(19) United States
(12) Patent Application Publication
(10) Pub. No.: US 2021/0228627 A1
(43) Pub. Date: Jul. 29, 2021

(54) FUSOSOME COMPOSITIONS AND USES THEREOF

(71) Applicant: FLAGSHIP PIONEERING INNOVATIONS V, INC., Cambridge, MA (US)

(72) Inventors: Geoffrey A. von Maltzahn, Somerville, MA (US); Jacob Rosenblum Rubens, Cambridge, MA (US); Michael Travis Mee, Montreal (CA); John Miles Milwid, Denver, CO (US); Neal Francis Gordon, Brookline, MA (US); Jagesh Vijaykumar Shah, Lexington, MA (US); Kyle Marvin Trudeau, Boston, MA (US); Brigham Jay Hartley, Long Island City, NY (US); Peter Anthony Jones, Medford, MA (US)

(21) Appl. No.: 17/055,077

(22) PCT Filed: May 15, 2019
(86) PCT No.: PCT/US2019/032488

§ 371 Date: Nov. 12, 2020

Related U.S. Application Data
(60) Provisional application No. 62/695,529, filed on Jul. 9, 2018.
(60) Provisional application No. 62/671,838, filed on May 15, 2018.

(51) Int. Cl. A61K 35/15; C07K 14/005; C12N 15/88; A61K 48/00

(52) U.S. Cl. CPC A61K 35/15; C07K 14/005; C12N 15/88; C12N 2830/007; C12N 2740/10043; C12N 2760/18034; A61K 48/0008

(57) ABSTRACT

The present disclosure provides, at least in part, methods and compositions for in vivo fusosome delivery. In some embodiments, the fusosome comprises a combination of elements that promote specificity for target cells, e.g., one or more of a re-targeted fusogen, a positive target cell-specific regulatory element, and a non-target cell-specific regulatory element. In some embodiments, the fusosome comprises one or more modifications that decrease an immune response against the fusosome.

### CROSS-REFERENCE TO RELATED APPLICATIONS

This application claims priority to U.S. Ser. No. 62/671,838 filed May 15, 2018 and U.S. Ser. No. 62/695,529 filed Jul. 9, 2018, each of which is incorporated herein by reference in its entirety.

### INCORPORATION BY REFERENCE OF SEQUENCE LISTING

The present application is being filed along with a Sequence Listing in electronic format. The Sequence Listing is provided as a file entitled V2050-7023WO Sequence Listing.TXT, created May 14, 2019, which is 651 kilobytes in size. The information in the electronic format of the Sequence Listing is incorporated by reference in its entirety.

### BACKGROUND

Complex biologics are promising therapeutic candidates for a variety of diseases. However, it is difficult to deliver large biologic agents into a cell because the plasma membrane acts as a barrier between the cell and the extracellular space. There is a need in the art for new methods of delivering complex biologics into cells in a subject.

### SUMMARY

The present disclosure provides, at least in part, fusosome methods and compositions for in vivo delivery. In some embodiments, the fusosome comprises a combination of elements that promote specificity for target cells, e.g., one or more of a re-targeted fusogen, a positive target cell-specific regulatory element, and a non-target cell-specific regulatory element. In some embodiments, the fusosome comprises one or more modifications that decrease an immune response against the fusosome.

### BRIEF DESCRIPTION OF DRAWINGS

FIGS. 1A-1C are a series of graphs showing results for cell lines, including target human hepatoma cell lines (HepG2) and non-target (non-hepatic) cell lines, transduced with lentivirus (LV) encoding nucleic acid constructs containing positive TCSREs or NTCSREs. FIG. 1A shows GFP expression in human hepatoma cell line (HepG2), human embryonic kidney cell line (293LX), human T-cell line of hematopoietic origin (Molt4.8) and endothelial cell line derived from mouse brain (bEND.3) transduced with LV generated with miRT sequences (hPGK-eGFP+miRT) or without miRT sequences (hPGK-eGFP), under the control of the PGK promoter. FIG. 1B shows GFP expression in HepG2 and 293LX cells transduced with LV generated under the control of the PGK promoter (hPGK-eGFP) or LVs containing mirT sequences and GFP under the control of the hepatocyte specific promoter ApoE (hApoE-eGFP+miRT). FIG. 1C shows quantification of Phenylalanine (Phe) in supernatant of HepG2 and 293LX cells transduced with LVs containing the transgene phenylalanine ammonia lyase (PAL) under the control of the SFFV promoter (SFFV-PAL), or LVs containing mirT sequences and under the control of the hApoE promoter (hApoE-PAL+miRT).

### ENUMERATED EMBODIMENTS

Provided herein are fusosomes, including retroviral vectors or particles, such as lentiviral vectors or particles, that generally result in increased expression of a desired exogenous agent (e.g., a therapeutic transgene) in target cells compared to non-target cells following introduction of the fusosomes into cells, e.g., in a subject. For example, in some cases, the increase in expression is following in vivo administration of a provided fusosome (e.g. a retroviral vector or particle) to a subject, e.g. human subject. In particular, one of the major challenges for successful gene therapy is the ability to maintain stable, long-term expression of a therapeutic transgene (e.g., an exogenous agent) from genetically modified cells in vivo. Transgene expression in non-target cells such as antigen-presenting cells (APCs) can, in some aspects, result in activation of the adaptive immune response leading to generation of neutralizing antibodies against the transgene product by B-cells and/or elimination of transgene producing cells by T-cells. Thus, limiting transgene expression to target cells may, in some embodiments, substantially impact the durability of transgene expression by avoiding immune clearance. Furthermore, cell-type specific transgene expression may be very relevant to disease biology such as, e.g., limiting expression of pro-apoptotic genes to tumor cells or other target cells (e.g., liver cells).

In particular, provided herein are fusosomes (e.g. retroviral vector pr particles) that, in some instances, include expression of nucleic acid sequences under the control of or that are regulated by a positive target cell-specific regulatory element (TCSRE, e.g., a tissue-specific promoter) and/or a negative target cell-specific regulatory element (negative TCSRE), e.g., a non-target cell-specific regulatory element (NTCSRE). In some embodiments, the negative TCSCRE, such as NCSRE, is by miRNA-mediated gene silencing, such as by nucleic acid sequences complementatry to miRNA sequences in a cell. In some embodiments, the provided fusosomes (e.g. retroviral vectors or particles) can specifically drive transgene (exogenous agent) expression in a target cell line (e.g. tumor or hepatic cell or other target cell) while restricting or limiting expression in non-target cells.

Among the provided embodiments are:

1. A fusosome comprising:

a) a lipid bilayer comprising a retargeted fusogen; and

b) a nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise fusosome lacking the positive tissue-specific regulatory element; or
(ii) a non-target cell-specific regulatory element (e.g., a tissue-specific miRNA recognition sequence), operatively linked to the nucleic acid encoding the exogenous agent, wherein the non-target cell-specific regulatory element decreases expression of the exogenous agent in a non-target cell or tissue relative to an otherwise similar fusosome lacking the non-target cell-specific regulatory element.
   2. A fusosome comprising:

a) a lipid bilayer comprising a retargeted fusogen;

b) a nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise retroviral vector lacking the positive tissue-specific regulatory element; or
(ii) a negative target cell-specific regulatory element (e.g., a tissue-specific miRNA recognition sequence), operatively linked to the nucleic acid encoding the exogenous agent, wherein the negative tissue-specific regulatory element decreases expression of the exogenous agent in a non-target cell or tissue relative to an otherwise similar retroviral vector lacking the negative tissue-specific regulatory element.
   3. A fusosome comprising:

a) a lipid bilayer comprising a fusogen (e.g., a re-targeted fusogen);

b) a nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise similar fusosome lacking the positive tissue-specific regulatory element; and
(ii) a non-target cell-specific regulatory element (e.g., a tissue-specific miRNA recognition sequence), operatively linked to the nucleic acid encoding the exogenous agent, wherein the non-target cell-specific regulatory element decreases expression of the exogenous agent in a non-target cell or tissue relative to an otherwise similar fusosome lacking the non-target cell-specific regulatory element.
   4. A fusosome, comprising:

a) a lipid bilayer comprising a fusogen (e.g., a re-targeted fusogen);

b) a nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise retroviral vector lacking the positive tissue-specific regulatory element; and
(ii) a negative target cell-specific regulatory element (e.g., a tissue-specific miRNA recognition sequence), operatively linked to the nucleic acid encoding the exogenous agent, wherein the negative tissue-specific regulatory element decreases expression of the exogenous agent in a non-target cell or tissue relative to an otherwise similar retroviral vector lacking the negative tissue-specific regulatory element.
   5. The fusosome of any of the preceding embodiments, wherein one or more of:
i) the fusosome fuses at a higher rate with a target cell than with a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
ii) the fusosome fuses at a higher rate with a target cell than with another fusosome, e.g., by at least 10%, 20%, 30%, 40%, 50%, 60%, 70%, 80%, or 90%, 2-fold, 3-fold, 4-fold, 5-fold, 10-fold, 20-fold, 50-fold, or 100-fold;
iii) the fusosome fuses with target cells at a rate such that an agent in the fusosome is delivered to at least 10%, 20%, 30%, 40%, 50%, 60%, 70%, 80%, or 90%, of target cells after 24, 48, or 72 hours;
iv) the fusosome delivers the nucleic acid to a target cell at a higher rate than to a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
v) the fusosome delivers the nucleic acid to a target cell at a higher rate than to another fusosome, e.g., by at least 10%, 20%, 30%, 40%, 50%, 60%, 70%, 80%, or 90%, 2-fold, 3-fold, 4-fold, 5-fold, 10-fold, 20-fold, 50-fold, or 100-fold; or
vi) the delivers the nucleic acid to a target cell at a rate such that an agent in the fusosome is delivered to at least 10%, 20%, 30%, 40%, 50%, 60%, 70%, 80%, or 90%, of target cells after 24, 48, or 72 hours.
   6. The fusosome of any of the preceding embodiments, wherein one or more of (e.g., 2 or all 3 of) the following apply: the fusosome is a retroviral vector, the lipid bilayer is comprised by an envelope, e.g., a viral envelope, and the nucleic acid is a retroviral nucleic acid.
   7. The fusosome of any of the preceding embodiments, wherein the nucleic acid comprises one or more of (e.g., all of) the following nucleic acid sequences: 5′ LTR (e.g., comprising U5 and lacking a functional U3 domain), Psi packaging element (Psi), Central polypurine tract (cPPT) Promoter operatively linked to the payload gene, e.g. nucleic acid encoding the exogenous agent, payload gene, e.g. nucleic acid encoding the exogenous agent (optionally comprising an intron before the open reading frame), Poly A tail sequence, WPRE, and 3′ LTR (e.g., comprising U5 and lacking a functional U3).
   8. The fusosome of any of the preceding embodiments, which comprises one or more of (e.g., all of) a polymerase (e.g., a reverse transcriptase, e.g., pol or a portion thereof), an integrase (e.g., pol or a portion thereof, e.g., a functional or non-functional variant), a matrix protein (e.g., gag or a portion thereof), a capsid protein (e.g., gag or a portion thereof), a nucleocaspid protein (e.g., gag or a portion thereof), and a protease (e.g., pro).
   9. The fusosome of any of the preceding embodiments, wherein, when the fusosome is administered to a subject, one or more of:
i) less than 10%, 5%, 4%, 3%, 2%, or 1% of the exogenous agent detectably present in the subject is in non-target cells;
ii) at least 90%, 95%, 96%, 97%, 98%, or 99% of the cells of the subject that detectably comprise the exogenous agent, are target cells (e.g., cells of a single cell type, e.g., T cells);
iii) less than 1,000,000, 500,000, 200,000, 100,000, 50,000, 20,000, or 10,000 cells of the cells of the subject that detectably comprise the exogenous agent are non-target cells;
iv) average levels of the exogenous agent in all target cells in the subject are at least 100-fold, 200-fold, 500-fold, or 1,000-fold higher than average levels of the exogenous agent in all non-target cells in the subject; or
v) the exogenous agent is not detectable in any non-target cell in the subject.
   10. The fusosome of any of the preceding embodiments, wherein the re-targeted fusogen comprises a sequence chosen from Nipah virus F and G proteins, measles virus F and H proteins, tupaia paramyxovirus F and H proteins, paramyxovirus F and G proteins or F and H proteins or F and HN proteins, Hendra virus F and G proteins, Henipavirus F and G proteins, Morbilivirus F and H proteins, respirovirus F and HN protein, a Sendai virus F and HN protein, rubulavirus F and HN proteins, or avulavirus F and HN proteins, or a derivative thereof, or any combination thereof.
   11. The fusosome of any of the preceding embodiments, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusogen has a sequence set forth in any of SEQ ID NOS: 1-132.
   12. The fusosome of embodiment 11, wherein the paramyxovirus is a Nipah virus, e.g., a henipavirus.
   13. The fusosome of any of the preceding embodiments, wherein the positive target cell-specific regulatory element comprises a tissue-specific promoter, a tissue-specific enhancer, a tissue-specific splice site, a tissue-specific site extending half-life of an RNA or protein, a tissue-specific mRNA nuclear export promoting site, a tissue-specific translational enhancing site, or a tissue-specific post-translational modification site.
   14. The fusosome of any of the preceding embodiments, wherein the non-target cell specific regulatory element or negative TCSRE comprises a tissue-specific miRNA recognition sequence, tissue-specific protease recognition site, tissue-specific ubiquitin ligase site, tissue-specific transcriptional repression site, or tissue-specific epigenetic repression site.
   15. The fusosome of any of the preceding embodiments, wherein the non-target cell specific regulatory element or negative TCSRE comprises a tissue-specific miRNA recognition sequence.
   16. The fusosome of embodiment 15, wherein the non-target cell specific regulatory element or negative TCSRE is situated or encoded within a transcribed region (e.g., the transcribed region encoding the exogenous agent), e.g., such that an RNA produced by the transcribed region comprises the miRNA recognition sequence within a UTR or coding region.
   17. The fusosome of any of the preceding embodiments, wherein the target cell is a cancer cell and the non-target cell is a non-cancerous cell.
   18. The fusosome of any of the preceding embodiments, wherein the nucleic acid, e.g., retroviral nucleic acid, encodes a positive TCSRE and/or a NTCSRE or negative TCSRE.
   19. The fusosome of any of the preceding embodiments, wherein the retroviral nucleic acid comprises the complement of a positive TCSRE and/or a NTCSRE or negative TCSRE.
   20. The fusosome of any of the preceding embodiments, which does not deliver nucleic acid to a non-target cell, e.g., an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell.
   21. The fusosome of any of the preceding embodiments, wherein less than 10%, 5%, 2.5%, 1%, 0.5%, 0.1%, 0.01%, 0.001%, 0.0001%, 0.00001%, or 0.000001% of a non-target cell type (e.g., one or more of an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell) comprise the nucleic acid, e.g., retroviral nucleic acid, e.g., using quantitative PCR, e.g., using an assay of Example 1.
   22. The fusosome of any of the preceding embodiments, wherein the target cells comprise 0.00001-10, 0.0001-10, 0.001-10, 0.01-10, 0.1-10, 0.5-5, 1-4, 1-3, or 1-2 copies of the nucleic acid, e.g., retroviral nucleic acid or a portion thereof, per host cell genome, e.g., wherein copy number of the nucleic acid is assessed after administration in vivo.
   23. The fusosome of any of the preceding embodiments, wherein:

less than 10%, 5%, 2.5%, 1%, 0.5%, 0.1%, 0.01% of the non-target cells (e.g., an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell) comprise the exogenous agent; or

the exogenous agent (e.g., protein) is not detectably present in a non-target cell, e.g an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell.

24. The fusosome of any of the preceding embodiments, wherein the fusosome delivers the nucleic acid, e.g., retroviral nucleic acid, to a target cell, e.g., a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.
25. The fusosome of any of the preceding embodiments, wherein at least 0.00001%, 0.0001%, 0.001%, 0.001%, 0.01%, 0.1%, 1%, 2%, 5%, 10%, 20%, 30%, 40%, 50%, 60%, 70%, 80%, or 90% of target cells (e.g., one or more of a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell) comprise the nucleic acid, e.g., using quantitative PCR, e.g., using an assay of Example 3.
26. The fusosome of any of the preceding embodiments, wherein at least 0.00001%, 0.0001%, 0.001%, 0.001%, 0.01%, 0.1%, 1%, 2%, 5%, 10%, 20%, 30%, 40%, 50%, 60%, 70%, 80%, or 90% of target cells (e.g., a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell) comprise the exogenous agent.
27. The fusosome of any of the preceding embodiments, wherein, upon administration, the ratio of target cells comprising the nucleic acid to non-target cells comprising the nucleic acid is at least 1.5, 2, 3, 4, 5, 10, 25, 50, 100, 500, 1000, 5000, 10,000, e.g., according to a quantitative PCR assay, e.g., using assays of Example 1 and Example 3.
28. The fusosome of any of the preceding embodiments, wherein the ratio of the average copy number of nucleic acid or a portion thereof in target cells to the average copy number of nucleic acid or a portion thereof in non-target cells is at least 1.5, 2, 3, 4, 5, 10, 25, 50, 100, 500, 1000, 5000, 10,000, e.g., according to a quantitative PCR assay, e.g., using assays of Example 1 and Example 3.
29. The fusosome of any of the preceding embodiments, wherein the ratio of the median copy number of of nucleic acid or a portion thereof in target cells to the median copy number of nucleic acid or a portion thereof in non-target cells is at least 1.5, 2, 3, 4, 5, 10, 25, 50, 100, 500, 1000, 5000, 10,000, e.g., according to a quantitative PCR assay, e.g., using assays of Example 1 and Example 3.
30. The fusosome of any of the preceding embodiments, wherein the ratio of target cells comprising the exogenous RNA agent to non-target cells comprising the exogenous RNA agent is at least 1.5, 2, 3, 4, 5, 10, 25, 50, 100, 500, 1000, 5000, 10,000, e.g., according to a reverse transcription quantitative PCR assay.
31. The fusosome of any of the preceding embodiments, wherein the ratio of the average exogenous RNA agent level of target cells to the average exogenous RNA agent level of non-target cells is at least 1.5, 2, 3, 4, 5, 10, 25, 50, 100, 500, 1000, 5000, 10,000, e.g., according to a reverse transcription quantitative PCR assay.
32. The fusosome of any of the preceding embodiments, wherein the ratio of the median exogenous RNA agent level of target cells to the median exogenous RNA agent level of non-target cells is at least 1.5, 2, 3, 4, 5, 10, 25, 50, 100, 500, 1000, 5000, 10,000, e.g., according to a reverse transcription quantitative PCR assay.
33. The fusosome of any of the preceding embodiments, wherein the ratio of target cells comprising the exogenous protein agent to non-target cells comprising the exogenous protein agent is at least 1.5, 2, 3, 4, 5, 10, 25, 50, 100, 500, 1000, 5000, 10,000, e.g., according to a FACS assay, e.g., using assays of Example 2 and/or Example 4.
34. The fusosome of any of the preceding embodiments, wherein the ratio of the average exogenous protein agent level of target cells to the average exogenous protein agent level of non-target cells is at least 1.5, 2, 3, 4, 5, 10, 25, 50, 100, 500, 1000, 5000, 10,000, e.g., according to a FACS assay, e.g., using assays of Example 2 and/or Example 4.
35. The fusosome of any of the preceding embodiments, wherein the ratio of the median exogenous protein agent level of target cells to the median exogenous protein agent level of non-target cells is at least 1.5, 2, 3, 4, 5, 10, 25, 50, 100, 500, 1000, 5000, 10,000, e.g., according to a FACS assay, e.g., using assays of Example 2 and/or Example 4.
36. The fusosome of any of the preceding embodiments, which comprises one or both of:
i) an exogenous or overexpressed immunosuppressive protein on the lipid bilayer, e.g., envelope; and
ii) an immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.
   37. The fusosome of any of the preceding embodiments, which comprises one or more of:
i) a first exogenous or overexpressed immunosuppressive protein on the lipid bilayer, e.g., envelope, and a second exogenous or overexpressed immunosuppressive protein on the lipid bilayer, e.g., envelope;
ii) a first exogenous or overexpressed immunosuppressive protein on the lipid bilayer, e.g., envelope, and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell; or
iii) a first immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.
   38. The fusosome of any of the preceding embodiments, wherein the nucleic acid comprises one or more insulator elements.
   39. A fusosome, comprising:

a) a lipid bilayer comprising a fusogen (e.g., a re-targeted fusogen), and

b) an exogenous agent (e.g., exogenous polypeptide or exogenous RNA) or a nucleic acid (e.g., a retroviral nucleic acid) encoding an exogenous agent; and

c) one or more of:
i) a first exogenous or overexpressed immunosuppressive protein on the lipid bilayer, e.g., envelope, and a second exogenous or overexpressed immunosuppressive protein on the lipid bilayer, e.g., envelope;
ii) a first exogenous or overexpressed immunosuppressive protein on the lipid bilayer, e.g., envelope, and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell; or
iii) a first immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell;

wherein, when administered to a subject (e.g., a human subject or a mouse), one or more of:

i) the fusosome does not produce a detectable antibody response (e.g., after a single administration or a plurality of administrations), or antibodies against the fusosome are present at a level of less than 10%, 5%, 4%, 3%, 2%, or 1% above a background level, e.g., by a FACS antibody detection assay, e.g., an assay of Example 13 or Example 14);

ii) the fusosome does not produce a detectable cellular immune response (e.g., T cell response, NK cell response, or macrophage response), or a cellular immune response against the fusosome is present at a level of less than 10%, 5%, 4%, 3%, 2%, or 1% above a background level, e.g., by a PBMC lysis assay (e.g., an assay of Example 5), by an NK cell lysis assay (e.g., an assay of Example 6), by a CD8 killer T cell lysis assay (e.g., an assay of Example 7), by a macrophage phagocytosis assay (e.g., an assay of Example 8);

iii) the fusosome does not produce a detectable innate immune response, e.g., complement activation (e.g., after a single administration or a plurality of administrations), or the innate immune response against the fusosome is present at a level of less than 10%, 5%, 4%, 3%, 2%, or 1% above a background level, e.g., by a complement activity assay (e.g., an assay of Example 9);

iv) less than 90%, 80%, 70%, 60%, 50%, 40%, 30%, 20%, 10%, 5%, 4%, 3%, 2%, 1%, 0.5%, 0.2%, 0.1%, 0.05%, 0.02%, 0.01%, 0.005%, 0.002%, or 0.001% of fusosomes are inactivated by serum, e.g., by a serum inactivation assay, e.g., an assay of Example 11 or Example 12;

v) a target cell that has received the exogenous agent from the fusosome does not produce a detectable antibody response (e.g., after a single administration or a plurality of administrations), or antibodies against the target cell are present at a level of less than 10%, 5%, 4%, 3%, 2%, or 1% above a background level, e.g., by a FACS antibody detection assay, e.g., an assay of Example 15; or

vi) a target cell that has received the exogenous agent from the fusosome do not produce a detectable cellular immune response (e.g., T cell response, NK cell response, or macrophage response), or a cellular response against the target cell is present at a level of less than 10%, 5%, 4%, 3%, 2%, or 1% above a background level, e.g., by a macrophage phagocytosis assay (e.g., an assay of Example 16), by a PBMC lysis assay (e.g., an assay of Example 17), by an NK cell lysis assay (e.g., an assay of Example 18), or by a CD8 killer T cell lysis assay (e.g., an assay of Example 19).

40. The fusosome of any of the preceding embodiments, wherein one or more of (e.g., 2 or all 3 of) the following apply: the fusosome is a retroviral vector, the lipid bilayer is comprised by an envelope, e.g., a viral envelope, and the nucleic acid is a retroviral nucleic acid.
41. The fusosome of embodiment 39 or 40, wherein the background level is the corresponding level in the same subject prior to administration of the particle or vector.
42. The fusosome of any of embodiments 39-41, wherein the immunosuppressive protein is a complement regulatory protein or CD47.
43. The fusosome of any of embodiments 39-42, wherein the immunostimulatory protein is an MHC (e.g., HLA) protein.
44. The fusosome of any of embodiments 39-43, wherein one or both of: the first exogenous or overexpressed immunosuppressive protein is other than CD47, and the second immunostimulatory protein is other than MHC.
45. A fusosome, comprising:

a) a lipid bilayer comprising a fusogen,

b) a nucleic acid encoding an exogenous agent (e.g., exogenous polypeptide or exogenous RNA); and

c) exogenous or overexpressed MHC, e.g., HLA (e.g., HLA-G or HLA-E), or a combination thereof, on the lipid bilayer.

46. A fusosome comprising:

a) a lipid bilayer comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence Table 4 or Table 5, optionally wherein the wild-type paramyxovius fusogen is set forth in any one of SEQ ID Nos: 1-132; and

b) a nucleic acid encoding an exogenous agent (e.g., exogenous polypeptide or exogenous RNA);

c) exogenous or overexpressed CD47 or a complement regulatory protein, or a combination thereof, on the envelope.

47. A fusosome, comprising:

a) a lipid bilayer comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusosen has a sequence of amino acids set forth in any one of SEQ ID NOS: 1-132, and

b) a nucleic acid encoding an exogenous agent (e.g., exogenous polypeptide or exogenous RNA); and

c) MHC I (e.g., HLA-A, HLA-B, or HLA-C) or MHC II (e.g., HLA-DP, HLA-DM, HLA-DOA, HLA-DOB, HLA-DQ, or HLA-DR) that is absent or present at reduced levels (e.g., reduced by at least 10%, 20%, 30%, 40%, 50%, 60%, 70%, 80%, or 90%) compared to a fusosome generated from an otherwise similar, unmodified source cell.

48. A fusosome, comprising:

a) a lipid bilayer comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-tpe pramyxovirus fusogen has a sequence set forth in any one of SEQ ID NOS: 1-132; and

b) a nucleic acid encoding an exogenous agent (e.g., exogenous polypeptide or exogenous RNA); and

c) one or both of an exogenous or overexpressed immunosuppressive protein or an immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.

49. The fusosome of any of embodiments 45-48, wherein one or more of (e.g., 2 or all 3 of) the following apply: the fusosome is a retroviral vector, the lipid bilayer is comprised by an envelope, e.g., a viral envelope, and the nucleic acid is a retroviral nucleic acid.
50. The fusosome of any of the preceding embodiments, wherein the fusosome is in circulation at least 0.5, 1, 2, 3, 4, 6, 12, 18, 24, 36, or 48 hours after administration to the subject.
51. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 30 minutes after administration.
52. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 1 hour after administration.
53. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 2 hours after administration.
54. The fusosome of any of the preceding embodiments, wherein at least odiments 0.001%, 0.01%, 0.1%, 1%, 10%, 20%, 30%, 40%, 50%, 60%, 70%, 80%, 90%, or 100% of fusosomes are in circulation 4 hours after administration.
55. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 8 hours after administration.
56. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 12 hours after administration.
57. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 18 hours after administration.
58. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 24 hours after administration.
59. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 36 hours after administration.
60. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 48 hours after administration.
61. The fusosome of any of the preceding embodiments, which has a reduction in immunogenicity as measured by a reduction in humoral response following one or more administration of the fusosome to an appropriate animal model, e.g., an animal model described herein, compared to reference retrovirus, e.g., an unmodified fusosome otherwise similar to the fusosome.
62. The fusosome of any of the preceding embodiments, wherein the reduction in humoral response is measured in a serum sample by an anti-cell antibody titre, e.g., anti-retroviral antibody titre, e.g., by ELISA.
63. The fusosome of any of the preceding embodiments, wherein a serum sample from animals administered the retrovirus composition has a reduction of 1%, 5%, 10%, 20%, 30%, 40%, 50%, 60%, 70%, 80%, 90%, or more of an anti-fusosome antibody titer compared to the serum sample from a subject administered an unmodified cell.
64. The fusosome of any of the preceding embodiments, wherein a serum sample from a subject administered the fusosome has an increased anti-cell antibody titre, e.g., increased by 1%, 2%, 5%, 10%, 20%, 30%, or 40% from baseline, e.g., wherein baseline refers to serum sample from the same subject before administration of the fusosome.
65. The fusosome of any of the preceding embodiments, wherein:

the subject to be administered the fusosome has, or is known to have, or is tested for, a pre-existing antibody (e.g., IgG or IgM) reactive with the fusosome;

the subject to be administered the fusosome does not have detectable levels of a pre-existing antibody reactive with the fusosome;

a subject that has received the fusosome has, or is known to have, or is tested for, an antibody (e.g., IgG or IgM) reactive with the fusosome;

the subject that received the fusosome (e.g., at least once, twice, three times, four times, five times, or more) does not have detectable levels of antibody reactive with the fusosome; or

levels of antibody do not rise more than 1%, 2%, 5%, 10%, 20%, or 50% between two timepoints, the first timepoint being before the first administration of the fusosome, and the second timepoint being after one or more administrations of the fusosome.

66. The fusosome of any of the preceding embodiments, wherein the fusosome is a retroviral vector produced by the methods of Example 5, 6, or 7, e.g., from cells transfected with HLA-G or HLA-E cDNA.
67. The fusosome of any of the preceding embodiments, wherein the fusosome is a retroviral vector generated from NMC-HLA-G cells and has a decreased percentage of lysis, e.g., PBMC mediated lysis, NK cell mediated lysis, and/or CD8+ T cell mediated lysis, at specific timepoints as compared to retroviral vectors generated from NMCs or NMC-empty vector.
68. The fusosome of any of the preceding embodiments, wherein the modified fusosome evades phagocytosis by macrophages.
69. The fusosome of any of the preceding embodiments, wherein the fusosome is produced by the method of Example 8, e.g., from cells transfected with CD47 cDNA.
70. The fusosome of any of the preceding embodiments, wherein the fusosome is a retroviral vector and wherein the phagocytic index is reduced when macrophages are incubated with retroviral vectors derived from NMC-CD47, versus those derived from NMC, or NMC-empty vector.
71. The fusosome of any of the preceding embodiments, which has a reduction in macrophage phagocytosis, e.g., a reduction of 1%, 5%, 10%, 20%, 30%, 40%, 50%, 60%, 70%, 80%, 90%, or more in macrophage phagocytosis compared to a reference fusosome, e.g., an unmodified fusosome otherwise similar to the fusosome, wherein the reduction in macrophage phagocytosis is determined by assaying the phagocytosis index in vitro, e.g., as described in Example 8.
72. The fusosome of any of the preceding embodiments, wherein a composition comprising a plurality of the fusosomes has a phagocytosis index of 0, 1, 10, 100, or more, e.g., as measured by an assay of Example 8, when incubated with macrophages in an in vitro assay of macrophage phagocytosis.
73. The fusosome of any of the preceding embodiments, which is modified and has reduced complement activity compared to an unmodified retroviral vector.
74. The fusosome of any of the preceding embodiments, which is produced by the methods of Example 9, e.g., from cells transfected with a cDNA coding for complement regulatory proteins, e.g., DAF.
75. The fusosome of any of the preceding embodiments, wherein the fusosome is a retroviral vector, and wherein the dose of retroviral vector at which 200 μg/ml of C3a is present is greater for the modified retroviral vector (e.g., HEK293-DAF) incubated with corresponding mouse sera (e.g., HEK-293 DAF mouse sera) than for the reference retroviral vector (e.g., HEK293 retroviral vector) incubated with corresponding mouse sera (e.g., HEK293 mouse sera).
76. The fusosome of any of the preceding embodiments, wherein the fusosome is a retroviral vector, and wherein the dose of retroviral vector at which 200 μg/ml of C3a is present is greater for for the modified retroviral vector (e.g., HEK293-DAF) incubated with naive mouse sera than for the reference retroviral vector (e.g., HEK293 retroviral vector) incubated with naive mouse sera.
77. The fusosome of any of the preceding embodiments, which is resistant to complement mediated inactivation in patient serum 30 minutes after administration according to an assay of Example 9.
78. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are resistant to complement mediated inactivation.
79. The fusosome of any of the preceding embodiments, wherein the complement regulatory protein comprises one or more of proteins that bind decay-accelerating factor (DAF, CD55), e.g. factor H (FH)-like protein-1 (FHL-1), e.g. C4b-binding protein (C4BP), e.g. complement receptor 1 (CD35), e.g. Membrane cofactor protein (MCP, CD46), eg. Protectin (CD59), e.g. proteins that inhibit the classical and alternative complement pathway CD/C5 convertase enzymes, e.g. proteins that regulate MAC assembly.
80. The fusosome of any of the preceding embodiments, which is produced by the methods of Example 10, e.g., from cells transfected with a DNA coding for an shRNA targeting MHC class I, e.g., wherein retroviral vectors derived from NMC-shMHC class I has lower expression of MHC class I compared to NMCs and NMC-vector control.
81. The fusosome of any of the preceding embodiments, wherein a measure of immunogenicity for fusosomes (e.g., retroviral vectors) is serum inactivation, e.g., serum inactivation measured as described herein, e.g., as described in Example 11.
82. The fusosome of any of the preceding embodiments, wherein the percent of cells which receive the exogenous agent is not different between fusosome samples that have been incubated with serum and heat-inactivated serum from fusosome naive mice.
83. The fusosome of any of the preceding embodiments, wherein the percent of cells which receive the exogenous agent is not different between fusosome samples that have been incubated with serum from fusosome naive mice and no-serum control incubations.
84. The fusosome of any of the preceding embodiments, wherein the percent of cells which receive the exogenous agent is less in fusosome samples that have been incubated with positive control serum than in fusosome samples that have been incubated with serum from fusosome naive mice.
85. The fusosome of any of the preceding embodiments, wherein a modified retroviral vector, e.g., modified by a method described herein, has a reduced (e.g., reduced compared to administration of an unmodified retroviral vector) serum inactivation following multiple (e.g., more than one, e.g., 2 or more), administrations of the modified retroviral vector.
86. The fusosome of any of the preceding embodiments, wherein a fusosome described herein is not inactivated by serum following multiple administrations.
87. The fusosome of any of the preceding embodiments, wherein a measure of immunogenicity for fusosome is serum inactivation, e.g., after multiple administrations, e.g., serum inactivation after multiple administrations measured as described herein, e.g., as described in Example 12.
88. The fusosome of any of the preceding embodiments, wherein the percent of cells which receive the exogenous agent is not different between fusosome samples that have been incubated with serum and heat-inactivated serum from mice treated with modified (e.g., HEK293-HLA-G) fusosome.
89. The fusosome of any of the preceding embodiments, wherein the percent of cells which receive the exogenous agent is not different between fusosome samples that have been incubated with serum from mice treated 1, 2, 3, 5 or 10 times with modified (e.g., HEK293-HLA-G) retroviral vectors.
90. The fusosome of any of the preceding embodiments, wherein the percent of cells which receive the exogenous agent is not different between fusosome samples that have been incubated with serum from mice treated with vehicle and from mice treated with modified (e.g., HEK293-HLA-G) fusosomes.
91. The fusosome of any of the preceding embodiments, wherein the percent of cells which receive the exogenous agent is less for fusosomes derived from a reference cell (e.g., HEK293) than for modified (e.g., HEK293-HLA-G) fusosomes.
92. The fusosome of any of the preceding embodiments, wherein a measure of immunogenicity for a fusosome is antibody responses.
93. The fusosome of any of the preceding embodiments, wherein a subject that receives a fusosome described herein has pre-existing antibodies which bind to and recognize the fusosome, e.g., measured as described herein, e.g., as described in Example 13.
94. The fusosome of any of the preceding embodiments, wherein serum from fusosome-naive mice shows more signal (e.g., fluorescence) than the negative control, e.g., serum from a mouse depleted of IgM and IgG, e.g., indicating that immunogenicity has occurred.
95. The fusosome of any of the preceding embodiments, wherein serum from fusosome-naive mice shows similar signal (e.g., fluorescence) compared to the negative control, e.g., indicating that immunogenicity did not detectably occur.
96. The fusosome of any of the preceding embodiments, which comprises a modified retroviral vector, e.g., modified by a method described herein, and which has a reduced (e.g., reduced compared to administration of an unmodified retroviral vector) humoral response following multiple (e.g., more than one, e.g., 2 or more), administrations of the modified retroviral vector e.g., measured as described herein, e.g., as described in Example 14.
97. The fusosome of any of the preceding embodiments, wherein the fusosome, e.g., retroviral vector, is produced by the methods of Example 5, 6, 7, or 14, e.g., from cells transfected with HLA-G or HLA-E cDNA.
98. The fusosome of any of the preceding embodiments, wherein humoral response is assessed by determining a value for the level of anti-fusosome antibodies (e.g., IgM, IgG1, and/or IgG2 antibodies).
99. The fusosome of any of the preceding embodiments, wherein modified (e.g., NMC-HLA-G) fusosomes, e.g., retroviral vectors, have decreased anti-viral IgM or IgG1/2 antibody titers (e.g., as measured by fluorescence intensity on FACS) after injections, as compared to a control, e.g., NMC retroviral vectors or NMC-empty retroviral vectors.
100. The fusosome of any of the preceding embodiments, wherein recipient cells are not targeted by an antibody response, or an antibody response will be below a reference level, e.g., measured as described herein, e.g., as described in Example 15.
101. The fusosome of any of the preceding embodiments, wherein signal (e.g., mean fluorescence intensity) is similar for recipient cells from mice treated with retroviral vectors and mice treated with PBS.
102. The fusosome of any of the preceding embodiments, wherein a measure of the immunogenicity of recipient cells is the macrophage response.
103. The fusosome of any of the preceding embodiments, wherein recipient cells are not targeted by macrophages, or are targeted below a reference level.
104. The fusosome of any of the preceding embodiments, wherein the phagocytic index, e.g., measured as described herein, e.g., as described in Example 16, is similar for recipient cells derived from mice treated with fusosomes and mice treated with PBS.
105. The fusosome of any of the preceding embodiments, wherein a measure of the immunogenicity of recipient cells is the PBMC response.
106. The fusosome of any of the preceding embodiments, wherein recipient cells do not elicit a PBMC response.
107. The fusosome of any of the preceding embodiments, wherein the percent of CD3+/CMG+ cells is similar for recipient cells derived from mice treated with fusosome and mice treated with PBS, e.g., as measured as described herein, e.g., as described in Example 17.
108. The fusosome of any of the preceding embodiments, wherein a measure of the immunogenicity of recipient cells is the natural killer cell response.
109. The fusosome of any of the preceding embodiments, wherein recipient cells do not elicit a natural killer cell response or elicit a lower natural killer cell response, e.g., lower than a reference value.
110. The fusosome of any of the preceding embodiments, wherein the percent of CD3+/CMG+ cells is similar for recipient cells derived from mice treated with fusosome and mice treated with PBS, e.g., as measured as described herein, e.g., as described in Example 18.
111. The fusosome of any of the preceding embodiments, wherein a measure of the immunogenicity of recipient cells is the CD8+ T cell response.
112. The fusosome of any of the preceding embodiments, wherein recipient cells do not elicit a CD8+ T cell response or elicit a lower CD8+ T cell response, e.g., lower than a reference value.
113. The fusosome of any of the preceding embodiments, wherein the percent of CD3+/CMG+ cells is similar for recipient cells derived from mice treated with fusosome and mice treated with PBS, e.g., as measured as described herein, e.g., as described in Example 19.
114. The fusosome of any of the preceding embodiments, wherein the fusogen is a re-targeted fusogen.
115. The fusosome of any of the preceding embodiments, which comprises a retroviral nucleic acid that encodes one or both of: (i) a positive target cell-specific regulatory element operatively linked to a nucleic acid encoding an exogenous agent, or (ii) a non-target cell-specific regulatory element or negative TCSRE operatively linked to the nucleic acid encoding the exogenous agent.
116. A fusosome, comprising:

a) a lipid bilayer comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type pramyxovirus has a sequence of amino acids set forth in any one of SEQ ID NOS: 1-132; and

b) a nucleic acid encoding an exogenous agent (e.g., exogenous polypeptide or exogenous RNA), wherein the retroviral nucleic acid comprises one or more insulator elements.

117. The fusosome of embodiment 116, wherein one or more of (e.g., 2 or all 3 of) the following apply: the fusosome is a retroviral vector, the lipid bilayer is comprised by an envelope, e.g., a viral envelope, e.g., a pseudotyped envelope, and the nucleic acid is a retroviral nucleic acid.
118. The fusosome of embodiment 116 or 117, wherein the nucleic acid comprises two insulator elements, e.g., a first insulator element upstream of a region encoding the exogenous agent and a second insulator element downstream of a region encoding the exogenous agent, e.g., wherein the first insulator element and second insulator element comprise the same or different sequences.
119. The fusosome of any of embodiments 116-118, wherein variation in the median exogenous agent level in a sample of cells isolated after administration of the fusosome to the subject at a first timepoint is at least, less than, or about 10,000%, 5,000%, 2,000%, 1,000%, 500%, 200%, 100%, 50%, 20%, 10%, or 5% of the median exogenous agent level in a sample of cells isolated after administration of the fusosome to the subject at a second, later timepoint.
120. The fusosome of embodiment 119, wherein the median expression level per cell is assessed only in cells that have a retroviral genome copy number of at least 1.0.
121. The fusosome of any of embodiments 116-120, wherein at least 10%, 20%, 30%, 40%, 50%, 60%, 70%, 80%, or 90% of target cells in the subject detectably comprise the exogenous agent.
122. The fusosome of any of embodiments 119-121, wherein the median payload gene expression level is assessed across cells isolated from the subject 7 days, 14 days, 28 days, 56 days, 112 days, 365 days, 730 days, 1095 days after administration of the fusosome to the subject.
123. The fusosome of any of embodiments 116-122, wherein at least 10%, 20%, 30%, 40%, 50%, 60%, 70%, 80%, or 90% of target cells in the subject that detectably comprised the exogenous agent at a first time point still detectably comprise the exogenous agent at a second, later timepoint, e.g., wherein the first time point is 7 days, 14 days, 28 days, 56 days, 112 days, 365 days, 730 days, 1095 days after administration of the fusosome to the subject.
124. The fusosome of embodiment 123, wherein the second time point is 7 days, 14 days, 28 days, 56 days, 112 days, 365 days, 730 days, 1095 days after the first time point.
125. The fusosome of any of embodiments 116-124, which is not genotoxic or does not increase the rate of tumor formation in target cells compared to target cells not treated with the fusosome.
126. The fusosome of any of embodiments 116-125, wherein the median exogenous agent level is assessed in a population of cells from a subject that has received the fusosome.
127. The fusosome of any of embodiments 116-126, wherein the median exogenous agent level assessed in populations of cells collected (e.g., isolated) from the subject at different days post administration is less than about 10,000% 1000%, 100%, or 10%, e.g., 10,000%-1000%, 1000%-100%, or 100%-10% different from the median exogenous agent level in the population of cells assessed at day 7, day 14, day 28, or day 56, wherein the cells in the population have a vector copy number of at least 1.0.
128. The fusosome of any of embodiments 116-127, wherein exogenous agent level is assessed across cells from a subject that has received the fusosome.
129. The fusosome of any of embodiments 116-128, wherein the percent of cells comprising the exogenous agent is assessed in a plurality of cells collected (e.g., isolated) from the subject 7 days, 14 days, 28 days, 56 days, 112 days, 365 days, 730 days, 1095 days after administration of the fusosome.
130. The fusosome of any of embodiments 116-129, wherein the difference in the percent of cells comprising the exogenous agent assessed in cells isolated at two different days post administration is less than 1%, 5%, 10%, 20%, 50%, 75%, 100%, 150%, 200%, 250%, 300%, 400%, 500%, 750%, 1000%, 1500%, or 2000%.
131. The fusosome of any of embodiments 116-130, wherein the percent of target cells that are positive for the exogenous agent is similar across cells collected at 7 days, 14 days, 28 days, 56 days, 112 days, 365 days, 730 days, or 1095 days.
132. The fusosome of any of embodiments 116-131, wherein:

at least as many target cells are positive for the exogenous agent 14 days, 28 days, 56 days, 112 days, 365 days, 730 days, or 1095 days as at 7 days;

at least as many target cells are positive for the exogenous agent at 28 days, 56 days, 112 days, 365 days, 730 days, or 1095 days as at 14 days;

at least as many target cells are positive for the exogenous agent at 56 days, 112 days, 365 days, 730 days, or 1095 days as at 28 days;

at least as many target cells are positive for the exogenous agent at 112 days, 365 days, 730 days, or 1095 days as at 56 days;

at least as many target cells are positive for the exogenous agent at 365 days, 730 days, or 1095 days as at 112 days;

at least as many target cells are positive for the exogenous agent at 730 days or 1095 days as at 365 days; or

at least as many target cells are positive for the exogenous agent at 1095 days as at 730 days.

133. The fusosome of any of embodiments 116-132, wherein:

the median exogenous agent level in target cells that comprise the exogenous agent is similar in cells collected at 7 days, 14 days, 28 days, 56 days, 112 days, 365 days, 730 days, or 1095 days;

the median exogenous agent level in target cells that comprise the exogenous agent at 14 days, 28 days, 56 days, 112 days, 365 days, 730 days, or 1095 days is at least as high as at 7 days;

the median exogenous agent level in target cells that comprise the exogenous agent at 28 days, 56 days, 112 days, 365 days, 730 days, or 1095 days is at least as high as at 14 days;

the median exogenous agent level in target cells that comprise the exogenous agent at 56 days, 112 days, 365 days, 730 days, or 1095 days is at least as high as at 28 days;

the median exogenous agent level in target cells that comprise the exogenous agent at 112 days, 365 days, 730 days, or 1095 days is at least as high as at 56 days;

the median exogenous agent level in target cells that comprise the exogenous agent at 365 days, 730 days, or 1095 days is at least as high as at 112 days;

the median exogenous agent level in target cells that comprise the exogenous agent at 730 days, or 1095 days is at least as high as at 365 days; or

the median exogenous agent level in target cells that comprise the exogenous agent at 1095 days is at least as high as at 730 days.

134. A method of delivering an exogenous agent to a subject (e.g., a human subject) comprising administering to the subject a fusosome of any of the preceding embodiments, thereby delivering the exogenous agent to the subject.
135. A method of modulating a function, in a subject (e.g., a human subject), target tissue or target cell, comprising contacting, e.g., administering to, the subject, the target tissue or the target cell a fusosome of any of the preceding embodiments.
136. The method of embodiment 135, wherein the target tissue or the target cell is present in a subject.
137. A method of treating or preventing a disorder, e.g., a cancer, in a subject (e.g., a human subject) comprising administering to the subject a fusosome of any of the preceding embodiments.
138. A method of making a fusosome of any of the preceding embodiments, comprising:

a) providing a source cell that comprises the nucleic acid and the fusogen (e.g., re-targeted fusogen);

b) culturing the source cell under conditions that allow for production of the fusosome, and

c) separating, enriching, or purifying the fusosome from the source cell, thereby making the fusosome.

139. A source cell for producing a fusosome, comprising:

a) a nucleic acid;

b) structural proteins that can package the nucleic acid, wherein at least one structural protein comprises a fusogen that binds a fusogen receptor; and

c) a fusogen receptor that is absent or present at reduced levels (e.g., reduced by at least 10%, 20%, 30%, 40%, 50%, 60%, 70%, 80%, or 90%) compared to an otherwise similar, unmodified source cell.

140. The method of embodiment 138 or the source cell of embodiment 139, wherein one or more of (e.g., 2 or all 3 of) the following apply: the fusosome is a retroviral vector, the nucleic acid is a retroviral nucleic acid, and the structural protein is a viral structural protein.
141. The source cell of embodiment 139 or 140, wherein the fusogen causes fusion of the fusosome with the target cell upon binding to the fusogen receptor.
142. The source cell of any of embodiments 139-141, which binds to the second similar source cell, e.g., the fusogen of the source cell binds to the fusogen receptor on the second source cell.
143. A population of source cells of any of embodiments 139-142.
144. The population of source cells of embodiment 143, wherein less than 10%, 5%, 4%, 3%, 2%, or 1% of cells in the population are multinucleated.
145. The source cell or population of source cells of any of embodiments 139-144, wherein a source cell is modified to have reduced fusion (e.g., to not fuse) with other source cells during manufacturing of a fusosome described herein.
146. The source cell or population of source cells of any of embodiments 139-145, wherein the fusogen (e.g., re-targeted fusogen) does not bind to a protein comprised by a source cell, e.g., to a protein on the surface of the source cell.
147. The source cell or population of source cells of any of embodiments 139-146, wherein the fusogen (e.g., re-targeted fusogen) binds to a protein comprised by a source cell, but does not fuse with the cell.
148. The source cell or population of source cells of any of embodiments 139-147, wherein the fusogen does not induce fusion with a source cell.
149. The source cell or population of source cells of any of embodiments 139-148, wherein the source cell does not express a protein (e.g., an antigen) that binds the fusogen.
150. The source cell or population of source cells of any of embodiments 139-149, a plurality of source cells do not form a syncytium when expressing the fusogen, or less than 50%, 40%, 30%, 20%, 10%, 5%, 4%, 3%, 2%, or 1% of cells in the population are multinucleated (e.g., comprise two or more nuclei).
151. The source cell or population of source cells of any of embodiments 139-150, wherein a plurality of source cells do not form a syncytium when producing fusosomes, or less than 50%, 40%, 30%, 20%, 10%, 5%, 4%, 3%, 2%, or 1% of cells in the population are multinucleated.
152. The source cell or population of source cells of any of embodiments 139-151, wherein less than 50%, 40%, 30%, 20%, 10%, 5%, 4%, 3%, 2%, or 1% of the nuclei in the population are in syncytia.
153. The source cell or population of source cells of any of embodiments 139-152, wherein at least 50%, 60%, 70%, 80%, 90%, 95%, 96%, 97%, 98%, 99% of nuclei in the population are in uninuclear cells.
154. The source cell or population of source cells of any of embodiments 139-153, wherein the percentage of cells that are multinucleated is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.
155. The source cell or population of source cells of any of embodiments 139-154, wherein the percent of nuclei present in syncytia is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.
156. The source cell or population of source cells of any of embodiments 139-155, wherein multinucleated cells (e.g., cells having two or more nuclei) are detected by a microscopy assay, e.g., using a DNA stain, e.g., an assay of Example 20.
157. The source cell or population of source cells of any of embodiments 139-156, wherein the functional fusosomes (e.g., viral particles) obtained from the modified source cells is at least 10%, 20%, 40%, 40%, 50%, 60%, 70%, 8-%, 90%, 2-fold, 5-fold, or 10-fold greater than the number of fusosomes obtained from otherwise similar unmodified source cells, e.g., using an assay of Example 20.
158. A fusosome that lacks a fusogen receptor or comprises a fusogen receptor that is present at reduced levels (e.g., reduced by at least 10%, 20%, 30%, 40%, 50%, 60%, 70%, 80%, or 90%) compared to an unmodified fusosome from an otherwise similar source cell.
159. A method of making a fusosome, comprising:

a) providing a source cell that comprises a fusogen (e.g., re-targeted fusogen), wherein the source cell lacks a fusogen receptor or comprises a fusogen receptor that is present at reduced levels (e.g., reduced by at least 10%, 20%, 30%, 40%, 50%, 60%, 70%, 80%, or 90%) compared to an otherwise similar, unmodified source cell;

b) culturing the source cell under conditions that allow for production of the fusosome, and

c) separating, enriching, or purifying the fusosome from the source cell, thereby making the fusosome.

160. The method of embodiment 159, wherein providing the source cell comprises knocking down or knocking out the fusogen receptor in the source cell or a precursor thereof.
161. The fusosome of embodiment 158 or the method of embodiment 159 or 160, wherein the fusosome is a retroviral vector or retrovirus like particle.
162. A retroviral vector (e.g., suitable for in vivo use in a human subject), comprising:

a) an envelope comprising a retargeted fusogen;

b) a retroviral nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise retroviral vector lacking the positive tissue-specific regulatory element; or
(ii) a negative target cell-specific regulatory element (e.g., a tissue-specific miRNA recognition sequence), operatively linked to the nucleic acid encoding the exogenous agent, wherein the negative tissue-specific regulatory element decreases expression of the exogenous agent in a non-target cell or tissue relative to an otherwise similar retroviral vector lacking the negative tissue-specific regulatory element.
   163. A retroviral vector (e.g., suitable for in vivo use in a human subject), comprising:

a) an envelope comprising a fusogen (e.g., a re-targeted fusogen);

b) a retroviral nucleic acid that comprises or encodes:
(i) a positive target cell-specific regulatory element (e.g., a tissue-specific promoter) operatively linked to a nucleic acid encoding an exogenous agent (e.g., exogenous polypeptide or exogenous RNA), wherein the positive tissue-specific regulatory element increases expression of the exogenous agent in a target cell or tissue relative to an otherwise retroviral vector lacking the positive tissue-specific regulatory element; and
(ii) a negative target cell-specific regulatory element (e.g., a tissue-specific miRNA recognition sequence), operatively linked to the nucleic acid encoding the exogenous agent, wherein the negative tissue-specific regulatory element decreases expression of the exogenous agent in a non-target cell or tissue relative to an otherwise similar retroviral vector lacking the negative tissue-specific regulatory element.
   164. The retroviral vector of either of the preceding embodiments, wherein, when administered to a subject, one or more of:
i) less than 10%, 5%, 4%, 3%, 2%, or 1% of the exogenous agent detectably present in the subject is in non-target cells;
ii) at least 90%, 95%, 96%, 97%, 98%, or 99% of the cells of the subject that detectably comprise the exogenous agent, are target cells (e.g., cells of a single cell type, e.g., T cells);
iii) less than 1,000,000, 500,000, 200,000, 100,000, 50,000, 20,000, or 10,000 cells of the cells of the subject that detectably comprise the exogenous agent are non-target cells;
iv) average levels of the exogenous agent in all target cells in the subject are at least 100-fold, 200-fold, 500-fold, or 1,000-fold higher than average levels of the exogenous agent in all non-target cells in the subject; or
v) the exogenous agent is not detectable in any non-target cell in the subject.
   165. The retroviral vector of any of the preceding embodiments, wherein the re-targeted fusogen comprises a sequence chosen from Nipah virus F and G proteins, measles virus F and H proteins, tupaia paramyxovirus F and H proteins, paramyxovirus F and G proteins or F and H proteins or F and HN proteins, Hendra virus F and G proteins, Henipavirus F and G proteins, Morbilivirus F and H proteins, respirovirus F and HN protein, a Sendai virus F and HN protein, rubulavirus F and HN proteins, or avulavirus F and HN proteins, or a derivative thereof, or any combination thereof.
   166. The retroviral vector of any of the preceding embodiments, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusogen has a sequence of amino acids set forth in any one of SEQ ID NOS: 1-132.
   167. The retroviral vector of embodiment 166, wherein the paramyxovirus is a Nipah virus, e.g., a henipavirus.
   168. The retroviral vector of any of the preceding embodiments, wherein the positive target cell-specific regulatory element comprises a tissue-specific promoter, a tissue-specific enhancer, a tissue-specific splice site, a tissue-specific site extending half-life of an RNA or protein, a tissue-specific mRNA nuclear export promoting site, a tissue-specific translational enhancing site, or a tissue-specific post-translational modification site.
   169. The retroviral vector of any of the preceding embodiments, wherein the negative target cell specific regulatory element comprises a tissue-specific miRNA recognition sequence, tissue-specific protease recognition site, tissue-specific ubiquitin ligase site, tissue-specific transcriptional repression site, or tissue-specific epigenetic repression site.
   170. The retroviral vector of any of the preceding embodiments, wherein the negative target cell specific regulatory element comprises a tissue-specific miRNA recognition sequence.
   171. The retroviral vector of embodiment 170, wherein the negative target cell specific regulatory element is situated or encoded within a transcribed region (e.g., the transcribed region encoding the exogenous agent), e.g., such that an RNA produced by the transcribed region comprises the miRNA recognition sequence within a UTR or coding region.
   172. The retroviral vector of any of the preceding embodiments, wherein the target cell is a cancer cell and the non-target cell is a non-cancerous cell.
   173. The retroviral vector of any of the preceding embodiments, wherein the retroviral nucleic acid encodes a positive TCSRE and/or a negative TCSRE.
   174. The retroviral vector of any of the preceding embodiments, wherein the retroviral nucleic acid comprises the complement of a positive TCSRE and/or a negative TCSRE.
   175. The retroviral vector of any of the preceding embodiments, which does not deliver nucleic acid to a non-target cell, e.g., an antigen presenting cell, an MHC class 11+ cell, a professional antigen presenting cell, an atypical antigen presenting cell, a macrophage, a dendritic cell, a myeloid dendritic cell, a plasmacyteoid dendritic cell, a CD I 1e+ cell, a CD11b+ cell, a splenocyte, a B cell, a hepatocyte, a endothelial cell, or a non-cancerous cell.
   176. The retroviral vector of any of the preceding embodiments, wherein less than 10%, 5%, 2.5%, 1%, 0.5%, 0.1%, 0.01%, 0.001%, 0.0001%, 0.00001%, or 0.000001% of a non-target cell type (e.g., one or more of an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD I 1e+ cell, a CD11b+ cell, a splenocyte, a B cell, a hepatocyte, a endothelial cell, or a non-cancerous cell) comprise the retroviral nucleic acid, e.g., using quantitative PCR, e.g., using an assay of Example 1.
   177. The retroviral vector of any of the preceding embodiments, wherein the target cells comprise 0.00001-10, 0.0001-10, 0.001-10, 0.01-10, 0.1-10, 0.5-5, 1-4, 1-3, or 1-2 copies of the retroviral nucleic acid or a portion thereof, per host cell genome, e.g., wherein copy number of the retroviral nucleic acid is assessed after administration in vivo.
   178. The retroviral vector of any of the preceding embodiments, wherein: less than 10%, 5%, 2.5%, 1%, 0.5%, 0.1%, 0.01% of the non-target cells (e.g., an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell) comprise the exogenous agent; or the exogenous agent (e.g., protein) is not detectably present in a non-target cell, e.g an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell.
   179. The retroviral vector of any of the preceding embodiments, wherein the retroviral vector delivers the retroviral nucleic acid to a target cell, e.g., a T cell, a CD3+ T cell, a CD4+ T cell, a CDS+ T cell, a hepatocyte, a haematepoietic stem cell, a CD34+ haematepoietic stem cell, a CD I05+ haematepoietic stem cell, a CD117+ haematepoietic stem cell, a CD I05+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLCIA3+ astrocyte, a SLC7AIO+ adipocyte, or a CD30+ lung epithelial cell.
   180. The retroviral vector of any of the preceding embodiments, wherein at least 0.00001%, 20 0.0001%, 0.001%, 0.001%, 0.01%, 0.1%, 1%, 2%, 5%, 10%, 20%, 30%, 40%, 50%, 60%, 70%, 80%, or 90% of target cells (e.g., one or more of a T cell, a CD3+ T cell, a CD4+ T cell, a CDS+ T cell, a hepatocyte, a haematepoietic stem cell, a CD34+ haematepoietic stem cell, a CD I05+ haematepoietic stem cell, a CD117+ haematepoietic stem cell, a CD I05+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLCIA3+ astrocyte, a SLC7AIO+ adipocyte, or a CD30+ lung epithelial cell) comprise the retroviral nucleic acid, e.g., using quantitative PCR, e.g., using an assay of Example 3.
   181. The retroviral vector of any of the preceding embodiments, wherein at least 0.00001%, 0.0001%, 0.001%, 0.001%, 0.01%, 0.1%, I %, 2%, 5%, 10%, 20%, 30%, 40%, 50%, 60%, 70%, 80%, or 90% of target cells (e.g., a T cell, a CD3+ T cell, a CD4+ T cell, a CDS+ T cell, a hepatocyte, a haematepoietic stem cell, a CD34+ haematepoietic stem cell, a CD I05+ haematepoietic stem cell, a CD117+ haematepoietic stem cell, a CD I05+ endothelial cell, a B cell, a CD20+ B cell, a CD19+ B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLCIA3+ astrocyte, a SLC7AIO+ adipocyte, or a CD30+ lung epithelial cell) comprise the exogenous agent.
   182. The retroviral vector of any of the preceding embodiments, wherein, upon administration, the ratio of target cells comprising the retroviral nucleic acid to non-target cells comprising the retroviral nucleic acid is at least 1.5, 2, 3, 4, 5, 10, 25, 50, 100, 500, 1000, 5000, 10,000, e.g., according to a quantitative PCR assay, e.g., using assays of Example 1 and Example 3.
   183. The retroviral vector of any of the preceding embodiments, wherein the ratio of the average copy number of retroviral nucleic acid or a portion thereof in target cells to the average copy number of retroviral nucleic acid or a portion thereof in non-target cells is at least 1.5, 2, 3, 4, 5, 10, 25, 50, 100, 500, 1000, 5000, 10,000, e.g., according to a quantitative PCR assay, e.g., using assays of Example 1 and Example 3.
   184. The retroviral vector of any of the preceding embodiments, wherein the ratio of the median copy number of of retroviral nucleic acid or a portion thereof in target cells to the median copy number of retroviral nucleic acid or a portion thereof in non-target cells is at least 1.5, 2, 3, 4, 5, 10, 25, 50, 100, 500, 1000, 5000, 10,000, e.g., according to a quantitative PCR assay, e.g., using assays of Example 1 and Example 3.
   185. The retroviral vector of any of the preceding embodiments, wherein the ratio of target cells comprising the exogenous RNA agent to non-target cells comprising the exogenous RNA agent is at least 1.5, 2, 3, 4, 5, 10, 25, 50, 100, 500, 1000, 5000, 10,000, e.g., according to a reverse transcription quantitative PCR assay.
   186. The retroviral vector of any of the preceding embodiments, wherein the ratio of the average exogenous RNA agent level of target cells to the average exogenous RNA agent level of non-target cells is at least 1.5, 2, 3, 4, 5, 10, 25, 50, 100, 500, 1000, 5000, 10,000, e.g., according to a reverse transcription quantitative PCR assay.
   187. The retroviral vector of any of the preceding embodiments, wherein the ratio of the median exogenous RNA agent level of target cells to the median exogenous RNA agent level of non-target cells is at least 1.5, 2, 3, 4, 5, 10, 25, 50, 100, 500, 1000, 5000, 10,000, e.g., according to a reverse transcription quantitative PCR assay.
   188. The retroviral vector of any of the preceding embodiments, wherein the ratio of target cells comprising the exogenous protein agent to non-target cells comprising the exogenous protein agent is at least 1.5, 2, 3, 4, 5, 10, 25, 50, 100, 500, 1000, 5000, 10,000, e.g., according to a FACS assay, e.g., using assays of Example 2 and/or Example 4.
   189. The retroviral vector of any of the preceding embodiments, wherein the ratio of the average exogenous protein agent level of target cells to the average exogenous protein agent level of non-target cells is at least 1.5, 2, 3, 4, 5, 10, 25, 50, 100, 500, 1000, 5000, 10,000, e.g., according to a FACS assay, e.g., using assays of Example 2 and/or Example 4.
   190. The retroviral vector of any of the preceding embodiments, wherein the ratio of the median exogenous protein agent level of target cells to the median exogenous protein agent level of non-target cells is at least 1.5, 2, 3, 4, 5, 10, 25, 50, 100, 500, 1000, 5000, 10,000, e.g., according to a FACS assay, e.g., using assays of Example 2 and/or Example 4.
   191. The retroviral vector of any of the preceding embodiments, which comprises one or both of:
i) an exogenous or overexpressed immunosuppressive protein on the envelope; and
ii) an immunostimulatory protein that is absent or present at reduced levels (e.g., reduced by at least 10%, 20%, 30%, 40%, 50%, 60%, 70%, 80%, or 90%) compared to a retroviral vector generated from an otherwise similar, unmodified source cell.
   192. The retroviral vector of any of the preceding embodiments, which comprises one or more of:
i) a first exogenous or overexpressed immunosuppressive protein on the envelope and a second exogenous or overexpressed immunosuppressive protein on the envelope;
ii) a first exogenous or overexpressed immunosuppressive protein on the envelope and a second immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell; or
iii) a first immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell and a second immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell.
   193. The retroviral vector of any of the preceding embodiments, wherein the retroviral nucleic acid comprises one or more insulator elements.
   194. A retrovirus-like particle or retroviral vector (e.g., a particle or vector suitable for in vivo use in a human subject), comprising:

a) an envelope comprising a fusogen (e.g., a re-targeted fusogen), and

b) an exogenous agent (e.g., exogenous polypeptide or exogenous RNA) or a nucleic acid (e.g., a retroviral nucleic acid) encoding an exogenous agent; and

c) one or more of:
i) a first exogenous or overexpressed immunosuppressive protein on the envelope and a second exogenous or overexpressed immunosuppressive protein on the envelope;
ii) a first exogenous or overexpressed immunosuppressive protein on the envelope and a second immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell; or
iii) a first immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell and a second immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell; wherein, when administered to a subject (e.g., a human subject or a mouse), one or more of:
   i) the particle or vector does not produce a detectable antibody response (e.g., after a single administration or a plurality of administrations), or antibodies against the particle or vector are present at a level of less than 10%, 5%, 4%, 3%, 2%, or I % above a background level, e.g., by a FACS antibody detection assay, e.g., an assay of Example 13 or Example 14);
   ii) the particle or vector does not produce a detectable cellular immune response (e.g., T cell response, NK cell response, or macrophage response), or a cellular immune response against the particle or vector is present at a level of less than 10%, 5%, 4%, 3%, 2%, or I % above a background level, e.g., by a PBMC lysis assay (e.g., an assay of Example 5), by an NK cell lysis assay (e.g., an assay of Example 6), by a CDS killer T cell lysis assay (e.g., an assay of Example 7), by a macrophage phagocytosis assay (e.g., an assay of Example 8);
   iii) the particle or vector does not produce a detectable innate immune response, e.g., complement activation (e.g., after a single administration or a plurality of administrations), or the innate immune response against the particle or vector is present at a level of less than 10%, 5%, 4%, 3%, 2%, or I % above a background level, e.g., by a complement activity assay (e.g., an assay of Example 9);
   iv) less than 90%, 80%, 70%, 60%, 50%, 40%, 30%, 20%, 10%, 5%, 4%, 3%, 2%, 1%, 0.5%, 0.2%, 0.1%, 0.05%, 0.02%, 0.01%, 0.005%, 0.002%, or 0.001% of viruses are inactivated by serum, e.g., by a serum inactivation assay, e.g., an assay of Example 11 or Example 12;
   v) a target cell that has received the exogenous agent from the particle or vector do not produce a detectable antibody response (e.g., after a single administration or a plurality of administrations), or antibodies against the target cell are present at a level of less than 10%, 5%, 4%, 3%, 2%, or I % above a background level, e.g., by a FACS antibody detection assay, e.g., an assay of Example 15; or
   vi) a target cell that has received the exogenous agent from the particle or vector do not produce a detectable cellular immune response (e.g., T cell response, NK cell response, or macrophage response), or a cellular response against the target cell is present at a level of less than 10%, 5%, 4%, 3%, 2%, or I % above a background level, e.g., by a macrophage phagocytosis assay (e.g., an assay of Example 16), by a PBMC lysis assay (e.g., an assay of Example 17), by an NK cell lysis assay (e.g., an assay of Example 18), or by a CDS killer T cell lysis assay (e.g., an assay of Example 19).
      195. The retrovirus-like particle or retroviral vector of embodiment 194, wherein the background level is the corresponding level in the same subject prior to administration of the particle or vector.
      196. The retrovirus-like particle or retroviral vector of embodiments 194 or 195, wherein the immunosuppressive protein is a complement regulatory protein or CD47.
      197. The retrovirus-like particle or retroviral vector of any of embodiments 194-196, wherein the immunostimulatory protein is an MHC (e.g., HLA) protein.
      198. The retrovirus-like particle or retroviral vector of any of embodiments 194-197, wherein one or both of: the first exogenous or overexpressed immunosuppressive protein is other than CD47, and the second immunostimulatory protein is other than MHC.
      199. A retrovirus-like particle or retroviral vector (e.g., a particle or vector suitable for in vivo use in a human subject), comprising:
a) an envelope comprising a fusogen, and
b) a retroviral nucleic acid encoding an exogenous agent (e.g., exogenous polypeptide or exogenous RNA);
c) exogenous or overexpressed MHC, e.g., HLA (e.g., HLA-G or HLA-E), or a combination thereof, on the envelope.
   200. A pseudotyped retrovirus-like particle or retroviral vector (e.g., a particle or vector suitable for in vivo use in a human subject), comprising:
a) a pseudotyped envelope comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence Table 4 or Table 5, optionally wherein the wild-type paramyxovirus fusogen has a sequence of amino acids set forth in any one of SEQ ID NOS: 1-132; and
b) a retroviral nucleic acid encoding an exogenous agent (e.g., exogenous polypeptide or exogenous RNA);
c) exogenous or overexpressed CD47 or a complement regulatory protein, or a combination thereof, on the envelope.
   201. A pseudotyped retrovirus-like particle or retroviral vector (e.g., a particle or vector suitable for in vivo use in a human subject), comprising:
a) a pseudotyped envelope comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusogen has a sequence of amino acids set forth in any one of SEQ ID NOS: 1-132, and
b) a retroviral nucleic acid encoding an exogenous agent (e.g., exogenous polypeptide or exogenous RNA); and
c) MHC I (e.g., HLA-A, HAL-B, or HLA-C) or MHC II (e.g., HLA-DP, HLA-DM, HLA-DOA, HLA-DOB, HLA-DQ, or HLA-DR) that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell.
   202. A pseudotyped retrovirus-like particle or retroviral vector (e.g., a particle or vector suitable for in vivo use in a human subject), comprising:
   a) a pseudotyped envelope comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusogen has a sequence of amino acids set forth in any one of SEQ ID NOS: 1-132; and
   b) a retroviral nucleic acid encoding an exogenous agent (e.g., exogenous polypeptide or exogenous RNA); and
   c) one or both of an exogenous or overexpressed immunosuppressive protein or an immunostimulatory protein that is absent or present at reduced levels (e.g., reduced by at least 10%, 20%, 30%, 40%, 50%, 60%, 70%, 80%, or 90%) compared to a retrovirus-like particle or retroviral vector generated from an otherwise similar, unmodified source cell.
   203. The retrovirus-like particle or retroviral vector of any of the preceding embodiments, wherein the retrovirus-like particle or retroviral vector is in circulation at least 0.5, 1, 2, 3, 4, 6, 12, 18, 24, 36, or 48 hours after administration to the subject.
   204. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 30 minutes after administration.
   205. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 1 hour after administration.
   206. The retrovirus-like particle or retroviral vector of any of the preceding embodiments, wherein at least 0.001%, 0.01%, 0.1%, I %, 10%, 20%, 30%, 40%, 50%, 60%, 70%, 80%, 90%, or 100% of retroviruses are in circulation 2 hours after administration.
   207. The retrovirus-like particle or retroviral vector of any of the preceding embodiments, wherein at least embodiments 0.001%, 0.01%, 0.1%, 1%, 10%, 20%, 30%, 40%, 50%, 60%, 70%, 80%, 90%, or 100% of retroviruses are in circulation 4 hours after administration.
   208. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 8 hours after administration.
   209. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 12 hours after administration.
   210. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 18 hours after administration.
   211. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 24 hours after administration.
   212. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 36 hours after administration.
   213. The retrovirus-like particle or retroviral vector of any of the preceding embodiments, wherein at least 0.001%, 0.01%, 0.1%, 1%, 10%, 20%, 30%, 40%, 50%, 60%, 70%, 80%, 90%, or 100% of retroviruses are in circulation 48 hours after administration.
   214. The retrovirus-like particle or retroviral vector of any of the preceding embodiments, which has a reduction in immunogenicity as measured by a reduction in humoral response following one or more administration of the retrovirus to an appropriate animal model, e.g., an animal model described herein, compared to reference retrovirus, e.g., an unmodified retrovirus otherwise similar to the retrovirus.
   215. The retrovirus-like particle or retroviral vector of any of the preceding embodiments, wherein the reduction in humoral response is measured in a serum sample by an anti-cell antibody titre, e.g., anti-retroviral antibody titre, e.g., by ELISA.
   216. The retrovirus-like particle or retroviral vector of any of the preceding embodiments, wherein a serum sample from animals administered the retrovirus composition has a reduction of 1%, 5%, 10%, 20%, 30%, 40%, 50%, 60%, 70%, 80%, 90%, or more of an anti-retrovirus antibody titer compared to the serum sample from a subject administered an unmodified cell.
   217. The retrovirus-like particle or retroviral vector of any of the preceding embodiments, wherein a serum sample from a subject administered the retorivirus composition has an increased anti-cell antibody titre, e.g., increased by 1%, 2%, 5%, 10%, 20%, 30%, or 40% from baseline, e.g., wherein baseline refers to serum sample from the same subject before administration of the retrovirus composition.
   218. The retrovirus-like particle or retroviral vector of any of the preceding embodiments, wherein: the subject to be administered the retrovirus or pharmaceutical composition has, or is known to have, or is tested for, a pre-existing antibody (e.g., IgG or IgM) reactive with the retrovirus; the subject to be administered the retrovirus composition does not have detectable levels of a pre-existing antibody reactive with the retrovirus; a subject that has received the retrovirus or pharmaceutical composition has, or is known to have, or is tested for, an antibody (e.g., IgG or IgM) reactive with the retrovirus; the subject that received the retrovirus or pharmaceutical composition (e.g., at least once, twice, three times, four times, five times, or more) does not have detectable levels of antibody reactive with the retrovirus; or levels of antibody do not rise more than 1%, 2%, 5%, 10%, 20%, or 50% between two timepoints, the first timepoint being before the first administration of the retrovirus, and the second timepoint being after one or more administrations of the retorivirus.
   219. The retrovirus-like particle or retroviral vector of any of the preceding embodiments, wherein the retroviral vector is produced by the methods of Example 5, 6, or 7, e.g., from cells transfected with HLA-G or HLA-E cDNA.
   220. The retrovirus-like particle or retroviral vector of any of the preceding embodiments, wherein retroviral vectors generated from NMC-HLA-G cells have a decreased percentage of lysis, e.g., PBMC mediated lysis, NK cell mediated lysis, and/or CDS+ T cell mediated lysis, at specific timepoints as compared to retroviral vectors generated from NMCs or NMC-empty vector.
   221. The retrovirus-like particle or retroviral vector of any of the preceding embodiments, wherein the modified retroviral vector evades phagocytosis by macrophages.
   222. The retrovirus-like particle or retroviral vector of any of the preceding embodiments, wherein the retroviral vector is produced by the methods of Example 8, e.g., from cells transfected with CD47 cDNA.
   223. The retrovirus-like particle or retroviral vector of any of the preceding embodiments, wherein the phagocytic index is reduced when macrophages are incubated with retroviral vectors derived from NMC-CD47, versus those derived from NMC, or NMC-empty vector.
   224. The retrovirus-like particle or retroviral vector of any of the preceding embodiments, which has a reduction in macrophage phagocytosis, e.g., a reduction of 1%, 5%, 10%, 20%, 30%, 40%, 50%, 60%, 70%, 80%, 90%, or more in macrophage phagocytosis compared to a reference retrovirus, e.g., an unmodified retrovirus otherwise similar to the retrovirus, wherein the reduction in macrophage phagocytosis is determined by assaying the phagocytosis index in vitro, e.g., as described in Example 8.
   225. The retrovirus-like particle or retroviral vector of any of the preceding embodiments, wherein the retrovirus composition has a phagocytosis index of 0, 1, 10, 100, or more, e.g., as measured by an assay of Example 8, when incubated with macrophages in an in vitro assay of macrophage phagocytosis.
   226. The retrovirus-like particle or retroviral vector of any of the preceding embodiments, which is modified and has reduced complement activity compared to an unmodified retroviral vector.
   227. The retrovirus-like particle or retroviral vector of any of the preceding embodiments, which is produced by the methods of Example 9, e.g., from cells transfected with a cDNA coding for complement regulatory proteins, e.g., DAF.
   228. The retrovirus-like particle or retroviral vector of any of the preceding embodiments, wherein the dose of retroviral vector at which 200 μg/ml of C3a is present is greater for the modified retroviral vector (e.g., HEK293-DAF) incubated with corresponding mouse sera (e.g., HEK-293 DAF mouse sera) than for the reference retroviral vector (e.g., HEK293 retroviral vector) incubated with corresponding mouse sera (e.g., HEK293 mouse sera).
   229. The retrovirus-like particle or retroviral vector of any of the preceding embodiments, wherein the dose of retroviral vector at which 200 μg/ml of C3a is present is greater for for the modified retroviral vector (e.g., HEK293-DAF) incubated with naive mouse sera than for the reference retroviral vector (e.g., HEK293 retroviral vector) incubated with naive mouse sera.
   230. The retrovirus-like particle or retroviral vector of any of the preceding embodiments, wherein the retrovirus composition is resistant to complement mediated inactivation in patient serum 30 minutes after administration according to an assay of Example 9.
   231. The retrovirus-like particle or retroviral vector of any of the preceding embodiments wherein at least 0.001%, 0.01%, 0.1%, 1%, 10%, 20%, 30%, 40%, 50%, 60%, 70%, 80%, 90%, or 100% of retroviruses are resistant to complement mediated inactivation.
   232. The retrovirus-like particle or retroviral vector of any of the preceding embodiments, wherein the complement regulatory protein comprises one or more of proteins that bind decay-accelerating factor (DAF, CD55), e.g. factor H (FH)-like protein-I (FHL-1), e.g. C4b-binding protein (C4BP), e.g. complement receptor I (CD35), e.g. Membrane cofactor protein (MCP, CD46), eg. Protectin (CD59), e.g. proteins that inhibit the classical and alternative complement pathway CD/C5 convertase enzymes, e.g. proteins that regulate MAC assembly.
   233. The retrovirus-like particle or retroviral vector of any of the preceding embodiments, which is produced by the methods of Example 10, e.g., from cells transfected with a DNA coding for an shRNA targeting MHC class I, e.g., wherein retroviral vectors derived from NMC-shMHC class I has lower expression of MHC class I compared to NMCs and NMC-vector control.
   234. The retrovirus-like particle or retroviral vector of any of the preceding embodiments, wherein a measure of immunogenicity for retroviral vectors is serum inactivation, e.g., serum inactivation measured as described herein, e.g., as described in Example 11.
   235. The retrovirus-like particle or retroviral vector of any of the preceding embodiments, wherein the percent of cells which receive the exogenous agent is not different between retroviral vector samples that have been incubated with serum and heat-inactivated serum from retroviral vector naive mice.
   236. The retrovirus-like particle or retroviral vector of any of the preceding embodiments, wherein the percent of cells which receive the exogenous agent is not different between retroviral vector samples that have been incubated with serum from retroviral vector nai:ve mice and no-serum control incubations.
   237. The retrovirus-like particle or retroviral vector of any of the preceding embodiments wherein the percent of cells which receive the exogenous agent is less in retroviral vector samples that have been incubated with positive control serum than in retroviral vector samples that have been incubated with serum from retroviral vector naive mice.
   238. The retrovirus-like particle or retroviral vector of any of the preceding embodiments, wherein a modified retroviral vector, e.g., modified by a method described herein, has a reduced (e.g., reduced compared to administration of an unmodified retroviral vector) serum inactivation following multiple (e.g., more than one, e.g., 2 or more), administrations of the modified retroviral vector.
   239. The retrovirus-like particle or retroviral vector of any of the preceding embodiments, wherein a retroviral vector described herein is not inactivated by serum following multiple administrations.
   240. The retrovirus-like particle or retroviral vector of any of the preceding embodiments, wherein a measure of immunogenicity for retroviral vector is serum inactivation, e.g., after multiple administrations, e.g., serum inactivation after multiple administrations measured as described herein, e.g., as described in Example 12.
   241. The retrovirus-like particle or retroviral vector of any of the preceding embodiments, wherein the percent of cells which receive the exogenous agent is not different between retroviral vector samples that have been incubated with serum and heat-inactivated serum from mice treated with modified (e.g., HEK293-HLA-G) retroviral vectors.
   242. The retrovirus-like particle or retroviral vector of any of the preceding embodiments, wherein the percent of cells which receive the exogenous agent is not different between retroviral vector samples that have been incubated from mice treated 1, 2, 3, 5 or 10 times with modified (e.g., HEK293-HLA-G) retroviral vectors.
   243. The retrovirus-like particle or retroviral vector of any of the preceding embodiments, wherein the percent of cells which receive the exogenous agent is not different between retroviral vector samples that have been incubated with serum from mice treated with vehicle and from mice treated with modified (e.g., HEK293-HLA-G) retroviral vectors.
   244. The retrovirus-like particle or retroviral vector of any of the preceding embodiments, wherein the percent of cells which receive the exogenous agent is less for retroviral vectors derived from a reference cell (e.g., HEK293) than for modified (e.g., HEK293-HLA-G) retroviral vectors.
   245. The retrovirus-like particle or retroviral vector of any of the preceding embodiments, wherein a measure of immunogenicity for a retroviral vector is antibody responses.
   246. The retrovirus-like particle or retroviral vector of any of the preceding embodiments, wherein a subject that receives a retroviral vector described herein has pre-existing antibodies which bind to and recognize retroviral vector, e.g., measured as described herein, e.g., as described in Example 13.
   247. The retrovirus-like particle or retroviral vector of any of the preceding embodiments, wherein serum from retroviral vector-nai:ve mice shows more signal (e.g., fluorescence) than the negative control, e.g., serum from a mouse depleted of IgM and IgG, e.g., indicating that in immunogenicity has occurred.
   248. The retrovirus-like particle or retroviral vector of any of the preceding embodiments, wherein serum from retroviral vector-nai:ve mice shows similar signal (e.g., fluorescence) compared to the negative control, e.g., indicating that immunogenicity did not detectably occur.
   249. The retrovirus-like particle or retroviral vector of any of the preceding embodiments, which is a modified retroviral vector, e.g., modified by a method described herein, and which has a reduced (e.g., reduced compared to administration of an unmodified retroviral vector) humoral response following multiple (e.g., more than one, e.g., 2 or more), administrations of the modified retroviral vector e.g., measured as described herein, e.g., as described in Example 14.
   250. The retrovirus-like particle or retroviral vector of any of the preceding embodiments, wherein the retroviral vector is produced by the methods of Example 5, 6, 7, or 14, e.g., from cells transfected with HLA-G or HLA-E cDNA.
   251. The retrovirus-like particle or retroviral vector of any of the preceding embodiments, wherein humoral response is assessed by determining a value for the level of anti-retroviral vector antibodies (e.g., IgM, IgG1, and/or IgG2 antibodies).
   252. The retrovirus-like particle or retroviral vector of any of the preceding embodiments, wherein modified (e.g., NMC-HLA-G) retroviral vectors have decreased anti-viral IgM or IgG1/2 antibody titers (e.g., as measured by fluorescence intensity on FACS) after injections, as compared to a control, e.g., NMC retroviral vectors or NMC-empty retroviral vectors.
   253. The retrovirus-like particle or retroviral vector of any of the preceding embodiments, wherein recipient cells are not targeted by an antibody response, or an antibody response will be below a reference level, e.g., measured as described herein, e.g., as described in Example 15.
   254. The retrovirus-like particle or retroviral vector of any of the preceding embodiments, signal (e.g., mean fluorescence intensity) is similar for recipient cells from mice treated with retroviral vectors and mice treated with PBS.
   255. The retrovirus-like particle or retroviral vector of any of the preceding embodiments, wherein a measure of the immunogenicity of recipient cells is the macrophage response.
   256. The retrovirus-like particle or retroviral vector of any of the preceding embodiments, wherein recipient cells are not targeted by macrophages, or are targeted below a reference level.
   257. The retrovirus-like particle or retroviral vector of any of the preceding embodiments, wherein the phagocytic index, e.g., measured as described herein, e.g., as described in Example 16, is similar for recipient cells derived from mice treated with retroviral vectors and mice treated with PBS.
   258. The retrovirus-like particle or retroviral vector of any of the preceding embodiments, wherein a measure of the immunogenicity of recipient cells is the PBMC response.
   259. The retrovirus-like particle or retroviral vector of any of the preceding embodiments, wherein recipient cells do not elicit a PBMC response.
   260. The retrovirus-like particle or retroviral vector of any of the preceding embodiments, wherein the percent of CD3+/CMG+ cells is similar for recipient cells derived from mice treated with retroviral vector and mice treated with PBS, e.g., as measured as described herein, e.g., as described in Example 17.
   261. The retrovirus-like particle or retroviral vector of any of the preceding embodiments, wherein a measure of the immunogenicity of recipient cells is the natural killer cell response.
   262. The retrovirus-like particle or retroviral vector of any of the preceding embodiments, wherein recipient cells do not elicit a natural killer cell response or elicit a lower natural killer cell response, e.g., lower than a reference value.
   263. The retrovirus-like particle or retroviral vector of any of the preceding embodiments, wherein the percent of CD3+/CMG+ cells is similar for recipient cells derived from mice treated with retroviral vector and mice treated with PBS, e.g., as measured as described herein, e.g., as described in Example 18.
   264. The retrovirus-like particle or retroviral vector of any of the preceding embodiments, wherein a measure of the immunogenicity of recipient cells is the CDS+ T cell response.
   265. The retrovirus-like particle or retroviral vector of any of the preceding embodiments wherein recipient cells do not elicit a CDS+ T cell response or elicit a lower CDS+ T cell response, e.g., lower than a reference value.
   266. The retrovirus-like particle or retroviral vector of any of the preceding embodiments, wherein the percent of CD3+/CMG+ cells is similar for recipient cells derived from mice treated with retroviral vector and mice treated with PBS, e.g., as measured as described herein, e.g., as described in Example 19.
   267. The retrovirus-like particle or retroviral vector of any of the preceding embodiments, wherein the fusogen is a re-targeted fusogen.
   268. The retrovirus-like particle or retroviral vector of any of the preceding embodiments, which comprises a retroviral nucleic acid that encodes one or both of: (i) a positive target cell-specific regulatory element operatively linked to a nucleic acid encoding an exogenous agent, or (ii) a negative target cell-specific regulatory element operatively linked to the nucleic acid encoding the exogenous agent.
   269. A pseudotyped retrovirus-like particle or retroviral vector (e.g., a particle or vector suitable for in vivo use in a human subject), comprising:
a) a pseudotyped envelope comprising a fusogen, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5, optionally wherein the wild-type paramyxovirus fusogen has a sequence of amino acids set forth in any one of SEQ ID NOS: 1-132; and
b) a retroviral nucleic acid encoding an exogenous agent (e.g., exogenous polypeptide or exogenous RNA), wherein the retroviral nucleic acid comprises one or more insulator elements.
   270. The pseudotyped retrovirus-like particle or retroviral vector of embodiment 269, wherein the retroviral nucleic acid comprises two insulator elements, e.g., a first insulator element upstream of a region encoding the exogenous agent and a second insulator element downstream of a region encoding the exogenous agent, e.g., wherein the first insulator element and second insulator element comprise the same or different sequences.
   271. The pseudotyped retrovirus-like particle or retroviral vector of embodiment 269 or 270, wherein variation in the median exogenous agent level in a sample of cells isolated after administration of the particle or vector to the subject at a first timepoint is at least, less than, or about 10,000%, 5,000%, 2,000%, 1,000%, 500%, 200%, 100%, 50%, 20%, 10%, or 5% of the median exogenous agent level in a sample of cells isolated after administration of the particle or vector to the subject at a second, later timepoint.
   272. The pseudotyped retrovirus-like particle or retroviral vector of embodiment 271, wherein the median expression level per cell is assessed only in cells that have a retroviral genome copy number of at least 1.0.
   273. The pseudotyped retrovirus-like particle or retroviral vector of any of embodiments 269-272, wherein at least 10%, 20%, 30%, 40%, 50%, 60%, 70%, 80%, or 90% of target cells in the subject detectably comprise the exogenous agent.
   274. The pseudotyped retrovirus-like particle or retroviral vector of any of embodiments 271-273, wherein the median payload gene expression level is assessed across cells isolated from the subject 7 days, 14 days, 28 days, 56 days, 112 days, 365 days, 730 days, 1095 days after administration of the retroviral composition to the subject.
   275. The pseudotyped retrovirus like particle or retroviral vector of any of embodiments 269-274, wherein at least 10%, 20%, 30%, 40%, 50%, 60%, 70%, 80%, or 90% of target cells in the subject that detectably comprised the exogenous agent at a first time point still detectably comprise the exogenous agent at a second, later timepoint, e.g., wherein the first time point is 7 days, 14 days, 28 days, 56 days, 112 days, 365 days, 730 days, 1095 days after administration of the retroviral composition to the subject.
   276. The pseudotyped retrovirus like particle or retroviral vector of embodiment 275, wherein the second time point is 7 days, 14 days, 28 days, 56 days, 112 days, 365 days, 730 days, 1095 days after the first time point.
   277. The pseudotyped retrovirus like particle or retroviral vector of any of embodiments 269-276, which is not genotoxic or does not increase the rate of tumor formation in target cells compared to target cells not treated with the retrovirus like particle or retroviral vector.
   278. The pseudotyped retrovirus like particle or retroviral vector of any of embodiments 269-277, wherein the median exogenous agent level is assessed in a population of cells from a subject that has received the retroviral vector or pharmaceutical composition.
   279. The pseudotyped retrovirus like particle or retroviral vector of any of embodiments 269-278, wherein the median exogenous agent level assessed in populations of cells collected (e.g., isolated) from the subject at different days post administration is less than about 10,000% 1000%, 100%, or 10%, e.g., 10,000%-1000%, 1000%-100%, or 100%-10% different from the median exogenous agent level in the population of cells assessed at day 7, day 14, day 28, or day 56, wherein the cells in the population have a vector copy number of at least 1.0.
   280. The pseudotyped retrovirus like particle or retroviral vector of any of embodiments 269-279, wherein exogenous agent level is assessed across cells from a subject that has received the retroviral vector or pharmaceutical composition.
   281. The pseudotyped retrovirus like particle or retroviral vector of any of embodiments 269-280, wherein the percent of cells comprising the exogenous agent is assessed in a plurality of cells collected (e.g., isolated) from the subject 7 days, 14 days, 28 days, 56 days, 112 days, 365 days, 730 days, 1095 days after administration of the retroviral vector or pharmaceutical composition.
   282. The pseudotyped retrovirus like particle or retroviral vector of any of embodiments 269-281, wherein the difference in the percent of cells comprising the exogenous agent assessed in cells isolated at two different days post administration is less than I %, 5%, 10%, 20%, 50%, 75%, 100%, 150%, 200%, 250%, 300%, 400%, 500%, 750%, 1000%, 1500%, or 2000%.
   283. The pseudotyped retrovirus like particle or retroviral vector of any of embodiments 269-282, wherein the percent of target cells that are positive for the exogenous agent is similar across cells collected at 7 days, 14 days, 28 days, 56 days, 112 days, 365 days, 730 days, or 1095 days.
   284. The pseudotyped retrovirus like particle or retroviral vector of any of embodiments 269-283, wherein: at least as many target cells are positive for the exogenous agent 14 days, 28 days, 56 days, 112 days, 365 days, 730 days, or 1095 days as at 7 days; at least as many target cells are positive for the exogenous agent at 28 days, 56 days, 112 days, 365 days, 730 days, or 1095 days as at 14 days; at least as many target cells are positive for the exogenous agent at 56 days, 112 days, 365 days, 730 days, or 1095 days as at 28 days; at least as many target cells are positive for the exogenous agent at 112 days, 365 days, 730 days, or 1095 days as at 56 days; at least as many target cells are positive for the exogenous agent at 365 days, 730 days, or 1095 days as at 112 days; at least as many target cells are positive for the exogenous agent at 730 days or 1095 days as at 365 days; or at least as many target cells are positive for the exogenous agent at 1095 days as at 730 days.
   285. The pseudotyped retrovirus like particle or retroviral vector of any of embodiments 269-284, wherein: the median exogenous agent level in target cells that comprise the exogenous agent is similar in cells collected at 7 days, 14 days, 28 days, 56 days, 112 days, 365 days, 730 days, or 1095 days; the median exogenous agent level in target cells that comprise the exogenous agent at 14 days, 28 days, 56 days, 112 days, 365 days, 730 days, or 1095 days is at least as high as at 7 days; the median exogenous agent level in target cells that comprise the exogenous agent at 28 days, 56 days, 112 days, 365 days, 730 days, or 1095 days is at least as high as at 14 days; the median exogenous agent level in target cells that comprise the exogenous agent at 56 days, 112 days, 365 days, 730 days, or 1095 days is at least as high as at 28 days; the median exogenous agent level in target cells that comprise the exogenous agent at 112 days, 365 days, 730 days, or 1095 days is at least as high as at 56 days; the median exogenous agent level in target cells that comprise the exogenous agent at 365 days, 730 days, or 1095 days is at least as high as at 112 days; the median exogenous agent level in target cells that comprise the exogenous agent at 730 days, or 1095 days is at least as high as at 365 days; or the median exogenous agent level in target cells that comprise the exogenous agent at 1095 days is at least as high as at 730 days.
   286. A method of delivering an exogenous agent to a subject (e.g., a human subject) comprising administering to the subject a retrovirus-like particle (e.g., a pseudotyped retrovirus-like particle) or retroviral vector (e.g., pseudotyped retroviral vector) of any of the preceding embodiments, thereby delivering the exogenous agent to the subject.
   287. A method of modulating a function, in a subject (e.g., a human subject), target tissue or target cell, comprising contacting, e.g., administering to, the subject, the target tissue or the target cell with a retrovirus-like particle (e.g., a pseudotyped retrovirus-like particle) or retroviral vector (e.g., pseudotyped retroviral vector) of any of the preceding embodiments.
   288. The method of embodiment 287, wherein the target tissue or the target cell is present in a subject.
   289. A method of treating or preventing a disorder, e.g., a cancer, in a subject (e.g., a human subject) comprising administering to the subject a retrovirus-like particle (e.g., a pseudotyped retrovirus-like particle) or retroviral vector (e.g., pseudotyped retroviral vector) of any of the preceding embodiments.
   290. A method of making a retroviral vector or retrovirus-like particle of any of the preceding embodiments, comprising:
a) providing a source cell that comprises the retroviral nucleic acid and the fusogen (e.g., re-targeted fusogen);
b) culturing the source cell under conditions that allow for production of the retroviral vector, and
c) separating, enriching, or purifying the retroviral vector from the source cell, thereby making the retroviral vector.
   291. A source cell for producing a retroviral vector, comprising:
a) a retroviral nucleic acid;
b) viral structural proteins that can package the retroviral nucleic acid, wherein at least one viral structural protein comprises a fusogen that binds a fusogen receptor; and
c) a fusogen receptor that is absent or present at reduced levels (e.g., reduced by at least 10%, 20%, 30%, 40%, 50%, 60%, 70%, 80%, or 90%) compared to an otherwise similar, unmodified source cell.
   292. The source cell of embodiment 291, wherein the fusogen causes fusion of the retroviral vector with the target cell upon binding to the fusogen receptor.
   293. The source cell of embodiment 291 or 292, which binds to the second similar source cell, e.g., the fusogen of the source cell binds to the fusogen receptor on the second source cell.
   294. A population of source cells of any of embodiments 291-293.
   295. The population of source cells of embodiment 294, wherein less than 10%, 5%, 4%, 3%, 2%, or 1% of cells in the population are multinucleated.
   296. The source cell or population of source cells of any of embodiments 291-295, wherein a source cell is modified to have reduced fusion (e.g., to not fuse) with other source cells during manufacturing of a retroviruse described herein.
   297. The source cell or population of source cells of any of embodiments 291-296, wherein the fusogen (e.g., re-targeted fusogen) does not bind to a protein comprised by a source cell, e.g., to a protein on the surface of the source cell.
   298. The source cell or population of source cells of any of embodiments 291-297, wherein the fusogen (e.g., re-targeted fusogen) binds to a protein comprised by a source cell, but does not fuse with the cell.
   299. The source cell or population of source cells of any of embodiments 291-298, wherein the fusogen does not induce fusion with a source cell.
   300. The source cell or population of source cells of any of embodiments 291-299, wherein the source cell does not express a protein, e.g., an antigen, that binds the fusogen.
   301. The source cell or population of source cells of any of embodiments 291-300, a plurality of source cells do not form a syncytium when expressing the fusogen, or less than 50%, 40%, 30%, 20%, 10%, 5%, 4%, 3%, 2%, or I % of cells in the population are multinucleated (e.g., comprise two or more nuclei).
   302. The source cell or population of source cells of any of embodiments 291-301, wherein a plurality of source cells do not form a syncytium when producing lentivirus, or less than 50%, 40%, 30%, 20%, 10%, 5%, 4%, 3%, 2%, or I % of cells in the population are multinucleated.
   303. The source cell or population of source cells of any of embodiments 291-302, wherein less than 50%, 40%, 30%, 20%, 10%, 5%, 4%, 3%, 2%, or I % of the nuclei in the population are in syncytia.
   304. The source cell or population of source cells of any of embodiments 291-303, wherein at least 50%, 60%, 70%, 80%, 90%, 95%, 96%, 97%, 98%, 99% of nuclei in the population are in uninuclear cells.
   305. The source cell or population of source cells of any of embodiments 291-304, wherein the percentage of cells that are multinucleated is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.
   306. The source cell or population of source cells of any of embodiments 291-305, wherein the percent of nuclei present in syncytia is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.
   307. The source cell or population of source cells of any of embodiments 291-306, wherein multinucleated cells (e.g., cells having two or more nuclei) are detected by a microscopy assay, e.g., using a DNA stain, e.g., an assay of Example 20.
   308. The source cell or population of source cells of any of embodiments 291-307, wherein the functional viral particles obtained from the modified source cells is at least 10%, 20%, 40%, 40%, 50%, 60%, 70%, 80%, 90%, 2-fold, 5-fold, or 10-fold greater than the number of functional viral particles obtained from otherwise similar unmodified source cells, e.g., using an assay of Example 20.
   309. A retroviral vector or retrovirus like particle that lacks a fusogen receptor or comprises a fusogen receptor that is present at reduced levels (e.g., reduced by at least 10%, 20%, 30%, 40%, 50%, 60%, 70%, 80%, or 90%) compared to an unmodified retroviral vector or retrovirus like particle from an otherwise similar source cell.
   310. A method of making a retroviral vector or retrovirus like particle, comprising:
a) providing a source cell that comprises a fusogen (e.g., re-targeted fusogen), wherein the source cell lacks a fusogen receptor or comprises a fusogen receptor that is present at reduced levels (e.g., reduced by at least 10%, 20%, 30%, 40%, 50%, 60%, 70%, 80%, or 90%) compared to an otherwise similar, unmodified source cell;
b) culturing the source cell under conditions that allow for production of the retroviral vector, and
c) separating, enriching, or purifying the retroviral vector from the source cell, thereby making the retroviral vector.
   311. The method of embodiment 310, wherein providing the source cell comprises knocking down or knocking out the fusogen receptor in the source cell or a precursor thereof.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of May 15, 2018. When one gene or protein references a plurality of sequence accession numbers, all of the sequence variants are encompassed. In addition, the materials, methods, and examples are illustrative only and not intended to be limiting.

### DETAILED DESCRIPTION

The present disclosure provides, at least in part, fusosome methods and compositions for in vivo delivery. In some embodiments, the fusosome comprises a combination of elements that promote specificity for target cells, e.g., one or more of a re-targeted fusogen, a positive target cell-specific regulatory element, and a non-target cell-specific regulatory element. In some embodiments, the fusosome comprises one or more modifications that decrease an immune response against the fusosome.

### Definitions

Terms used in the claims and specification are defined as set forth below unless otherwise specified.

As used herein, “detectably present”, when used in the context of an exogenous agent being detectably present, means that the exogenous agent itself is detectably present. For instance, if the exogenous agent is a protein, the exogenous protein agent can be detectably present regardless of whether a nucleic acid that encodes it is detectably present or not.

As used herein, “fusosome” refers to a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “fusogen receptor” refers to an entity (e.g., a protein) comprised by a target cell, wherein binding of a fusogen on a fusosome (e.g., retrovirus) to a fusogen receptor on a target cell promotes delivery of a nucleic acid (e.g., retroviral nucleic acid) (and optionally also an exogenous agent encoded therein) to the target cell.

As used herein, an “insulator element” refers to a nucleotide sequence that blocks enhancers or prevents heterochromatin spreading. An insulator element can be wild-type or mutant.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a virus, VLP, or fusosome, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

The term “pharmaceutically acceptable” as used herein, refers to excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “positive target cell-specific regulatory element” (or positive TCSRE) refers to a nucleic acid sequence that increases the level of an exogenous agent in a target cell compared to in a non-target cell, wherein the nucleic acid encoding the exogenous agent is operably linked to the positive TCSRE. In some embodiments, the positive TCSRE is a functional nucleic acid sequence, e.g., the positive TCSRE can comprise a promoter or enhancer.

In some embodiments, the positive TCSRE encodes a functional RNA sequence, e.g., the positive TCSRE can encode a splice site that promotes correct splicing of the RNA in the target cell. In some embodiments, the positive TCSRE encodes a functional protein sequence, or the positive TCSRE can encode a protein sequence that promotes correct post-translational modification of the protein. In some embodiments, the positive TCSRE decreases the level or activity of a downregulator or inhibitor of the exogenous agent.

As used herein, a “negative target cell-specific regulatory element” (or negative TCSRE) refers to a nucleic acid sequence that decreases the level of an exogenous agent in a non-target cell compared to in a target cell, wherein the nucleic acid encoding the exogenous agent is operably linked to the negative TCSRE. In some embodiments, the negative TCSRE is a functional nucleic acid sequence, e.g., a miRNA recognition site that causes degradation or inhibition of the retroviral nucleic acid in a non-target cell. In some embodiments, the nucleic acid sequence encodes a functional RNA sequence, e.g., the nucleic acid encodes an miRNA sequence present in an mRNA encoding an exogenous protein agent, such that the mRNA is degraded or inhibited in a non-target cell. In some embodiments, the negative TCSRE increases the level or activity of a downregulator or inhibitor of the exogenous agent.

As used herein, a “non-target cell-specific regulatory element” (or NTCSRE) refers to a nucleic acid sequence that decreases the level of an exogenous agent in a non-target cell compared to in a target cell, wherein the nucleic acid encoding the exogenous agent is operably linked to the NTCSRE. In some embodiments, the NTCSRE is a functional nucleic acid sequence, e.g., a miRNA recognition site that causes degradation or inhibition of the retroviral nucleic acid in a non-target cell. In some embodiments, the nucleic acid sequence encodes a functional RNA sequence, e.g., the nucleic acid encodes an miRNA sequence present in an mRNA encoding an exogenous protein agent, such that the mRNA is degraded or inhibited in a non-target cell. In some embodiments, the NTCSRE increases the level or activity of a downregulator or inhibitor of the exogenous agent. The terms “negative TCSRE” and “NTCSRE” are used interchangeably herein.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a fusosome (e.g., lentiviral vector) deliver an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen (alone or in combination with the positive TCSRE, NTCSRE, negative TCSRE, or any combination thereof) leads to preferential delivery of the exogenous agent to a target cell compared to a non-target cell.

As used herein a “non-target cell” refers to a cell of a type to which it is not desired that a fusosome (e.g., lentiviral vector) deliver an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen (alone or in combination with the positive TCSRE, NTCSRE, negative TCSRE or any combination thereof) leads to lower delivery of the exogenous agent to a non-target cell compared to a target cell.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

### Fusosomes, e.g., Cell-Derived Fusosomes

Fusosomes can take various forms. For example, in some embodiments, a fusosome described herein is derived from a source cell. A fusosome may comprise, e.g., an extracellular vesicle, a microvesicle, a nanovesicle, an exosome, an apoptotic body (from apoptotic cells), a microparticle (which may be derived from, e.g., platelets), an ectosome (derivable from, e.g., neutrophiles and monocytes in serum), a prostatosome (obtainable from prostate cancer cells), a cardiosome (derivable from cardiac cells), or any combination thereof. In some embodiments, a fusosome is released naturally from a source cell, and in some embodiments, the source cell is treated to enhance formation of fusosomes. In some embodiments, the fusosome is between about 10-10,000 nm in diameter, e.g., about 30-100 nm in diameter. In some embodiments, the fusosome comprises one or more synthetic lipids.

In some embodiments, the fusosome is or comprises a virus, e.g., a retrovirus, e.g., a lentivirus. In some embodiments, a fusosome comprising a lipid bilayer comprises a retroviral vector comprising an envelope. For instance, in some embodiments, the fusosome's bilayer of amphipathic lipids is or comprises the viral envelope. The viral envelope may comprise a fusogen, e.g., a fusogen that is endogenous to the virus or a pseudotyped fusogen. In some embodiments, the fusosome's lumen or cavity comprises a viral nucleic acid, e.g., a retroviral nucleic acid, e.g., a lentiviral nucleic acid. The viral nucleic acid may be a viral genome. In some embodiments, the fusosome further comprises one or more viral non-structural proteins, e.g., in its cavity or lumen.

Fusosomes may have various properties that facilitate delivery of a payload, such as, e.g., a desired transgene or exogenous agent, to a target cell. For instance, in some embodiments, the fusosome and the source cell together comprise nucleic acid(s) sufficient to make a particle that can fuse with a target cell. In embodiments, these nucleic acid(s) encode proteins having one or more of (e.g., all of) the following activities: gag polyprotein activity, polymerase activity, integrase activity, protease activity, and fusogen activity.

Fusosomes may also comprise various structures that facilitate delivery of a payload to a target cell. For instance, in some embodiments, the fusosome (e.g., virus, e.g., retrovirus, e.g., lentivirus) comprises one or more of (e.g., all of) the following proteins: gag polyprotein, polymerase (e.g., pol), integrase (e.g., a functional or non-functional variant), protease, and a fusogen. In some embodiments, the fusosome further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the fusosome nucleic acid (e.g., retroviral nucleic acid) comprises one or more of (e.g., all of) the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the fusosome nucleic acid (e.g., retroviral nucleic acid) further comprises one or more insulator element. In some embodiments the fusosome nucleic acid (e.g., retroviral nucleic acid) further comprises one or more miRNA recognition sites. In some embodiments, one or more of the miRNA recognition sites are situated downstream of the poly A tail sequence, e.g., between the poly A tail sequence and the WPRE.

In some embodiments, a fusosome provided herein is administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease. In some embodiments, the fusosome contains nucleic acid sequences encoding an exogenous agent for treating the disease or condition.

### Lentiviral Components and Helper Cells

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

A viral vector can comprise, e.g., a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

Codon optimization has a number of other advantages. By virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

Derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

Due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

The strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

As described above, the packaging components for a retroviral vector can include expression products of gag, pol and env genes. In addition, packaging can utilize a short sequence of 4 stem loops followed by a partial sequence from gag and env as a packaging signal.

Thus, inclusion of a deleted gag sequence in the retroviral vector genome (in addition to the full gag sequence on the packaging construct) can be used. In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

The retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

The gag and pol coding sequences are generally organized as the Gag-Pol Precursor in native lentivirus. The gag sequence codes for a 55-kD Gag precursor protein, also called p55.

The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

Native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In various examples, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

A packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### Vectors Engineered to Remove Splice Sites

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

Retroviral Production Methods

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides.

Packaging cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

A source cell line includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

Packaging Plasmids and Cell Lines

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

### Strategies for Packaging a Retroviral Nucleic Acid

Typically, modern retroviral vector systems consist of viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis-and trans-acting vector sequences completely, the virus is unable to maintain replication for more than one cycle of infection. Generation of live virus can be avoided by a number of strategies, e.g., by minimizing the overlap between the cis-and trans-acting sequences to avoid recombination.

A viral vector particle which comprises a sequence that is devoid of or lacking viral RNA may be the result of removing or eliminating the viral RNA from the sequence. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The vector particles could be used to deliver therapeutic RNA, in which case functional integrase and/or reverse transcriptase is not required. These vector particles could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognising a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1.

Repression of a Gene Encoding an Exogenous Agent in a Source Cell

(Over-)expressed protein in the source cell may have an indirect or direct effect on vector virion assembly and/or infectivity. Incorporation of the exogenous agent into vector virions may also impact downstream processing of vector particles.

In some embodiments, a tissue-specific promoter is used to limit expression of the exogenous agent in source cells. In some embodiments, a heterologous translation control system is used in eukaryotic cell cultures to repress the translation of the exogenous agent in source cells. More specifically, the retroviral nucleic acid may comprise a binding site operably linked to the gene encoding the exogenous agent, wherein the binding site is capable of interacting with an RNA-binding protein such that translation of the exogenous agent is repressed or prevented in the source cell.

In some embodiments, the RNA-binding protein is tryptophan RNA-binding attenuation protein (TRAP), for example bacterial tryptophan RNA-binding attenuation protein. The use of an RNA-binding protein (e.g. the bacterial trp operon regulator protein, tryptophan RNA-binding attenuation protein, TRAP), and RNA targets to which it binds, will repress or prevent transgene translation within a source cell. This system is referred to as the Transgene Repression In vector Production cell system or TRIP system.

In embodiments, the placement of a binding site for an RNA binding protein (e.g., a TRAP-binding sequence, tbs) upstream of the NOI translation initiation codon allows specific repression of translation of mRNA derived from the internal expression cassette, while having no detrimental effect on production or stability of vector RNA. The number of nucleotides between the tbs and translation initiation codon of the gene encoding the exogenous agent may be varied from 0 to 12 nucleotides. The tbs may be placed downstream of an internal ribosome entry site (IRES) to repress translation of the gene encoding the exogenous agent in a multicistronic mRNA.

Kill Switch Systems and Amplification

In some embodiments, a polynucleotide or cell harboring the gene encoding the exogenous agent utilizes a suicide gene, e.g., an inducible suicide gene, to reduce the risk of direct toxicity and/or uncontrolled proliferation. In specific aspects, the suicide gene is not immunogenic to the host cell harboring the exogenous agent. Examples of suicide genes include caspase-9, caspase-8, or cytosine deaminase. Caspase-9 can be activated using a specific chemical inducer of dimerization (CID).

In certain embodiments, vectors comprise gene segments that cause target cells, e.g., immune effector cells, e.g., T cells, to be susceptible to negative selection in vivo. For instance, the transduced cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, transduced cells, e.g., immune effector cells, such as T cells, comprise a polynucleotide further comprising a positive marker that enables the selection of cells of the negative selectable phenotype in vitro. The positive selectable marker may be a gene which, upon being introduced into the target cell, expresses a dominant phenotype permitting positive selection of cells carrying the gene. Genes of this type include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In some embodiments, the positive selectable marker and the negative selectable element are linked such that loss of the negative selectable element necessarily also is accompanied by loss of the positive selectable marker. For instance, the positive and negative selectable markers can be fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In addition, in embodiments, the polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT U591/08442 and PCT/US94/05601, describing the use of bifunctional selectable fusion genes derived from fusing a dominant positive selectable markers with negative selectable markers.

Suitable positive selectable markers can be derived from genes selected from the group consisting of hph, nco, and gpt, and suitable negative selectable markers can be derived from genes selected from the group consisting of cytosine deaminase, HSV-I TK, VZV TK, HPRT, APRT and gpt. Other suitable markers are bifunctional selectable fusion genes wherein the positive selectable marker is derived from hph or neo, and the negative selectable marker is derived from cytosine deaminase or a TK gene or selectable marker.

### Strategies for Regulating Lentiviral Integration

Retroviral and lentiviral nucleic acids are disclosed which are lacking or disabled in key proteins/sequences so as to prevent integration of the retroviral or lentiviral genome into the target cell genome. For instance, viral nucleic acids lacking each of the amino acids making up the highly conserved DDE motif (Engelman and Craigie (1992) J. Virol. 66:6361-6369; Johnson et al. (1986) Proc. Nat. Acad. Sci. USA 83:7648-7652; Khan et al. (1991) Nucleic Acids Res. 19:851-860) of retroviral integrase enables the production of integration defective retroviral nucleic acids.

For instance, in some embodiments, a retroviral nucleic acid herein comprises a lentiviral integrase comprising a mutation that causes said integrase to be unable to catalyze the integration of the viral genome into a cell genome. In some embodiments, said mutations are type I mutations which affect directly the integration, or type II mutations which trigger pleiotropic defects affecting virion morphogenesis and/or reverse transcription. Illustrative non-limitative examples of type I mutations are those mutations affecting any of the three residues that participate in the catalytic core domain of the integrase: DX₃₉₋₅₈DX₃₅E (D64, D116 and E152 residues of the integrase of the HIV-1). In a particular embodiment, the mutation that causes said integrase to be unable to catalyze the integration of the viral genome into a cell genome is the substitution of one or more amino acid residues of the DDE motif of the catalytic core domain of the integrase, preferably the substitution of the first aspartic residue of said DEE motif by an asparagine residue. In some embodiment the retroviral vector does not comprise an integrase protein.

In some embodiments the retrovirus integrates into active transcription units. In some embodiments the retrovirus does not integrate near transcriptional start sites, the 5′ end of genes, or DNAsel cleavage sites. In some embodiments the retrovirus integration does not active proto-oncogenes or inactive tumor suppressor genes. In some embodiments the retrovirus is not genotoxic. In some embodiments the lentivirus integrates into introns.

In some embodiments, the retroviral nucleic acid integrates into the genome of a target cell with a particular copy number. The average copy number may be determined from single cells, a population of cells, or individual cell colonies. Exemplary methods for determining copy number include polymerase chain reaction (PCR) and flow cytometry.

In some embodiments DNA encoding the exogenous agent is integrated into the genome. In some embodiments DNA encoding the exogenous agent is maintained episomally. In some embodiments the ratio of integrated to episomal DNA encoding the exogenous agent is at least 0.01, 0.1, 0.5, 1.0, 2, 5, 10, 100.

In some embodiments DNA encoding the exogenous agent is linear. In some embodiments DNA encoding the exogenous agent is circular. In some embodiments the ratio of linear to circular copies of DNA encoding the exogenous agent is at least 0.01, 0.1, 0.5, 1.0, 2, 5, 10, 100.

In embodiments the DNA encoding the exogenous agent is circular with 1 LTR. In some embodiments the DNA encoding the exogenous agent is circular with 2 LTRs. In some embodiments the ratio of circular, 1 LTR-comprising DNA encoding the exogenous agent to circular, 2 LTR-comprising DNA encoding the exogenous agent is at least 0.1, 0.5, 1.0, 2, 5, 10, 20, 50, 100.

### Maintenance of an Episomal Virus

In retroviruses deficient in integration, circular cDNA off-products of the retrotranscription (e.g., 1-LTR and 2-LTR) can accumulate in the cell nucleus without integrating into the host genome (see Yinez-Munoz R J et al., Nat. Med. 2006, 12: 348-353). Like other exogenous DNA those intermediates can then integrate in the cellular DNA at equal frequencies (e.g., 10³ to 10⁵/cell).

In some embodiments, episomal retroviral nucleic acid does not replicate. Episomal virus DNA can be modified to be maintained in replicating cells through the inclusion of eukaryotic origin of replication and a scaffold/matrix attachment region (S/MAR) for association with the nuclear matrix.

Thus, in some embodiments, a retroviral nucleic acid described herein comprises a eukaryotic origin of replication or a variant thereof. Examples of eukaryotic origins of replication of interest are the origin of replication of the β-globin gene as have been described by Aladjem et al (Science, 1995, 270: 815-819), a consensus sequence from autonomously replicating sequences associated with alpha-satellite sequences isolated previously from monkey CV-1 cells and human skin fibroblasts as has been described by Price et al Journal of Biological Chemistry, 2003, 278 (22): 19649-59, the origin of replication of the human c-myc promoter region has have been described by McWinney and Leffak (McWinney C. and Leffak M., Nucleic Acid Research 1990, 18(5): 1233-42). In embodiments, the variant substantially maintains the ability to initiate the replication in eukaryotes. The ability of a particular sequence of initiating replication can be determined by any suitable method, for example, the autonomous replication assay based on bromodeoxyuridine incorporation and density shift (Araujo F. D. et al., supra; Frappier L. et al., supra).

In some embodiments, the retroviral nucleic acid comprises a scaffold/matrix attachment region (S/MAR) or variant thereof, e.g., a non-consensus-like AT-rich DNA element several hundred base pairs in length, which organizes the nuclear DNA of the eukaryotic genome into chromatin domains, by periodic attachment to the protein scaffold or matrix of the cell nucleus. They are typically found in non-coding regions such as flanking regions, chromatin border regions, and introns. Examples of S/MAR regions are 1.8 kbp S/MAR of the human IFN-γ gene (hIFN-γ^{large}) as described by Bode et al (Bode J. et al., Science, 1992, 255: 195-7), the 0.7 Kbp minimal region of the S/MAR of the human IFN-γ gene (hIFN-γ^{short}) as has have been described by Ramezani (Ramezani A. et al., Blood 2003, 101: 4717-24), the 0.2 Kbp minimal region of the S/MAR of the human dehydrofolate reductase gene (hDHFR) as has been described by Mesner L. D. et al., Proc Natl Acad Sci USA, 2003, 100: 3281-86). In embodiments, the functionally equivalent variant of the S/MAR is a sequence selected based on the set six rules that together or alone have been suggested to contribute to S/MAR function (Kramer et al (1996) Genomics 33, 305; Singh et al (1997) Nucl. Acids Res 25, 1419). These rules have been merged into the MAR-Wiz computer program freely available at genomecluster.secs.oakland.edu/MAR-Wiz. In embodiments, the variant substantially maintains the same functions of the S/MAR from which it derives, in particular, the ability to specifically bind to the nuclear the matrix. The skilled person can determine if a particular variant is able to specifically bind to the nuclear matrix, for example by the in vitro or in vivo MAR assays described by Mesner et al. (Mesner L. D. et al, supra). In some embodiments, a specific sequence is a variant of a S/MAR if the particular variant shows propensity for DNA strand separation. This property can be determined using a specific program based on methods from equilibrium statistical mechanics. The stress-induced duplex destabilization (SIDD) analysis technique “[ . . . ] calculates the extent to which the imposed level of superhelical stress decreases the free energy needed to open the duplex at each position along a DNA sequence. The results are displayed as an SIDD profile, in which sites of strong destabilization appear as deep minima [ . . . ]” as defined in Bode et al (2005) J. Mol. Biol. 358,597. The SIDD algorithm and the mathematical basis (Bi and Benham (2004) Bioinformatics 20, 1477) and the analysis of the SIDD profile can be performed using the freely available internet resource at WebSIDD (www.genomecenter.ucdavis.edu/benham). Accordingly, in some embodiment, the polynucleotide is considered a variant of the S/MAR sequence if it shows a similar SIDD profile as the S/MAR.

### Fusogens and Pseudotyping

Fusogens, which include, viral envelope proteins (env), generally determine the range of host cells which can be infected and transformed by fusosomes. In the case of lentiviruses, such as HIV-1, HIV-2, SIV, FIV and EIV, the native env proteins include gp41 and gp120. In some embodiments, the viral env proteins expressed by source cells described herein are encoded on a separate vector from the viral gag and pol genes, as has been previously described.

Illustrative examples of retroviral-derived env genes which can be employed include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In some embodiments, envelope proteins for display on a fusosome include, but are not limited to any of the following sources: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In some embodiments, a source cell described herein produces a fusosome, e.g., recombinant retrovirus, e.g., lentivirus, pseudotyped with the VSV-G glycoprotein.

A fusosome or pseudotyped virus generally has a modification to one or more of its envelope proteins, e.g., an envelope protein is substituted with an envelope protein from another virus. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In some embodiments, lentiviral envelope proteins are pseudotyped with VSV-G. In one embodiment, source cells produce recombinant retrovirus, e.g., lentivirus, pseudotyped with the VSV-G envelope glycoprotein.

Furthermore, a fusogen or viral envelope protein can be modified or engineered to contain polypeptide sequences that allow the transduction vector to target and infect host cells outside its normal range or more specifically limit transduction to a cell or tissue type. For example, the fusogen or envelope protein can be joined in-frame with targeting sequences, such as receptor ligands, antibodies (using an antigen-binding portion of an antibody or a recombinant antibody-type molecule, such as a single chain antibody), and polypeptide moieties or modifications thereof (e.g., where a glycosylation site is present in the targeting sequence) that, when displayed on the transduction vector coat, facilitate directed delivery of the virion particle to a target cell of interest. Furthermore, envelope proteins can further comprise sequences that modulate cell function. Modulating cell function with a transducing vector may increase or decrease transduction efficiency for certain cell types in a mixed population of cells. For example, stem cells could be transduced more specifically with envelope sequences containing ligands or binding partners that bind specifically to stem cells, rather than other cell types that are found in the blood or bone marrow. Non-limiting examples are stem cell factor (SCF) and Flt-3 ligand. Other examples, include, e.g., antibodies (e.g., single-chain antibodies that are specific for a cell-type), and essentially any antigen (including receptors) that binds tissues as lung, liver, pancreas, heart, endothelial, smooth, breast, prostate, epithelial, vascular cancer, etc.

### Exemplary Fusogens

In some embodiments, the retroviral vector or VLP includes one or more fusogens, e.g., to facilitate the fusion of the retroviral vector or VLP to a membrane, e.g., a cell membrane.

In some embodiments, the retroviral vector or VLP comprises one or more fusogens on its envelope to target a specific cell or tissue type. Fusogens include without limitation protein based, lipid based, and chemical based fusogens. In some embodiments, the retroviral vector or VLP includes a first fusogen which is a protein fusogen and a second fusogen which is a lipid fusogen or chemical fusogen. The fusogen may bind a fusogen binding partner on a target cells' surface. In some embodiments, the retroviral vector or VLP comprising the fusogen will integrate the membrane into a lipid bilayer of a target cell.

In some embodiments, one or more of the fusogens described herein may be included in the retroviral vector or VLP.

### Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen results in mixing between lipids in the retroviral vector or VLP and lipids in the target cell. In some embodiments, the fusogen results in formation of one or more pores between the interior of the retroviral vector or VLP and the cytosol of the target cell.

### Mammalian Proteins

In some embodiments, the fusogen may include a mammalian protein, see Table 1. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties (see Table 3), a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

Table 1: Non-limiting examples of human and non-human fusogens.

TABLE 1
Non-limiting examples of human and non-human fusogens.
Human and Non-Human Fusogen Classes

| Fusogen Class | Uniprot Protein Family ID | # of sequences |
|---|---|---|
| EFF-AFF | PF14884 | 191 |
| SNARE | PF05739 | 5977 |
| DC-STAMP | PF07782 | 633 |
| ENV | PF00429 | 312 |

TABLE 2
Genes that encode proteins with fusogen properties.
Human genes with the gene ontology annotation of:
Syncytium formation by plasma membrane fusion proteins

| ID | Symbol |
|---|---|
| A0A024R0I0 | DYRK1B |
| A0A024R1N1 | MYH9 |
| A0A024R2D8 | CAV3 |
| A0A096LNV2 | FER1L5 |
| A0A096LPA8 | FER1L5 |
| A0A096LPB1 | FER1L5 |
| A0AVI2 | FER1L5 |
| A6NI61 | TMEM8C (myomaker) |
| B3KSL7 | — |
| B7ZLI3 | FER1L5 |
| H0YD14 | MYOF |
| O43184 | ADAM12 |
| O60242 | ADGRB3 |
| O60500 | NPHS1 |
| O95180 | CACNA1H |
| O95259 | KCNH1 |
| P04628 | WNT1 |
| P15172 | MYOD1 |
| P17655 | CAPN2 |
| P29475 | NOS1 |
| P35579 | MYH9 |
| P56539 | CAV3 |
| Q2NNQ7 | FER1L5 |
| Q4KMG0 | CDON |
| Q53GL0 | PLEKHO1 |
| Q5TCZ1 | SH3PXD2A |
| Q6YHK3 | CD109 |
| Q86V25 | VASH2 |
| Q99697 | PITX2 |
| Q9C0D5 | TANC1 |
| Q9H295 | DCSTAMP |
| Q9NZM1 | MYOF |
| Q9Y463 | DYRK1B |

TABLE 3
Human Fusogen Candidates

| Fusogen Class | Gene ID |
|---|---|
| SNARE | O15400 |
| | Q16623 |
| | K7EQB1 |
| | Q86Y82 |
| | E9PN33 |
| | Q96NA8 |
| | H3BT82 |
| | Q9UNK0 |
| | P32856 |
| | Q13190 |
| | O14662 |
| | P61266 |
| | O43752 |
| | O60499 |
| | Q13277 |
| | B7ZBM8 |
| | A0AVG3 |
| | Q12846 |
| DC-STAMP | Q9H295 |
| | Q5T1A1 |
| | Q5T197 |
| | E9PJX3 |
| | Q9BR26 |
| ENV | Q9UQF0 |
| | Q9N2K0 |
| | P60507 |
| | P60608 |
| | B6SEH9 |
| | P60508 |
| | B6SEH8 |
| | P61550 |
| | P60509 |
| | Q9N2J8 |
| Muscle Fusion (Myomaker) | H0Y5B2 |
| | H7C1S0 |
| | Q9HCN3 |
| | A6NDV4 |
| | K4DI83 |
| Muscle Fusion (Myomixer) | NP_001302423.1 |
| | ACT64390.1 |
| | XP_018884517.1 |
| | XP_017826615.1 |
| | XP_020012665.1 |
| | XP_017402927.1 |
| | XP_019498363.1 |
| | ELW65617.1 |
| | ERE90100.1 |
| | XP_017813001.1 |
| | XP_017733785.1 |
| | XP_017531750.1 |
| | XP_020142594.1 |
| | XP_019649987.1 |
| | XP_019805280.1 |
| | NP_001170939.1 |
| | NP_001170941.1 |
| | XP_019590171.1 |
| | XP_019062106.1 |
| | EPQ04443.1 |
| | EPY76709.1 |
| | XP_017652630.1 |
| | XP_017459263.1 |
| | OBS58441.1 |
| | XP_017459262.1 |
| | XP_017894180.1 |
| | XP_020746447.1 |
| | ELK00259.1 |
| | XP_019312826.1 |
| | XP_017200354.1 |
| | BAH40091.1 |
| HA | P03452 |
| | Q9Q0U6 |
| | P03460 |
| GAP JUNCTION | P36382 |
| | P17302 |
| | P36383 |
| | P08034 |
| | P35212 |
| Other | FGFRL1 |
| | GAPDH |

In some embodiments, the retroviral vector or VLP comprises a curvature-generating protein, e.g., Epsin1, dynamin, or a protein comprising a BAR domain. See, e.g., Kozlovet al, CurrOp StrucBio 2015, Zimmerberget al. Nat Rev 2006, Richard et al, Biochem J 2011.

### Non-Mammalian Proteins

### Viral Proteins

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gpl60 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

In some embodiments the fusogen is a paramyxovirus fusogen. In some embodiments the fusogen is a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein.

In some embodiments, the fusogen is a poxviridae fusogen.

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, a fusogen described herein comprises an amino acid sequence of Table 4, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a fusogen described herein comprises an amino acid sequence having at least 80% identity to any amino acid sequence of Table 4. In some embodiments, a nucleic acid sequence described herein encodes an amino acid sequence of Table 4, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

In some embodiments, a fusogen described herein comprises an amino acid sequence set forth in any one of SEQ ID NOS: 1-56, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a fusogen described herein comprises an amino acid sequence having at least 80% identity to an amino acid sequence set forth in any one of SEQ ID NOS: 1-56. In some embodiments, a nucleic acid sequence described herein encodes an amino acid sequence set forth in any one of SEQ ID NOS: 1-56, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

TABLE 4
Paramyxovirus F sequence clusters. Column 1, Genbank ID includes the Genbank ID
of the whole genome sequence of the virus that is the centroid sequence of the
cluster. Column 2, Nucleotides of CDSprovides the nucleotides corresponding
to the CDSof the gene in the whole genome. Column 3, Full Gene Name, provides
the full name of the gene including Genbank ID, virus species, strain, and protein
name. Column 4, Sequence, provides the amino acid sequence of the gene. Column 5,
#Sequences/Cluster, provides the number of sequences
that cluster with this centroid sequence.

| | Nucleo- | | | | |
|---|---|---|---|---|---|
| Genbank | tides | | | #Sequences/ | SEQ |
| ID | of CDS | Full Gene Name | Sequence | Cluster | ID NO |
| KP317927 | 5630-7399 | gb: KP317927: | MIPQARTELNLGQITMELLIHRSSAIFLTLAINALYL | 993 | 1 |
| | | 5630-73pp\| | TSSQNITEEFYQSTCSAVSRGYLSALRTGWYTSVITI | | |
| | | Organism: | ELSNIKETKCNGTDTKVKLIKQELDKYKNAVTELQLL | | |
| | | Human respriatory | MQNTPAANNRARREAPQYMNYTINTTGSLNVSISKKR | | |
| | | syncytial virus\| | KRRFLGFLLGVGSAIASGIAVSKVLHLEGEVNKIKNA | | |
| | | Strain Name: | LLSTNKAVVSLSNGVSVLTSKVLDLKNYINNQLLPIV | | |
| | | Kilifi_9465_7_ | NQQSCRISNIETVIEFQQKNSRLLEITREFSVNAGVT | | |
| | | RSVB_2011\| | TPLSTYMLTNSELLSLINDMPITNDQKKLMSSNVQIV | | |
| | | Protein Name: | RQQSYSIMSIIKEEVLAYVVQLPIYGVIDTPCWKLHT | | |
| | | fusion glycoprotein\| | SPLCTTNIKEGSNICLTRTDRGWYCDNAGSVSFFPQA | | |
| | | Gene Symbol: F | DTCKVQSNRVFCDTMNSLTLPSEVSLCNTDIFNSKYD | | |
| | | | CKIMTSKTDISSSVITSLGAIVSCYGKTKCTASNKNR | | |
| | | | GIIKTFSNGCDYVSNKGVDTVSVGNTLYYVNKLEGKN | | |
| | | | LYVKGEPIINYYDPLVFPSDEFDASISQVNEKINQSL | | |
| | | | AFIRRSDELLHNVNTGKSTTNIMITAIIIVIIVVLLS | | |
| | | | LIAIGLLLYCKAKNTPVTLSKDQLSGINNIAFSK | | |
| AB524405 | 4556-6217 | gb: AB524405: | MDPKPSTSYLHAFPLIFVAISLVFMAGRASALDGRPL | 418 | 2 |
| | | 4556-6217\| | AAAGIVVTGDKAVNIYTSSQTGTIIIKLLPNMPKDKE | | |
| | | Organism: | QCAKSPLDAYNRTLTTLLAPLGDSIRRIQESVTTSGG | | |
| | | Newcastle disease | ERQERLVGAIIGGVALGVATAAQITAASALIQANQNA | | |
| | | virus\|Strain | ANILKLKESIAATNEAVHEVTSGLSQLAVAVGKMQQF | | |
| | | Name:Goose/ | VNDQFNKTAQEIDCIKITQQVGVELNLYLTELTTVFG | | |
| | | Alaska/415/91\| | PQITSPALTQLTIQALYNLAGGNMDYMLTKLGVGNNQ | | |
| | | Protein Name: | LSSLISSGLISGNPILYDSQTQLLGIQVTLPSVGNLN | | |
| | | fusion protein\| | NMRATYLETLSVSTNKGFASALVPKVVTQVGSVIEEL | | |
| | | Gene Symbol: F | DTSYCIETDLDLYCTRIVTFPMSPGIFSCLGGNTSAC | | |
| | | | MYSKTEGALTTPYMTLKGSVIANCKMTTCRCADPPGI | | |
| | | | ISQNYGEAVSLIDKKVCNILTLDGITLRLSGEFDATY | | |
| | | | QKNISIQDSQVVITGNLDISTELGNVNNSISNALDKL | | |
| | | | EESNSKLDKVNVRLTSTSALITYIVLTTIALICGIVS | | |
| | | | LVLACYIMYKQKAQQKTLLWLGNNTLDQMRATTKM | | |
| AF266286 | 4875-7247 | gb: AF266286: | MSIMGLKVNVSAIFMAVLLTLQTPTGQIHWGNLSKIG | 128 | 3 |
| | | 4875-7247\| | VVGIGSASYKVMTRSSHQSLVIKLMPNITLLNNCTRV | | |
| | | Organism: | EIAEYRRLLRTVLEPIRDALNAMTQNIRPVQSVASSR | | |
| | | Measles virus | RHKRFAGVVLAGAALGVATAAQITAGIALHQSMLNSQ | | |
| | | strain AIK-C\| | AIDNLRASLETTNQAIEAIRQAGQEMILAVQGVQDYI | | |
| | | Strain Name: | NNELIPSMNQLSCDLIGQKLGLKLLRYYTEILSLFGP | | |
| | | Measles virus | SLRDPISAEISIQALSYALGGDINKVLEKLGYSGGDL | | |
| | | strain Edmonston | LGILESRGIKARITHVDTESYFIVLSIAYPTLSEIKG | | |
| | | (AIK-C vaccine)\| | VIVHRLEGVSYNIGSQEWYTTVPKYVATQGYLISNFD | | |
| | | Protein Name: | ESSCTFMPEGTVCSQNALYPMSPLLQECLRGYTKSCA | | |
| | | fusion protein\| | RTLVSGSFGNRFILSQGNLIANCASILCKCYTTGTII | | |
| | | Gene Symbol: F | NQDPDKILTYIAADNCPVVEVNGVTIQVGSRRYPDAV | | |
| | | | YLHRIDLGPPILLERLDVGTNLGNAIAKLEDAKELLE | | |
| | | | SSDQILRSMKGLSSTCIVYILIAVCLGGLIGIPALIC | | |
| | | | CCRGRCNKKGEQVGMSRPGLKPDLTGTSKSYVRSL | | |
| AB503857 | 3068-4687 | gb: AB503857: | MSWKVVIIFSLLITPQHGLKESYLEESCSTITEGYLS | 125 | 4 |
| | | 3068-4687\| | VLRTGWYTNVFTLEVGDVENLTCSDGPSLIKTELDLT | | |
| | | Organism: Human | KSALRELKTVSADQLAREEQIEKPRQSRFVLGAIALG | | |
| | | metapneumovirus\| | VATAAAVTAGVAIAKTIRLESEVTAIKNALKTTNEAV | | |
| | | Strain Name: Jpn03- | STLGNGVRVLATAVRELKDFVSKNLTRAINKNKCDID | | |
| | | 1\|Protein Name: | DLKMAVSFSQFNRRFLNVVRQFSDNAGITPAISLDLM | | |
| | | fusion glycoprotein | TDAELARAVSNMPTSAGQIKLMLENRAMVRRKGFGIL | | |
| | | precursor\|Gene | IGVYGSSVIYMVQLPIFGVIDTPCWIVKAAPSCSEKK | | |
| | | Symbol: F | GNYACLLREDQGWYCQNAGSTVYYPNEKDCETRGDHV | | |
| | | | FCDTAAGINVAEQSKECNINISTTNYPCKVSTGRHPI | | |
| | | | SMVALSPLGALVACYKGVSCSIGSNRVGIIKQLNKGC | | |
| | | | SYITNQDADTVTIDNTVYQLSKVEGEQHVIKGRPVSS | | |
| | | | SFDPIKFPEDQFNVALDQVFENIENSQALVDQSNRIL | | |
| | | | SSAEKGNTGFIIVIILIAVLGSSMILVSIFIIIKKTK | | |
| | | | KPTGAPPELSGVTNNGFIPHS | | |
| EU277658 | 5078-6700 | gb: EU277658: | MIIIVITMILSLTPSSLCQIDITKLQSVGVLVNSPKG | 93 | 5 |
| | | 5078-6700\| | IKISQNFETRYLILSLIPKIEDSHSCGNQQIDQYKKL | | |
| | | Organism: Bovine | LDRLIIPLYDGLKLQKDVIVVNHESHNNTNLRTKRFF | | |
| | | parainfluenza virus | GEIIGTIAIGIATSAQITAAVALVEAKQARSDIDKLK | | |
| | | 3\|Strain Name: | EAIKDTNKAVQSIQSSVGNLIVAVKSVQDYVNNEIVP | | |
| | | Q5592\|Protein Name: | SITRLGCEAAGLQLGIALTQHYSELTNIFGDNIGTLG | | |
| | | fusion protein\| | EKGVKLQGIASLYRTNITEVFTTSTVDQYDIYDLLFT | | |
| | | Gene Symbol: F | ESIKMRVIDVDLSDYSITLQVRLPLLTKVSNTQIYKV | | |
| | | | DSISYNIQGKEWYIPLPHHIMTKGAFLGGADIKECIE | | |
| | | | SFSNYICPSDPGFILNHEMENCLSGNITQCPKTIVTS | | |
| | | | DIVPRYAFVDGGVIANCIPTTCTCNGIDNRINQSPDQ | | |
| | | | GIKIITYKECQIVGINGMLFKTNQEGTLAKYTFDNIK | | |
| | | | LNNSVALNPIDISLELNKAKSDLEESKRWIEKSNQKL | | |
| | | | DSIGSWHQSSVTIIIIIVMIVVLLIINAIIIMIMIRY | | |
| | | | LRDRNRHLNNKDSEPYVLTNRQ | | |
| AB040874 | 4546-6162 | gb: AB040874: | MKVFLVTCLGFAVFSSSVCVNINILQQIGYIKQQVRQ | 89 | 6 |
| | | 4546-6162\| | LSYYSQSSSSYIVVKLLPNIQPTDNSCEFKSVTQYNK | | |
| | | Orgamism: Mumps | TLSNLLLPIAENINNIASPSSGSRRHKRFAGIAIGIA | | |
| | | virus\|Strain Name: | ALGVATAAQVTAAVSLVQAQTNARAIAAMKNSIQATN | | |
| | | Miyaharal Protein | RAVFEVKEGTQRLAIAVQAIQDHINTIMNTQLNNMSC | | |
| | | Name: fusion | QILDNQLATSLGLYLTELTTVFQPQLINPALSPISIQ | | |
| | | protein\|Gene | ALRSLLGSMTPAVVQATLSTSISAAEILSAGLMEGQI | | |
| | | Symbol: F | VSVLLDEMQMIVKINIPTIVTQSNALVIDFYSISSFI | | |
| | | | NNQESIIQLPDRILEIGNEQWSYPAKNCKLTRHHIFC | | |
| | | | QYNEAERLSLESKLCLAGNISACVFSPIAGSYMRRFV | | |
| | | | ALDGTIVANCRSLTCLCKSPSYPIYQPDHHAVTTIDL | | |
| | | | TACQTLSLDGLDFSIVSLSNITYAENLTISLSQTINT | | |
| | | | QPIDISTELSKVNASLQNAVKYIKESNHQLQSVNVNS | | |
| | | | KIGAIIVAALVLSILSIIISLLFCCWAYVATKEIRRI | | |
| | | | NFKTNHINTISSSVDDLIRY | | |
| AB475097 | 4908-6923 | gb: AB475097: | MNPHEQTIPMHEKIPKRSKTQTHTQQDLPQQHSTKSA | 46 | 7 |
| | | 4908-6923\| | ESKTSRARHSITSAQRSTHYDPRTADWPDYYIMKRTR | | |
| | | Organism: Canine | SCKQASYRSDNIPAHGDHDGIIHHTPESVSQGAKSRL | | |
| | | distemper virus\| | KMGQSNAVKSGSQCTWLVLWCIGVASLFLCSKAQIHW | | |
| | | Strain Name: M25CR\| | NNLSTIGIIGTDSVHYKIMTRPSHQYLVIKLMPNVSL | | |
| | | Protein Name: fusion | IDNCTKAELDEYEKLLSSILEPINQALTLMTKNVKPL | | |
| | | protein\|Gene | QSVGSGRRQRRFAGVVLAGAALGVATAAQITAGIALH | | |
| | | Symbol: F | QSNLNAQAIQSLRTSLEQSNKAIEEIREATQETVIAV | | |
| | | | QGVQDYVNNELVPAMQHMSCELVGQRLGLKLLRYYTE | | |
| | | | LLSIFGPSLRDPISAEISIQALSYALGGEIHKILEKL | | |
| | | | GYSGNDMIAILESRGIKTKITHVDLPGKFIILSVSYP | | |
| | | | TLSEVKGVIVHRLEAVSYNIGSQEWYTTVPRYVATNG | | |
| | | | YLISNFDESSCVFVSESAICSQNSLYPMSPLLQQCIR | | |
| | | | GDTSSCARTLVSGTMGNKFILSKGNIVANCASILCKC | | |
| | | | YSTSTIINQSPDKLLTFIASDTCPLVEIDGVTIQVGS | | |
| | | | RQYPDMVYESKVALGPAISLERLDVGTNLGNALKKLD | | |
| | | | DAKVLIDSSNQILETVRRSSFNFGSLLSVPILSCTAL | | |
| | | | ALLLLICCCKRRYQQTHKQNTKVDPTFKPDLTGTSRS | | |
| | | | YVRSL | | |
| AJ849636 | 5526-7166 | gb: AJ849636: | MTRVAILTFLFLFPNAVACQIHWGNLSKIGIVGTGSA | 34 | 8 |
| | | 5526-7166\| | SYKVMTRPSHQTLVIKLMPNITAIDNCTKSEIAEYKR | | |
| | | Organism: Peste-des- | LLITVLKPVEDALSVITKNVRPIQTLTPGRRTRRFAG | | |
| | | petits-ruminants | AVLAGVALGVATAAQITAGVALHQSLMNSQAIESLKT | | |
| | | virus\|Strain Name: | SLEKSNQAIEEIRLANKETILAVQGVQDYINNELVPS | | |
| | | Turkey 2000\|Protein | VHRMSCELVGHKLGLKLLRYYTEILSIFGPSLRDPIA | | |
| | | Name: fusion | AEISIQALSYALGGDINRILDKLGYSGGDFLAILESK | | |
| | | protein\| Gene | GIKARVTYVDTRDYFIILSIAYPTLSEIKGVIVHKIE | | |
| | | Symbol: F | AITYNIGAQEWYTTIPKYVATQGYLISNFDETSCVFT | | |
| | | | PDGTVCSQNALYPMSPLLQECFQGSTKSCARTLVSGT | | |
| | | | ISNRFILSKGNLIANCASVLCKCYTTETVISQDPDKL | | |
| | | | LTVVASDKCPVVEVDGVTIQVGSREYPDSVYLHKIDL | | |
| | | | GPAISLEKLDVGTNLGNAVTRLENAKELLDASDQILK | | |
| | | | TVKGVPFGGNMYIALAACIGVSLGLVTLICCCKGRCK | | |
| | | | NKEVPISKINPGLKPDLTGTSKSYVRSL | | |
| AF017149 | 6618-8258 | gb: AF017149\| | MATQEVRLKCLLCGIIVLVLSLEGLGILHYEKLSKIG | 29 | 9 |
| | | Organism: Hendra | LVKGITRKYKIKSNPLTKDIVIKMIPNVSNVSKCTGT | | |
| | | virus\|Strain Name: | VMENYKSRLTGILSPIKGAIELYNNNTHDLVGDVKLA | | |
| | | UNKNOWN-AF017149\| | GVVMAGIAIGIATAAQITAGVALYEAMKNADNINKLK | | |
| | | Protein Name: | SSIESTNEAVVKLQETAEKTVYVLTALQDYINTNLVP | | |
| | | fusion\|Gene Symbol: | TIDQISCKQTELALDLALSKYLSDLLFVFGPNLQDPV | | |
| | | F | SNSMTIQAISQAFGGNYETLLRTLGYATEDFDDLLES | | |
| | | | DSIAGQIVYVDLSSYYIIVRVYFPILTEIQQAYVQEL | | |
| | | | LPVSFNNDNSEWISIVPNFVLIRNTLISNIEVKYCLI | | |
| | | | TKKSVICNQDYATPMTASVRECLTGSTDKCPRELVVS | | |
| | | | SHVPRFALSGGVLFANCISVTCQCQTTGRAISQSGEQ | | |
| | | | TLLMIDNTTCTTVVLGNIIISLGKYLGSINYNSESIA | | |
| | | | VGPPVYTDKVDISSQISSMNQSLQQSKDYIKEAQKIL | | |
| | | | DTVNPSLISMLSMIILYVLSIAALCIGLITFISFVIV | | |
| | | | EKKRGNYSRLDDRQVRPVSNGDLYYIGT | | |
| AB005795 | 4866-6563 | gb: AB005795: | MATYIQRVQCISALLSVVLTTLVSCQIPRDRLSNIGV | 23 | 10 |
| | | 4866-6563\| | IVDEGKSLKIAGSHESRYIVLSLVPGIDLENGCGTAQ | | |
| | | Organism: Sendai | VIQYKSLLNRLLIPLRDALDLQEALITVTNDTMTGAD | | |
| | | virus\|Strain Name: | VPQSRFFGAVIGTIALGVATSAQITAGIALAEAREAK | | |
| | | Ohita\|Protein Name: | RDIALIKESMTKTHKSIELLQNAVGEQILALKTLQDF | | |
| | | fusion protein\| | VNDEIKPAISELGCETAALRLGIKLTQHYSELLTAFG | | |
| | | Gene Symbol: F | SNFGTIGEKSLTLQALSSLYSANITEIMTTIRTGQSN | | |
| | | | IYDVIYTEQIKGTVIDVDLERYMVTLSVKIPILSEVP | | |
| | | | GVLIHKASSISYNIDGEEWYVTVPSHILSRASFLGGA | | |
| | | | NIADCVESRLTYICPRDPAQLIPDSQQKCILGDTTRC | | |
| | | | PVTKVVDNIIPKFAFVNGGVVANCIASTCTCGTGRRP | | |
| | | | ISQDRSKGVVFLTHDNCGLIGVNGIELYANRKGHDAT | | |
| | | | WGVQNLTVGPAIAIRPVDISLNLAAATDFLQDSRAEL | | |
| | | | EKARKILSEVGRWYNSGATLITIIVVMIVVLVVIIVI | | |
| | | | VIVLYRLRRSMLMSNPAGRISRDTYTLEPKIRHMYTN | | |
| | | | GGFDAMTEKR | | |
| AF457102 | 5088-6755 | gb: AF457102\| | MQKSEILFLVYSSLLLSSSLCQIPVEKLSNVGVIINE | 21 | 11 |
| | | Organism: Human | GKLLKIAGSYESRYIVLSLVPSIDLQDGCGTTQIIQY | | |
| | | parainfluenza virus | KNLLNRLLIPLKDALDLQESLITITNDTTVTNDNPQT | | |
| | | 1 strain Washington/ | RFFGAVIGTIALGVATAAQITAGIALAEAREARKDIA | | |
| | | 1964\|Strain Name: | LIKDSIVKTHNSVELIQRGIGEQIIALKTLQDFVNDE | | |
| | | Washington 1964\| | IRPAIGELRCETTALKLGIKLTQHYSELATAFSSNLG | | |
| | | Protein Name: F | TIGEKSLTLQALSSLYSANITEILSTTKKDKSDIYDI | | |
| | | glycoprotein\|Gene | IYTEQVKGTVIDVDLEKYMVTLLVKIPILSEIPGVLI | | |
| | | Symbol: F | YRASSISYNIEGEEWHVAIPNYIINKASSLGGADVTN | | |
| | | | CIESKLAYICPRDPTQLIPDNQQKCILGDVSKCPVTK | | |
| | | | VINNLVPKFAFINGGVVANCIASTCTCGTNRIPVNQD | | |
| | | | RSRGVTFLTYTNCGLIGINGIELYANKRGRDTTWGNQ | | |
| | | | IIKVGPAVSIRPVDISLNLASATNFLEESKTELMKAR | | |
| | | | AIISAVGGWHNTESTQIIMIIIVCILIIIICGILYYL | | |
| | | | YRVRRLLVMINSTHNSPVNAYTLESRMRNPYMGNNSN | | |
| AB910309 | 4951-6582 | gb: AB910309: | MGKIRVIIISSLLLSNITTAQVGWDNLTSIGVISTKQ | 12 | 12 |
| | | 4951-6582\| | YDYKITTLNTNQLMVIKMVPNISSIINCTKPELMKYR | | |
| | | Organism: Feline | ELVLGVIRPINESLELMNSYINMRAGSERFIGAVIAG | | |
| | | morbillivirus\|Strain | VALGVATAAQITSGIALHNSIMNKRQIQELRKALSTT | | |
| | | Name: SS1\|Protein | NKAIDEIRIAGERTLIAVQGVQDYINNIIIPMQDKLQ | | |
| | | Name: fusion | CDILSSQLAIALLRYYTNILTVFGPSIRDPVTSIISI | | |
| | | protein\|Gene Symbol: | QALSQAFNGNLQALLDGLGYTGRDLRDLLESRSITGQ | | |
| | | F | IIHADMTDLFLVLRINYPSITEMQGVTIYELNSITYH | | |
| | | | IGPEEWYTIMPNFIAVQGFLTSNFDERKCSITKSSIL | | |
| | | | CQQNSIYPMSTEMQRCIKGEIRFCPRSKAVGTLVNRF | | |
| | | | ILTKGNLMANCLGVICRCYSSGQIITQDPSKLITIIS | | |
| | | | QEECKEVGVDGIRIMVGPRKLPDVIFNARLEVGVPIS | | |
| | | | LSKLDVGTDLAIASAKLNNSKALLEQSDKILDSMSKL | | |
| | | | DSINSRITGLILAIMAIFIITVTIIWIIYKRCRNKDN | | |
| | | | KFSTSIEPLYIPPSYNSPHSVVKSI | | |
| KT071755 | 4310-6070 | gb: KT071755: | MIAALFISLFATCGALDNSVLAPVGIASAQEWQLAAY | 12 | 13 |
| | | 4310-6070\| | TNTLSGTIAVRFVPVLPGNLSTCAQATLAEYNKTVTN | | |
| | | Organism: Avian | ILGPLKENLETLLSEPTKTAARFVGAIIGTVALGVAT | | |
| | | paramyxovirus 2\| | SAQITAAVALNQAQENARNIWRLKESIRKTNEAVLEL | | |
| | | Strain Name: APMV- | KDGLASTAIALDKVQKFINEDIIPQIKEIDCQVVANK | | |
| | | 2/Procarduelis | LGVYLSLYLTELTTIFGAQITNPALTPLSYQALYNLC | | |
| | | nipalensis/China/ | GGDMGKLTELIGVKAKDINSLYEANLITGQVIGYDSE | | |
| | | Suiling/53/2013\| | SQIILIQVSYPSVSEVTGVRATELVTVSVTTPKGEGR | | |
| | | Protein Name: | AIAPKYVAQSRVVTEELDTSTCRFSKTTLYCRSIITR | | |
| | | fusion protein\|Gene | PLPPLIANCLNGLYQDCQYTTEIGALSSRFITVNGGI | | |
| | | Symbol: F | IANCRATICKCVNPPKIIVQSDASSLTVIDSAICKDV | | |
| | | | VLDNVQLRLEGKLSAQYFTNITIDLSQITTSGSLDIS | | |
| | | | SEIGSINNTVNKVEELIAESNAWLQAVNPHLVNNTSI | | |
| | | | IVLCVLAAIFVVWLVALTGCLAYYIKKSSATRMVGIG | | |
| | | | SSPAGNPYVAQSATKM | | |
| AY029299 | 4598-6265 | gb: AY029299\| | MGARLGPLAMAPGRYVIIFNLILLHRVVSLDNSRLLQ | 11 | 14 |
| | | Orgamism: Avian | QGIMSATEREIKVYTNSITGSIAVRLIPNLPQEVLKC | | |
| | | paramyxovirus 6\| | SAGQIKSYNDTLNRIFTPIKANLERLLATPSMLEDNQ | | |
| | | Strain Name: APMV- | NPAPEPRLIGAIIGTAALGLATAAQVTAALALNQAQD | | |
| | | 6/duck/Taiwan/Y1/ | NAKAILNLKESITKTNEAVLELKDATGQIAIALDKTQ | | |
| | | 98\|Protein Name: | RFINDNILPAINNLTCEVAGAKVGVELSLYLTELSTV | | |
| | | fusion protein\| | FGSQITNPALSTLSIQALMSLCGNDFNYLLNLMGAKH | | |
| | | Gene Symbol: F | SDLGALYEANLINGRIIQYDQASQIMVIQVSVPSISS | | |
| | | | ISGLRLTELFTLSIETPVGEGKAVVPQFVVESGQLLE | | |
| | | | EIDTQACTLTDTTAYCTIVRTKPLPELVAQCLRGDES | | |
| | | | RCQYTTGIGMLESRFGVFDGLVIANCKATICRCLAPE | | |
| | | | MIITQNKGLPLTVISQETCKRILIDGVTLQIEAQVSG | | |
| | | | SYSRNITVGNSQIAPSGPLDISSELGKVNQSLSNVED | | |
| | | | LIDQSNQLLNRVNPNIVNNTAIIVTIVLLVLLVLWCL | | |
| | | | ALTISILYVSKHAVRMIKTVPNPYVMQAKSPGSATQF | | |
| AY141760 | 5028-6665 | gb: AY141660\| | MTRITILQIILTLTLPVMCQVSFDNLEQVGVMFDKPK | 8 | 15 |
| | | Organism: Fer-de- | FLKITGPASTATMIIKLIPTLGTMESCGTSAVNEYKK | | |
| | | Lance paramyxovirus\| | TLDTILVPLRDTINKLSTDITVVEGTSNISNKREKRF | | |
| | | Strain Name: ATCC | VGIAIAVGAVALATSAQITAGIALSNTIKNAEAIESI | | |
| | | VR-895\|Protein Name: | KSSIQASNQAIQKVIDAQGRTVTVINGIQDHINSVIN | | |
| | | fusion protein F\| | PALNQLGCDVAKNTLAISLTQYFSKLSLLFGPNLRNP | | |
| | | Gene Symbol: F | VEQPLSVQAIAGLMDGDINAVVSQLGYTQSDLLDLLS | | |
| | | | TESIVGTVTAIDMVNYMIQIEMSFPQYITIPDTKVLE | | |
| | | | GHKITFNDKGSEWQTQVPSTIAVRDILIAGVDPDGCS | | |
| | | | ITSTSYICKNDPTYAMSEVLTNCFRGNTQECPRARIT | | |
| | | | STFATRFAIARSTVIANCVAAVCLCGDPGIPVVQKAE | | |
| | | | VTLTAMTLDQCSLITVDGLQIKPSKSIANVTANFGNI | | |
| | | | TLGPVVSVGDLDLSAELTKVQSDLKEAQDKLDESNAI | | |
| | | | LQGINNKILTAPTSIALIVVSVVVILLIIGMISWLVW | | |
| | | | LTKAVRRSNTRSERVTPSAYNNLGFIK | | |
| EU877976 | 4330-6410 | gb: EU877976: | MRLSRTILTLILGTLTGYLMGAHSTNVNEGPKSEGIR | 8 | 16 |
| | | 4330-6410\| | GDLIPGAGIFVTQVRQLQIYQQSGYHDLVIRLLPLLP | | |
| | | Organism: Avian | AELNDCQREVVTEYNNTVSQLLQPIKTNLDTLLADGG | | |
| | | paramyxovirus 4\| | TRDADIQPRFIGAIIATGALAVATVAEVTAAQALSQS | | |
| | | Strain Name: APMV- | KTNAQNILKLRDSIQATNQAVFEISQGLEATATVLSK | | |
| | | 4/KR/YJ/06\|Protein | LQTELNENIIPSLNNLSCAAMGNRLGVSLSLYLTLMT | | |
| | | Name: fusion | TLFGDQITNPVLTPISYSTLSAMAGGHIGPVMSKILA | | |
| | | protein\|Gene Symbol: | GSVTSQLGAEQLIASGLIQSQVVGYDSQYQLLVIRVN | | |
| | | F | LVRIQEVQNTRVVSLRTLAVNRDGGLYRAQVPPEVVE | | |
| | | | RSGIAERFYADDCVLTTTDYICSSIRSSRLNPELVKC | | |
| | | | LSGALDSCTFERESALLSTPFFVYNKAVVANCKAATC | | |
| | | | RCNKPPSIIAQYSASALVTITTDTCADLEIEGYRFNI | | |
| | | | QTESNSWVAPNFTVSTSQIVSVDPIDISSDIAKINSS | | |
| | | | IEAAREQLELSNQILSRINPRIVNDESLIAIIVTIVV | | |
| | | | LSLLVIGLIVVLGVMYKNLKKVQRAQAAMMMQQMSSS | | |
| | | | QPVTTKLGTPF | | |
| AB176531 | 4793-6448 | gb: AB176531: | MHHLHPMIVCIFVMYTGIVGSDAIAGDQLLNIGVIQS | 7 | 17 |
| | | 4793-6448\| | KIRSLMYYTDGGASFIVVKLLPNLPPSNGTCNITSLD | | |
| | | Organism: Human | AYNVTLFKLLTPLIENLSKISTVTDTKTRQKRFAGVV | | |
| | | parainfluenza virus | VGLAALGVATAAQITAAVAIVKANANAAAINNLASSI | | |
| | | 2\|Strain Name: | QSTNKAVSDVIDASRTIATAVQAIQDRINGAIVNGIT | | |
| | | Nishio\|Protein Name: | SASCRAHDALIGSILNLYLTELTTIFHNQITNPALTP | | |
| | | fusion protein\| | LSIQALRILLGSTLPIVIESKLNTNFNTAELLSSGLL | | |
| | | Gene Symbol: F | TGQIISISPMYMQMLIQINVPTFIMQPGAKVIDLIAI | | |
| | | | SANHKLQEVVVQVPNRILEYANELQNYPANDCVVTPN | | |
| | | | SVFCRYNEGSPIPESQYQCLRGNLNSCTFTPIIGNFL | | |
| | | | KRFAFANGVLYANCKSLLCRCADPPHVVSQDDTQGIS | | |
| | | | IIDIKRCSEMMLDTFSFRITSTFNATYVTDFSMINAN | | |
| | | | IVHLSPLDLSNQINSINKSLKSAEDWIADSNFFANQA | | |
| | | | RTAKTLYSLSAIALILSVITLVVVGLLIAYIIKLVSQ | | |
| | | | IHQFRSLAATTMFHRENPAFFSKNNHGNIYGIS | | |
| BK005918 | 4677-6302 | gb: BK005918\| | MPQQQVAHTCVMLWGIISTVSGINTEALSQYGVVVTN | 7 | 18 |
| | | Organism: Porcine | VRQLTYYTQAGSTYLAVRLLPSLASPDQSCALHSIIN | | |
| | | rubulavirus\|Strain | YNATLQAILSPIAENLNLISTALREQHRKKRFAGVAI | | |
| | | Name: UNKNOWN- | GLTALGVATAAQATAAVALVRANKNAEKVEQLSQALG | | |
| | | BK005918\|Protein | ETNAAISDLIDATKNLGFAVQAIQNQINTAILPQIHN | | |
| | | Name: fusion | LSCQVIDAQLGNILSLYLTELTTVFQPQLTNPALSPL | | |
| | | protein\|Gene Symbol: | TIQALRAVLGTTLPALLSEKLKSNIPLGDLMSSGLLK | | |
| | | F | GQLVGLNLQNMLMIIELYIPTLSTHSTAKVLDLVTIS | | |
| | | | SHVNGREVEIQVPNRVLELGSEVLGYGGSECALTMSH | | |
| | | | ILCPFNDARVLSTDMKYCLQGNITHCIFSPVVGSFLR | | |
| | | | RFALVNGVVIANCADMSCVCFDPQEIIYQNFQEPTTV | | |
| | | | IDIKKCGKVQLDTLTFTISTFANRTYGPPAYVPPDNI | | |
| | | | IQSEPLDISGNLIAVNNSLSSALNHLATSEILRNEQI | | |
| | | | WTSSLGISTIVALVIIGILIICLVVTWAALWALLKEV | | |
| | | | RGLNSAVNSQLSSYVMGDKFIRY | | |
| KC237063 | 4530-6185 | gb: KC237063: | MGTRIQFLMVSCLLAGTGSLDPAALMQIGVIPTNVRQ | 7 | 19 |
| | | 4530-6185\| | LMYYTEASSAFIVVKLMPTIDSPISGCNITSISSYNA | | |
| | | Orgamins: | TMTKLLQPIGENLETIRYQLIPTRRRRRFVGVVIGLA | | |
| | | Parainfluenza virus | ALGVATAAQVTAAVALVKANKNAAAILNLKNAIQKTN | | |
| | | 5\|Strain Name: | AAVADVVQATQSLGTAVQAVQDHINSVVSPAITAANC | | |
| | | 08-1990\|Protein | KAQDAIIGSILNLYLTELTTIFHNQITNPALSPITIQ | | |
| | | Name: fusion | ALRILLGSTLPTVVRKSFNTQISAAELLSSGLLTGQI | | |
| | | protein\|Gene Symbol: | VGLDLTYMQMVIKIELPTLTVQPATQIIDLVTISAFI | | |
| | | F\|Segment: 4 | NNREVMAQLPTRIIVTGSLIQAYPASQCTITPNTVYC | | |
| | | | RYNDAQVLSDDTMACLQGNLTRCTFSPVVGSFLTRFV | | |
| | | | LFDGIVYANCRSMLCKCMQPAAVILQPSSSPVTVIDM | | |
| | | | HKCVSLQLDNLRFTITQLANITYNSTIKLETSQILPI | | |
| | | | DPLDISQNLAAVNKSLSDALQHLAQSDTYLSAITSAT | | |
| | | | TTSVLSIIAICLGSLGLILIILISVVVWKLLTIVAAN | | |
| | | | RNRMENFVYHNSAFHHSRSDLSEKNQPATLGTR | | |
| AY729016 | 5862-7523 | gb: AY729016: | MIPGRIFLVLLVIFNTKPIHPNTLTEKFYESTCSVET | 6 | 20 |
| | | 5862-7523\| | AGYKSALRTGWHMTVMSIKLSQINIESCKSSNSLLAH | | |
| | | Organism: Murine | ELAIYSSAVDELRTLSSNALKSKRKKRFLGLILGLGA | | |
| | | pneumonia virus\| | AVTAGVALAKTVQLESEIALIRDAVRNTNEAVVSLTN | | |
| | | Strain Name: 15; | GMSVLAKVVDDLKNFISKELLPKINRVSCDVHDITAV | | |
| | | ATCC VR-25\|Protein | IRFQQLNKRLLEVSREFSSNAGLTHTVSSFMLTDREL | | |
| | | Name: fusion | TSIVGGMAVSAGQKEIMLSSKAIMRRNGLAILSSVNA | | |
| | | glycoprotein | DTLVYVIQLPLFGVMDTDCWVIRSSIDCHNIADKYAC | | |
| | | precursor\|Gene | LARADNGWYCHNAGSLSYFPSPTDCEIHNGYAFCDTL | | |
| | | Symbol: F | KSLTVPVTSRECNSNMYTTNYDCKISTSKTYVSTAVL | | |
| | | | TTMGCLVSCYGHNSCTVINNDKGIIRTLPDGCHYISN | | |
| | | | KGVDRVQVGNTVYYLSKEVGKSIVVRGEPLVLKYDPL | | |
| | | | SFPDDKFDVAIRDVEHSINQTRTFLKASDQLLDLSEN | | |
| | | | RENKNLNKSYILTTLLFVVMLIIIMAVIGFILYKVLK | | |
| | | | MIRDNKLKSKSTPGLTVLS | | |
| AB543336 | 5174-6805 | gb: AB543336: | MGVKGLSLIMIGLLISPITNLDITHLMNLGTVPTAIR | 5 | 21 |
| | | 5174-6805\| | SLVYYTYTKPSYLTVDLIPNLKNLDQNCNYSSLNYYN | | |
| | | Organism: Human | KTALSLIQPIADNINRLTKPITSSEIQSRFFGAVIGT | | |
| | | parainfluenza | IALGVATAAQVTAAIGLAKAQENAKLILTLKKAATET | | |
| | | virus 4a\|Strain | NEAVRDLANSNKIVVKMISAIQNQINTIIQPAIDQIN | | |
| | | Name: M-25\|Protein | CQIKDLQVANILNLYLTEITTVFHNQLTNPALESISI | | |
| | | Name: fusion | QALKSLLGPTLPEVLSKLDLNNISAASVMASGLIKGQ | | |
| | | protein\|Gene Symbol: | IIAVDIPTMTLVLMVQIPSISPLRQAKIIDLTSITIH | | |
| | | F | TNSQEVQAVVPARFLEIGSEILGFDGSVCQITKDTIF | | |
| | | | CPYNDAYELPIQQKRCLQGQTRDCVFTPVAGTFPRRF | | |
| | | | LTTYGTIVANCRDLVCSCLRPPQIIYQPDENPVTIID | | |
| | | | KDLCTTLTLDSITIEIQKSINSTFRREVVLESTQVRS | | |
| | | | LTPLDLSTDLNQYNQLLKSAEDHIQRSTDYLNSINPS | | |
| | | | IVNNNAIIILIILCILLILTVTICIIWLKYLTKEVKN | | |
| | | | VARNQRLNRDADLFYKIPSQIPVPR | | |
| AF298895 | 4834-6450 | gb: AF298895\| | MRIALTAVIVSIHFDLAFPMNKNSLLSVGLVHKSVKN | 5 | 22 |
| | | Organism\| Tioman | LYFYSQGSPSYIVVKLVPTLGNVPGNCTLNSLVRYKS | | |
| | | virus\|Strain Name: | TVSSLLSPLAENLEYLQKTLTVSRGGRRRRFAGVAIG | | |
| | | UNKNOWN-AF298895\| | LAALGVAAAAQATAAVALVEARQNAAQIQSLSEAIQN | | |
| | | Protein Name: | TNLAVNELKTAIGASATAIQAIQTQINEVINPAINRL | | |
| | | fusion protein\|Gene | SCEILDAQLASMLNLYLIHLTTVFQNQLTNPALTPLS | | |
| | | Symbol: F | IQSLQSLLQGTSSVLTNITSSSKLALNDALVTGLITG | | |
| | | | QVVGLNMTSLQIVIAAYVPSVAKLSNAVVHNFIRITT | | |
| | | | SVNGTEVIIQSPTIIMEQNEVMYDLKTGHCTESDLNI | | |
| | | | YCPYVDAQLLSPGMTNCINGRLNDCTFSKVVGSFPTR | | |
| | | | FAAVEGAILANCKYLQCNCLTPPYIITPLNGEMISMI | | |
| | | | DLSKCQRLDLGTIVFDINNPVNVTFNGNYRADVGQMI | | |
| | | | VTNPLDISAELNQINTSLSNAQGFLSKSDAWLHVSQW | | |
| | | | VTNSGTIFIILIIGLIVGIVYMIINTYVVVQIIKEIN | | |
| | | | RMRTSDRAHLLKGSISSIST | | |
| FJ215863 | 4499-6130 | gb: FJ215863: | MGQISVYLINSVLLLLVYPVNSIDNTLIAPIGVASAN | 5 | 23 |
| | | 4499-6130\| | EWQLAAYTTSLSGTIAVRFLPVLPDNMTTCLRETITT | | |
| | | Organism: Avian | YNNTVNNILGPLKSNLDALLSSETYPQTRLIGAVIGS | | |
| | | paramyxovirus 8\| | IALGVATSAQITAAVALKQAQDNARNILALKEALSKT | | |
| | | Strain Name: goose/ | NEAVKELSSGLQQTAIALGKIQSFVNEEILPSINQLS | | |
| | | Delaware/1053/76\| | CEVTANKLGVYLSLYLTELTTIFGAQLTNPALTSLSY | | |
| | | Protein Name: fusion | QALYNLCGGNMAMLTQKIGIKQQDVNSLYEAGLITGQ | | |
| | | protein\|Gene | VIGYDSQYQLLVIQVNYPSISEVTGVRATELVTVSVT | | |
| | | Symbol: F | TDKGEGKAIVPQFVAESRVTIEELDVASCKFSSTTLY | | |
| | | | CRQVNTRALPPLVASCLRGNYDDCQYTTEIGALSSRY | | |
| | | | ITLDGGVLVNCKSIVCRCLNPSKIISQNTNAAVTYVD | | |
| | | | ATICKTIQLDDIQLQLEGSLSSVYARNISIEISQVTT | | |
| | | | SGSLDISSEIGNINNTVNRVEDLIHQSEEWLAKVNPH | | |
| | | | IVNNTTLIVLCVLSALAVIWLAVLTAIIIYLRTKLKT | | |
| | | | ISALAVTNTIQSNPYVNQTKRESKF | | |
| JN689227 | 4689-6521 | gb: JN689227: | MKLSVVYTTLLVSTFYSDLARSQLALSELTKIGVIPG | 5 | 24 |
| | | 4689-6521\| | RSYDLKISTQASYQYMVVKLIPNLTGLNNCTNGTIEA | | |
| | | Organism: Tailam | YKKMLNRLLSPIDAALRKMKDAVNDKPPESVGNVKFW | | |
| | | virus\|Strain Name: | GAVIGGVALGVATSAQITAGVALHNSIQNANAILALK | | |
| | | TL8K\|Protein Name: | DSIRQSNKAIQELQTAMSTTVVVLNALQDQINNQLVP | | |
| | | fusion protein\|Gene | AINSLGCQVVANTLGLKLNQYFSEISLVFGPNLRDPT | | |
| | | Symbol: F | SETLSIQALSRAFNGDFDSMLSKLKYDDSDFLDLLES | | |
| | | | DSIRGRIIDVSLSDYLITIQIEYPALLSIKDAVIQTF | | |
| | | | NLISYNTRGTEWISIFPKQLLVRGTYISNIDISQCVI | | |
| | | | AATSIICKSDTSTPISSATWSCATGNITNCARTRVVN | | |
| | | | AHVPRFALYGGVVFANCAPVVCKCQDPLYSINQEPKV | | |
| | | | TNVMVDVDACKEMYLDGLYITLGKTQISRAMYAEDVS | | |
| | | | LGGPISVDPIDLGNEINSINSAINRSEEHLNHANELL | | |
| | | | DKVNPRIVNVKTFGVMIGLLVLVVLWCVITLVWLICL | | |
| | | | TKQLARTAYAGSMGSRASTVNSLSGFVG | | |
| JX957409 | 4831-6615 | gb: JX857409: | MQVTTLRPAIILSIALLVTGQVPRDKLANLGIIIKDS | 5 | 25 |
| | | 4831-6615\| | KALKIAGSYENRYIVLSLVPTIDNVNGCGSIQIAKYK | | |
| | | Organism: Procine | EMLERLLIPIKDALDLQESLIVIDNETVNNNYSPQYR | | |
| | | parainfluenza virus | FVGAIIGTIALGVATAAQVTAGVALMEAREAKRDISM | | |
| | | 1\|Strain Name: | LKEAIEKTQNSIEKLQNSAGEQILALKMLQDYVNGEI | | |
| | | S206N\|Protein Name: | KPAIEELGCETAALKLGIALTQHYTELTNAFGSNLGS | | |
| | | fusion protein\|Gene | IGEKSLTLQALSSLYKTNITNILTATNLGKTDIYDII | | |
| | | Symbol: F | YAEQVKGRVIDVDLKRYMVTISVKIPILSEIPGVLIY | | |
| | | | EVSSISYNIDGAEWYAAVPDHILSKSAYIGGADISDC | | |
| | | | IESRLTYICPQDPAQIIADNQQQCFFGHLDKCPITKV | | |
| | | | IDNLVPKFAFINGGVVANCIASTCTCGEERIQVSQDR | | |
| | | | NKGVTFLTHNNCGLIGINGIEFHANKKGSDATWNVSP | | |
| | | | IGVGPAVSLRPVDISLQIVAATNFLNSSRKDLMKAKE | | |
| | | | ILNQVGNLKDLTTITIINIVIIIILLICVIGLGILYH | | |
| | | | QLRSALGMRDKMSVLNNSSYSLEPRTAQVQVIKPTSF | | |
| | | | MG | | |
| AY640317 | 2932-4571 | gb: AY640317: | MDVRICLLLFLISNPSSCIQETYNEESCSTVTRGYKS | 4 | 26 |
| | | 2932-4571\| | VLRTGWYTNVFNLEIGNVENITCNDGPSLIDTELVLT | | |
| | | Organism: Avian | KNALRELKTVSADQVAKESRLSSPRRRRFVLGAIALG | | |
| | | metapneumovirus\| | VATAAAVTAGVALAKTIRLEGEVKAIKNALRNTNEAV | | |
| | | Strain Name: LAHA\| | STLGNGVRVLATAVNDLKEFISKKLTPAINQNKCNIA | | |
| | | Protein Name: F\| | DIKMAISFGQNNRRFLNVVRQFSDSAGITSAVSLDLM | | |
| | | Gene Symbol: F | TDDELVRAINRMPTSSGQISLMLNNRAMVRRKGFGIL | | |
| | | | IGVYDGTVVYMVQLPIFGVIETPCWRVVAAPLCRKRR | | |
| | | | GNYACILREDQGWYCTNAGSTAYYPNKDDCEVRDDYV | | |
| | | | FCDTAAGINVALEVDQCNYNISTSKYPCKVSTGRHPV | | |
| | | | SMVALTPLGGLVSCYESVSCSIGSNKVGIIKQLGKGC | | |
| | | | THIPNNEADTITIDNTVYQLSKVVGEQRTIKGAPVVN | | |
| | | | NFNPILFPVDQFNVALDQVFESIDRSQDLIDKSNDLL | | |
| | | | GADAKSKAGIAIAIVVLVILGIFFLLAVIYYCSRVRK | | |
| | | | TKPKHDYPATTGHSSMAYVS | | |
| KU636513 | 4641-6498 | gb: KU646513: | MARFSWEIFRLSTILLIAQTCQGSIDGRLTLAAGIVP | 4 | 27 |
| | | 4641-6498\| | VGDRPISIYTSSQTGIIVVKLIPNLPDNKKDCAKQSL | | |
| | | Organism: Avian | QSYNETLSRILTPLATAMSAIRGNSTTQVRENRLVGA | | |
| | | paramyxovirus 13 | IIGSVALGVATAAQITAATALIQANQNAANIARLANS | | |
| | | goose/Kazakhstan/ | IAKTNEAVTDLTEGLGTLAIGVGKLQDYVNEQFNNTA | | |
| | | 5751/2013\|Strain | VAIDCLTLESRLGIQLSLYLTELMGVFGNQLTSPALT | | |
| | | Name: APMV-13/ | PITIQALYNLAGGNLNALLSRLGASETQLGSLINSGL | | |
| | | white fronted | IKGMPIMYDDANKLLAVQVELPSIGKLNGARSTLLET | | |
| | | goose/Northern | LAVDTTRGPSSPIIPSAVIEIGGAMEELDLSPCITTD | | |
| | | Kazakhstan/5751/ | LDMFCTKIISYPLSQSTLSCLNGNLSDCVFSRSEGVL | | |
| | | 2013\|Protein Name: | STPYMTIKGKIVANCKQVICRCMDPPQILSQNYGEAL | | |
| | | fusion protein\| | LLIDENTCRSLELSGVILKLAGTYESEYTRNLTVDPS | | |
| | | Gene Symbol: F | QVIITGPLDISAELSKVNQSIDSAKENIAESNKFLSQ | | |
| | | | VNVKLLSSSAMITYIVATVVCLIIAITGCVIGIYTLT | | |
| | | | KLKSQQKTLLWLGNNAEMHGSRSKTSF | | |
| AF326114 | 4818-6482 | gb: AF326114\| | MMPRVLGMIVLYLTHSQILCINRNTLYQIGLIHRSVK | 3 | 28 |
| | | Organism: Menangle | KVNFYSQGSPSYIVVKLVPTLAAIPPNCSIKSLQRYK | | |
| | | virus\|Strain Name: | ETVTSLVQPISDNLGYLQDKLVTGQSRRRRRFAGVAI | | |
| | | UNKNOWN-AF326114\| | GLAALGVAAAAQATAAVALVETRENAGKIQALSESIQ | | |
| | | Protein Name: fusion | NTNQAVHSLKTALGFSATAIQAIQNQVNEVINPAINK | | |
| | | protein\|Gene | LSCEVLDSQLASMLNLYLIHLTTVFQTQLTNPALTPL | | |
| | | Symbol: F | SIQALTSVLQGTSGVLMNSTNSTLTQPIDLLATGLIT | | |
| | | | GQIISVNMTSLQLIIATFMPSIAELPNAVLHSFFRIT | | |
| | | | TSVNLTEVMIQSPEFIMEQNGVFYDFNTAHCQLGDNN | | |
| | | | VYCPYIDAARLSSMMTNCINGNLGECVFSRVIGSFPS | | |
| | | | RFVSLNGAILANCKFMRCNCLSPEKIITPLDGEMISL | | |
| | | | IDLRVCQKLTLGTITFEISQPVNVSFQGGFVANAGQI | | |
| | | | IVTNPFDISAELGQINNSLNDAQGFLDQSNNWLKVSG | | |
| | | | WINNSGSLFIAGIVVIGLIVLCIVIIIYINVQIIREV | | |
| | | | NRLRSFIYRDYVLDHDKAPYSPESSSPHRKSLKTVS | | |
| GU206351 | 5441-7468 | gb: GU206351: | MLQLPLTILLSILSAHQSLCLDNSKLIHAGIMSTTER | 3 | 29 |
| | | 5441-7468\| | EVNVYAQSITGSIVVRLIPNIPSNHKSCATSQIKLYN | | |
| | | Organism: Avian | DTLTRLLTPIKANLEGLISAVSQDQSQNSGKRKKRFV | | |
| | | paramyxovirus 5\| | GAVIGAAALGLATAAQVTATVALNQAQENARNILRLK | | |
| | | Strain Name: | NSIQKTNEAVMELKDAVGQTAVAIDKTQAFINNQILP | | |
| | | budgerigar/ | AISNLSCEVLGNKIGVQLSLYLTELTTVFGNQLTNPA | | |
| | | Kunitachi/74/Protein | LTTLSLQALYNLCGDDFNYLINLLNAKNRNLASLYEA | | |
| | | Name: fusion | NLIQGRITQYDSMNQLLIIQVQIPSISTVSGMRVTEL | | |
| | | protein\|Gene | FTLSVDTPIGEGKALVPKYVLSSGRIMEEVDLSSCAI | | |
| | | Symbol: F | TSTSVFCSSIISRPLPLETINCLNGNVTQCQFTANTG | | |
| | | | TLESRYAVIGGLVIANCKAIVCRCLNPPGVIAQNLGL | | |
| | | | PITIISSNTCQRINLEQITLSLGNSILSTYSANLSQV | | |
| | | | EMNLAPSNPLDISVELNRVNTSLSKVESLIKESNSIL | | |
| | | | DSVNPQILNVKTVIILAVIIGLIVVWCFILTCLIVRG | | |
| | | | FMLLVKQQKFKGLSVQNNPYVSNNSH | | |
| JQ001776 | 6129-8166 | gb: JQ001776: | MSNKRTTVLIIISYTLFYLNNAAIVGFDFDKLNKIGV | 3 | 30 |
| | | 6129-8166\| | VQGRVLNYKIKGDPMTKDLVLKFIPNIVNITECVREP | | |
| | | Organism: Cedar | LSRYNETVRRLLLPIHNMLGLYLNNTNAKMTGLMIAG | | |
| | | virus\|Strain Name: | VIMGGIAIGIATAAQITAGFALYEAKKNTENIQKLTD | | |
| | | CG1a\|Protein Name: | SIMKTQDSIDKLTDSVGTSILILNKLQTYINNQLVPN | | |
| | | fusion glycoprotein\| | LELLSCRQNKIEFDLMLTKYLVDLMTVIGPNINNPVN | | |
| | | Gene Symbol: F | KDMTIQSLSLLFDGNYDIMMSELGYTPQDFLDLIESK | | |
| | | | SITGQIIYVDMENLYVVIRTYLPTLIEVPDAQIYEFN | | |
| | | | KITMSSNGGEYLSTIPNFILIRGNYMSNIDVATCYMT | | |
| | | | KASVICNQDYSLPMSQNLRSCYQGETEYCPVEAVIAS | | |
| | | | HSPRFALTNGVIFANCINTICRCQDNGKTITQNINQF | | |
| | | | VSMIDNSTCNDVMVDKFTIKVGKYMGRKDINNINIQI | | |
| | | | GPQIIIDKVDLSNEINKMNQSLKDSIFYLREAKRILD | | |
| | | | SVNISLISPSVQLFLIIISVLSFIILLIIIVYLYCKS | | |
| | | | KHSYKYNKFIDDPDYYNDYKRERINGKASKSNNIYYV | | |
| | | | GD | | |
| LC168749 | 4869-7235 | gb: LC168749: | MGILFAALLAMTNPHLATGQIHWGNLSKIGVVGTGSA | 2 | 31 |
| | | 4869-7235\| | SYKVMTQSSHQSLVIKLMPNVTAIDNCTKTEIMEYKR | | |
| | | Organism: Rinderpest | LLGTVLKPIREALNAITKNIKPIQSSTTSRRHKRFAG | | |
| | | morbillivirus\| | VVLAGAALGVATAAQITAGIALHQSMMNSQAIESLKA | | |
| | | Strain Name: Lv\| | SLETTNQAIEEIRQAGQEMVLAVQGVQDYINNELVPA | | |
| | | Protein Name: F | MGQLSCEIVGQKLGLKLLRYYTEILSLFGPSLRDPVS | | |
| | | protein\|Gene Symbol: | AELSIQALSYALGGDINKILEKLGYSGSDLLAILESK | | |
| | | F | GIKAKITYVDIESYFIVLSIAYPSLSEIKGVIVHRLE | | |
| | | | SVSYNIGSQEWYTTVPRYVATQGYLISNFDDTPCAFT | | |
| | | | PEGTICSQNALYPMSPLLQECFRGSTRSCARTLVSGS | | |
| | | | IGNRFILSKGNLIANCASILCKCYTTGSIISQDPDKI | | |
| | | | LTYIAADQCPVVEVGGVTIQVGSREYSDAVYLHEIDL | | |
| | | | GPPISLEKLDVGTNLWNAVTKLEKAKDLLDSSDLILE | | |
| | | | NIKGVSVTNTGYILVGVGLIAVVGILIITCCCKKRRS | | |
| | | | DNKVSTMVLNPGLRPDLTGTSKSYVRSL | | |
| LC187310 | 6250-7860 | gb: LC187310: | MTRTRLLFLLTCYIPGAVSLDNSILAPAGIISASERQ | 2 | 32 |
| | | 6250-7860\| | IAIYTQTLQGTIALRFIPVLPQNLSSCAKDTLESYNS | | |
| | | Organism: Avian | TVSNLLLPIAENLNALLKDADKPSQRIIGAIIGSVAL | | |
| | | paramyxovirus 10\| | GVATTAQVTAALAMTQAQQNARNIWKLKESIKNTNQA | | |
| | | Strain Name: rAPMV- | VLELKDGLQQSAIALDKVQSFINSEILPQINQLGCEV | | |
| | | 10-FI324/YmHA\| | AANKLGIFLSLYLTEITTVFKNQITNPALSTLSYQAL | | |
| | | Protein Name: fusion | YNLCGGNMAALTKQIGIKDTEINSLYEAELITGQVIG | | |
| | | protein\|Gene | YDSADQILLIQVSYPSVSRVQGVRAVELLTVSVATPK | | |
| | | Symbol: F | GEGKAIAPSFIAQSNIIAEELDTQPCKFSKTTLYCRQ | | |
| | | | VNTRTLPVRVANCLKGKYNDCQYTTEIGALASRYVTI | | |
| | | | TNGVVANCRSIICRCLDPEGIVAQNSDAAITVIDRST | | |
| | | | CKLIQLGDITLRLEGKLSSSYSKNITIDISQVTTSGS | | |
| | | | LDISSELGSINNTITKVEDLISKSNDWLSKVNPTLIS | | |
| | | | NDTIIALCVIAGIVVIWLVIITILSYYILIKLKNVAL | | |
| | | | LSTMPKKDLNPYVNNTKF | | |
| NC_005283 | 5277-6935 | gb: NC_005283: | MAASNGGVMYQSFLTIIILVIMTEGQIHWGNLSKIGI | 2 | 33 |
| | | 5277-6935\| | VGTGSASYKVMTRPNHQYLVIKLMPNVTMIDNCTRTE | | |
| | | Organism: Dolphin | VTEYRKLLKTVLEPVKNALTVITKNIKPIQSLTTSRR | | |
| | | morbillivirus\| | SKRFAGVVLAGVALGVATAAQITAGVALHQSIMNSQS | | |
| | | Strain Name: | IDNLRTSLEKSNQAIEEIRQASQETVLAVQGVQDFIN | | |
| | | UNKNOWN-NC_005283\| | NELIPSMHQLSCEMLGQKLGLKLLRYYTEILSIFGPS | | |
| | | Protein Name: fusion | LRDPVSAEISIQALSYALGGDINKILEKLGYSGADLL | | |
| | | protein\|Gene | AILESRGIKAKVTHVDLEGYFIVLSIAYPTLSEVKGV | | |
| | | Symbol: F | IVHKLEAVSYNLGSQEWYTTLPKYVATNGYLISNFDE | | |
| | | | SSCAFMSEVTICSQNALYPMSPLLQQCLRGSTASCAR | | |
| | | | SLVSGTIGNRFILSKGNLIANCASVLCKCYSTGTIIS | | |
| | | | QDPDKLLTFVAADKCPLVEVDGITIQVGSREYPDSVY | | |
| | | | VSRIDLGPAISLEKLDVGTNLGSALTKLDNAKDLLDS | | |
| | | | SNQILENVRRSSFGGAMYIGILVCAGALVILCVLVYC | | |
| | | | CRRHCRKRVQTPPKATPGLKPDLTGTTKSYVRSL | | |
| NC_005339 | 5374-7602 | gb: NC_005339: | MSNYFPARVIIIVSLITAVSCQISFQNLSTIGVFKFK | 2 | 34 |
| | | 5374-7602\| | EYDYRVSGDYNEQFLAIKMVPNVTGVENCTASLIDEY | | |
| | | Organism: Mossman | RHVIYNLLQPINTTLTASTSNVDPYAGNKKFFGAVIA | | |
| | | virus\|Strain Name: | GVALGVATAAQVTAGVALYEARQNAAAIAEIKESLHY | | |
| | | UNKNOWN-NC_005339\| | THKAIESLQISQKQTVVAIQGIQDQINTNIIPQINAL | | |
| | | Protein Name: | TCEIANQRLRLMLLQYYTEMLSSFGPIIQDPLSGHIT | | |
| | | fusion protein\| | VQALSQAAGGNITGLMRELGYSSKDLRYILSVNGISA | | |
| | | Gene Symbol: F | NIIDADPEIGSIILRIRYPSMIKIPDVAVMELSYLAY | | |
| | | | HAAGGDWLTVGPRFILKRGYSLSNLDITSCTIGEDFL | | |
| | | | LCSKDVSSPMSLATQSCLRGDTQMCSRTAVQDREAPR | | |
| | | | FLLLQGNLIVNCMSVNCKCEDPEETITQDPAYPLMVL | | |
| | | | GSDTCKIHYIDGIRIKLGKVQLPPITVLNTLSLGPIV | | |
| | | | VLNPIDVSNQLSLVETTVKESEDHLKNAIGALRSQSR | | |
| | | | VGGVGIVAIVGLIIATVSLVVLVISGCCLVKYFSRTA | | |
| | | | TLESSLTTIEHGPTLAPKSGPIIPTYINPVYRHD | | |
| NC_007454 | 4635-6384 | gb: NC_007454: | MKPVALIYLTILAFTVKVRSQLALSDLTKIGIIPAKS | 2 | 35 |
| | | 4635-6384\| | YELKISTQAAQQLMVIKLIPNVNGLTNCTIPVMDSYK | | |
| | | Organism: J-virus\| | KMLDRILKPIDDALNHVKNAIQDKQGDGVPGVRFWGA | | |
| | | Strain Name: | IIGGVALGVATSAQITAGVALHNSIQNANAILQLKES | | |
| | | UNKNOWN-NC_007454\| | IRNSNKAIEELQAGLQSTVLVINALQDQINSQLVPAI | | |
| | | Protein Name: fusion | NTLGCSVIANTLGLRLNQYFSEISLVFGPNLRDPTSQ | | |
| | | protein\|Gene Symbol: | TLSIQAIAKAFNGDFDSMMKKMHYTDSDFLDLLESDS | | |
| | | F | IRGRIISVSLEDYLIIIQIDYPGLTTIPNSVVQTFNL | | |
| | | | ITYNYKGTEWESIFPRELLIRGSYISNIDISQCVGTS | | |
| | | | KSMICKSDTSTTISPATWACATGNLTSCARTRVVNSH | | |
| | | | STRFALSGGVLFANCAPIACRCQDPQYSINQEPKTTN | | |
| | | | VMVTSEDCKELYIDGFYLTLGKKWILDRAMYAEDVAL | | |
| | | | GGSVSVDPIDIGNELNSINESINKSHEYLDKANELLE | | |
| | | | QVNPNIVNVSSFSFILVISILLIIWFIVTLVWLIYLT | | |
| | | | KHMNFIVGKVAMGSRSSTVNSLSGFVG | | |
| NC_009489 | 4620-6500 | gb: NC_009489: | MRSSLFLVLTLLVPFAHSIDSITLEQYGTVITSVRSL | 2 | 36 |
| | | 4620-6500\| | AYFLETNPTYISVRLMPAIQTDSSHCSYHSIENYNLT | | |
| | | Organism: Mapuera | LTKLLLPLQENLHQITDSLSSRRRKKRFAGVAVGLAA | | |
| | | virus\|Strain Name: | LGVATAAQVTAAIAVVKAKENSAKIAQLTSAISETNR | | |
| | | BeAnn 370284\|Protein | AVQDLIEGSKQLAVAVQAIQDQINNVIQPQLTNLSCQ | | |
| | | Name: fusion | VADAQVGTILNMYLTELTTVFHPQITNSALTPITIQA | | |
| | | protein\|Gene Symbol: | LRSLLGSTLPQVVTSTIKTDVPLQDLLTSGLLKGQIV | | |
| | | F | YLDLQSMIMVVSVSVPTIALHSMAKVYTLKAISAHVN | | |
| | | | NAEVQMQVPSRVMELGSEIMGYDIDQCEETSRYLFCP | | |
| | | | YNGGSILSATMKMCLNGNISQCVFTPIYGSFLQRFVL | | |
| | | | VDGVIVANCRDMTCACKSPSKIITQPDSLPVTIIDST | | |
| | | | SCSNLVLDTLELPIISINNATYRPVQYVGPNQIIFSQ | | |
| | | | PLDLLSQLGKINSSLSDAIEHLAKSDEILEQIQWDSP | | |
| | | | QGYTLIALTSVLAFVVVAIVGLLISTRYLIFEIRRIN | | |
| | | | TTLTQQLSSYVLSNKIIQY | | |
| NC_017937 | 4534-6330 | gb: NC_017937: | MAEQEKTPLRYKILLIIIVINHYNITNVFGQIHLANL | 2 | 37 |
| | | 4534-6330\| | SSIGVFVTKTLDYRTTSDPTEQLLVINMLPNISNIQD | | |
| | | Organism: Nariva | CAQGVVNEYKHLISSLLTPINDTLDLITSNINPYSGR | | |
| | | virus\|Strain Name: | NKLFGEIIAGAALTVATSAQITAGVALYEARQNAKDI | | |
| | | UNKNOWN-NC_017937\| | AAIKESLGYAYKAIDKLTTATREITVVINELQDQINN | | |
| | | Protein Name: fusion | RLIPRINDLACEVWATRLQAMLLQYYAEIFSVIGPNL | | |
| | | protein\|Gene | QDPLSGKISIQALARAAGGNIKLMVDELNYSGQDLSR | | |
| | | Symbol: F | LVKVGAIKGQIIDADPSLGVVIIKMRYPNIIKIPNVA | | |
| | | | ISELSYVSYSSDGQDWITTGPNYIVTRGYSIANIQTS | | |
| | | | SCSVGDDFVLCDRDMTYPMSQVTQDCLRGNIALCSRM | | |
| | | | VVRDREAPRYLILQGNMVANCMSITCRCEEPESEIYQ | | |
| | | | SPDQPLTLLTRDTCDTHVVDGIRIRLGVRKLPTISVI | | |
| | | | NNITLGPIITTDPIDVSNQLNAVVSTIDQSAELLHQA | | |
| | | | QRVLSERARGARDHILATAAIVICVVLAVLILVLLIG | | |
| | | | LVYLYRTQNEILVKTTMLEQVPTFAPKSFPMESQIYS | | |
| | | | GKTNKGYDPAE | | |
| NC_025256 | 6865-8853 | gb: NC_025256: | MKKKTDNPTISKRGHNHSRGIKSRALLRETDNYSNGL | 2 | 38 |
| | | 6865-8853\| | IVENLVRNCHHPSKNNLNYTKTQKRDSTIPYRVEERK | | |
| | | Organism: Bat | GHYPKIKHLIDKSYKHIKRGKRRNGHNGNIITIILLL | | |
| | | Paramyxovirus | ILILKTQMSEGAIHYETLSKIGLIKGITREYKVKGTP | | |
| | | Eid_hel/GH-M74a/ | SSKDIVIKLIPNVTGLNKCTNISMENYKEQLDKILIP | | |
| | | GHA/2009\|Strain | INNIIELYANSTKSAPGNARFAGVIIAGVALGVAAAA | | |
| | | Name: BatPV/ | QITAGIALHEARQNAERINLLKDSISATNNAVAELQE | | |
| | | Eid_hel/GH-M74a/ | ATGGIVNVITGMQDYINTNLVPQIDKLQCSQIKTALD | | |
| | | GHA/2009\|Protein | ISLSQYYSEILTVFGPNLQNPVTTSMSIQAISQSFGG | | |
| | | Name: fusion | NIDLLLNLLGYTANDLLDLLESKSITGQITYINLEHY | | |
| | | protein\|Gene | FMVIRVYYPIMTTISNAYVQELIKISFNVDGSEWVSL | | |
| | | Symbol: F | VPSYILIRNSYLSNIDISECLITKNSVICRHDFAMPM | | |
| | | | SYTLKECLTGDTEKCPREAVVTSYVPRFAISGGVIYA | | |
| | | | NCLSTTCQCYQTGKVIAQDGSQTLMMIDNQTCSIVRI | | |
| | | | EEILISTGKYLGSQEYNTMHVSVGNPVFTDKLDITSQ | | |
| | | | ISNINQSIEQSKFYLDKSKAILDKINLNLIGSVPISI | | |
| | | | LFIIAILSLILSIITFVIVMIIVRRYNKYTPLINSDP | | |
| | | | SSRRSTIQDVYIIPNPGEHSIRSAARSIDRDRD | | |
| NC_025347 | 4471-6386 | gb: NC_025347: | MRVRPLIIILVLLVLLWLNILPVIGLDNSKIAQAGII | 2 | 39 |
| | | 4471-6386\| | SAQEYAVNVYSQSNEAYIALRTVPYIPPHNLSCFQDL | | |
| | | Organism: Avian | INTYNTTIQNIFSPIQDQITSITSASTLPSSRFAGLV | | |
| | | paramyxovirus 7\| | VGAIALGVATSAQITAAVALTKAQQNAQEIIRLRDSI | | |
| | | Strain Name: APMV- | QNTINAVNDITVGLSSIGVALSKVQNYLNDVINPALQ | | |
| | | 7/dove/Tennessee/ | NLSCQVSALNLGIQLNLYLTEITTIFGPQITNPSLTP | | |
| | | 4/75\|Protein Name: | LSIQALYTLAGDNLMQFLTRYGYGETSVSSILESGLI | | |
| | | fusion protein\| | SAQIVSFDKQTGIAILYVTLPSIATLSGSRVTKLMSV | | |
| | | Gene Symbol: F | SVQTGVGEGSAIVPSYVIQQGTVIEEFIPDSCIFTRS | | |
| | | | DVYCTQLYSKLLPDSILQCLQGSMADCQFTRSLGSFA | | |
| | | | NRFMTVAGGVIANCQTVLCRCYNPVMIIPQNNGIAVT | | |
| | | | LIDGSLCKELELEGIRLTMADPVFASYSRDLIINGNQ | | |
| | | | FAPSDALDISSELGQLNNSISSATDNLQKAQESLNKS | | |
| | | | IIPAATSSWLIILLFVLVSISLVIGCISIYFIYKHST | | |
| | | | TNRSRNLSSDIISNPYIQKAN | | |
| NC_025348 | 4790-6570 | gb: NC_025348: | MAPCVLFLSSLLLISTISPSHGINQPALRRIGAIVSS | 2 | 40 |
| | | 4790-6570\| | VKQLKFYSKTKPNYIIVKLLPTINLSKSNCNLTSINR | | |
| | | Organism: Tuhoko | YKESVIEIIKPLADNIDNLNQKLLPKNRRKRMAGVAI | | |
| | | virus 2\|Strain | GLAALGVAAAAQATAAVALVEARKNTQMIQSLADSIQ | | |
| | | Name: UNKNOWN- | DTNAAVQAVNIGLQNSAVAIQAIQNQINNVINPALDR | | |
| | | NC_025348\|Protein | LNCEVLDAQIASILNLYLIKSVTIFQNQLTNPALQQL | | |
| | | Name: fusion | SIQMLSIVMQDTAKILGNFTIGDKFDQHDLLGSGLIT | | |
| | | protein\|Gene | GQVVGVNLTNLQLIIAAFIPSIAPLPQAYIIDLISIT | | |
| | | Symbol: F | ISVNDTEAVIQIPERIMEHGSSIYQFGGKQCVYGQFS | | |
| | | | AYCPFSDAVLMTQDLQLCMKGNIEHCIFSSVLGSFPN | | |
| | | | RFASVDGVFYANCKYMSCACSDPLQVIHQDDSVNLMV | | |
| | | | IDSSVCRSLTLGHVTFPIIAFSNVSYQMKTNISIEQM | | |
| | | | IVTSPLDLSTELKQINNSVNIANTFLDSSNRALKTSI | | |
| | | | FGTSSQIILIVLLIFTCLLILYVIFLTYIIKILIKEV | | |
| | | | KRLRDGNSRTGSKLSFINPDV | | |
| NC_025350 | 4663-6428 | gb: NC_025350: | MLWLTILIALVGNHESTCMNINFLQSLGQINSQKRFL | 2 | 41 |
| | | 4663-6428\| | NFYTQQPPSYMVIRLVPTLQLSANNCTLGSIVRYRNA | | |
| | | Organism: Tuhoko | IKELIQPMDENLRWLSSNLIPQRRGKRFAGVAVGLAA | | |
| | | virus 3\|Strain Name: | LGVAVAAQATAAVALVEARANAEKIASMSQSIQETNK | | |
| | | UNLNOWN-NC_025350\| | AVTSLSQAVSASGIAIQAIQNEINNVIHPILNQVQCD | | |
| | | Protein Name: fusion | VLDARVGNILNLYLIKVTTIFQNQLTNPALQRLSTQA | | |
| | | protein\|Gene | LSMLMQSTSSYLRNLSSSESAINADLSMTNLIEAQIV | | |
| | | Symbol: F | GINMTNLQLVLAVFIPSIARLNGALLYDFISITISSN | | |
| | | | QTEVMLQIPHRVLEIGNSLYTFEGTQCEMTKLNAYCL | | |
| | | | YSDAIPVTESLRDCMNGLFSQCGFVRIIGSFANRFAS | | |
| | | | VNGVIYANCKHLTCSCLQPDEIITQDTNVPLTIIDTK | | |
| | | | RCTKISLGHLTFTIREYANVTYSLRTEIANSQITVVS | | |
| | | | PLDLSSQLTTINNSLADATNHIMNSDRILDRLNSGLY | | |
| | | | SKWVIIFLICASIVSLIGLVFLGFLIRGLILELRSKH | | |
| | | | RSNLNKASTYSIDSSIGLT | | |
| NC_025352 | 5950-8712 | gb: NC_025352: | MALNKNMFSSLFLGYLLVYATTVQSSIHYDSLSKVGV | 2 | 42 |
| | | 5950-8712\| | IKGLTYNYKIKGSPSTKLMVVKLIPNIDSVKNCTQKQ | | |
| | | Organism: Mojiang | YDEYKNLVRKALEPVKMAIDTMLNNVKSGNNKYRFAG | | |
| | | virus\|Strain Name: | AIMAGVALGVATAATVTAGIALHRSNENAQAIANMKS | | |
| | | Tongguan 1\|Protein | AIQNTNEAVKQLQLANKQTLAVIDTIRGEINNNIIPV | | |
| | | Name: fusion | INQLSCDTIGLSVGIRLTQYYSEIITAFGPALQNPVN | | |
| | | protein\|Gene | TRITIQAISSVFNGNFDELLKIMGYTSGDLYEILHSE | | |
| | | Symbol: F | LIRGNIIDVDVDAGYIALEIEFPNLTLVPNAVVQELM | | |
| | | | PISYNIDGDEWVTLVPRFVLTRTTLLSNIDTSRCTIT | | |
| | | | DSSVICDNDYALPMSHELIGCLQGDTSKCAREKVVSS | | |
| | | | YVPKFALSDGLVYANCLNTICRCMDTDTPISQSLGAT | | |
| | | | VSLLDNKRCSVYQVGDVLISVGSYLGDGEYNADNVEL | | |
| | | | GPPIVIDKIDIGNQLAGINQTLQEAEDYIEKSEEFLK | | |
| | | | GVNPSIITLGSMVVLYIFMILIAIVSVIALVLSIKLT | | |
| | | | VKGNVVRQQFTYTQHVPSMENINYVSH | | |
| NC_025363 | 4622-6262 | gb: NC_025363: | MAIPVPSSTALMIFNILVSLAPASALDGRLLLGAGIV | 2 | 43 |
| | | 4622-6262\| | PTGDRQVNVYTSSQTGIIALKLLPNLPKDKENCAEVS | | |
| | | Organism: Avian | IRSYNETLTRILTPLAQSMAAIRGNSTVSTRGREPRL | | |
| | | paramyxovirus 12\| | VGAIIGGVALGVATAAQITAATALIQANQNAENIARL | | |
| | | Strain Name: | AKGLAATNEAVTDLTKGVGSLAIGVGKLQDYVNEQFN | | |
| | | Wigeion/Italy/ | RTGEAIECLTIESRVGVQLSLYLTEVIGVFGDQITSP | | |
| | | 3920_1/2005\|Protein | ALSDISIQALYNLAGGNLNVLLQKMGIEGTQLGSLIN | | |
| | | Name: fusion | SGLIKGRPIMYDDGNKILGIQVTLPSVGRINGARATL | | |
| | | protein\|Gene | LEAIAVATPKGNASPLIPRAVISVGSLVEELDMTPCV | | |
| | | Symbol: F | LTPTDIFCTRILSYPLSDSLTTCLKGNLSSCVFSRTE | | |
| | | | GALSTPYVSVHGKIVANCKSVVCRCVEPQQIISQNYG | | |
| | | | EALSLIDESLCRILELNGVILKMDGQFTSEYTKNITI | | |
| | | | DPVQVIISGPIDISSELSQVNQSLDSALENIKESNSY | | |
| | | | LSKVNVKLISSSAMITYIVITVICLILTFVALVLGIY | | |
| | | | SYTKIRSQQKTLIWMGNNIARSKEGNRF | | |
| NC_025373 | 4617-6582 | gb: NC_025373: | MASPMVPLLIITVVPALISSQSANIDKLIQAGIIMGS | 2 | 44 |
| | | 4617-6582\| | GKELHIYQESGSLDLYLRLLPVIPSNLSHCQSEVITQ | | |
| | | Organism: Avian | YNSTVTRLLSPIAKNLNHLLQPRPSGRLFGAVIGSIA | | |
| | | paramyxovirus 3\| | LGVATSAQISAAIALVRAQQNANDILALKAAIQSSNE | | |
| | | Strain Name: | AIKQLTYGQEKQLLAISKIQKAVNEQVIPALTALDCA | | |
| | | turkey/Wisconsin/ | VLGNKLAAQLNLYLIEMTTIFGDQINNPVLTPIPLSY | | |
| | | 68\|Protein Name: | LLRLTGSELNDVLLQQTRSSLSLIHLVSKGLLSGQII | | |
| | | fusion protein\| | GYDPSVQGIIIRIGLIRTQRIDRSLVFXPYVLPITIS | | |
| | | Gene Symbol: F | SNIATPIIPDCVVKKGVIIEGMLKSNCIELERDIICK | | |
| | | | TINTYQITKETRACLQGNITMCKYQQSRTQLSTPFIT | | |
| | | | YNGVVIANCDLVSCRCIRPPMIITQVKGYPLTIINRN | | |
| | | | LCTELSVDNLILNIETNHNFSLNPTIIDSQSRLIATS | | |
| | | | PLEIDALIQDAQHHAAAALLKVEESNAHLLRVTGLGS | | |
| | | | SSWHIILILTLLVCTIAWLIGLSIYVCRIKNDDSTDK | | |
| | | | EPTTQSSNRGIGVGSIQYMT | | |
| NC_025386 | 5548-7206 | gb: NC_025386: | MNPLNQTLIAKVLGFLLLSSSFTVGQIGFENLTRIGV | 2 | 45 |
| | | 5548-7206\| | HQVKQYGYKLAHYNSHQLLLIRMIPTVNGTHNCTHQV | | |
| | | Organism: Salem | ITRYREMVREIITPIKGALDIMKKAVSPDLVGARIFG | | |
| | | virus\|Strain Name: | AIVAGAALGIATSAQITAGVALHRTKLNGQEISKLKE | | |
| | | UNKNOWN-NC_025386\| | AVSLTNEAVEQLQYSQGKSILAIQGIQDFINFNVVPL | | |
| | | Protein Name\|fusion | LEEHTCGIAKLHLEMALMEYFQKLILVFGPNLRDPIG | | |
| | | protein\|Gene Symbol: | STIGIQALATLFQNNMFEVSLRLGYAGDDLEDVLQSN | | |
| | | F | SIRANIIEAEPDSGFIVLAIRYPTLTLVEDQVITELA | | |
| | | | HITFNDGPQEWVATIPQFVTYRGLVLANIDVSTCTFT | | |
| | | | ERNVICARDQTYPMIIDLQLCMRGNIAKCGRTRVTGS | | |
| | | | TASRFLLKDGNMYANCIATMCRCMSSSSIINQEPSHL | | |
| | | | TTLIVKETCSEVMIDTIRITLGERKHPPIDYQTTITL | | |
| | | | GQPIALAPLDVGTELANAVSYLNKSKVLLEHSNEVLS | | |
| | | | SVSTAHTSLTATIVLGIVVGGLAILIVVMFLFLEAQV | | |
| | | | IKVQRAMMLCPITNHGYLPNEDLLTRGHSIPTIG | | |
| NC_025390 | 4805-6460 | gb: NC_025390: | MGYFHLLLILTAIAISAHLCYTTTLDGRKLLGAGIVI | 2 | 46 |
| | | 4805-6460\| | TEEKQVRVYTAAQSGTIVLRSFRVVSLDRYSCMESTI | | |
| | | Organism: Avian | ESYNKTVYNILAPLGDAIRRIQASGVSVERIREGRIF | | |
| | | paramyxovirus 9\| | GAILGGVALGVATAAQITAAIALIQANENAKNILRIK | | |
| | | Strain Name: duck/ | DSITKTNEAVRDVTNGVSQLTIAVGKLQDFVNKEFNK | | |
| | | New York/22/1978\| | TTEAINCVQAAQQLGVELSLYLTEITTVFGPQITSPA | | |
| | | Protein Name: | LSKLTIQALYNLAGVSLDVLLGRLGADNSQLSSLVSS | | |
| | | fusion protein\| | GLITGQPILYDSESQILALQVSLPSISDLRGVRATYL | | |
| | | Gene Symbol: F | DTLAVNTAAGLASAMIPKVVIQSNNIVEELDTTACIA | | |
| | | | AEADLYCTRITTFPIASAVSACILGDVSQCLYSKTNG | | |
| | | | VLTTPYVAVKGKIVANCKHVTCRCVDPTSIISQNYGE | | |
| | | | AATLIDDQLCKVINLDGVSIQLSGTFESTYVRNVSIS | | |
| | | | ANKVIVSSSIDISNELENVNSSLSSALEKLDESDAAL | | |
| | | | SKVNVHLTSTSAMATYIVLTVIALILGFVGLGLGCFA | | |
| | | | MIKVKSQAKTLLWLGAHADRSYILQSKPAQSST | | |
| NC_025403 | 4826-6649 | gb: NC_025403: | MWIMIILSLFQIIPGVTPINSKVLTQLGVITKHTRQL | 2 | 47 |
| | | 4826-6649\| | KFYSHSTPSYLVVKLVPTINTESTVCNFTSLSRYKDS | | |
| | | Organism: Achimota | VRELITPLAKNIDNLNSILTIPKRRKRMAGVVIGLAA | | |
| | | virus 1\|Strain Name: | LGVAAAAQATAAVALIEAKKNTEQIQALSESIQNTNK | | |
| | | UNKNOWN-NC_025403\| | AVSSIEKGLSSAAIAVQAIQNQINNVINPALTALDCG | | |
| | | Protein Name: fusion | VTDAQLGNILNLYLIKTLTVFQKQITNPALQPLSIQA | | |
| | | protein\|Gene Symbol: | LNIIMQETSSVLRNFTKTDEIEHTDLLTSGLITGQVV | | |
| | | F | GVNLTNLQLIIAAFIPSIAPLNQAYILDFIRITVNIN | | |
| | | | NSESMIQIPERIMEHGISLYQFGGDQCTFSDWSAYCP | | |
| | | | YSDATLMAPGLQNCFRGQAADCVFSTVMGSFPNRFVS | | |
| | | | VQGVFYVNCKFIRCACTQPQRLITQDDSLSLTQIDAK | | |
| | | | TCRMLTLGFVQFSINEYANVTYSFKNNVTAGQLIMTN | | |
| | | | PIDLSTEIKQMNDSVDEAARYIEKSNAALNKLMYGGR | | |
| | | | SDIVTTVLLVGFILLVVYVIFVTYILKILMKEVARLR | | |
| | | | NSNHPDLIKPYNYPM | | |
| NC_025404 | 4772-6647 | gb: NC_025404: | MLNSFYQIICLAVCLTTYTVISIDQHNLLKAGVIVKS | 2 | 48 |
| | | 4772-6647\| | IKGLNFYSRGQANYIIVKLIPNVNVTDTDCDIGSIKR | | |
| | | Organism\| Achimota | YNETVYSLIKPLADNIDYLRTQFAPTKRKKRFAGVAI | | |
| | | virus 2\|Strain Name: | GLTALGVATAAQVTAAVALVKAQENARKLDALADSIQ | | |
| | | UNKNOWN-NC_025404\| | ATNEAVQDLSTGLQAGAIAIQAIQSEINHVINPALER | | |
| | | Protein Name: | LSCEIIDTRVASILNLYLIRLTTVFHRQLVNPALTPL | | |
| | | fusion protein\| | SIQALNHLLQGETEGLVKNESKMTDSKIDLLMSGLIT | | |
| | | Gene Symbol: F | GQVVGVNIKHMQLMIAVFVPTTAQLPNAYVINLLTIT | | |
| | | | ANINNSEVLVQLPNQILERSGIIYQFRGKDCVSSPNH | | |
| | | | MYCPYSDASILSPELQLCLQGRLEMCLFTQVVGSFPT | | |
| | | | RFASDKGIVYANCRHLQCACSEPEGIIYQDDTSAITQ | | |
| | | | IDASKCSTLKLDMLTFKLSTYANKTFDASFSVGKDQM | | |
| | | | LVTNLLDLSAELKTMNASVAHANKLIDKSNLLIQSNA | | |
| | | | LIGHSNTIFIVVIVILAVMVLYLIIVTYIIKVIMVEV | | |
| | | | SRLKRMNIYSIDK | | |
| NC_025410 | 4958-6751 | gb: NC_025410: | MVTIIKPLILLVTVILQISGHIDTTALTSIGAVIASS | 2 | 49 |
| | | 4958-6751\| | KEIMYYAQSTPNYIVIKLIPNLPNIPSQCNFSSIAYY | | |
| | | Organism: Tihoko | NKTLLDLFTPISDNINMLHQRLSNTGRNRRFAGVAIG | | |
| | | virus 1\|Strain Name: | LAALGVATAAQVTAAFALVEAKSNTAKIAQIGQAIQN | | |
| | | UNKNOWN-NC_025410\| | TNAAINSLNAGIGGAVTAIQAIQTQINGIITDQINAA | | |
| | | Protein Name: | TCTALDAQIGTLLNMYLLQLTTTFQPQIQNPALQPLS | | |
| | | fusion protein\| | IQALHRIMQGTSIVLSNLTDSSKYGLNDALSAGLITG | | |
| | | Gene Symbol: F | QIVSVDLRLMQITIAANVPTLSRLENAIAHDIMRITT | | |
| | | | NVNNTEVIVQLPETIMEHAGRLYQFNKDHCLSSTQRF | | |
| | | | FCPYSDAKLLTSKISSCLSGIRGDCIFSPVVGNFATR | | |
| | | | FISVKGVIIANCKFIRCTCLQPEGIISQLDDHTLTVI | | |
| | | | DLKLCNKLDLGLIQFDLQVLSNISYEMTLNTSQNQLI | | |
| | | | LTDPLDLSSELQTMNQSINNAANFIEKSNSLLNSSTY | | |
| | | | EFNRSVALLVALILLSLTILYVIVLTCVVKLLVHEVS | | |
| | | | KNRRHIQDLESHHK | | |
| NC_028249 | 4850-7055 | gb: NC_028249: | MTRVKKLPVPTNPPMHHSLDSPFLNPEHATGKISITD | 2 | 50 |
| | | 4850-7055\| | DTSSQLTNFLYHKYHKTTINHLSRTISGTDPPSAKLN | | |
| | | Organism: Phocine | KFGSPILSTYQIRSALWWIAMVILVHCVMGQIHWTNL | | |
| | | distemper virus\| | STIGIIGTDSSHYKIMTRSSHQYLVLKLMPNVSIIDN | | |
| | | Strain Name: PDV/ | CTKAELDEYEKLLNSVLEPINQALTLMTKNVKSLQSL | | |
| | | Wadden_Sea.NLD/ | GSGRRQRRFAGVVIAGAALGVATAAQITAGVALYQSN | | |
| | | 1988\|Protein Name: | LNAQAIQSLRASLEQSNKAIDEVRQASQNIIIAVQGV | | |
| | | fusion protein\|Gene | QDYVNNEIVPALQHMSCELIGQRLGLKLLRYYTELLS | | |
| | | Symbol: F | VFGPSLRDPVSAEISIQALSYALGGEIHKILEKLGYS | | |
| | | | GNDMVAILETKGIRAKITHVDLSGKFIVLSISYPTLS | | |
| | | | EVKGVVVHRLEAVSYNIGSQEWYTTVPRYVATNGYLI | | |
| | | | SNFDESSCVFVSESAICSQNSLYPMSPILQQCLRGET | | |
| | | | ASCARTLVSGTLGNKFILSKGNIIANCASILCKCHST | | |
| | | | SKIINQSPDKLLTFIASDTCSLVEIDGVTIQVGSRQY | | |
| | | | PDVVYASKVILGPAISLERLDVGTNLGSALKKLNDAK | | |
| | | | VLIESSDQILDTVKNSYLSLGTLIALPVSIGLGLILL | | |
| | | | LLICCCKKRYQHLFSQSTKVAPVFKPDLTGTSKSYVR | | |
| | | | SL | | |
| NC_028362 | 5217-6842 | gb: NC_028362: | MIKKIICIFSMPILLSFCQVDIIKLQRVGILVSKPKS | 2 | 51 |
| | | 5217-6842\| | IKISQNFETRYLVLNLIPNIENAQSCGDQQIKQYKKL | | |
| | | Organism: Caprine | LDRLIIPLYDGLRLQQDIIVVDNNLKNNTNHRAKRFF | | |
| | | parainfluenza virus | GEIIGTIALGVATSAQITAAVALVEAKQARSDIERVK | | |
| | | 3\|Strain Name: | NAVRDTNKAVQSIQGSVGNLIVAVKSVQDYVNNEIVP | | |
| | | JS2013\|Protein Name: | SIKRLGCEAAGLQLGIALTQHYSELTNIFGDNIGTLK | | |
| | | fusion protein\| | EKGIKLQGIASLYHTNITEIFTTSTVDQYDIYDLLFT | | |
| | | Gene Symbol: F | ESIKMRVIDVDLNDYSITLQVRLPLLTKISDAQIYNV | | |
| | | | DSVSYNIGGTEWYIPLPRNIMTKGAFLGGANLQDCIE | | |
| | | | SFSDYICPSDPGFILNRDIENCLSGNITQCPKTLVIS | | |
| | | | DIVPRYAFVDGGVIANCLSTTCTCNGIDNRINQAPDQ | | |
| | | | GIKIITYKDCQTIGINGMLFKTNQEGTLAAYTPVDIT | | |
| | | | LNNSVNLDPIDLSIELNRARSDLAESKEWIKRSEAKL | | |
| | | | DSVGSWYQSSTTEIIQIVMIIVLFIINIIVLIVLIKY | | |
| | | | SRSQNQSMNNHMNEPYILTNKVQ | | |
| AF079780 | 5919-7580 | gb: AF079780\| | MASLLKTICYIYLITYAKLEPTPKSQLDLDSLASIGV | 1 | 52 |
| | | Organism: Tupaia | VDAGKYNYKLMTTGSEKLMVIKLVPNITYATNCNLTA | | |
| | | paramyxovirus\| | HTAYTKMIERLLTPINQSLYEMRSVITERDGGTIFWG | | |
| | | Strain Name: | AIIAGAALGVATAAAITAGVALHRAEQNARNIAALKD | | |
| | | UNKNOWN-AF079780\| | ALRNSNEAIQHLKDAQGHTVLAIQGLQEQINNNIIPK | | |
| | | Protein Name: | LKESHCLGVNNQLGLLLNQYYSEILTVFGPNLQNPVS | | |
| | | fusion protein\| | ASLTIQAIAKAFNGDFNSLMTNLNYDPTDLLDILESN | | |
| | | Gene Symbol: F | SINGRIIDVNLNEKYIALSIEIPNFITLTDAKIQTFN | | |
| | | | RITYGYGSNEWLTLIPDNILEYGNLISNVDLTSCVKT | | |
| | | | KSSYICNQDTSYPISSELTRCLRGDTSSCPRTPVVNS | | |
| | | | RAPTFALSGGHIYANCAKAACRCEKPPMAIVQPATST | | |
| | | | LTFLTEKECQEVVIDQINIQLAPNRLNKTIITDGIDL | | |
| | | | GPEVIINPIDVSAELGNIELEMDKTQKALDRSNKILD | | |
| | | | SMITEVTPDKLLIAMIVVFGILLLWLFGVSYYAFKIW | | |
| | | | SKLHFLDSYVYSLRNPSHHRSNGHQNHSFSTDISG | | |
| EU403085 | 4664-6585 | gb: EU403085: | MQPGSALHLPHLYIIIALVSDGTLGQTAKIDRLIQAG | 1 | 53 |
| | | 4664-6585\| | IVLGSGKELHISQDSGTLDLFVRLLPVLPSNLSHCQL | | |
| | | Organism: Avian | EAITQYNKTVTRLLAPIGKNLEQVLQARPRGRLFGPI | | |
| | | paramyxovirus 14\| | IGSIALGVATSAQITAAIALVRAQQNANDILALKNAL | | |
| | | Strain Name: | QSSNEAIRQLTYGQDKQLLAISKIQKAVNEQILPALD | | |
| | | APMV14/duck/Japan/ | QLDCAVLGTKLAVQLNLYLIEMTTIFGEQINNPVLAT | | |
| | | 11OG0352/2011\| | IPLSYILRLTGAELNNVLMKQARSSLSLVQLVSKGLL | | |
| | | Protein Name: fusion | SGQVIGYDPSVQGLIIRVNLMRTQKIDRALVYQPYVL | | |
| | | protein\|Gene | PITLNSNIVTPIAPECVIQKGTIIEGMSRKDCTELEQ | | |
| | | Symbol: F | DIICRTVTTYTLARDTRLCLQGNISSCRYQQSGTQLH | | |
| | | | TPFITYNGAVIANCDLVSCRCLRPPMIITQVKGYPLT | | |
| | | | IITRSVCQELSVDNLVLNIETHHNFSLNPTIIDPLTR | | |
| | | | VIATTPLEIDSLIQEAQDHANAALAKVEESDKYLRAV | | |
| | | | TGGNYSNWYIVLVIVLLFGNLGWSLLLTVLLCRSRKQ | | |
| | | | QRRYQQDDSVGSERGVGVGTIQYMS | | |
| KX258200 | 4443-6068 | gb: KX258200: | MEKGTVLFLAALTLYNVKALDNTKLLGAGIASGKEHE | 1 | 54 |
| | | 4443-6068\| | LKIYQSSVNGYIAVKLIPFLPSTKRECYNEQLKNYNA | | |
| | | Organism: Avian | TINRLMGPINDNIKLVLSGVKTRTREGKLIGAIIGTA | | |
| | | paramyxovirus 14\| | ALGLATAAQVTAAIALEQAQDNARAILTLKESIRNTN | | |
| | | Strain Name: APMV14/ | NAVSELKTGLSEVSIALSKTQDYINTQIMPALSNLSC | | |
| | | duck/Japan/ | EIVGLKIGIQLSQYLTEVTAVFGNQITNPALQPLSMQ | | |
| | | 11OG0352/2011\| | ALYQLCGGDFSLLLDKIGADRNELESLYEANLVTGRI | | |
| | | Protein Name: fusion | VQYDTADQLVIIQVSIPSVSTLSGYRVTELQSISVDM | | |
| | | protein\|Gene | DHGEGKAVIPRYIVTSGRVIEEMDISPCVLTATAVYC | | |
| | | Symbol: F | NRLLTTSLPESVLKCLDGDHSSCTYTSNSGVLETRYI | | |
| | | | AFDGMLIANCRSIVCKCLDPPYIIPQNKGKPLTIISK | | |
| | | | EVCKKVTLDGITLLIDAEFTGEYGLNITIGPDQFAPS | | |
| | | | GALDISTELGKLNNSINKAEDYIDKSNELLNRVNVDI | | |
| | | | VNDTAVIVLCVMSALVVVWCIGLTVGLIYVSKNTLRA | | |
| | | | VAIKGTSIENPYVSSGKHAKNSS | | |
| KY511044 | 4592-6247 | gb: KY511044: | MIFTMYHVTVLLLLSLLTLPLGIQLARASIDGRQLAA | 1 | 55 |
| | | 4592-6247\| | AGIVVTGEKAINLYTSSQTGTIVVKLLPNVPQGREAC | | |
| | | Organism: Avian | MRDPLTSYNKTLTSLLSPLGEAIRRIHESTTETAGLV | | |
| | | paramyxovirus | QARLVGAIIGSVALGVATSAQITAAAALIQANKNAEN | | |
| | | UPO216\|Strain Name: | ILKLKQSIAATNEAVHEVTDGLSQLAVAVGKMQDFIN | | |
| | | APMV-15/WB/Kr/ | TQFNNTAQEIDCIRISQQLGVELNLYLTELTTVFGPQ | | |
| | | UPO216/2014\|Protein | ITSPALSPLSIQALYNLAGGNLDVLLSKIGVGNNQLS | | |
| | | Name: fusion | ALISSGLISGSPILYDSQTQLLGIQVTLPSVSSLNNM | | |
| | | protein\|Gene | RAIFLETLSVSTDKGFAAALIPKVVTTVGTVTEELDT | | |
| | | Symbol: F | SYCIETDIDLFCTRIVTFPMSPGIYACLNGNTSECMY | | |
| | | | SKTQGALTTPYMSVKGSIVANCKMTTCRCADPASIIS | | |
| | | | QNYGEAVSLIDSSVCRVITLDGVTLRLSGSFDSTYQK | | |
| | | | NITIRDSQVIITGSLDISTELGNVNNSINNALDKIEE | | |
| | | | SNQILESVNVSLTSTNALIVYIICTALALICGITGLI | | |
| | | | LSCYIMYKMRSQQKTLMWLGNNTLDQMRAQTKM | | |
| NC_025360 | 6104-8123 | gb: NC_025360: | MDGPKFRFVLLILLTAPARGQVDYDKLLKVGIFEKGT | 1 | 56 |
| | | 6104-8123\| | ANLKISVSSQQRYMVIKMMPNLGPMNQCGIKEVNLYK | | |
| | | Organism: Atlantic | ESILRLITPISTTLNYIKSEIQVEREVALQPNGTIVR | | |
| | | salmon paramyxo- | FFGLIVAAGALTLATSAQITAGIALHNSLENAKAIKG | | |
| | | virus\|Strain Name: | LTDAIKESNLAIQKIQDATAGTVIALNALQDQVNTNI | | |
| | | ASPV/Yrkje371/95\| | IPAINTLGCTAAGNTLGIALTRYYSELIMIFGPSLGN | | |
| | | Protein Name: fusion | PVEAPLTIQALAGAFNGDLHGMIREYGYTPSDIEDIL | | |
| | | protein\|Gene | RTNSVTGRVIDVDLVGMNIVLEINLPTLYTLRDTKIV | | |
| | | Symbol: F | NLGKITYNVDGSEWQTLVPEWLAIRNTLMGGVDLSRC | | |
| | | | VVSSRDLICKQDPVFSLDTSIISCLNGNTESCPRNRV | | |
| | | | VNSVAPRYAVIRGNILANCISTTCLCGDPGVPIIQKG | | |
| | | | DNTLTAMSINDCKLVGVDGYVFRPGPKAVNVTFNLPH | | |
| | | | LNLGPEVNVNPVDISGALGKVEQDLASSRDHLAKSEK | | |
| | | | ILSGINPNIINTEMVLVAVILSLVCAMVVIGIVCWLS | | |
| | | | ILTKWVRSCRADCRRPNKGPDLGPIMSSQDNLSF | | |

In some embodiments, a fusogen described herein comprises an amino acid sequence of Table 5, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a fusogen described herein comprises an amino acid sequence having at least 80% identity to any amino acid sequence of Table 5. In some embodiments, a nucleic acid sequence described herein encodes an amino acid sequence of Table 5, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

In some embodiments, a fusogen described herein comprises an amino acid sequence set forth in any one of SEQ ID NOS: 57-132, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a fusogen described herein comprises an amino acid sequence having at least 80% identity to an amino acid sequence set forth in any one of SEQ ID NOS: 57-132. In some embodiments, nucleic acid sequence described herein encodes an amino acid sequence set forth in anyone of SEQ ID NOS: 57-132, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

TABLE 5
Paramyxovirus protein G, H, and HN sequence clusters. Column 1, Genbank ID
includes the Genbank ID of the whole genome sequence of the virus that is
the centroid sequence of the cluster. Column 2, nucleotides of CDS provides
the nucleotides corresponding to the CDS of the gene in the whole genome.
Column 3, Full Gene Name, provides the full name of the gene including Genbank
ID, virus species, strain, and protein name. Column 4, Sequence, provides the
amino acid sequence of the gene. Column 5, #Sequences/Cluster, provides the
number of sequences that cluster with this centroid sequence.

| | Nucleo- | | | | SEQ |
|---|---|---|---|---|---|
| Genbank | tides | | | #Sequences/ | ID |
| ID | of CDS | Ful sequence ID | Sequence | Cluster | NO |
| KU950686 | 4643-5638 | gb: KU950686: | MSKTKDQRTAKTLERTWDTLNHLLFISSCLYKLN | 706 | 57 |
| | | 4643-5638\|Organism: | LKSIAQITLSILAMIISTSLIIAAIIFIASANHK | | |
| | | Human respiratory | VTLTTAIIQDATNQIKNTTPTYLTQNPQLGISFS | | |
| | | syncytial virus\|Strain | NLSGTTLQSTTILASTTPSAESTPQSTTVKIINT | | |
| | | Name: RSVA/Homo | TTTQILPSKPTTKQRQNKPQNKPNNDFHFEVFNF | | |
| | | sapiens/USA/TH_10506/ | VPCSICSNNPTCWAICKRIPNKKPGKKTTTKPTK | | |
| | | 2014/Protein Name: | KPTLKTTKKDPKPQTTKPKEALTTKPTGKPTINT | | |
| | | attachment glyco- | TKTNIRTTLLTSNTKGNPEHTSQEETLHSTTSEG | | |
| | | protein\|Gene Symbol: G | YLSPSQVYTTSGQEETLHSTTSEGYLSPSQVYTT | | |
| | | | SEYLSQSLSSSNTTK | | |
| AB524405 | 6424-8274 | gb: AB524405: | MERGVSQVALENDEREAKNTWRLVFRVTVLFLTI | 418 | 58 |
| | | 6424-8274\|Organism: | VTLAISAAALAFSMNASTPQDLEGIPVAISKVED | | |
| | | Newcastle disease | KITSALGASQDVMDRIYKQVALESPLALLNTEST | | |
| | | virus\|Strain Name: | IMNALTSLSYQINGAANASGCGAPVPDPDYIGGI | | |
| | | Goose/Alaska/415/91\| | GKELIVDDTSDVTSFYPSAFQEHLNFIPAPTTGS | | |
| | | Protein Name: | GCTRIPSFDMSATHYCYTHNVILSGCRDHSHSHQ | | |
| | | hemagglutinin- | YLALGVLRTSATGRVFFSTLRSINLDDTQNRKSC | | |
| | | neuraminidase protein\| | SVSATPLGCDMLCSKVTETEEEDYQSTDPTLMVH | | |
| | | Gene Symbol: HN | GRLGFDGQYHERDLDVHTLFGDWVANYPGVGGGS | | |
| | | | FINNRVWFPVYGGLKPGSPTDKRQEGQYAIYKRY | | |
| | | | NDTCPDDQEYQVRMAKSAYKPNRFGGKRVQQAIL | | |
| | | | SIGVSTTLADDPVLTVTSNTITLMGAEGRVMTVG | | |
| | | | TSHYLYQRGSSYYSPAILYPLTIANKTATLQDPY | | |
| | | | KFNAFTRPGSVPCQASARCPNSCVTGVYTDPYPI | | |
| | | | VFHKNHTLRGVFGTMLDDEQARLNPVSAVFDSIA | | |
| | | | RSRVTRVSSSSTKAAYTTSTCFKVVKTGKVYCLS | | |
| | | | IAEISNTLFGEFRIVPLLVEILRDEGRSEARSAL | | |
| | | | TTQGHPGWNDEVVDPIFCAVTNQTDHRQKLEEYA | | |
| | | | QSWP | | |
| JQ582844 | 4686-5636 | gb: JQ582844: | MSKNKNQRTARTLEKTWDTLNHLIVISSCLYKLN | 278 | 59 |
| | | 4686-5636\|Organism: | LKSIAQIALSVLAMIISTSLIIAAIIFIISANHK | | |
| | | Human respiratory | VTLTTVTVQTIKNHTEKNITTYLTQVSPERVSPS | | |
| | | syncytial virus\|Strain | KQPTTTPPIHTNSATISPNTKSETHHTTAQTKGR | | |
| | | Name: NH1067\|Protein | TTTPTQNNKPSTKPRPKNPPKKPKDDYHFEVFNF | | |
| | | Name: receptor-binding | VPCSICGNNQLCKSICKTIPNNKPKKKPTTKPTN | | |
| | | glycoprotein\|Gene | KPPTKTTNKRDPKTPAKTLKKETTINPTTKKPTP | | |
| | | Symbol: G | KTTERDTSTPQSTVLDTTTSKHTERDTSTPQSTV | | |
| | | | LDTTTSKHTIQQQSLHSITPENTPNSTQTPTASE | | |
| | | | PSTSNSTQKL | | |
| AB256456 | 7271-9136 | gb: AB254456: | MSPHRDRINAFYRDNPHPKGSRIVINREHLMIDR | 128 | 60 |
| | | 7271-9136\|Organism: | PYVLLAVLFVMFLSLIGLLAIAGIRLHRAAIYTA | | |
| | | Measles virus\|Strain | EIHKSLSTNLDVTNSIEHQVKDVLTPLFKIIGDE | | |
| | | Name: SSPE-Kobe-1\| | VGLRTPQRFTDLVKFISDKIKFLNPDREYDFRDL | | |
| | | Protein Name: | TWCINPPERIKLDYDQYCADVAAEELMNALVNST | | |
| | | Hemagglutinin\|Gene | LLEARATNQFLAVSKGNCSGPTTIRGQFSNMSLS | | |
| | | Symbol: H | LLDLYLSRGYNVSSIVTMTSQGMYGGTYLVGKPN | | |
| | | | LSSKGSELSQLSMHRVFEVGVIRNPGLGAPVFHM | | |
| | | | TNYFEQPVSNDFSNCMVALGELRFAALCHREDSV | | |
| | | | TVPYQGSGKGVSFQLVKLGVWKSPTDMQSWVPLS | | |
| | | | TDDPVIDRLYLSSHRGVIADNQAKWAVPTTRTDD | | |
| | | | KLRMETCFQQACKGKNQALCENPEWAPLKDNRIP | | |
| | | | SYGVLSVNLSLTVELKIKIASGFGPLITHGSGMD | | |
| | | | LYKTNHDNVYWLTIPPMKNLALGVINTLEWIPRF | | |
| | | | KVSPNLFTVPIKEAGEDCHAPTYLPAEVDGDVKL | | |
| | | | SSNLVILPGQDLQYVLATYDTSRVEHAVVYYVYS | | |
| | | | PSRSFSYFYPFRLPIKGVPIELQVECFTWDQKLW | | |
| | | | CRHFCVLADSESGGHITHSGMVGMGVSCTVTRED | | |
| | | | GTNRRQGCQ | | |
| AB040874 | 6614-8362 | gb: AB040774: | MEPSKLFTMSDNATFAPGPVINAADKKTFRTCFR | 87 | 61 |
| | | 6614-8362\|Organism: | ILVLSVQAVTLILVIVTLGELVRMINDQGLSNQL | | |
| | | Mumps virus\|Strain | SSIADKIRESATMIASAVGVMNQVIHGVTVSLPL | | |
| | | Name: Miyaharal\| | QIEGNQNQLLSTLATICTGKKQVSNCSTNIPLVN | | |
| | | Protein Name: | DLRFINGINKFIIEDYATHDFSIGHPLNMPSFIP | | |
| | | hemagglutinin- | TATSPNGCTRIPSFSLGKTHWCYTHNVINANCKD | | |
| | | neuraminidase\|Gene | HTSSNQYISMGILVQTASGYPMFKTLKIQYLSDG | | |
| | | Symbol: HN | LNRKSCSIATVPDGCAMYCYVSTQLETDDYAGSS | | |
| | | | PPTQKLTLLFYNDTVTERTISPTGLEGNWATLVP | | |
| | | | GVGSGIYFENKLIFPAYGGVLPNSSLGVKSAREF | | |
| | | | FRPVNPYNPCSGPQQDLDQRALRSYFPSYFSNRR | | |
| | | | VQSAFLVCAWNQILVTNCELVVPSNNQTLMGAEG | | |
| | | | RVLLINNRLLYYQRSTSWWPYELLYEISFTFTNS | | |
| | | | GQSSVNMSWIPIYSFTRPGSGNCSGENVCPTACV | | |
| | | | SGVYLDPWPLTPYSHQSGINRNFYFTGALLNSST | | |
| | | | TRVNPTLYVSALNNLKVLAPYGNQGLFASYTTTT | | |
| | | | CFQDTGDASVYCVYIMELASNIVGEFQILPVLTR | | |
| | | | LTIT | | |
| AB736166 | 6709-8427 | gb: AB736166: | MEYWKHTNHGKDAGNELETATATHGNRLTNKITY | 78 | 62 |
| | | 6709-8427\|Organism: | ILWTITLVLLSIVFIIVLINSIKSEKAHESLLQD | | |
| | | Human respirovirus 3\| | INNEFMEVTEKIQVASDNTNDLIQSGVNTRLLTI | | |
| | | Strain Name: ZMLS/ | QSHVQNYIPISLTQQISDLRKFISEITIRNDNQE | | |
| | | 2011\|Protein Name: | VPPQRITHDVGIKPLNPDDFWRCTSGLPSLMRTP | | |
| | | hemagglutinin- | KIRLMPGPGLLAMPTTVDGCVRTPSLVINDLIYA | | |
| | | neuraminidase\|Gene | YTSNLITRGCQDIGKSYQVLQIGIITVNSDLVPD | | |
| | | Symbol: HN | LNPRISHTFNINDNRKSCSLALLNTDVYQLCSTP | | |
| | | | KVDERSDYASSGIEDIVLDIVNYDGSISTTRFKN | | |
| | | | NNISFDQPYAALYPSVGPGIYYKGKIIFLGYGGL | | |
| | | | EHPINENAICNTTGCPGKTQRDCNQASHSPWFSD | | |
| | | | RRMVNSIIVVDKGLNSVPKLKVWTISMRQNYWGS | | |
| | | | EGRLLLLGNKIYIYTRSTSWHSKLQLGIIDITDY | | |
| | | | SDIRIKWTWHNVLSRPGNNECPWGHSCPDGCITG | | |
| | | | VYTDAYPLNPTGSIVSSVILDSQKSRVNPVITYS | | |
| | | | TATERVNELAIRNKTLSAGYTTTSCITHYNKGYC | | |
| | | | FHIVEINHKSLNTFQPMLFKTEIPKSCS | | |
| KJ627396 | 6166-6885 | gb: KJ627396: | MEVKVENIRAIDMLKARVKNRVARSKCFKNASLI | 71 | 63 |
| | | 6166-6885\|Organism: | LIGITTLSIALNIYLIINYTIQKTTSESEHHTSS | | |
| | | Human metapneumovirus\| | PPTESNKETSTIPIDNPDITPNSQHPTQQSTESL | | |
| | | Strain Name: HMPV/ | TLYPASSMSPSETEPASTPGITNRLSLADRSTTQ | | |
| | | Homo sapiens/PER/ | PSESRTKTNSTVHKKNKKNISSTISRTQSPPRTT | | |
| | | FLI1305/2010/A\| | AKAVSRTTALRMSSTGERPTTTSVQSDSSTTAQN | | |
| | | Protein Name: | HEETGPANPQASVSTM | | |
| | | attachment glyco- | | | |
| | | protein G\|Gene | | | |
| | | Symbol: G | | | |
| AB475097 | 7079-8902 | gb: AB475097: | MLSYQDKVGAFYKDNARANSSKLSLVTEEQGGRR | 45 | 64 |
| | | 7079-8902\|Organism: | PPYLLFVLLILLVGILALLAIAGVRFRQVSTSNV | | |
| | | Canine distemper | EFGRLLKDDLEKSEAVHHQVMDVLTPLFKIIGDE | | |
| | | virus\|Strain Name: | IGLRLPQKLNEIKQFILQKTNFFNPNREFDFRDL | | |
| | | M25CR\|Protein Name: | HWCINPPSKIKVNFTNYCDAIGVRKSIASAANPI | | |
| | | hemagglutinin\|Gene | LLSALSGGRGDIFPPYRCSGATTSVGRVFPLSVS | | |
| | | Symbol: H | LSMSLISKTSEIISMLTAISDGVYGKTYLLVPDY | | |
| | | | IEREFDTQKIRVFEIGFIKRWLNDMPLLQTTNYM | | |
| | | | VLPENSKAKVCTIAVGELTLASLCVDESTVLLYH | | |
| | | | DSNGSQDSILVVTLGIFGATPMNQVEEVIPVAHP | | |
| | | | SVERIHITNHRGFIKDSVATWMVPALVSEQQEGQ | | |
| | | | KNCLESACQRKSYPMCNQTSWEPFGGVQLPSYGR | | |
| | | | LTLPLDASIDLQLNISFTYGPVILNGDGMDYYEN | | |
| | | | PLLDSGWLTIPPKNGTILGLINKASRGDQFTVTP | | |
| | | | HVLTFAPRESSGNCYLPIQTSQIMDKDVLTESNL | | |
| | | | VVLPTQNFRYVVATYDISRENHAIVYYVYDPIRT | | |
| | | | ISYTYPFRLTTKGRPDFLRIECFVWDDDLWCHQF | | |
| | | | YRFESDITNSTTSVEDLVRIRFSCNRSKP | | |
| AJ849636 | 7326-9155 | gb: AJ849636: | MSAQRERINAFYKDNPHNKNHRVILDRERLVIER | 34 | 65 |
| | | 7326-9155\|Organism: | PYILLGVLLVMFLSLIGLLAIAGIRLHRATVGTS | | |
| | | Pestes-des-petits- | EIQSRLNTNIELTESIDHQTKDVLTPLFKIIGDE | | |
| | | ruminants virus\|Strain | VGIRIPQKFSDLVKFISDKIKFLNPDREYDFRDL | | |
| | | Name: Turkey 2000\| | RWCMNPPERVKINFDQFCEYKAAVKSIEHIFESP | | |
| | | Protein Name: | LNKSKKLQSLTLGPGTGCLGRTVTRAHFSELTLT | | |
| | | hemagglutinin\|Gene | LMDLDLEMKHNVSSVFTVVEEGLFGRTYTVWRSD | | |
| | | Symbol: H | ARDPSTDLGIGHFLRVFEIGLVRDLGLGPPVFHM | | |
| | | | TNYLTVNMSDDYRRCLLAVGELKLTALCSSSETV | | |
| | | | TLGERGVPKREPLVVVILNLAGPTLGGELYSVLP | | |
| | | | TSDLMVEKLYLSSHRGIIKDDEANWVVPSTDVRD | | |
| | | | LQNKGECLVEACKTRPPSFCNGTGSGPWSEGRIP | | |
| | | | AYGVIRVSLDLASDPGVVITSVFGPLIPHLSGMD | | |
| | | | LYNNPFSRAVWLAVPPYEQSFLGMINTIGFPNRA | | |
| | | | EVMPHILTTEIRGPRGRCHVPIELSRRVDDDIKI | | |
| | | | GSNMVILPTIDLRYITATYDVSRSEHAIVYYIYD | | |
| | | | TGRSSSYFYPVRLNFKGNPLSLRIECFPWRHKVW | | |
| | | | CYHDCLIYNTITDEEVHTRGLTGIEVTCNPV | | |
| AB005795 | 6693-8420 | gb: AB005795: | MDGDRSKRDSYWSTSPGGSTTKLVSDSERSGKVD | 23 | 66 |
| | | 6693-8420\|Organism: | TWLLILAFTQWALSIATVIICIVIAARQGYSMER | | |
| | | Sendai virus\|Strain | YSMTVEALNTSNKEVKESLTSLIRQEVITRAANI | | |
| | | Name: Ohita\|Protein | QSSVQTGIPVLLNKNSRDVIRLIEKSCNRQELTQ | | |
| | | Name: hemagglutinin- | LCDSTIAVHHAEGIAPLEPHSFWRCPAGEPYLSS | | |
| | | neuraminidase | DPEVSLLPGPSLLSGSTTISGCVRLPSLSIGEAI | | |
| | | protein\| Gene Symbol: | YAYSSNLITQGCADIGKSYQVLQLGYISLNSDMF | | |
| | | HN | PDLNPVVSHTYDINDNRKSCSVVATGTRGYQLCS | | |
| | | | MPIVDERTDYSSDGIEDLVLDILDLKGRTKSHRY | | |
| | | | SNSEIDLDHPFSALYPSVGSGIATEGSLIFLGYG | | |
| | | | GLTTPLQGDTKCRIQGCQQVSQDTCNEALKITWL | | |
| | | | GGKQVVSVLIQVNDYLSERPRIRVTTIPITQNYL | | |
| | | | GAEGRLLKLGDQVYIYTRSSGWHSQLQIGVLDVS | | |
| | | | HPLTISWTPHEALSRPGNEDCNWYNTCPKECISG | | |
| | | | VYTDAYPLSPDAANVATVTLYANTSRVNPTIMYS | | |
| | | | NTTNIINMLRIKDVQLEAAYTTTSCITHFGKGYC | | |
| | | | FHIIEINQKSLNTLQPMLFKTSIPKLCKAES | | |
| AF457102 | 6903-8630 | gb: AF457102\| | MAEKGKTNSSYWSTTRNDNSTVNTHINTPAGRTH | 21 | 67 |
| | | Organism: Human | IWLLIATTMHTVLSFIIMILCIDLIIKQDTCMKT | | |
| | | parainfluenza virus 1 | NIMTVSSMNESAKIIKETITELIRQEVISRTINI | | |
| | | strain Washington/ | QSSVQSGIPILLNKQSRDLTQLIEKSCNRQELAQ | | |
| | | 1964\|Strain Name: | ICENTIAIHHADGISPLDPHDFWRCPVGEPLLSN | | |
| | | Washington 1964\| | NPNISLLPGPSLLSGSTTISGCVRLPSLSIGDAI | | |
| | | Protein Name: HN | YAYSSNLITQGCADIGKSYQVLQLGYISLNSDMY | | |
| | | glycoprotein\|Gene | PDLNPVISHTYDINDNRKSCSVIAAGTRGYQLCS | | |
| | | Symbol: HN | LPTVNETTDYSSEGIEDLVFDILDLKGKTKSHRY | | |
| | | | KNEDITFDHPFSAMYPSVGSGIKIENTLIFLGYG | | |
| | | | GLTTPLQGDTKCVINRCTNVNQSVCNDALKITWL | | |
| | | | KKRQVVNVLIRINNYLSDRPKIVVETIPITQNYL | | |
| | | | GAEGRLLKLGKKIYIYTRSSGWHSNLQIGSLDIN | | |
| | | | NPMTIKWAPHEVLSRPGNQDCNWYNRCPRECISG | | |
| | | | VYTDAYPLSPDAVNVATTTLYANTSRVNPTIMYS | | |
| | | | NTSEIINMLRLKNVQLEAAYTTTSCITHFGKGYC | | |
| | | | FHIVEINQASLNTLQPMLFKTSIPKICKITS | | |
| KJ627397 | 6146-6888 | gb: KJ627397: | MEVRVENIRAIDMFKAKMKNRIRSSKCYRNATLI | 21 | 68 |
| | | 6146-6888\|Organism: | LIGLTALSMALNIFLIIDYATLKNMTKVEHCVNM | | |
| | | Human metapneumovirus\| | PPVEPSKKSPMTSAADLNTKLNPQQATQLTTEDS | | |
| | | Strain Name: HPMPV/ | TSLAATSENHLHTETTPTSDATISQQATDEHTTL | | |
| | | Homo sapeins/PER/ | LRPINRQTTQTTTEKKPTGATTKKDKEKETTTRT | | |
| | | FPP00098/2010/B\| | TSTAATQTLNTTNQTSNGREATTTSARSRNGATT | | |
| | | Protein Name: | QNSDQTIQAADPSSKPYHTQTNTTTAHNTDTSSL | | |
| | | attachment | SS | | |
| | | attachment glyco- | | | |
| | | prtein G\|Gene | | | |
| | | Symbol: G | | | |
| AF017149 | 8913-10727 | gb: AF017149\| | MMADSKLVSLNNNLSGKIKDQGKVIKNYYGTMDI | 14 | 69 |
| | | Organism: Hendra | KKINDGLLDSKILGAFNTVIALLGSIIIIVMNIM | | |
| | | virus\|Strain Name: | IIQNYTRTTDNQALIKESLQSVQQQIKALTDKIG | | |
| | | UNKNOWN-AF017149\| | TEIGPKVSLIDTSSTITIPANIGLLGSKISQSTS | | |
| | | Protein Name: | SINENVNDKCKFTLPPLKIHECNISCPNPLPFRE | | |
| | | glycoprotein\|Gene | YRPISQGVSDLVGLPNQICLQKTTSTILKPRLIS | | |
| | | Symbol: G | YTLPINTREGVCITDPLLAVDNGFFAYSHLEKIG | | |
| | | | SCTRGIAKQRIIGVGEVLDRGDKVPSMFMTNVWT | | |
| | | | PPNPSTIHHCSSTYHEDFYYTLCAVSHVGDPILN | | |
| | | | STSWTESLSLIRLAVRPKSDSGDYNQKYIAITKV | | |
| | | | ERGKYDKVMPYGPSGIKQGDTLYFPAVGFLPRTE | | |
| | | | FQYNDSNCPIIHCKYSKAENCRLSMGVNSKSHYI | | |
| | | | LRSGLLKYNLSLGGDIILQFIEIADNRLTIGSPS | | |
| | | | KIYNSLGQPVFYQASYSWDTMIKLGDVDTVDPLR | | |
| | | | VQWRNNSVISRPGQSQCPRFNVCPEVCWEGTYND | | |
| | | | AFLIDRLNWVSAGVYLNSNQTAENPVFAVFKDNE | | |
| | | | ILYQVPLAEDDTNAQKTITDCFLLENVIWCISLV | | |
| | | | EIYDTGDSVIRPKLFAVKIPAQCSES | | |
| AF212302 | 8943-10751 | gb: AF213302\| | MPAENKKVRFENTTSDKGKIPSKVIKSYYGTMDI | 14 | 70 |
| | | Oganism: Nipah virus\| | KKINEGLLDSKILSAFNTVIALLGSIVIIVMNIM | | |
| | | Strain Name: | IIQNYTRSTDNQAVIKDALQGIQQQIKGLADKIG | | |
| | | UNKNOWN-AF212302\| | TEIGPKVSLIDTSSTITIPANIGLLGSKISQSTA | | |
| | | Protein Name: | SINENVNEKCKFTLPPLKIHECNISCPNPLPFRE | | |
| | | attachment glyco- | YRPQTEGVSNLVGLPNNICLQKTSNQILKPKLIS | | |
| | | protein\|Gene Symbol: | YTLPVVGQSGTCITDPLLAMDEGYFAYSHLERIG | | |
| | | G | SCSRGVSKQRIIGVGEVLDRGDEVPSLFMTNVWT | | |
| | | | PPNPNTVYHCSAVYNNEFYYVLCAVSTVGDPILN | | |
| | | | STYWSGSLMMTRLAVKPKSNGGGYNQHQLALRSI | | |
| | | | EKGRYDKVMPYGPSGIKQGDTLYFPAVGFLVRTE | | |
| | | | FKYNDSNCPITKCQYSKPENCRLSMGIRPNSHYI | | |
| | | | LRSGLLKYNLSDGENPKVVFIEISDQRLSIGSPS | | |
| | | | KIYDSLGQPVFYQASFSWDTMIKFGDVLTVNPLV | | |
| | | | VNWRNNTVISRPGQSQCPRFNTCPEICWEGVYND | | |
| | | | AFLIDRINWISAGVFLDSNQTAENPVFTVFKDNE | | |
| | | | ILYRAQLASEDTNAQKTITNCFLLKNKIWCISLV | | |
| | | | EIYDTGDNVIRPKLFAVKIPEQCT | | |
| EU439428 | 6751-8638 | gb: EU439428: | MEYWKHTNSTKDTNNELGTTRDRHSSKATNIIMY | 14 | 71 |
| | | 6751-8638\|Organism: | IFWTTTSTILSVIFIMILINLIQENNHNKLMLQE | | |
| | | Swine parainfluenza | IKKEFAVIDTKIQKTSDDISTSIQSGINTRLLTI | | |
| | | virus 3\|Strain Name: | QSHVQNYIPLSLTQQMSDLRKFINDLTTKREHQE | | |
| | | 92-7783_ISU-92\| | VPIQRMTHDSGIEPLNPDKFWRCTSGNPSLTSSP | | |
| | | Protein Name: | KIRLIPGPGLLATSTTVNGCIRIPSLAINNLIYA | | |
| | | hemagglutinin- | YTSNLITQGCQDIGKSYQVLQIGIITINSDLVPD | | |
| | | neuraminidase HN\| | LNPRVTHTFNIDDNRKSCSLALLNTDVYQLCSTP | | |
| | | Gene Symbol: HN | KVDERSDYASTGIEDIVLDIVTSNGLIITTRFTN | | |
| | | | NNITFDKPYAALYPSVGPGIYYKDKVIFLGYGGL | | |
| | | | EHEENGDVICNTTGCPGKTQRDCNQASYSPWFSN | | |
| | | | RRMVNSIIVVDKSIDTTFSLRVWTIPMRQNYWGS | | |
| | | | EGRLLLLGDRIYIYTRSTSWHSKLQLGVIDISDY | | |
| | | | NNIRINWTWHNVLSRPGNDECPWGHSCPDGCITG | | |
| | | | VYTDAYPLNPSGSVVSSVILDSQKSRENPIITYS | | |
| | | | TATNRVNELAIYNRTLPAAYTTTNCITHYDKGYC | | |
| | | | FHIVEINHRSLNTFQPMLFKTEVPKNCS | | |
| KF530164 | 6157-6906 | gb: KF530164: | MEVRVENIRAIDMFKAKIKNRIRSSRCYRNATLI | 14 | 72 |
| | | 6157-6906\|Organism: | LIGLTALSMALNIFLIIDHATLRNMIKTENCANM | | |
| | | Human metapneumovirus\| | PSAEPSKKTPMTSTAGPSTKPNPQQATQWTTENS | | |
| | | Strain Name: HPMV/ | TSPAATLEGHPYTGTTQTPDTTAPQQTTDKHTAL | | |
| | | AUS/172832788/2004/B\| | PKSTNEQITQTTTEKKTTRATTQKREKRKENTNQ | | |
| | | Protein Name: | TTSTAATQTTNTTNQTRNASETITTSDGPRIDTT | | |
| | | attachment glyco- | TQSSEQTARATEPGSSPYHARRGAGPR | | |
| | | protein G\|Gene | | | |
| | | Symbol: G | | | |
| AB910309 | 6960-8747 | gb: AB910309: | MKNINIKYYKDSNRYLGKILDEHKIVNSQLYSLS | 12 | 73 |
| | | 6960-8747\|Organism: | IKVITIIAIIVSLIATIMTIINATSGRTTLNSNT | | |
| | | Feline morbillivirus\| | DILLNQRDEIHSIHEMIFDRVYPLITAMSTELGL | | |
| | | Strain Name: SS1\| | HIPTLLDELTKAIDQKIKIMNPPVDTVTSDLSWC | | |
| | | Protein Name: | IKPPNGIIIDPKGYCESMELSKTYKLLLDQLDVS | | |
| | | hemagglutinin protein\| | RKKSLTINRKNINQCQLVDDSEIIFATVNIQSTP | | |
| | | Gene Symbol: H | RFLNFGHTVSNQRITFGQGTYSSTYILTIQEDGI | | |
| | | | TDVQYRVFEIGYISDQFGVFPSLIVSRVLPIRMV | | |
| | | | LGMESCTLTSDRQGGYFLCMNTLTRSIYDYVNIR | | |
| | | | DLKSLYITLPHYGKVNYTYFNFGKIRSPHEIDKL | | |
| | | | WLTSDRGQIISGYFAAFVTITIRNYNNYPYKCLN | | |
| | | | NPCFDNSENYCRGWYKNITGTDDVPILAYLLVEM | | |
| | | | YDEEGPLITLVAIPPYNYTAPSHNSLYYDDKINK | | |
| | | | LIMTTSHIGYIQINEVHEVIVGDNLKAILLNRLS | | |
| | | | DEHPNLTACRLNQGIKEQYKSDGMIISNSALIDI | | |
| | | | QERMYITVKAIPPVGNYNFTVELHSRSNTSYILL | | |
| | | | PKQFNAKYDKLHLECFNWDKSWWCALIPQFSLSW | | |
| | | | NESLSVDTAIFNLINCK | | |
| AB759118 | 7116-8957 | gb: AB759118: | MASPSELNRSQATLYEGDPNSKRTWRTVYRASTL | 11 | 74 |
| | | 7116-8957\|Organism: | ILDLAILCVSIVAIVRMSTLTPSDVTDSISSSIT | | |
| | | Avian paramyxovirus 6\| | SLSDTYQSVWSDTHQKVNSIFKEVGISIPVTLDK | | |
| | | Strain Name: red- | MQVEMGTAVNIITDAVRQLQGVNGSAGFSITNSP | | |
| | | necked stint/Japan/ | EYSGGIDALIYPQKSLNGKSLAISDLLEHPSFIP | | |
| | | 8KS0813/2008\|Protein | APTTSHGCTRIPTFHLGYRHWCYSHNTIESGCHD | | |
| | | Name: hemaglutinin- | AGESIMYLSMGAVGVGHQGKPVFTTSAAVILDDG | | |
| | | neuraminidase\|Gene | KNRKSCSVVANPNGCDVLCSLVKQTEDQDYADPT | | |
| | | Symbol: HN | PTPMIHGRLHFNGTYTESMLDQSLFTGHWVAQYP | | |
| | | | AVGSGSVSHGRLFFPLYGGISKSSSLFPKLRAHA | | |
| | | | YFTHNEELECKNLTSKQREDLFNAYMPGKIAGSL | | |
| | | | WAQGIVICNLTTLADCKIAVANTSTMMMAAEGRL | | |
| | | | QLVQDKVVLYQRSSSWWPVLIYYDILVSELVNAR | | |
| | | | HLDIVNWVPYPQSKFPRPTWTKGLCEKPSICPAV | | |
| | | | CVTGVYQDVWVVSVGDFSNETVVIGGYLEAASER | | |
| | | | KDPWIAAANQYNWLTRRQLFTAQTEAAYSSTTCF | | |
| | | | RNTHQDKVFCLTIMEVTDNLLGDWRIAPLLYEVT | | |
| | | | VVDRQQSSRKAVAMSEAHRTRFKYYSPENKFTPQ | | |
| | | | H | | |
| AY141760 | 6791-8485 | gb: AY141760\| | MDPKSYYCNEDLRSDGGEKSPGGDLYKGIILVST | 8 | 75 |
| | | Organism: Fer-de- | VISLIIAIISLAFIIDNKINIQSLDPLRGLEDSY | | |
| | | Lance paramyxovirus\| | LVPIKDKSESISQDIQEGIFPRLNLITAATTTTI | | |
| | | Strain Name: ATCC | PRSIAIQTKDLSDLIMNRCYPSVVNNDTSCDVLA | | |
| | | VR-895\|Protein Name: | GAIHSNLFSQLDPSTYWTCSSGTPTMNQTVKLLP | | |
| | | hemagglutinin- | DNSQIPGSTYSTGCVRIPTFSLGSMIYSYSHNVI | | |
| | | neuraminidase protein | YEGCNDHSKSSQYWQLGYISTSKTGEPLQQVSRT | | |
| | | HN\|Gene Symbol: HN | LTLNNGLNRKSCSTVAQGRGAYLLCTNVVEDERT | | |
| | | | DYSTEGIQDLTLDYIDIFGAERSYRYTNNEVDLD | | |
| | | | RPYAALYPSVGSGTVYNDRILFLGYGGLMTPYGD | | |
| | | | QAMCQAPECTSATQEGCNSNQLIGYFSGRQIVNC | | |
| | | | IIEIITVGTEKPIIRVRTIPNSQVWLGAEGRIQT | | |
| | | | LGGVLYLYIRSSGWHALAQTGIILTLDPIRISWI | | |
| | | | VNTGYSRPGNGPCSASSRCPAQCITGVYTDIFPL | | |
| | | | SQNYGYLATVTLLSGVDRVNPVISYGTSTGRVAD | | |
| | | | SQLTSSSQVAAYTTTTCFTFNQKGYCYHIIELSP | | |
| | | | ATLGIFQPVLVVTEIPKICS | | |
| EU877976 | 6248-8161 | gb: EU877976: | MQGNMEGSRDNLTVDDELKTTWRLAYRVVSLLLM | 8 | 76 |
| | | 6248-8161\|Organism: | VSALIISIVILTRDNSQSIITAINQSSDADSKWQ | | |
| | | Avian paramyxovirus | TGIEGKITSIMTDTLDTRNAALLHIPLQLNTLEA | | |
| | | 4\|Strain Name: APMV-4/ | NLLSALGGNTGIGPGDLEHCRYPVHDTAYLHGVN | | |
| | | KR/YJ/06\|Protein Name: | RLLINQTADYTAEGPLDHVNFIPAPVTTTGCTRI | | |
| | | hemagglutinin- | PSFSVSSSIWCYTHNVIETGCNDHSGSNQYISMG | | |
| | | neuraminidase protein | VIKRAGNGLPYFSTVVSKYLTDGLNRKSCSVAAG | | |
| | | HN\|Gene Symbol: HN | SGHCYLLCSLVSEPEPDDYVSPDPTPMRLGVLTW | | |
| | | | DGSYTEQAVPERIFKNIWSANYPGVGSGAIVGNK | | |
| | | | VLFPFYGGVRNGSTPEVMNRGRYYYIQDPNDYCP | | |
| | | | DPLQDQILRAEQSYYPTRFGRRMVMQGVLACPVS | | |
| | | | NNSTIASQCQSYYFNNSLGFIGAESRIYYLNGNI | | |
| | | | YLYQRSSSWWPHPQIYLLDSRIASPGTQNIDSGV | | |
| | | | NLKMLNVTVITRPSSGFCNSQSRCPNDCLFGVYS | | |
| | | | DIWPLSLTSDSIFAFTMYLQGKTTRIDPAWALFS | | |
| | | | NHAIGHEARLFNKEVSAAYSTTTCFSDTIQNQVY | | |
| | | | CLSILEVRSELLGAFKIVPFLYRVL | | |
| AB176531 | 6821-8536 | gb: AB176531: | MEDYSNLSLKSIPKRTCRIIFRTATILGICTLIV | 7 | 77 |
| | | 6821-8536\|Organism: | LCSSILHEIIHLDVSSGLMDSDDSQQGIIQPIIE | | |
| | | Human parainfluenza | SLKSLIALANQILYNVAIIIPLKIDSIETVIFSA | | |
| | | virus 2\|Strain Name: | LKDMHTGSMSNTNCTPGNLLLHDAAYINGINKFL | | |
| | | Nishio\|Protein Name: | VLKSYNGTPKYGPLLNIPSFIPSATSPNGCTRIP | | |
| | | hemagglutinin- | SFSLIKTHWCYTHNVMLGDCLDFTTSNQYLAMGI | | |
| | | neuraminidase protein | IQQSAAAFPIFRTMKTIYLSDGINRKSCSVTAIP | | |
| | | HN\|Gene Symbol: HN | GGCVLYCYVATRSEKEDYATTDLAELRLAFYYYN | | |
| | | | DTFIERVISLPNTTGQWATINPAVGSGIYHLGFI | | |
| | | | LFPVYGGLISGTPSYNKQSSRYFIPKHPNITCAG | | |
| | | | NSSEQAAAARSSYVIRYHSNRLIQSAVLICPLSD | | |
| | | | MHTARCNLVMFNNSQVMMGAEGRLYVIDNNLYYY | | |
| | | | QRSSSWWSASLFYRINTDFSKGIPPIIEAQWVPS | | |
| | | | YQVPRPGVMPCNATSFCPANCITGVYADVWPLND | | |
| | | | PEPTSQNALNPNYRFAGAFLRNESNRTNPTFYTA | | |
| | | | SASALLNTTGFNNTNHKAAYTSSTCFKNTGTQKI | | |
| | | | YCLIIIEMGSSLLGEFQIIPFLRELIP | | |
| AF052755 | 6584-8281 | gb: AF052755\| | MVAEDAPVRATCRVLFRTTTLIFLCTLLALSISI | 7 | 78 |
| | | Organism: | LYESLITQKQIMSQAGSTGSNSGLGSITDLLNNI | | |
| | | Parainfluenza virus 5\| | LSVANQIIYNSAVALPLQLDTLESTLLTAIKSLQ | | |
| | | Strain Name: W3A\| | TSDKLEQNCSWSAALINDNRYINGINQFYFSIAE | | |
| | | Protein Name: | GRNLTLGPLLNMPSFIPTATTPEGCTRIPSFSLT | | |
| | | hemagglutinin- | KTHWCYTHNVILNGCQDHVSSNQFVSMGIIEPTS | | |
| | | neuraminidase protein | AGFPFFRTLKTLYLSDGVNRKSCSISTVPGGCMM | | |
| | | HN\|Gene Symbol: HN | YCFVSTQPERDDYFSAAPPEQRIIIMYYNDTIVE | | |
| | | | RIINPPGVLDVWATLNPGTGSGVYYLGWVLFPIY | | |
| | | | GGVIKGTSLWNNQANKYFIPQMVAALCSQNQATQ | | |
| | | | VQNAKSSYYSSWFGNRMIQSGILACPLRQDLTNE | | |
| | | | CLVLPFSNDQVLMGAEGRLYMYGDSVYYYQRSNS | | |
| | | | WWPMTMLYKVTITFTNGQPSAISAQNVPTQQVPR | | |
| | | | PGTGDCSATNRCPGFCLTGVYADAWLLTNPSSTS | | |
| | | | TFGSEATFTGSYLNTATQRINPTMYIANNTQIIS | | |
| | | | SQQFGSSGQEAAYGHTTCFRDTGSVMVYCIYIIE | | |
| | | | LSSSLLGQFQIVPFIRQVTLS | | |
| BK005918 | 6560-8290 | gb: BK005918\| | MSQLGTDQIMHLAQPAIARRTWRLCFRIFALFIL | 7 | 79 |
| | | Organism: Porcine | IAIVITQIFMLTFDHTLLTTTQFLTSIGNLQSTI | | |
| | | rubulavirus\|Strain | TSWTPDVQAMLSISNQLIYTTSITLPLKISTTEM | | |
| | | Name: UNKNOWN- | SILTAIRDHCHCPDCSSACPTRQMLLNDPRYMSG | | |
| | | BK005918\|Protein Name: | VNQFIGAPTESINITFGPLFGIPSFIPTSTTTQG | | |
| | | attachemnt protein\| | CTRIPSFALGPSHWCYTHNFITAGCADGGHSNQY | | |
| | | Gene Symbol: HN | LAMGTIQSASDGSPLLITARSYYLSDGVNRKSCS | | |
| | | | IAVVPGGCAMYCYVATRSETDYYAGNSPPQQLLT | | |
| | | | LVFSNDTIIERTIHPTGLANGWVMLVPGVGSGTL | | |
| | | | YNEYLLFPAYGGMQQILANQSGEINQFFTPYNAT | | |
| | | | VRCAMAQPQFSQRAAASYYPRYFSNRWIRSAIVA | | |
| | | | CPYRAIYQTQCTLIPLPNRMVMMGSEGRIFTLGD | | |
| | | | RLFYYQRSSSWWPYPLLYQVGLNFLTTPPSVSSM | | |
| | | | TQVPLEHLARPGKGGCPGNSHCPATCVTGVYADV | | |
| | | | WPLTDPRSGVGGTSLVAAGGLDSTSERMAPVNYL | | |
| | | | AIGESLLSKTYLLSKTQPAAYTTTTCFRDTDTGK | | |
| | | | IYCITIAELGKVLLGEFQIVPFLREIKIQSRY | | |
| EU338-414 | 6015-7913 | gb: EU338414: | MDFPSRENLAAGDISGRKTWRLLFRILTLSIGVV | 7 | 80 |
| | | 6015-7913\|Organism: | CLAINIATIAKLDHLDNMASNTWTTTEADRVISS | | |
| | | Avian prarmyxoviurs 2\| | ITTPLKVPVNQINDMFRIVALDLPLQMTSLQKEI | | |
| | | Strain Name: APMV-2/ | TSQVGFLAESINNVLSKNGSAGLVLVNDPEYAGG | | |
| | | Chicken/California/ | IAVSLYQGDASAGLNFQPISLIEHPSFVPGPTTA | | |
| | | Yucaipa/56\|Protein | KGCIRIPTFHMGPSHWCYSHNIIASGCQDASHSS | | |
| | | Name: hemagglutinin- | MYISLGVLKASQTGSPIFLTTASHLVDDNINRKS | | |
| | | neuraminidase protein | CSIVASKYGCDILCSIVIETENEDYRSDPATSMI | | |
| | | HN\|Gene Symbol: HN | IGRLFFNGSYTESKINTGSIFSLFSANYPAVGSG | | |
| | | | IVVGDEAAFPIYGGVKQNTWLFNQLKDFGYFTHN | | |
| | | | DVYKCNRTDIQQTILDAYRPPKISGRLWVQGILL | | |
| | | | CPVSLRPDPGCRLKVFNTSNVMMGAEARLIQVGS | | |
| | | | TVYLYQRSSSWWVVGLTYKLDVSEITSQTGNTLN | | |
| | | | HVDPIAHTKFPRPSFRRDACARPNICPAVCVSGV | | |
| | | | YQDIWPISTATNNSNIVWVGQYLEAFYSRKDPRI | | |
| | | | GIATQYEWKVTNQLFNSNTEGGYSTTTCFRNTKR | | |
| | | | DKAYCVVISEYADGVFGSYRIVPQLIEIRTTTGK | | |
| | | | SE | | |
| KC403973 | 6234-6964 | gb: KC403973: | MEVKVENIRTIDMLKARVKNRVARSKCFKNASLI | 6 | 81 |
| | | 6234-6964\|Organism: | LIGITTLSIALNIYLIINYTMQENTSESEHHTSS | | |
| | | Human metapneumovirus\| | SPMESSRETPTVPIDNSDTNPSSQYPTQQSTEGS | | |
| | | Strain Name: HMPV/USA/ | TLYFAASASSPETEPTSTPDTTSRPPFVDTHTTP | | |
| | | TN-82-518/1982/A\| | PSASRTKTSPAVHTKNNPRISSRTHSPPWAMTRT | | |
| | | Protein Name: | VRRTTTLRTSSIRKRSSTASVQPDSSATTHKHEE | | |
| | | attachment glyco- | ASPVSPQTSASTTRPQRKSMEASTSTTYNQTS | | |
| | | protein G\|Gene | | | |
| | | Symbol: G\|Segment: 8 | | | |
| KF015281 | 4511-5844 | gb: KF015281: | MRPAEQLIQENYKLTSLSMGRNFEVSGSTTNLNF | 6 | 82 |
| | | 4511-5844\|Organism: | ERTQYPDTFRAVVKVNQMCKLIAGVLTSAAVAVC | | |
| | | Canine pneumovirus\| | VGVIMYSVFTSNHKANSMQNATIRNSTSAPPQPT | | |
| | | Strain Name: dog/ | AGPPTTEQGTTPKFTKPPTKTTTHHEITEPAKMV | | |
| | | Bari/100-12/ITA/2012\| | TPSEDPYQCSSNGYLDRPDLPEDFKLVLDVICKP | | |
| | | Protein Name: | PGPEHHSTNCYEKREINLGSVCPDLVTMKANMGL | | |
| | | attachment protein\| | NNGGGEEAAPYIEVITLSTYSNKRAMCVHNGCDQ | | |
| | | Gene Symbol: G | GFCFFLSGLSTDQKRAVLELGGQQAIMELHYDSY | | |
| | | | WKHYWSNSNCVVPRTNCNLTDQTVILFPSFNNKN | | |
| | | | QSQCTTCADSAGLDNKFYLTCDGLSRNLPLVGLP | | |
| | | | SLSPQAHKAALKQSTGTTTAPTPETRNPTPAPRR | | |
| | | | SKPLSRKKRALCGVDSSREPKPTMPYWCPMLQLF | | |
| | | | PRRSNS | | |
| KF973339 | 4624-5310 | gb: KF973339: | MSKTKDQRAAKTLEKTWDTLNHLLFISSCLYKSN | 6 | 83 |
| | | 4624-5310\|Organism: | LKSIAQITLSILAMTIPTSLIIVATTFIASANNK | | |
| | | Respiratory syncytial | VTPTTAIIQDATSQIKNTTPTHLTQNPQPGISFF | | |
| | | virus type A\|Strain | NLSGTISQTTAILAPTTPSVEPILQSTTVKTKNT | | |
| | | Name: RSV-A/US/BID- | TTTQIQPSKLTTKQRQNKPPNKPNDDFHFEVFNF | | |
| | | V7358/2002\|Protein | VPCSICSNNPTCWAICKRIPSKKPGKKTTTKPTK | | |
| | | Name: truncated | KQTIKTTKKDLKPQTTKPKEAPTT | | |
| | | attachment glyco- | | | |
| | | protein\|Gene Symbol: G | | | |
| FJ215864 | 6383-8116 | gb: FJ215864: | MSNIASSLENIVEQDSRKTTWRAIFRWSVLLITT | 5 | 84 |
| | | 6383-8116\|Organism: | GCLALSIVSIVQIGNLKIPSVGDLADEVVTPLKT | | |
| | | Avian paramyxovirus 8\| | TLSDTLRNPINQINDIFRIVALDIPLQVTSIQKD | | |
| | | Strain Name: pintail/ | LASQFSMLIDSLNAIKLGNGTNLIIPTSDKEYAG | | |
| | | Wakuya/20/78\|Protein | GIGNPVFTVDAGGSIGFKQFSLIEHPSFIAGPTT | | |
| | | Name: hemagglutinin- | TRGCTRIPTFHMSESHWCYSHNIIAAGCQDASAS | | |
| | | neuraminidase | SMYISMGVLHVSSSGTPIFLTTASELIDDGVNRK | | |
| | | protein\|Gene Symbol: | SCSIVATQFGCDILCSIVIEKEGDDYWSDTPTPM | | |
| | | HN | RHGRFSFNGSFVETELPVSSMFSSFSANYPAVGS | | |
| | | | GEIVKDRILFPIYGGIKQTSPEFTELVKYGLFVS | | |
| | | | TPTTVCQSSWTYDQVKAAYRPDYISGRFWAQVIL | | |
| | | | SCALDAVDLSSCIVKIMNSSTVMMAAEGRIIKIG | | |
| | | | IDYFYYQRSSSWWPLAFVTKLDPQELADTNSIWL | | |
| | | | TNSIPIPQSKFPRPSYSENYCTKPAVCPATCVTG | | |
| | | | VYSDIWPLTSSSSLPSIIWIGQYLDAPVGRTYPR | | |
| | | | FGIANQSHWYLQEDILPTSTASAYSTTTCFKNTA | | |
| | | | RNRVFCVTIAEFADGLFGEYRITPQLYELVRNN | | |
| JX857409 | 6619-8542 | gb: JX857409: | MEETKVKTSEYWARSPQIHATNHPNVQNREKIKE | 5 | 85 |
| | | 6619-8542\|Organism: | ILTILISFISSLSLVLVIAVLIMQSLHNGTILRC | | |
| | | Porcine parainfluenza | KDVGLESINKSTYSISNAILDVIKQELITRIINT | | |
| | | virus 1\|Strain Name: | QSSVQVALPILINKKIQDLSLIIEKSSKVHQNSP | | |
| | | S206NJ\|Protein Name: | TCSGVAALTHVEGIKPLDPDDYWRCPSGEPYLED | | |
| | | hemagglutinin protein\| | ELTLSLIPGPSMLAGTSTIDGCVRLPSLAIGKSL | | |
| | | Gene Symbol: H | YAYSSNLITKGCQDIGKSYQVLQLGIITLNSDLH | | |
| | | | PDLNPIISHTYDINDNRKSCSVAVSETKGYQLCS | | |
| | | | MPRVNEKTDYTSDGIEDIVFDVLDLKGSSRSFKF | | |
| | | | SNNDINFDHPFSALYPSVGSGIIWKNELYFLGYG | | |
| | | | ALTTALQGNTKCNLMGCPGATQDNCNKFISSSWL | | |
| | | | YSKQMVNVLIQVKGYLSSKPSIIVRTIPITENYV | | |
| | | | GAEGKLVGTRERIYIYTRSTGWHTNLQIGVLNIN | | |
| | | | HPITITWTDHRVLSRPGRSPCAWNNKCPRNCTTG | | |
| | | | VYTDAYPISPDANYVATVTLLSNSTRNNPTIMYS | | |
| | | | SSDRVYNMLRLRNTELEAAYTTTSCIVHFDRGYC | | |
| | | | FHIIEINQKELNTLQPMLFKTAIPKACRISNL | | |
| KF908238 | 7510-9249 | gb: KF908228: | MQDSRGNTQIFSQANSMVKRTWRLLFRIVTLILL | 5 | 86 |
| | | 7510-9249\|Organism: | ISIFVLSLIIVLQSTPGNLQSDVDIIRKELDELM | | |
| | | Human parainfluenza | ENFETTSKSLLSVANQITYDVSVLTPIRQEATET | | |
| | | virus 4b\|Strain Name: | NIIAKIKDHCKDRVVKGESTCTLGHKPLHDVSFL | | |
| | | QLD-01\|Protein Name: | NGFNKFYFTYRDNVQIRLNPLLDYPNFIPTATTP | | |
| | | hemagglutinin- | HGCIRIPSFSLSQTHWCYTHNTILRGCEDTASSK | | |
| | | neuraminidase | QYVSLGTLQTLENGDPYFKVEYSHYLNDRKNRKS | | |
| | | protein\|Gene Symbol: | CSVVAVLDGCLLYCVIMTKNETENFKDPQLATQL | | |
| | | HN | LTYISYNGTIKERIINPPGSSRDWVHISPGVGSG | | |
| | | | ILYSNYIIFPLYGGLMENSMIYNNQSGKYFFPNS | | |
| | | | TKLPCSNKTSEKITGAKDSYTITYFSKRLIQSAF | | |
| | | | LICDLRQFLSEDCEILIPSNDHMLVGAEGRLYNI | | |
| | | | ENNIFYYQRGSSWWPYPSLYRIKLNSNKKYPRII | | |
| | | | EIKFTKIEIAPRPGNKDCPGNKACPKECITGVYQ | | |
| | | | DIWPLSYPNTAFPHKKRAYYTGFYLNNSLARRNP | | |
| | | | TFYTADNLDYHQQERLGKFNLTAGYSTTTCFKQT | | |
| | | | TTARLYCLYILEVGDSVIGDFQIFPFLRSIDQAI | | |
| | | | T | | |
| KT071757 | 6066-7962 | gb: KT071757: | MDALSRENLTEISQGGRRTWRMLFRILTLVLTLV | 5 | 87 |
| | | 6066-7962\|Organism: | CLAINIATIAKLDSIDTSKVQTWTTTESDRVIGS | | |
| | | Avian paramyxovirus 2\| | LTDTLKIPINQVNDMFRIVALDLPLQMTTLQKEI | | |
| | | Strain Name: APMV-2/ | ASQVGFLAESINNFLSKNGSAGSVLVNDPEYAGG | | |
| | | Emberiza spodocephala/ | IGTSLFHGDSASGLDFEAPSLIEHPSFIPGPTTA | | |
| | | China/Daxing'anling/ | KGCIRIPTFHMSASHWCYSHNIIASGCQDAGHSS | | |
| | | 974/2013\|Protein Name: | MYISMGVLKATQAGSPSFLTTASQLVDDKLNRKS | | |
| | | hemagglutinin- | CSIISTTYGCDILCSLVVENEDADYRSDPPTDMI | | |
| | | neuraminidase | LGRLFFNGTYSESKLNTSAIFQLFSANYPAVGSG | | |
| | | protein\|Gene Symbol: | IVLGDEIAFPVYGGVKQNTWLFNQLKDYGYFAHN | | |
| | | HN | NVYKCNNSNIHQTVLNAYRPPKISGRLWSQVVLI | | |
| | | | CPMRLFINTDCRIKVFNTSTVMMGAEARLIQVGS | | |
| | | | DIYLYQRSSSWWVVGLTYKLDFQELSSKTGNILN | | |
| | | | NVSPIAHAKFPRPSYSRDACARPNICPAVCVSGV | | |
| | | | YQDIWPISTAHNLSQVVWVGQYLEAFYARKDPWI | | |
| | | | GIATQYDWKKNVRLFNANTEGGYSTTTCFRNTKR | | |
| | | | DKAFCVIISEYADGVFGSYRIVPQLIEIRTTSKK | | |
| | | | GLPS | | |
| LC041132 | 6605-8437 | gb: LC041132: | MQPGISEVSFVNDERSERGTWRLLFRILTIVLCL | 4 | 88 |
| | | 6605-8437\|Organism: | TSIGIGIPALIYSKEAATSGDIDKSLEAVKTGMS | | |
| | | Avain paramyxovirus | TLSSKIDESINTEQKIYRQVILEAPVSQLNMESN | | |
| | | goose/Shimane/67/ | ILSAITSLSYQIDGTSNSSGCGSPMHDQDFVGGI | | |
| | | 2000\|Strain Name: | NKEIWTTDNVNLGEITLTPFLEHLNFIPAPTTGN | | |
| | | goose/Shimane/67/2000\| | GCTRIPSFDLGLTHWCYTHNVILSGCQDYSSSFQ | | |
| | | Protein Name: | YIALGVLKISATGHVFLSTMRSINLDDERNRKSC | | |
| | | hemagglutinin- | SISATSIGCDIICSLVTEREVDDYNSPAATPMIH | | |
| | | neuraminidase | GRLDFSGKYNEVDLNVGQLFGDWSANYPGVGGGS | | |
| | | protein\|Gene Symbol: | FLNGRVWFPIYGGVKEGTPTFKENDGRYAIYTRY | | |
| | | HN | NDTCPDSESEQVSRAKSSYRPSYFGGKLVQQAVL | | |
| | | | SIKIDDTLGLDPVLTISNNSITLMGAESRVLQIE | | |
| | | | EKLYFYQRGTSWFPSLIMYPLTVDDKMVRFEPPT | | |
| | | | IFDQFTRPGNHPCSADSRCPNACVTGVYTDGYPI | | |
| | | | VFHNNHSIAAVYGMQLNDVTNRLNPRSAVWYGVS | | |
| | | | MSNVIRVSSSTTKAAYTTSTCFKVKKTQRVYCLS | | |
| | | | IGEIGNTLFGEFRIVPLLLEVYSEKGKSLKSSFD | | |
| | | | GWEDISINNPLRPLDNHRVDPILISNYTSSWP | | |
| AF092942 | 4705-5478 | gb: AF092942\| | MSNHTHHLKFKTLKRAWKASKYFIVGLSCLYKFN | 3 | 89 |
| | | Organism: Bovine | LKSLVQTALTTLAMITLTSLVITAIIYISVGNAK | | |
| | | respiratory syncytial | AKPTSKPTIQQTQQPQNHTSPFFTEHNYKSTHTS | | |
| | | virus\|Strain Name: | IQSTTLSQLPNTDTTRETTYSHSINETQNRKIKS | | |
| | | ATue51908\|Protein | QSTLPATRKPPINPSGSNPPENHQDHNNSQTLPY | | |
| | | Name: attachment | VPCSTCEGNLACLSLCQIGPERAPSRAPTITLKK | | |
| | | glycoprotein\|Gene | TPKPKTTKKPTKTTIHHRTSPEAKLQPKNNTAAP | | |
| | | Symbol: G | QQGILSSPEHHTNQSTTQI | | |
| AF326114 | 6691-847 | gb: AF326114\| | MWNSIPQLVSDHEEAKGKFTDIPLQDDTDSQHPS | 3 | 90 |
| | | Organism: Menangle | GSKSTCRTLFRTVSIILSLVILVLGVTSTMFSAK | | |
| | | virus\|Strain Name: | YSGGCATNSQLLGVSNLINQIQKSIDSLISEVNQ | | |
| | | UNKNOWN-AF326114\| | VSITTAVTLPIKIMDFGKSVTDQVTQMIRQCNTV | | |
| | | Protein Name: | CKGPGQKPGSQNVRIMPSNNLSTFQNINMSARGI | | |
| | | attachment protein\| | AYQDVPLTFVRPIKNPQSCSRFPSYSVSFGVHCF | | |
| | | Gene Symbol: HN | ANAVTDQTCELNQNTFYRVVLSVSKGNISDPSSL | | |
| | | | ETKAETRTPKGTPVRTCSIISSVYGCYLLCSKAT | | |
| | | | VPESEEMKTIGFSQMFILYLSMDSKRIIYDNIVS | | |
| | | | STSAIWSGLYPGEGAGIWHMGQLFFPLWGGIPFL | | |
| | | | TPLGQKILNSTLDIPEVGSKCKSDLTSNPAKTKD | | |
| | | | MLFSPYYGENVMVFGFLTCYLLSNVPTNCHADYL | | |
| | | | NSTVLGFGSKAQFYDYRGIVYMYIQSAGWYPFTQ | | |
| | | | IFRITLQLKQNRLQAKSIKRIEVTSTTRPGNREC | | |
| | | | SVLRNCPYICATGLFQVPWIVNSDAITSKEVDNM | | |
| | | | VFVQAWAADFTEFRKGILSLCSQVSCPINDLLSK | | |
| | | | DNSYMRDTTTYCFPQTVPNILSCTSFVEWGGDSG | | |
| | | | NPINILEIHYEVIFVAS | | |
| GU206351 | 7500-9714 | gb: GU206351: | MDKSYYTEPEDQRGNSRTWRLLFRLIVLTLLCLI | 3 | 91 |
| | | 7500-9714\|Organism: | ACTSVSQLFYPWLPQVLSTLISLNSSIITSSNGL | | |
| | | Avian paramyxovirus | KKEILNQNIKEDLIYREVAINIPLTLDRVTVEVG | | |
| | | 5\|Strain Name: | TAVNQITDALRQLQSVNGSAAFALSNSPDYSGGI | | |
| | | gudgerigar/Kunitachi/ | EHLVFQRNTLINRSVSVSDLIEHPSFIPTPTTQH | | |
| | | 74\|Protein Name: | GCTRIPTFHLGTRHWCYSHNIIGQGCADSGASMM | | |
| | | heamgglutinin | YISMGALGVSSLGTPTFTTSATSILSDSLNRKSC | | |
| | | neuraminidase protein\| | SIVATTEGCDVLCSIVTQTEDQDYADHTPTPMIH | | |
| | | Gene Symbol: HN | GRLWFNGTYTERSLSQSLFLGTWAAQYPAVGSGI | | |
| | | | MTPGRVIFPFYGGVIPNSPLFLDLERFALFTHNG | | |
| | | | DLECRNLTQYQKEAIYSAYKPPKIRGSLWAQGFI | | |
| | | | VCSVGDMGNCSLKVINTSTVMMGAEGRLQLVGDS | | |
| | | | VMYYQRSSSWWPVGILYRLSLVDIIARDIQVVIN | | |
| | | | SEPLPLSKFPRPTWTPGVCQKPNVCPAVCVTGVY | | |
| | | | QDLWAISAGETLSEMTFFGGYLEASTQRKDPWIG | | |
| | | | VANQYSWFMRRRLFKTSTEAAYSSSTCFRNTRLD | | |
| | | | RNFCLLIFELTDNLLGDWRIVPLLFELTIV | | |
| JQ001776 | 8170-10275 | gb: JQ001776: | MLSQLQKNYLDNSNQQGDKMNNPDKKLSVNFNPL | 3 | 92 |
| | | 8170-10275\|Organism: | ELDKGQKDLNKSYYVKNKNYNVSNLLNESLHDIK | | |
| | | Cedar virus\|Strain | FCIYCIFSLLIIITIINIITISIVITRLKVHEEN | | |
| | | Name: CG1a\|Protein | NGMESPNLQSIQDSLSSLTNMINTEITPRIGILV | | |
| | | Name: attachment | TATSVTLSSSINYVGTKTNQLVNELKDYITKSCG | | |
| | | glycoprotein\|Gene | FKVPELKLHECNISCADPKISKSAMYSTNAYAEL | | |
| | | Symbol: G | AGPPKIFCKSVSKDPDFRLKQIDYVIPVQQDRSI | | |
| | | | CMNNPLLDISDGFFTYIHYEGINSCKKSDSFKVL | | |
| | | | LSHGEIVDRGDYRPSLYLLSSHYHPYSMQVINCV | | |
| | | | PVTCNQSSFVFCHISNNTKTLDNSDYSSDEYYIT | | |
| | | | YFNGIDRPKTKKIPINNMTADNRYIHFTFSGGGG | | |
| | | | VCLGEEFIIPVTTVINTDVFTHDYCESFNCSVQT | | |
| | | | GKSLKEICSESLRSPTNSSRYNLNGIMIISQNNM | | |
| | | | TDFKIQLNGITYNKLSFGSPGRLSKTLGQVLYYQ | | |
| | | | SSMSWDTYLKAGFVEKWKPFTPNWMNNTVISRPN | | |
| | | | QGNCPRYHKCPEICYGGTYNDIAPLDLGKDMYVS | | |
| | | | VILDSDQLAENPEITVFNSTTILYKERVSKDELN | | |
| | | | TRSTTTSCFLFLDEPWCISVLETNRFNGKSIRPE | | |
| | | | IYSYKIPKYC | | |
| KP271123 | 6644-8431 | gb: KP271123: | MWSTQASKHPAMVNSATNLVDIPLDHPSSAQFPI | 3 | 93 |
| | | 6644-8431\|Organism: | NRKRTGRLIYRLFSILCNLILISILISLVVIWSR | | |
| | | Teviot virus/Strain | SSRDCAKSDGLSSVDNQLSSLSRSINSLITEVNQ | | |
| | | Name: Geelong\|Protein | ISVTTAINLPIKLSEFGKSVVDQVTQMIRQCNAA | | |
| | | Name: attachment | CKGPGEKPGIQNVRINIPNNFSTYSELNRTANSL | | |
| | | protein\|Gene Symbol: | NFQSRTALFARPNPYPKTCSRFPSYSVYFGIHCF | | |
| | | HN | SHAVTDSSCELSDSTYYRLVIGVADKNLSDPADV | | |
| | | | KYIGETTTPVRVQTRGCSVVSSIYGCYLLCSKSN | | |
| | | | QDYQDDFREQGFHQMFILFLSRELKTTFFDDMVS | | |
| | | | STTVTWNGLYPGEGSGIWHMGHLVFPLWGGIRFG | | |
| | | | THASEGILNSTLELPPVGPSCKRSLADNGLINKD | | |
| | | | VLFSPYFGDSVMVFAYLSCYMLSNVPTHCQVETM | | |
| | | | NSSVLGFGSRAQFYDLKGIVYLYIQSAGWFSYTQ | | |
| | | | LFRLSLQSKGYKLSVKQIKRIPISSTSRPGTEPC | | |
| | | | DIIHNCPYTCATGLFQAPWIVNGDSIRDRDVRNM | | |
| | | | AFVQAWSGAINTFQRPFMSICSQYSCPLSELLDS | | |
| | | | ESSIMRSTTTYCFPSLTESILQCVSFIEWGGPVG | | |
| | | | NPISINEVYSSISFRPD | | |
| AY286409 | 7644-9542 | gb: AY286409\| | MVDPPAVSYYTGTGRNDRVKVVTTQSTNPYWAHN | 2 | 94 |
| | | Organism: Mossman | PNQGLRRLIDMVVNVIMVTGVIFALINIILGIVI | | |
| | | virus\|Strain Name: | ISQSAGSRQDTSKSLDIIQHVDSSVAITKQIVME | | |
| | | UNKNOWN-AY286409\| | NLEPKIRSILDSVSFQIPKLLSSLLGPGKTDPPI | | |
| | | Protein Name: | ALPTKASTPVIPTEYPSLNTTTCLRIEESVTQNA | | |
| | | attachment glyco- | AALFNISFDLKTVMYELVTRTGGCVTLPSYSELY | | |
| | | protein\|Gene Symbol: G | TRVRTFSTAIRNPKTCQRAGQETDLNLIPAFIGT | | |
| | | | DTGILINSCVRQPVIATGDGIYALTYLTMRGTCQ | | |
| | | | DHRHAVRHFEIGLVRRDAWWDPVLTPIHHFTEPG | | |
| | | | TPVFDGCSLTVQNQTALALCTLTTDGPETDIHNG | | |
| | | | ASLGLALVHFNIRGEFSKHKVDPRNIDTQNQGLH | | |
| | | | LVTTAGKSAVKKGILYSFGYMVTRSPEPGDSKCV | | |
| | | | TEECNQNNQEKCNAYSKTTLDPDKPRSMIIFQID | | |
| | | | VGAEYFTVDKVVVVPRTQYYQLTSGDLFYTGEEN | | |
| | | | DLLYQLHNKGWYNKPIRGRVTFDGQVTLHEHSRT | | |
| | | | YDSLSNQRACNPRLGCPSTCELTSMASYFPLDKD | | |
| | | | FKAAVGVIALRNGMTPIITYSTDDWRNHWKYIKN | | |
| | | | ADLEFSESSLSCYSPNPPLDDYVLCTAVITAKVM | | |
| | | | SNTNPQLLATSWYQYDKCHT | | |
| AY900001 | 7809-9938 | gb: AY900001\| | MNPVAMSNFYGINQADHLREKGDQPEKGPSVLTY | 2 | 95 |
| | | Organism: J-virus\| | VSLITGLLSLFTIIALNVTNIIYLTGSGGTMATI | | |
| | | Strain Name: UNKNOWN- | KDNQQSMSGSMRDISGMLVEDLKPKTDLINSMVS | | |
| | | AY900001\|Protein Name: | YTIPSQISAMSAMIKNEVLRQCTPSFMFNNTICP | | |
| | | attachment glyco- | IAEHPVHTSYFEEVGIEAISMCTGTNRKLVVNQG | | |
| | | protein\|Gene Symbol: G | INFVEYPSFIPGSTKPGGCVRLPSFSLGLEVFAY | | |
| | | | AHAITQDDCTSSSTPDYYFSVGRIADHGTDVPVF | | |
| | | | ETLAEWFLDDKMNRRSCSVTAAGKGGWLGCSILV | | |
| | | | GSFTDELTSPEVNRISLSYMDTFGKKKDWLYTGS | | |
| | | | EVRADQSWSALFFSVGSGVVIGDTVYFLVWGGLN | | |
| | | | HPINVDAMCRAPGCQSPTQSLCNYAIKPQEWGGN | | |
| | | | QIVNGILHFKHDTNEKPTLHVRTLSPDNNWMGAE | | |
| | | | GRLFHFHNSGKTFIYTRSSTWHTLPQVGILTLGW | | |
| | | | PLSVQWVDITSISRPGQSPCEYDNRCPHQCVTGV | | |
| | | | YTDLFPLGVSYEYSVTAYLDQVQSRMNPKIALVG | | |
| | | | AQEKIYEKTITTNTQHADYTTTSCFAYKLRVWCV | | |
| | | | SIVEMSPGVITTRQPVPFLYHLNLGCQDTSTGSL | | |
| | | | TPLDAHGGTYLNTDPVGNKVDCYFVLHEGQIYFG | | |
| | | | MSVGPINYTYSIVGRSREIGANMNVSLNQLCHSV | | |
| | | | YTEFLKEKEHPGTRNNIDVEGWLLKRIETLNGTK | | |
| | | | IFGLDDLEGSGPGHQSGPEDPSIAPIGHN | | |
| EF199772 | 6150-6944 | gb: EF199772: | MEVKVENVGKSQELKVKVKNFIKRSDCKKKLFAL | 2 | 96 |
| | | 6150-6944\|Organism: | ILGLVSFELTMNIMLSVMYVESNEALSLCRIQGT | | |
| | | Avian metapneumovirus\| | PAPRDNKTNTENATKETTLHTTTTTRDPEVRETK | | |
| | | Strain Name: PL-2\| | TTKPQANEGATNPSRNLTTKGDKHQTTRATTEAE | | |
| | | Protein Name: | LEKQSKQTTEPGTSTQKHTPARPSSKSPTTTQAT | | |
| | | attachment glyco- | AQPTTPTAPKASTAPKNRQATTKKTETDTTTASR | | |
| | | protein\|Gene Symbol: G | ARNTNNPTETATTTPKATTETGKGKEGPTQHTTK | | |
| | | | EQPETTARETTTPQPRRTAGASPRAS | | |
| JF424833 | 5981-7156 | gb: JF424833: | MGSKLYMVQGTSAYQTAVGFWLDIGRRYILAIVL | 2 | 97 |
| | | 5981-7156\|Organism: | SAFGLTCTVTIALTVSVIVEQSVLEECRNYNGGD | | |
| | | Avian metapneumovirus\| | RDWWSTTQEQPTTAPSATPAGNYGGLQTARTRKS | | |
| | | Strain Name: IT/Ty/A/ | ESCLHVQISYGDMYSRSDTVLGGFDCMGLLVLCK | | |
| | | 259-01/03\|Protein | SGPICQRDNQVDPTALCHCRVDLSSVDCCKVNKI | | |
| | | Name: attachment | STNSSTTSEPQKTNPAWPSQDNTDSDPNPQGITT | | |
| | | protein\|Gene Symbol: G | STATLLSTSLGLMLTSKTGTHKSGPPQALPGSNT | | |
| | | | NGKTTTDRELGSTNQPNSTTNGQHNKHTQRMTLP | | |
| | | | PSYDNTRTILQHTTPWEKTFSTYKPTHSPTNESD | | |
| | | | QSLPTTQNSINCEHFDPQGKEKICYRVGSYNSNI | | |
| | | | TKQCRIDVPLCSTYNTVCMKTYYTEPFNCWRRIW | | |
| | | | RCLCDDGVGLVEWCCTS | | |
| JN689227 | 7918-12444 | gb: JN689227: | MSQLAAHNLAMSNFYGIHQGGQSTSQKEEEQPVQ | 2 | 98 |
| | | 7918-12444\|Organism: | GVIRYASMIVGLLSLFTIIALNVTNIIYMTESGG | | |
| | | Tailam virus\|Strain | TMQSIKNAQGSIDGSMKDLSGTIMEDIKPKTDLI | | |
| | | Name: TL8K\|Protein | NSMVSYNIPAQLSMIHQIIKNDVLKQCTPSFMFN | | |
| | | Name: attachment | NTICPLAENPTHSRYFEEVNLDSISECSGNEMSL | | |
| | | glycoprotein\|Gene | ELGTEPEFIEYPSFAPGSTKPGSCVRLPSFSLSS | | |
| | | Symbol: G | TVFAYTHTIMGHGCSELDVGDHYLAIGRIADAGH | | |
| | | | EIPQFETISSWFINDKINRRSCTVAAGVMETWMG | | |
| | | | CVIMTETFYDDLDSLDTGKITISYLDVFGRKKEW | | |
| | | | IYTRSEILYDYTYTSVYFSIGSGVVVGDTVYFLL | | |
| | | | WGSLSSPIEETAYCYAPGCSNYNQRMCNEAQRPA | | |
| | | | KFGHRQMANAILRFKTNSMGKPSISVRTLSPTVI | | |
| | | | PFGTEGRLIYSDFTKIIYLYLRSTSWYVLPLTGL | | |
| | | | LILGPPVSISWVTQEAVSRPGEYPCGASNRCPKD | | |
| | | | CITGVYTDLFPLGARYEYAVTVYLNAETYRVNPT | | |
| | | | LALIDRSKIIARKKITTESQKAGYTTTTCFVFKL | | |
| | | | RIWCMSVVELAPATMTAFEPVPFLYQLDLTCKRN | | |
| | | | NGTTAMQFSGQDGMYKSGRYKSPRNECFFEKVSN | | |
| | | | KYYFVVSTPEGIQPYEVRDLTPERVSHVIMYISD | | |
| | | | VCAPALSAFKKLIPAMRPITTLTIGNWQFRPVDI | | |
| | | | SGGLRVNIYRNLTRYGDLSMSAPEDPGTDTFPGT | | |
| | | | HAPSKGHEEVGHYTLPNEKLSEVTTAAVKTKESL | | |
| | | | NLIPDTKDTRGEEENGSGLNEIITGHTTPGHIKT | | |
| | | | HPAETKVTKHTVIIPQIEEDGSGATTSTELQDET | | |
| | | | GYHTEDYNTTNTNGSLTAPNERNNYTSGDHTVSG | | |
| | | | EDITHTITVSDRTKTTQTLPTDNTFNQTPTKIQE | | |
| | | | GSPKSESTPKDYTAIESEDSHFTDPTLIRSTPEG | | |
| | | | TIVQVIGDQFHSAVTQLGESNAIGNSEPIDQGNN | | |
| | | | LIPTTDRGTMDNTSSQSHSSTTSTQGSHSAGHGS | | |
| | | | QSNMNLTALADTDSVTDQSTSTQEIDHEHENVSS | | |
| | | | ILNPLSRHTRVMRDTVQEALTGAWGFIRGMIP | | |
| KC562242 | 6178-6926 | gb: KC562242: | MEVRVENIRAIDMFKAKIKNRIRNSRCYRNATLI | 2 | 99 |
| | | 6178-6926\|Organism: | LIGLTALSMALNIFLIIDHATLRNMIKTENCANM | | |
| | | Human metapneumovirus\| | PSAEPSKKTPMTSIAGPSTKPNPQQATQWTTENS | | |
| | | Strain Name: HMPV/USA/ | TSPAATLEGHPYTGTTQTPDTTAPQQTTDKHTAL | | |
| | | C1-334/2004/B\|Protein | PKSTNEQITQTTTEKKTTRATTQKRKKEKKTQTK | | |
| | | Name: attachment | PQVQLQPKQPTPPTKSEMQVRQSQHPTDPELTPL | | |
| | | glycoprotein G\|Gene | PKAVNRQPGQQNQAPHHIMHGEVQDPGERNTQVS | | |
| | | Symbol: G | HPSS | | |
| KC915036 | 6154-7911 | gb: KC915036: | MEVKIENVGKSQELRVKVKNFIKRSDCKKKLFAL | 2 | 100 |
| | | 6154-7911\|Organism: | ILGLISFDITMNIMLSVMYVESNEALSSCRVQGT | | |
| | | Avian metapneumovirus | PAPRDNRTNTENTAKETTLHTMTTTRNTEAGGTK | | |
| | | type C\|Strain Name: | TTKPQADERATSPSKNPTIGADKHKTTRATTEAE | | |
| | | GDY\|Protein Name: | QEKQSKQTTEPGTSTPKHIPARPSSKSPATTKTT | | |
| | | attachment glyco- | TQPTTPTVAKGGTAPKNRQTTTKKTEADTPTTSR | | |
| | | protein\|Gene Symbol: G | AKQTNKPTGTETTPPRATTETDKDKEGPTQHTTK | | |
| | | | EQPETTAGGTTTPQPRRTTSRPAPTTNTKEGAET | | |
| | | | TGTRTTKSTQTSASPPRPTRSTPSKTATGTNKRA | | |
| | | | TTTKGPNTASTDRRQQTRTTPKQDQQTQTKAKTT | | |
| | | | TNKAHAKAATTPEHNTDTTDSMKENSKEDKTTRD | | |
| | | | PSSKATTKQENTSKGTTATNLGNNTEAGARTPPT | | |
| | | | TTPTRHTTEPATSTAGGHTKARTTRWKSTAARQP | | |
| | | | TRNNTTADTKTAQSKQTTPAQLGNNTTPENTTPP | | |
| | | | DNKSNSQTNVAPTEEIEIGSSLWRRRYVYGPCRE | | |
| | | | NALEHPMNPCLKDNTTWIYLDNGRNLPAGYYDSK | | |
| | | | TDKIICYGIYRGNSYCYGRIECTCKNGTGLLSYC | | |
| | | | CNSYNWS | | |
| LC168749 | 7239-9196 | gb: LC168749: | MSSPRDRVNAFYKDNLQFKNTRVVLNKEQLLIER | 2 | 101 |
| | | 7239-9196\|Organism: | PYMLLAVLFVMFLSLVGLLAIAGIRLHRAAVNTA | | |
| | | Rinderpest morbilli- | EINSGLTTSIDITKSIEYQVKDVLTPLFKIIGDE | | |
| | | virus\|Strain Name: | VGLRTPQRFTDLTKFISDKIKFLNPDKEYDFRDI | | |
| | | Lv\|Protein Name: | NWCISPPERIKINYDQYCAHTAAEELITMLVNSS | | |
| | | H protein\|Gene Symbol: | LAGTAVLRTSLVNLGRSCTGSTTTKGQFSNMSLA | | |
| | | H | LSGIYSGRGYNISSMITITEKGMYGSTYLVGKHN | | |
| | | | QGARRPSTAWQRDYRVFEVGIIRELGVGTPVFHM | | |
| | | | TNYLELPRQPELEICMLALGEFKLAALCLADNSV | | |
| | | | ALHYGGLRDDHKIRFVKLGVWPSPADSDTLATLS | | |
| | | | AVDPTLDGLYITTHRGIIAAGKAVWAVPVTRTDD | | |
| | | | QRKMGQCRREACREKPPPFCNSTDWEPLEAGRIP | | |
| | | | AYGILTIRLGLADKPEIDIISEFGPLITHDSGMD | | |
| | | | LYTPLDGNEYWLTIPPLQNSALGTVNTLVLEPSL | | |
| | | | KISPNILTLPIRSGGGDCYTPTYLSDLADDDVKL | | |
| | | | SSNLVILPSRNLQYVSATYDTSRVEHAIVYYIYS | | |
| | | | TGRLSSYYYPVKLPIKGDPVSLQIGCFPWGLKLW | | |
| | | | CHHFCSVIDSGTGKQVTHTGAVGIEITCNSR | | |
| LC187310 | 8144-9871 | gb: LC187310: | MDSSQMNILDAMDRESSKRTWRGVFRVTTIIMVV | 2 | 102 |
| | | 8144-9871\|Organism: | TCVVLSAITLSKVAHPQGFDTNELGNGIVDRVSD | | |
| | | Avian paramyxovirus | KITEALTVPNNQIGEIFKIVALDLHVLVSSSQQA | | |
| | | 10\|Strain Name: | IAGQIGMLAESINSILSQNGSASTILSSSPEYAG | | |
| | | APMV-10-FI324/YmHA\| | GIGVPLFSNKLTNGTVIKPITLIEHPSFIPGPTT | | |
| | | Protein Name: | IGGCTRIPTFHMASSHWCYSHNIIEKGCKDSGIS | | |
| | | hemagglutinin- | SMYISLGVLQVLKKGTPVFLVTASAVLSDDRNRK | | |
| | | neuraminidase\|Gene | SCSITSSRFGCEILCSLVTEAESDDYKSDTPTGM | | |
| | | Symbol: HN | VHGRLYFNGTYREGLVDTETIFRDFSANYPGVGS | | |
| | | | GEIVEGHIHFPIYGGVKQNTGLYNSLTPYWLDAK | | |
| | | | NKYDYCKLPYTNQTIQNSYKPPFIHGRFWAQGIL | | |
| | | | SCELDLFNLGNCNLKIIRSDKVMMGAESRLMLVG | | |
| | | | SKLLMYQRASSWWPLGITQEIDIAELHSSNTTIL | | |
| | | | REVKPILSSKFPRPSYQPNYCTKPSVCPAVCVTG | | |
| | | | VYTDMWPISITGNISDYAWISHYLDAPTSRQQPR | | |
| | | | IGIANQYFWIHQTTIFPTNTQSSYSTTTCFRNQV | | |
| | | | RSRMFCLSIAEFADGVFGEFRIVPLLYELRV | | |
| NC_004074 | 6590-8563 | gb: NC_004084: | MWATSESKAPIPANSTLNLVDVPLDEPQTITKHR | 2 | 103 |
| | | 6590-8563\|Organism: | KQKRTGRLVFRLLSLVLSLMTVILVLVILASWSQ | | |
| | | Tioman virus\|Strain | KINACATKEGFNSLDLQISGLVKSINSLITEVNQ | | |
| | | Name: UNKNOWN- | ISITTAINLPIKLSDFGKSIVDQVTQMIRQCNAV | | |
| | | NC_004074\|Protein | CKGPGEKPGIQNIRINIPNNFSTYLELNNTVKSI | | |
| | | Name: attachment | ELQRRPALLARPNPIPKSCSRFPSYSVNFGIHCF | | |
| | | protein\|Gene Symbol: | AHAITDQSCELSDKTYYRLAIGISDKNLSDPSDV | | |
| | | HN | KYIGEAFTPMGLQARGCSVISSIYGCYLLCSKSN | | |
| | | | QGYEADFQTQGFHQMYILFLSRDLKTTLFNDMIS | | |
| | | | STTVVWNGLYPGEGAGIWHMGYLIFPLWGGIKIG | | |
| | | | TPASTSILNSTLDLPLVGPSCKSTLEENNLINKD | | |
| | | | VLFSPYFGESVMVFGFLSCYMLSNVPTHCQVEVL | | |
| | | | NSSVLGFGSRSQLMDLKGIVYLYIQSAGWYSYTQ | | |
| | | | LFRLSLQSRGYKLTVKQIRRIPISSTTRPGTAPC | | |
| | | | DVVHNCPYTCATGLFQAPWIVNGDSILDRDVRNL | | |
| | | | VFVQAWSGNFNTFQKGLISICNQYTCPLTTLLDN | | |
| | | | DNSIMRSTTTYCYPSLSEYNLQCQSFIEWGGPVG | | |
| | | | NPIGILEVHYIIKFK | | |
| NC_005283 | 7091-8905 | gb: NC_005283: | MSSPRDKVDAFYKDIPRPRNNRVLLDNERVIIER | 2 | 104 |
| | | 7091-8905\|Organism: | PLILVGVLAVMFLSLVGLLAIAGVRLQKATTNSI | | |
| | | Dolphin morbillivirus\| | EVNRKLSTNLETTVSIEHHVKDVLTPLFKIIGDE | | |
| | | Strain Name: UNKNOWN- | VGLRMPQKLTEIMQFISNKIKFLNPDREYDFNDL | | |
| | | NC_005283\|Protein | HWCVNPPDQVKIDYAQYCNHIAAEELIVTKFKEL | | |
| | | Name: haemagglutinin | MNHSLDMSKGRIFPPKNCSGSVITRGQTIKPGLT | | |
| | | protein\|Gene Symbol: H | LVNIYTTRNFEVSFMVTVISGGMYGKTYFLKPPE | | |
| | | | PDDPFEFQAFRIFEVGLVRDVGSREPVLQMTNFM | | |
| | | | VIDEDEGLNFCLLSVGELRLAAVCVRGRPVVTKD | | |
| | | | IGGYKDEPFKVVTLGIIGGGLSNQKTEIYPTIDS | | |
| | | | SIEKLYITSHRGIIRNSKARWSVPAIRSDDKDKM | | |
| | | | EKCTQALCKSRPPPSCNSSDWEPLTSNRIPAYAY | | |
| | | | IALEIKEDSGLELDITSNYGPLIIHGAGMDIYEG | | |
| | | | PSSNQDWLAIPPLSQSVLGVINKVDFTAGFDIKP | | |
| | | | HTLTTAVDYESGKCYVPVELSGAKDQDLKLESNL | | |
| | | | VVLPTKDFGYVTATYDTSRSEHAIVYYVYDTARS | | |
| | | | SSYFFPFRIKARGEPIYLRIECFPWSRQLWCHHY | | |
| | | | CMINSTVSNEIVVVDNLVSINMSCSR | | |
| NC_007803 | 7978-12504 | gb: NC_007803: | MSQLAAHNLAMSNFYGTHQGDLSGSQKGEEQQVQ | 2 | 105 |
| | | 7978-12504\|Organism: | GVIRYVSMIVSLLSLFTIIALNVTNIIYMTESGG | | |
| | | Beilong virus\|Strain | TMQSIKTAQGSIDGSMREISGVIMEDVKPKTDLI | | |
| | | Name: Li\|Protein | NSMVSYNIPAQLSMIHQIIKNDVPKQCTPSFMFN | | |
| | | Name: attachment | NTICPLAENPTHSRYFEEVNLDSISECSGPDMHL | | |
| | | glycoprotein\|Gene | GLGVNPEFIEFPSFAPGSTKPGSCVRLPSFSLST | | |
| | | Symbol: G | TVFAYTHTIMGHGCSELDVGDHYFSVGRIADAGH | | |
| | | | EIPQFETISSWFINDKINRRSCTVAAGAMEAWMG | | |
| | | | CVIMTETFYDDRNSLDTGKLTISYLDVFGRKKEW | | |
| | | | IYTRSEILYDYTYTSVYFSVGSGVVVGDTVYFLI | | |
| | | | WGSLSSPIEETAYCFAPDCSNYNQRMCNEAQRPS | | |
| | | | KFGHRQMVNGILKFKTTSTGKPLLSVGTLSPSVV | | |
| | | | PFGSEGRLMYSEITKIIYLYLRSTSWHALPLTGL | | |
| | | | FVLGPPTSISWIVQRAVSRPGEFPCGASNRCPKD | | |
| | | | CVTGVYTDLFPLGSRYEYAATVYLNSETYRVNPT | | |
| | | | LALINQTNIIASKKVTTESQRAGYTTTTCFVFKL | | |
| | | | RVWCISVVELAPSTMTAYEPIPFLYQLDLTCKGK | | |
| | | | NGSLAMRFAGKEGTYKSGRYKSPRNECFFEKVSN | | |
| | | | KYYFIVSTPEGIQPYEIRDLTPDRMPHIIMYISD | | |
| | | | VCAPALSAFKKLLPAMRPITTLTIGNWQFRPVEV | | |
| | | | SGGLRVNIGRNLTKEGDLTMSAPEDPGSNTFPGN | | |
| | | | HIPGNGILDAGYYTVEYPKE | | |
| NC_009489 | 6559-8512 | gb: NC_009489: | MASLQSEPGSQKPHYQSDDQLVKRTWRSFFRFSV | 2 | 106 |
| | | 6559-8512\|Organism: | LVVTITSLALSIITLIGVNRISTAKQISNAFAAI | | |
| | | Mapuera virus\|Strain | QANILSSIPDIRPINSLLNQLVYTSSVTLPLRIS | | |
| | | Name: BeAnn 370284\| | SLESNVLAAIQEACTYRDSQSSCSATMSVMNDQR | | |
| | | Protein Name: | YIEGIQVYSGSFLDLQKHTLSPPIAFPSFIPTST | | |
| | | attachment protein\| | TTVGCTRIPSFSLTKTHWCYTHNYIKTGCRDATQ | | |
| | | Gene Symbol: HN | SNQYIALGTIYTDPDGTPGFSTSRSQYLNDGVNR | | |
| | | | KSCSISAVPMGCALYCFISVKEEVDYYKGTVPPA | | |
| | | | QTLILFFFNGTVHEHRIVPSSMNSEWVMLSPGVG | | |
| | | | SGVFYNNYIIFPLYGGMTKDKAEKRGELTRFFTP | | |
| | | | KNSRSLCKMNDSVFSNAAQSAYYPPYFSSRWIRS | | |
| | | | GLLACNWNQIITTNCEILTFSNQVMMMGAEGRLI | | |
| | | | LINDDLFYYQRSTSWWPRPLVYKLDIELNYPDSH | | |
| | | | IQRVDQVEVTFPTRPGWGGCVGNNFCPMICVSGV | | |
| | | | YQDVWPVTNPVNTTDSRTLWVGGTLLSNTTRENP | | |
| | | | ASVVTSGGSISQTVSWFNQTVPGAYSTTTCFNDQ | | |
| | | | VQGRIFCLIIFEVGGGLLGEYQIVPFLKELKYQG | | |
| | | | AVHA | | |
| NC_017937 | 6334-8544 | gb: NC_017937: | MAPINYPASYYTNNAERPVVITTKSTESKGQRPL | 2 | 107 |
| | | 6334-8544\|Organism: | PLGNARFWEYFGHVCGTLTFCMSLIGIIVGIIAL | | |
| | | Nariva virus\|Strain | ANYSSDKDWKGRIGGDIQVTRMATEKTVKLILED | | |
| | | Name: UNKNOWN- | TTPKLRNILDSVLFQLPKMLASIASKINTQTPPP | | |
| | | NC_017937\|Protein | PTTSGHSTALATQCSSNCENRPEIGYDYLRQVEQ | | |
| | | Name: attachment | SLQRITNISIQLLEASEIHSMAGAYPNALYKIRT | | |
| | | protein\|Gene Symbol: H | QDSWSVTAKECPLQAFQPNLNLIPAMIGTATGAL | | |
| | | | IRNCVRQPVIVVDDGVYMLTYLAMRGSCQDHQKS | | |
| | | | VRHFEMGVITSDPFGDPVPTPLRHWTKRALPAYD | | |
| | | | GCALAVKGHAGFALCTETSVGPLRDRTAKRKPNI | | |
| | | | VLFKASLVGELSERVIPPQSWLSGFSFFSVYTVA | | |
| | | | GKGYAYHSKFHAFGNVVRVGQSEYQAKCRGTGCP | | |
| | | | TANQDDCNTAQRVSQEDNTYLHQAILSVDIDSVI | | |
| | | | DPEDVVYVIERDQYYQASAGDLYRVPETGEILYN | | |
| | | | LHNGGWSNEVQVGRIQPSDRFYMREIQLTSTRVP | | |
| | | | APNGCNRVKGCPGGCVAVISPAFTPMHPEFNVGV | | |
| | | | GIFPMNQPHNPSIMHVQQQTELFWKPIVGGNITL | | |
| | | | HESSIACYSTVPPNPSYDLCIGVMTLLLHQGQLP | | |
| | | | QFQALSWYQPTMCNGNAPQNRRALIPVIVEDSKA | | |
| | | | MSVSSDAPRTP | | |
| NC_025256 | 9117-11015 | gb: NC025256: | MPQKTVEFINMNSPLERGVSTLSDKKTLNQSKIT | 2 | 108 |
| | | 9117-11015\|Organism: | KQGYFGLGSHSERNWKKQKNQNDHYMTVSTMILE | | |
| | | Bat Paramyxovirus | ILVVLGIMFNLIVLTMVYYQNDNINQRMAELTSN | | |
| | | Eid_hel/GH-M74a/GHA/ | ITVLNLNLNQLTNKIQREIIPRITLIDTATTITI | | |
| | | 2009\|Strain Name: | PSAITYILATLTTRISELLPSINQKCEFKTPTLV | | |
| | | BatPV/Eid_hel/GH- | LNDCRINCTPPLNPSDGVKMSSLATNLVAHGPSP | | |
| | | M74a/GHA/2009\|Protein | CRNFSSVPTIYYYRIPGLYNRTALDERCILNPRL | | |
| | | Name: glycoprotein\| | TISSTKFAYVHSEYDKNCTRGFKYYELMTFGEIL | | |
| | | Gene Symbol: G | EGPEKEPRMFSRSFYSPTNAVNYHSCTPIVTVNE | | |
| | | | GYFLCLECTSSDPLYKANLSNSTFHLVILRHNKD | | |
| | | | EKIVSMPSFNLSTDQEYVQIIPAEGGGTAESGNL | | |
| | | | YFPCIGRLLHKRVTHPLCKKSNCSRTDDESCLKS | | |
| | | | YYNQGSPQHQVVNCLIRIRNAQRDNPTWDVITVD | | |
| | | | LTNTYPGSRSRIFGSFSKPMLYQSSVSWHTLLQV | | |
| | | | AEITDLDKYQLDWLDTPYISRPGGSECPFGNYCP | | |
| | | | TVCWEGTYNDVYSLTPNNDLFVTVYLKSEQVAEN | | |
| | | | PYFAIFSRDQILKEFPLDAWISSARTTTISCFMF | | |
| | | | NNEIWCIAALEITRLNDDIIRPIYYSFWLPTDCR | | |
| | | | TPYPHTGKMTRVPLRSTYNY | | |
| NC_025347 | 6398-8418 | gb: NC_025347: | MESIGKGTWRTVYRVLTILLDVVIIILSVIALIS | 2 | 109 |
| | | 6398-8418\|Organism: | LGLKPGERIINEVNGSIHNQLVPLSGITSDIQAK | | |
| | | Avian paramyxovirus | VSSIYRSNLLSIPLQLDQINQAISSSARQIADTI | | |
| | | 7\|Strain Name: APMV- | NSFLALNGSGTFIYTNSPEFANGFNRAMFPTLNQ | | |
| | | 7/dove/Tennessee/4/ | SLNMLTPGNLIEFTNFIPTPTTKSGCIRIPSFSM | | |
| | | 75\|\|Protein Name: | SSSHWCYTHNIIASGCQDHSTSSEYISMGVVEVT | | |
| | | hemagglutinin- | DQAYPNFRTTLSITLADNLNRKSCSIAATGFGCD | | |
| | | neuraminidase\|Gene | ILCSVVTETENDDYQSPEPTQMIYGRLFFNGTYS | | |
| | | Symbol: HN | EMSLNVNQMFADWVANYPAVGSGVELADFVIFPL | | |
| | | | YGGVKITSTLGASLSQYYYIPKVPTVNCSETDAQ | | |
| | | | QIEKAKASYSPPKVAPNIWAQAVVRCNKSVNLAN | | |
| | | | SCEILTFNTSTMMMGAEGRLLMIGKNVYFYQRSS | | |
| | | | SYWPVGIIYKLDLQELTTFSSNQLLSTIPIPFEK | | |
| | | | FPRPASTAGVCSKPNVCPAVCQTGVYQDLWVLYD | | |
| | | | LGKLENTTAVGLYLNSAVGRMNPFIGIANTLSWY | | |
| | | | NTTRLFAQGTPASYSTTTCFKNTKIDTAYCLSIL | | |
| | | | ELSDSLLGSWRITPLLYNITLSIMS | | |
| NC_025348 | 6590-8548 | gb: NC_025348: | MPPVPTVSQSIDEGSFTDIPLSPDDIKHPLSKKT | 2 | 110 |
| | | 6590-8548\|Organism: | CRKLFRIVTLIGVGLISILTIISLAQQTGILRKV | | |
| | | Tuhoko virus 2\|Strain | DSSDFQSYVQESFKQVLNLMKQFSSNLNSLIEIT | | |
| | | Name: UNKNOWN- | SVTLPFRIDQFGTDIKTQVAQLVRQCNAVCRGPI | | |
| | | NC_025348\|Protein | KGPTTQNIVYPALYETSLNKTLETKNVRIQEVRQ | | |
| | | Name: hemagglutinin- | EVDPVPGPGLSNGCTRNPSFSVYHGVWCYTHATS | | |
| | | neuraminidase\|Gene | IGNCNGSLGTSQLFRIGNVLEGDGGAPYHKSLAT | | |
| | | Symbol: HN | HLLTTRNVSRQCSATASYYGCYFICSEPVLTERD | | |
| | | | DYETPGIEPITIFRLDPDGNWVVFPNINRFTEYS | | |
| | | | LKALYPGIGSGVLFQGKLIFPMYGGIDKERLSAL | | |
| | | | GLGNIGLIERRMADTCNHTEKELGRSFPGAFSSP | | |
| | | | YYHDAVMLNFLLICEMIENLPGDCDLQILNPTNM | | |
| | | | SMGSESQLSVLDNELFLYQRSASWWPYTLIYRLN | | |
| | | | MRYTGKYLKPKSIIPMVIKSNTRPGYEGCNHERV | | |
| | | | CPKVCVTGVFQAPWILSIGRDHKERVSNVTYMVA | | |
| | | | WSMDKSDRTYPAVSVCGSDTCKLTVPLGDSKVHS | | |
| | | | AYSVTRCYLSRDHMSAYCLVIFELDARPWAEMRI | | |
| | | | QSFLYKLILT | | |
| NC_025350 | 6451-8341 | gb: NC_025350: | MHNRTQSVSSIDTSSDVYLPRRKKAVTKFTFKKI | 2 | 111 |
| | | 6451-8341\|Organism: | FRVLILTLLLSIIIIIAVIFPKIDHIRETCDNSQ | | |
| | | Tuhoko virus 3\|Strain | ILETITNQNSEIKNLINSAITNLNVLLTSTTVDL | | |
| | | Name: UNKNOWN- | PIKLNNFGKSIVDQVTMMVRQCNAVCRGPGDRPT | | |
| | | NC_025350\|Protein | QNIELFKGLYHTSPPSNTSTKLSMITEASNPDDI | | |
| | | Name: hemagglutinin- | VPRPGKLLGCTRFPSFSVHYGLWCYGHMASTGNC | | |
| | | neuraminidase\|Gene | SGSSPSVQIIRIGSIGTNKDGTPKYVIIASASLP | | |
| | | Symbol: HN | ETTRLYHCSVTMTSIGCYILCTTPSVSETDDYST | | |
| | | | MGIEKMSISFLSLDGYLTQLGQPTGLDNQNLYAL | | |
| | | | YPGPGSGVIFRDFLIFPMMGGIRLMDAQKMLNRN | | |
| | | | ITYRGFPPSETCTESELKLKQEVANMLTSPYYGE | | |
| | | | VLVLNFLYVCSLLDNIPGDCSVQLIPPDNMTLGA | | |
| | | | ESRLYVLNGSLIMYKRGSSWWPYTELYQINYRVN | | |
| | | | NRAFRVRESVRINTTSTSRPGVQGCNLEKVCPKV | | |
| | | | CVSGIYQSPGIISAPVNPTRQEEGLLYFLVWTSS | | |
| | | | MSSRTGPLSSLCDHSTCRITYPIGDDTIFIGYTD | | |
| | | | SSCFMSSIKEGIYCIAFLELDNQPYSMMAIRSLS | | |
| | | | YIIN | | |
| NC_025352 | 8716-11257 | gb: NC_025352: | MATNRDNTITSAEVSQEDKVKKYYGVETAEKVAD | 2 | 112 |
| | | 8716-11257\|Organism: | SISGNKVFILMNTLLILTGAIITITLNITNLTAA | | |
| | | Mojiang virus\|Strain | KSQQNMLKIIQDDVNAKLEMFVNLDQLVKGEIKP | | |
| | | Name: tongguan1\| | KVSLINTAVSVSIPGQISNLQTKFLQKYVYLEES | | |
| | | Protein Name: | ITKQCTCNPLSGIFPTSGPTYPPTDKPDDDTTDD | | |
| | | attachment glyco- | DKVDTTIKPIEYPKPDGCNRTGDHFTMEPGANFY | | |
| | | protein\|Gene Symbol: G | TVPNLGPASSNSDECYTNPSFSIGSSIYMFSQEI | | |
| | | | RKTDCTAGEILSIQIVLGRIVDKGQQGPQASPLL | | |
| | | | VWAVPNPKIINSCAVAAGDEMGWVLCSVTLTAAS | | |
| | | | GEPIPHMFDGFWLYKLEPDTEVVSYRITGYAYLL | | |
| | | | DKQYDSVFIGKGGGIQKGNDLYFQMYGLSRNRQS | | |
| | | | FKALCEHGSCLGTGGGGYQVLCDRAVMSFGSEES | | |
| | | | LITNAYLKVNDLASGKPVIIGQTFPPSDSYKGSN | | |
| | | | GRMYTIGDKYGLYLAPSSWNRYLRFGITPDISVR | | |
| | | | STTWLKSQDPIMKILSTCTNTDRDMCPEICNTRG | | |
| | | | YQDIFPLSEDSEYYTYIGITPNNGGTKNFVAVRD | | |
| | | | SDGHIASIDILQNYYSITSATISCFMYKDEIWCI | | |
| | | | AITEGKKQKDNPQRIYAHSYKIRQMCYNMKSATV | | |
| | | | TVGNAKNITIRRY | | |
| NC_025362 | 6503-8347 | gb: NC_025363: | MESATSQVSFENDKTSDRRTWRAVFRVLMIILAL | 2 | 113 |
| | | 6503-8347\|Organism: | SSLCVTVAALIYSAKAAIPGNIDASEQRILSSVE | | |
| | | Avian paramyxovirus | AVQVPVSRLEDTSQKIYRQVILEAPVTQLNMETN | | |
| | | 12\|Strain Name: | ILNAITSLSYQIDASANSSGCGAPVHDSDFTGGV | | |
| | | Wigeion/Italy/3920_1/ | GRELLQEAEVNLTIIRPSKFLEHLNFIPAPTTGN | | |
| | | 2005\|Protein Name: | GCTRIPSFDLGQTHWCYTHNVVLNGCRDRGHSFQ | | |
| | | hemagglutinin- | YVALGILRTSATGSVFLSTLRSVNLDDDRNRKSC | | |
| | | neuraminidase\|Gene | SVSATPIGCEMLCSLVTETEEGDYDSIDPTPMVH | | |
| | | Symbol: HN | GRLGFDGKYREVDLSEKEIFADWRANYPAVGGGA | | |
| | | | FFGNRVWFPVYGGLKEGTQSERDAEKGYAIYKRF | | |
| | | | NNTCPDDNTTQIANAKASYRPSRFGGRFIQQGIL | | |
| | | | SFKVEGNLGSDPILSLTDNSITLMGAEARVMNIE | | |
| | | | NKLYLYQRGTSWFPSALVYPLDVANTAVKVRAPY | | |
| | | | IFDKFTRPGGHPCSASSRCPNVCVTGVYTDAYPL | | |
| | | | VFSRSHDIVAVYGMQLAAGTARLDPQAAIWYGNE | | |
| | | | MSTPTKVSSSTTKAAYTTSTCFKVTKTKRIYCIS | | |
| | | | IAEIGNTLFGEFRIVPLLIEVQKTPLTRRSELRQ | | |
| | | | QMPQPPIDLVIDNPFCAPSGNLSRKNAIDEYANS | | |
| | | | WP | | |
| NC_025373 | 6619-8605 | gb: NC_025373: | MEPTGSKVDIVPSQGTKRTCRTFYRLLILILNLI | 2 | 114 |
| | | 6619-8605\|Organism: | IIILTIISIYVSISTDQHKLCNNEADSLLHSIVE | | |
| | | Avian paramyxovirus | PITVPLGTDSDVEDELREIRRDTGINIPIQIDNT | | |
| | | 3\|Strain Name: turkey/ | ENIILTTLASINSNIARLHNATDESPTCLSPVND | | |
| | | Wisconsin/68\|Protein | PRFIAGINKITKGSMIYRNFSNLIEHVNFIPSPT | | |
| | | Name: hemagglutinin- | TLSGCTRIPSFSLSKTHWCYSHNVISTGCQDHAA | | |
| | | neuraminidase\|Gene | SSQYISIGIVDTGLNNEPYLRTMSSRLLNDGLNR | | |
| | | Symbol: HN | KSCSVTAGAGVCWLLCSVVTESESADYRSRAPTA | | |
| | | | MILGRFNFYGDYTESPVPASLFSGRFTANYPGVG | | |
| | | | SGTQLNGTLYFPIYGGVVNDSDIELSNRGKSFRP | | |
| | | | RNPTNPCPDPEVTQSQRAQASYYPTRFGRLLIQQ | | |
| | | | AILACRISDTTCTDYYLLYFDNNQVMMGAEARIY | | |
| | | | YLNNQMYLYQRSSSWWPHPLFYRFSLPHCEPMSV | | |
| | | | CMITDTHLILTYATSRPGTSICTGASRCPNNCVD | | |
| | | | GVYTDVWPLTEGTTQDPDSYYTVFLNSPNRRISP | | |
| | | | TISIYSYNQKISSRLAVGSEIGAAYTTSTCFSRT | | |
| | | | DTGALYCITIIEAVNTIFGQYRIVPILVQLISD | | |
| NC_025386 | 7541-9403 | gb: NC_025386: | MKAMHYYKNDFADPGTNDNSSDLTTNPFISNQIK | 2 | 115 |
| | | 7541-9403\|Organism: | SNLSPPVLAEGHLSPSPIPKFRKILLTISFVSTI | | |
| | | Salem virus\|Strain | VVLTVILLVLTIRILTIIEASAGDEKDIHTILSS | | |
| | | Name: UNKNOWN- | LLNTFMNEYIPVFKNLVSIISLQIPQMLIDLKTS | | |
| | | NC_025386\|Protein | STQMMQSLKTFPRDLETLSTVTQSVAVLLEKAKS | | |
| | | Name: attachment | TIPDINKFYKNVGKVTFNDPNIKVLTLEVPAWLP | | |
| | | glycoprotein\|Gene | IVRQCLKQDFRQVISNSTGFALIGALPSQLFNEF | | |
| | | Symbol: G | EGYPSLAIVSEVYAITYLKGVMFENQENFLYQYF | | |
| | | | EIGTISPDGYNKPYFLRHTSVMLSTFKLSGKCTA | | |
| | | | AVDYRGGIFLCTPSPKIPKILQNPPDLPTLTVVS | | |
| | | | IPFDGRYTIRNISLMLTDEADIIYDLDTLQGRGV | | |
| | | | LQAMRFYALVRVISSSSPRHFPFCKNSWCPTADD | | |
| | | | KICDQSRRLGADGNYPVMYGLISIPAHSSYQGNV | | |
| | | | SLKLIDPKYYAYTRDASLFYNSMTDTYHYSFGTR | | |
| | | | GWVSRPIIGELLLGDDIVLTRYTVRSVSRATAGD | | |
| | | | CTTVSMCPQACSGGMNSIFYPLNFDKPQVTGVAI | | |
| | | | RQYERQQEGIIVVTMNDHYYYSVPIIKNGTLLIS | | |
| | | | SVTDCFWLMGDLWCMSLMEKNNLPLGVRSLAHLT | | |
| | | | WNIHWSCS | | |
| NC_025390 | 6647-8386 | gb: NC_025396: | MESGISQASLVNDNIELRNTWRTAFRVVSLLLGF | 2 | 116 |
| | | 6647-8386\|Organism: | TSLVLTACALHFALNAATPADLSSIPVAVDQSHH | | |
| | | Avian paramyxovirus | EILQTLSLMSDIGNKIYKQVALDSPVALLNTEST | | |
| | | 9\|Strain Name: duck/ | LMSAITSLSYQINNAANNSGCGAPVHDKDFINGV | | |
| | | New York/22/1978\| | AKELFVGSQYNASNYRPSRFLEHLNFIPAPTTGK | | |
| | | Protein Name: | GCTRIPSFDLAATHWCYTHNVILNGCNDHAQSYQ | | |
| | | hemagglutinin- | YISLGILKVSATGNVFLSTLRSINLDDDENRKSC | | |
| | | neuraminidase\|Gene | SISATPLGCDLLCAKVTEREEADYNSDAATRLVH | | |
| | | Symbol: HN | GRLGFDGVYHEQALPVESLFSDWVANYPSVGGGS | | |
| | | | YFDNRVWFGVYGGIRPGSQTDLLQSEKYAIYRRY | | |
| | | | NNTCPDNNPTQIERAKSSYRPQRFGQRLVQQAIL | | |
| | | | SIRVEPSLGNDPKLSVLDNTVVLMGAEARIMTFG | | |
| | | | HVALMYQRGSSYFPSALLYPLSLTNGSAAASKPF | | |
| | | | IFEQYTRPGSPPCQATARCPNSCVTGVYTDAYPL | | |
| | | | FWSEDHKVNGVYGMMLDDITSRLNPVAAIFDRYG | | |
| | | | RSRVTRVSSSSTKAAYTTNTCFKVVKTKRVYCLS | | |
| | | | IAEIENTLFGEFRITPLLSEIIFDPNLEPSDTSR | | |
| | | | N | | |
| NC_025403 | 6692-8645 | gb: NC_025403: | MATNLSTITNGKFSQNSDEGSLTELPFFEHNRKV | 2 | 117 |
| | | 6692-8645\|Organism: | ATTKRTCRFVFRSVITLCNLTILIVTVVVLFQQA | | |
| | | Achimota virus 1\| | GFIKRTESNQVCETLQNDMHGVVTMSKGVITTLN | | |
| | | Strain Name: UNKNOWN- | NLIEITSVNLPFQMKQFGQGIVTQVTQMVRQCNA | | |
| | | NC_025403\|Protein | VCKGPTIGPDIQNIVYPASYESMIKHPVNNSNIL | | |
| | | Name: attachment | LSEIRQPLNFVPNTGKLNGCTRTPSFSVYNGFWC | | |
| | | protein\|Gene Symbol: | YTHAESDWNCNGSSPYMQVFRVGVVTSDYDYNVI | | |
| | | HN | HKTLHTKTSRLANVTYQCSTISTGYECYFLCSTP | | |
| | | | NVDEITDYKTPGIESLQIYKIDNRGTFAKFPITD | | |
| | | | QLNKELLTALYPGPGNGVLYQGRLLFPMHGGMQS | | |
| | | | SELNKVNLNNTVLSQFNDNKGCNATEIKLESEFP | | |
| | | | GTFTSPYYSNQVMLNYILICEMIENLPGNCDLQI | | |
| | | | VAPKNMSMGSESQLYSINNKLYLYQRSSSRWPYP | | |
| | | | LIYEVGTRLTNRQFRLRAINRFLIKSTTRPGSEG | | |
| | | | CNIYRVCPKVCVTGVYQAPWILHVSKAGSQSIAK | | |
| | | | VLYAVAWSKDHMSRKGPLFSICDNDTCFLTKSLA | | |
| | | | SEHVHSGYSITRCYLENSERHIICVVIMELDASP | | |
| | | | WAEMRIQSVIYNITLPS | | |
| NC_025402 | 6655-8586 | gb: NC_025404: | MDNSMSISTISLDAQPRIWSRHESRRTWRNIFRI | 2 | 118 |
| | | 6655-8586\|Organism: | TSLVLLGVTVIICIWLCCEVARESELELLASPLG | | |
| | | Achimota virus 2\| | ALIMAINTIKSSVVKMTTELNQVTFTTSIILPNK | | |
| | | Strain Name: UNKNOWN- | VDQFGQNVVSQVAQLVKQCNAVCRGHQDTPELEQ | | |
| | | NC_025404\|Protein | FINQKNPTWILQPNYTTKLTNLHEIDSIIPLVDY | | |
| | | Name: attachment | PGFSKSCTRFPSFSEGSKFWCFTYAVVKEPCSDI | | |
| | | protein: Gene Symbol: | SSSIQVVKYGAIKANHSDGNPYLVLGTKVLDDGK | | |
| | | HN | FRRGCSITSSLYGCYLLCSTANVSEVNDYAHTPA | | |
| | | | YPLTLELISKDGITTDLSPTYTVQLDKWSALYPG | | |
| | | | IGSGVIFKGYLMFPVYGGLPFKSPLISASWVGPG | | |
| | | | NKWPVDFSCSEDQYSTFNFSNPYSALYSPHFSNN | | |
| | | | IVVSALFVCPLNENLPYSCEVQVLPQGNLTIGAE | | |
| | | | GRLYVIDQDLYYYQRSTSWWPYLQLYKLNIRITN | | |
| | | | RVFRVRSLSLLPIKSTTRPGYGNCTYFKLCPHIC | | |
| | | | VTGVYQSPWLISIRDKRPHEEKNILYFIGWSPDE | | |
| | | | QIRQNPLVSLCHETACFINRSLATNKTHAGYSES | | |
| | | | HCVQSFERNKLTCTVFYELTAKPWAEMRVQSLLF | | |
| | | | QVDFL | | |
| NC_025410 | 6799-8869 | gb: NC_025410: | MDSRSDSFTDIPLDNRIERTVTSKKTWRSIFRVT | 2 | 119 |
| | | 6799-8869\|Organism: | AIILLIICVVVSSISLNQHNDAPLNGAGNQATSG | | |
| | | Tuhoko virus 1\| | FMDAIKSLEKLMSQTINELNQVVMTTSVQLPNRI | | |
| | | Strain Name: UNKNOWN- | TKFGQDILDQVTQMVRQCNAVCRGPGVGPSIQNY | | |
| | | NC_025410\|Protein | VIQGHAPTVSFDPISAEYQKFVFGITEKTLITAY | | |
| | | Name: hemagglutinin- | HNPWECLRFPSQHLFDTTWCVSYQILTQNCSDHG | | |
| | | neuraminidase\|Gene | PRITVIQLGEIMIANNLSTVFRDPVIKYIRHHIW | | |
| | | Symbol: HN | LRSCSVVAYYSQCTIFCTSTNKSEPSDYADTGYE | | |
| | | | QLFLATLQSDGTFTEHSMHGVNIVHQWNAIYGGV | | |
| | | | GNGVIIGRNMLIPLYGGINYYDHNTTIVQTVDLR | | |
| | | | PYPIPDSCSQTDNYQTNYLPSMFTNSYYGTNLVV | | |
| | | | SGYLSCRLMAGTPTSCSIRVIPIENMTMGSEGQF | | |
| | | | YLINNQLYYYKRSSNWIRDTQVYLLSYSDKGNII | | |
| | | | EITSAERYIFKSVTSPDEGDCVTNHGCPSNCIGG | | |
| | | | LFQAPWILNDFKLCGSNITCPKIVTVWADQPDKR | | |
| | | | SNPMLSIAETDKLLLHKSYINYHTAVGYSTVLCF | | |
| | | | DSPKLNLKTCVVLQELMSDDKLLIRISYSIVSIM | | |
| | | | VE | | |
| NC_028249 | 7059-9010 | gb: NC_028249: | MFSHQDKVGAFYKNNARANSSKLSLVTDEVEERR | 2 | 120 |
| | | 7059-9010\|Organism: | SPWFLSILLILLVGILILLAITGIRFHQVVKSNL | | |
| | | Phocine distemper | EFNKLLIEDMEKTKAVHHQVKDVLTPLFKIIGDE | | |
| | | virus\|Strain Name: | VGLRLPQKLNEIKQFIVQKTNFFNPNREFDFREL | | |
| | | PDV/Wadden_Sea.NLD/ | HWCINPPSKVKVNFTQYCEITEFKEATRSVANSI | | |
| | | 1988\|Protein Name: | LLLTLYRGRDDIFPPYKCRGATTSMGNVFPLAVS | | |
| | | hemagglutinin- | LSMSLISKPSEVINMLTAISEGIYGKTYLLVTDD | | |
| | | neuraminidase\|Gene | TEENFETPEIRVFEIGFINRWLGDMPLFQTTNYR | | |
| | | Symbol: HN | IISNNSNTKICTIAVGELALASLCTKESTILLNL | | |
| | | | GDEESQNSVLVVILGLFGATHMDQLEEVIPVAHP | | |
| | | | SIEKIHITNHRGFIKDSVATWMVPALALSEQGEQ | | |
| | | | INCLRSACKRRTYPMCNQTSWEPFGDKRLPSYGR | | |
| | | | LTLSLDVSTDLSINVSVAQGPIIFNGDGMDYYEG | | |
| | | | TLLNSGWLTIPPKNGTILGLINQASKGDQFIVTP | | |
| | | | HILTFAPRESSTDCHLPIQTYQIQDDDVLLESNL | | |
| | | | VVLPTQSFEYVVATYDVSRSDHAIVYYVYDPART | | |
| | | | VSYTYPFRLRTKGRPDILRIECFVWDGHLWCHQF | | |
| | | | YRFQLDATNSTSVVENLIRIRFSCDRLDP | | |
| NC_028362 | 6951-8675 | gb: NC_028362: | MEYWGHTNNPDKINRKVGVDQVRDRSKTLKIITF | 2 | 121 |
| | | 6951-8675\|Organism: | IISMMTSIMSTVALILILIMFIQNNNNNRIILQE | | |
| | | Caprine parainfluenza | LRDETDAIEARIQKASNDIGVSIQSGINTRLLTI | | |
| | | virus 3\|Strain Name: | QNHVQNYIPLALTQQVSSLRESINDVITKREETQ | | |
| | | JS2013\|Protein Name: | SKMPIQRMTHDDGIEPLIPDNFWKCPSGIPTISA | | |
| | | hemagglutinin- | SPKIRLIPGPGLLATSTTINGCIRLPSLVINNLI | | |
| | | neuraminidase\|Gene | YAYTSNLITQGCQDIGKSYQVLQIGIITINSDLV | | |
| | | Symbol: HN | PDLNPRITHTFDIDDNRKSCSLALRNADVYQLCS | | |
| | | | TPKVDERSDYSSIGIEDIVLDIVTSEGTVSTTRF | | |
| | | | TNNNITFDKPYAALYPSVGPGIYYDNKIIFLGYG | | |
| | | | GLEHEENGDVICNITGCPGKTQHDCNQASYSPWF | | |
| | | | SNRRMVNAIILVNKGLNKVPSLQVWTIPMRQNYW | | |
| | | | GSEGRLLLLGNKIYIYTRSTSWHSKLQLGTLDIS | | |
| | | | NYNDIRIRWTHHDVLSRPGSEECPWGNTCPRGCI | | |
| | | | TGVYNDAYPLNPSGSVVSSVILDSRTSRENPIIT | | |
| | | | YSTDTSRVNELAIRNNTLSAAYTTTNCVTHYGKG | | |
| | | | YCFHIIEINHKSLNTLQPMLFKTEIPKSCN | | |
| AB548428 | 5999-7261 | gb: AB548428: | MGSELYIIEGVSSSEIVLKQVLRRSKKILLGLVL | 1 | 122 |
| | | 5999-7261\|Organism: | SALGLTLTSTIVISICISVEQVKLRQCVDTYWAE | | |
| | | Avian metapneumovirus\| | NGSLHPGQSTENTSTRGKTTTKDPRRLQATGAGK | | |
| | | Strain Name: VCO3/ | FESCGYVQVVDGDMHDRSYAVLGGVDCLGLLALC | | |
| | | 60616\|Protein Name: | ESGPICQGDTWSEDGNFCRCTFSSHGVSCCKKPK | | |
| | | attachment glyco- | SKATTAQRNSKPANSKSTPPVHSDRASKEHNPSQ | | |
| | | protein\|Gene Symbol: G | GEQPRRGPTSSKTTIASTPSTEDTAKPTISKPKL | | |
| | | | TIRPSQRGPSGSTKAASSTPSHKTNTRGTSKTTD | | |
| | | | QRPRTGPTPERPRQTHSTATPPPTTPIHKGRAPT | | |
| | | | PKPTTDLKVNPREGSTSPTAIQKNPTTQSNLVDC | | |
| | | | TLSDPDEPQRICYQVGTYNPSQSGTCNIEVPKCS | | |
| | | | TYGHACMATLYDTPFNCWRRTRRCICDSGGELIE | | |
| | | | WCCTSQ | | |
| AF079780 | 8118-10115 | gb: AF079780\| | MDYHSHTTQTGSNETLYQDPLQSQSGSRDTLDGP | 1 | 123 |
| | | Organism: Tupaia | PSTLQHYSNPPPYSEEDQGIDGPQRSQPLSTPHQ | | |
| | | paramyxovirus\|Strain | YDRYYGVNIQHTRVYNHLGTIYKGLKLAFQILGW | | |
| | | Name: UNKNOWN- | VSVIITMIITVTTLKKMSDGNSQDSAMLKSLDEN | | |
| | | AF079780\|Protein | FDAIQEVANLLDNEVRPKLGVTMTQTTFQLPKEL | | |
| | | Name: hemagglutinin\| | SEIKRYLLRLERNCPVCGTEATPQGSKGNASGDT | | |
| | | Gene Symbol: H | AFCPPCLTRQCSEDSTHDQGPGVEGTSRNHKGKI | | |
| | | | NFPHILQSDDCGRSDNLIVYSINLVPGLSFIQLP | | |
| | | | SGTKHCIIDVSYTFSDTLAGYLIVGGVDGCQLHN | | |
| | | | KAIIYLSLGYYKTKMIYPPDYIAIATYTYDLVPN | | |
| | | | LRDCSIAVNQTSLAAICTSKKTKENQDFSTSGVH | | |
| | | | PFYIFTLNTDGIFTVTVIEQSQLKLDYQYAALYP | | |
| | | | ATGPGIFIGDHLVFLMWGGLMTKAEGDAYCQASG | | |
| | | | CNDAHRTSCNIAQMPSAYGHRQLVNGLLMLPIKE | | |
| | | | LGSHLIQPSLETISPKINWAGGHGRLYYNWEINT | | |
| | | | TYIYIEGKTWRSRPNLGITSWSKPLSIRWIDHSV | | |
| | | | ARRPGARPCDSANDCPEDCLVGGYYDMFPMSSDY | | |
| | | | KTAITIIPTHHQWPSSPALKLFNTNREVRVVMIL | | |
| | | | RPPNNVKKTTISCIRIMQTNWCLGFIIFKEGNNA | | |
| | | | WGQIYSYIYQVESTCPNTK | | |
| AY590688 | 6138-7935 | gb: AY590688: | MEVKVENVGKSQELKVKVKNFIKRSDCKKKLFAL | 1 | 124 |
| | | 6138-7935\|Organism: | ILGLVSFELTMNIMLSVMYVESNEALSLCRIQGT | | |
| | | Avian metapneumovirus\| | PAPRDNKTNTENATKETTLHTTTTTRDPEVRETK | | |
| | | Strain Name: Colorado\| | TTKPQANEGATNPSRNLTTKGDKHQTTRATTEAE | | |
| | | Protein Name: | LEKQSKQTTEPGTSTQKHTPTRPSSKSPTTTQAI | | |
| | | attachment glyco- | AQLTTPTTPKASTAPKNRQATTKKTETDTTTASR | | |
| | | protein\|Gene Symbol: G | ARNTNNPTETATTTPKATTETGKSKEGPTQHTTK | | |
| | | | EQPETTAGETTTPQPRRTASRPAPTTKIEEEAET | | |
| | | | TKTRTTKSTQTSTGPPRPTGGAPSGAATEGSGRA | | |
| | | | AAAGGPSAASAGGRRRTEAAAERDRRTRAGAGPT | | |
| | | | AGGARARTAAASERGADTAGSAGGGPGGDGATGG | | |
| | | | LSGGAPAEREDASGGTAAAGPGDGTEADGRAPPA | | |
| | | | AALAGRTTESAAGAAGDSGRAGTAGWGSAADGRS | | |
| | | | TGGNAAAEAGAAQSGRAAPRQPSGGTAPESTAPP | | |
| | | | NSGGSGRADAAPTEEVGVGSGLWRGRYVCGPCGE | | |
| | | | SVPEHPMNPCFGDGTAWICSDDGGSLPAGCYDGG | | |
| | | | TDGVVCCGVCGGNSCCCGRVECTCGGGAGLLSCC | | |
| | | | CGSYSWS | | |
| EU403085 | 6620-8593 | gb: EU403085: | MESPPSGKDAPAFREPKRTCRLCYRATTLSLNLT | 1 | 125 |
| | | 6620-8593\|Organism: | IVVLSIISIYVSTQTGANNSCVNPTIVTPDYLTG | | |
| | | Avian paramyxovirus | STTGSVEDLADLESQLREIRRDTGINLPVQIDNT | | |
| | | 3\|Strain Name: APMV3/ | ENLILTTLASINSNLRFLQNATTESQTCLSPVND | | |
| | | PKT/Netherland/449/ | PRFVAGINRIPAGSMAYNDFSNLIEHVNFIPSPT | | |
| | | 75\|Protein Name: | TLSGCTRIPSFSLSKTHWCYTHNVISNGCLDHAA | | |
| | | hemagglutinin- | SSQYISIGIVDTGLNNEPYFRTMSSKSLNDGLNR | | |
| | | neuraminidase\|Gene | KSCSVTAAANACWLLCSVVTEYEAADYRSRTPTA | | |
| | | Symbol: HN | MVLGRFDFNGEYTEIAVPSSLFDGRFASNYPGVG | | |
| | | | SGTQVNGTLYFPLYGGVLNGSDIETANKGKSFRP | | |
| | | | QNPKNRCPDSEAIQSFRAQDSYYPTRFGKVLIQQ | | |
| | | | AIIACRISNKSCTDFYLLYFDNNRVMMGAEARLY | | |
| | | | YLNNQLYLYQRSSSWWPHPLFYSISLPSCQALAV | | |
| | | | CQITEAHLTLTYATSRPGMSICTGASRCPNNCVD | | |
| | | | GVYTDVWPLTKNDAQDPNLFYTVYLNNSTRRISP | | |
| | | | TISLYTYDRRIKSKLAVGSDIGAAYTTSTCFGRS | | |
| | | | DTGAVYCLTIMETVNTIFGQYRIVPILLRVTSR | | |
| FJ977568 | 6139-7936 | gb: FJ977568: | MEVKVENVGKSQELKVKVKNFIKRSDCKKKLFAL | 1 | 126 |
| | | 6139-7936\|Organism: | ILGLVSFELTMNIMLSVMYVESNEALSLCRIQGT | | |
| | | Avian metapneumovirus\| | PAPRDNKTNTENATKETTLHTTTTTRDPEVRETK | | |
| | | Strain Name: aMPV/MN/ | TTKPQANEGATNPSRNLTTKGDKHQTTRATTEAE | | |
| | | turkey/2a/97\|Protein | LEKQSKQTTEPGTSTQKHTPARPSSKSPTTTQAT | | |
| | | Name: attachment | AQPTTPTAPKASTAPKNRQATTKKTETDTTTASR | | |
| | | glycoprotein\|Gene | ARNTNNPTETATTTPKATTETGKGKEGPTQHTTK | | |
| | | Symbol: G | EQPETTARETTTPQPRRTASRPAPTTKIEEEAET | | |
| | | | TKTRTTKNTQTSTGPPRPTRSTPSKTATENNKRT | | |
| | | | TTTKRPNTASTDSRQQTRTTAEQDQQTQTRAKPT | | |
| | | | TNGAHPQTTTTPEHNTDTTNSTKGSPKEDKTTRD | | |
| | | | PSSKTPTEQEDASKGTAAANPGGSAEADRRAPPA | | |
| | | | TTPTGRTTESAAGTTGDDSGAETTRRRSAADRRP | | |
| | | | TGGSTAAEAGTAQSGRATPKQPSGGTAAGNTAPP | | |
| | | | NNESSGRADAAPAEEAGVGPSIRRGRHACGPRRE | | |
| | | | SAPEHPTNPCPGDGTAWTRSDGGGNLPAGRHDSG | | |
| | | | ADGAARRGARGGNPRRRGRAERTRGGGAGPPSCR | | |
| | | | CGSHNRS | | |
| HG934339 | 5997-7166 | gb: HG934339: | MGAKLYAISGASDAQLMKKTCAKLLEKVVPIIIL | 1 | 127 |
| | | 5997-7166\|Organism: | AVLGITGTTTIALSISISIERAVLSDCTTQLRNG | | |
| | | Avian metapneumovirus | TTSGSLSNPTRSTTSTAVTTRDIRGLQTTRTREL | | |
| | | type D\|Strain Name: | KSCSNVQIAYGYLHDSSNPVLDSIGCLGLLALCE | | |
| | | Turkey/1985/Fr85.1\| | SGPFCQRNYNPRDRPKCRCTLRGKDISCCKEPPT | | |
| | | Protein Name: | AVTTSKTTPWGTEVHPTYPTQVTPQSQPATMAHQ | | |
| | | attachment glyco- | TATANQRSSTTEPVGSQGNTTSSNPEQQTEPPPS | | |
| | | protein\|Gene Symbol: G | PQHPPTTTSQDQSTETADGQEHTPTRKTPTATSN | | |
| | | | RRSPTPKRQETGRATPRNTATTQSGSSPPHSSPP | | |
| | | | GVDANMEGQCKELQAPKPNSVCKGLDIYREALPR | | |
| | | | GCDKVLPLCKTSTIMCVDAYYSKPPICFGYNQRC | | |
| | | | FCMETFGPIEFCCKS | | |
| JN032116 | 4659-5252 | gb: JN032116: | MSKNKNQRTARTLEKTWDTLNHLIVISSCLYKLN | 1 | 128 |
| | | 4659-5252\|Organism: | LKSIAQIALSVLAMIISTSLIIAAIIFIISANHK | | |
| | | Respiratory syncytial | VTLTTVTVQTIKNHTEKNITTYLTQVSPERVSPS | | |
| | | virus\|Strain Name: | KQPTTTPPIHTNSATISPNTKSEIHHTTAQTKGR | | |
| | | B/WI/629-12/06-07\| | TSTPTQNNKPNTKPRPKNPPKKDDYHFEVFNFVP | | |
| | | Protein Name: | CSICGNNQLCKSICKTIPSNKPRKNQP | | |
| | | attachment glyco- | | | |
| | | protein\|Gene Symbol: G | | | |
| KX258200 | 6254-7996 | gb: KX258200: | MEGSRTVIYQGDPNEKNTWRLVFRTLTLILNLAI | 1 | 129 |
| | | 6254-7996\|Organism: | LSVTIASIIITSKITLSEVTTLKTEGVEEVITPL | | |
| | | Avian paramyxovirus | MATLSDSVQQEKMIYKEVAISIPLVLDKIQTDVG | | |
| | | 14\|Strain Name: | TSVAQITDALRQIQGVNGTQAFALSNAPEYSGGI | | |
| | | APMV14/duck/Japan/ | EVPLFQIDSFVNKSMSISGLLEHASFIPSPTTLH | | |
| | | 11OG0352/2011\|Protein | GCTRIPSFHLGPRHWCYTHNIIGSRCRDEGFSSM | | |
| | | Name: hemagglutinin- | YISIGAITVNRDGNPLFITTASTILADDNNRKSC | | |
| | | neuraminidase protein\| | SIIASSYGCDLLCSIVTESENDDYANPNPTKMVH | | |
| | | Gene Symbol: HN | GRFLYNGSYVEQALPNSLFQDKWVAQYPGVGSGI | | |
| | | | TTHGKVLFPIYGGIKKNTQLFYELSKYGFFAHNK | | |
| | | | ELECKNMTEEQIRDIKAAYLPSKTSGNLFAQGII | | |
| | | | YCNISKLGDCNVAVLNTSTTMMGAEGRLQMMGEY | | |
| | | | VYYYQRSSSWWPVGIVYKKSLAELMNGINMEVLS | | |
| | | | FEPIPLSKFPRPTWTAGLCQKPSICPDVCVTGVY | | |
| | | | TDLFSVTIGSTTDKDTYFGVYLDSATERKDPWVA | | |
| | | | AADQYEWRNRVRLFESTTEAAYTTSTCFKNTVNN | | |
| | | | RVFCVSIVELRENLLGDWKIVPLLFQIGVSQGPP | | |
| | | | PK | | |
| KX940961 | 7978-12504 | gb: KX940961: | MSQLAAHNLAMSNFYGTHQGDLSGSQKGEEQQVQ | 1 | 130 |
| | | 7978-12504\|Organism: | GVIRYVSMIVGLLSLFTIIALNVTNIIYMTESGG | | |
| | | Beilong virus\|Strain | TMQSIKTAQGSIDGSMREISGVIMEDVKPKTDLI | | |
| | | Name: ERN081008_1S\| | NSMVSYNIPAQLSMIHQIIKNDVLKQCTPSFMFN | | |
| | | Protein Name: | NTICPLAENPTHSRYFEEVNLDSISECSGPDMHL | | |
| | | attachment glyco- | GLGVNPEFIEFPSFAPGSTKPGSCVRLPSFSLST | | |
| | | protein\|Gene Symbol: G | TVFAYTHTIMGHGCSELDVGDHYFSVGRIADAGH | | |
| | | | EIPQFETISSWFINDKINRRSCTVAAGAMEAWMG | | |
| | | | CVIMTETFYDDLNSLDTGKLTISYLDVFGRKKEW | | |
| | | | IYTRSEILYDYTYTSVYFSVGSGVVVGDTVYFLI | | |
| | | | WGSLSSPIEETAYCFAPDCSNYNQRMCNEAQRPS | | |
| | | | KFGHRQMVNGILKFKTTSTGKPLLSVGTLSPSVV | | |
| | | | PFGSEGRLMYSEITKIIYLYLRSTSWHALPLTGL | | |
| | | | FVLGPPTSISWIVQRAVSRPGEFPCGASNRCPKD | | |
| | | | CVTGVYTDLFPLGSRYEYAATVYLNSETYRVNPT | | |
| | | | LALINQTNIIASKKVTTESQRAGYTTTTCFVFKL | | |
| | | | RVWCISVVELAPSTMTAYEPIPFLYQLDLTCKGK | | |
| | | | NGSLAMRFTGKEGTYKSGRYKSPRNECFFEKVSN | | |
| | | | KYYFIVSTPEGIQPYEIRDLTPDRMPHIIMYISD | | |
| | | | VCAPALSAFKKLLPAMRPITTLTIGNWQFRPVEV | | |
| | | | SGGLRVSIGRNLTKEGDLTMSAPEDPGSNTFPGG | | |
| | | | HIPGNGLFDAGYYTVEYPKEWKQTTPKPSEGGNI | | |
| | | | IDKNKTPVIPSRDNPTSDSSIPHRESIEPVRPTR | | |
| | | | EVLKSSDYVTIVSTDSGSGSGDFATGVPWTGVSP | | |
| | | | KAPQNGINLPGTELPHPTVLDRINTPAPSDPKVS | | |
| | | | ADSDHTRDTIDPTALSKPLNHDTTGDTDTRINTG | | |
| | | | TATYGFTPGREATSSGKLANDLTNSTSVPSEAHP | | |
| | | | SASTSEASKPEKNTDNRVTQDPTSGTAERPTTNA | | |
| | | | PVDGKHSTQLTDARPNTADPERTSQHSSSTTRDE | | |
| | | | VKPSLPSTTEASTHQRTEAATPPELVNNTLNPPS | | |
| | | | TQVRSVRSLMQDAIAQAWNFVRGVTP | | |
| KY511044 | 6454-8310 | gb: KY511044: | MERGISEVALANDRTEEKNTWRLIFRITVLVVSV | 1 | 131 |
| | | 6454-8310\|Organism: | ITLGLTAASLVYSMNAAQPADFDGIIPAVQQVGT | | |
| | | Avian paramyxovirus | SLTNSIGGMQDVLDRTYKQVALESPLTLLNMEST | | |
| | | UPO216\|Strain Name: | IMNAITSLSYKINNGGNSSGCGAPIHDPEYIGGI | | |
| | | APMV-15/WB/Kr/UPO216/ | GKELLIDDNVDVTSFYPSAFKEHLNFIPAPTTGA | | |
| | | 2014/Protein Name: | GCTRIPSFDLSATHYCYTHNVILSGCQDHSHSHQ | | |
| | | hemagglutinin- | YIALGVLKLSDTGNVFFSTLRSINLDDTANRKSC | | |
| | | neuraminidase protein\| | SISATPLGCDILCSKVTETELEDYKSEEPTPMVH | | |
| | | Gene Symbol: HN | GRLSFDGTYSEKDLDVNNLFSDWTANYPSVGGGS | | |
| | | | YIGNRVWYAVYGGLKPGSNTDQSQRDKYVIYKRY | | |
| | | | NNTCPDPEDYQINKAKSSYTPSYFGSKRVQQAIL | | |
| | | | SIAVSPTLGSDPVLTPLSNDVVLMGAEGRVMHIG | | |
| | | | GYTYLYQRGTSYYSPALLYPLNIQDKSATASSPY | | |
| | | | KFDAFTRPGSVPCQADARCPQSCVTGVYTDPYPL | | |
| | | | IFAKDHSIRGVYGMMLNDVTARLNPIAAVFSNIS | | |
| | | | RSQITRVSSSSTKAAYTTSTCFKVIKTNRIYCMS | | |
| | | | IAEISNTLFGEFRIVPLLVEILSNGGNTARSAGG | | |
| | | | TPVKESPKGWSDAIAEPLFCTPTNVTRYNADIRR | | |
| | | | YAYSWP | | |
| NC_025360 | 8127-10158 | gb: NC_025360: | MPPAPSPVHDPSSFYGSSLFNEDTASRKGTSEEI | 1 | 132 |
| | | 8127-10158\|Organism: | HLLGIRWNTVLIVLGLILAIIGIGIGASSFSASG | | |
| | | Atlantic salmon | ITGNTTKEIRLIVEEMSYGLVRISDSVRQEISPK | | |
| | | paramyxovirus\|Strain | VTLLQNAVLSSIPALVTTETNTIINAVKNHCNSP | | |
| | | Name: ASPV/Yrkje371/ | PTPPPPTEAPLKKHETGMAPLDPTTYWTCTSGTP | | |
| | | p5\|Protein Name: | RFYSSPNATFIPGPSPLPHTATPGGCVRIPSMHI | | |
| | | hemagglutinin- | GSEIYAYTSNLIASGCQDIGKSYQNVQIGVLDRT | | |
| | | neuraminidase protein\| | PEGNPEMSPMLSHTFPINDNRKSCSIVTLKRAAY | | |
| | | Gene Symbol: HN | IYCSQPKVTEFVDYQTPGIEPMSLDHINANGTTK | | |
| | | | TWIYSPTEVVTDVPYASMYPSVGSGVVIDGKLVF | | |
| | | | LVYGGLLNGIQVPAMCLSPECPGIDQAACNASQY | | |
| | | | NQYLSGRQVVNGIATVDLMNGQKPHISVETISPS | | |
| | | | KNWFGAEGRLVYMGGRLYIYIRSTGWHSPIQIGV | | |
| | | | IYTMNPLAITWVTNTVLSRPGSAGCDWNNRCPKA | | |
| | | | CLSGVYTDAYPISPDYNHLATMILHSTSTRSNPV | | |
| | | | MVYSSPTNMVNYAQLTTTAQIAGYTTTSCFTDNE | | |
| | | | VGYCATALELTPGTLSSVQPILVMTKIPKECV | | |

### Other Proteins

In some embodiments, the fusogen may include a pH dependent protein, a homologue thereof, a fragment thereof, and a protein fusion comprising one or more proteins or fragments thereof. Fusogens may mediate membrane fusion at the cell surface or in an endosome or in another cell-membrane bound space.

In some embodiments, the fusogen includes a EFF-1, AFF-1, gap junction protein, e.g., a connexin (such as Cn43, GAP43, CX43) (DO: 10.1021/jacs.Wb5191), other tumor connection proteins, a homologue thereof, a fragment thereof, a variant thereof, and a protein fusion comprising one or more proteins or fragments thereof.

### Lipid Fusogens

In some embodiments, the retroviral vector or VLP can comprise one or more fusogenic lipids, such as saturated fatty acids. In some embodiments, the saturated fatty acids have between 10-14 carbons. In some embodiments, the saturated fatty acids have longer-chain carboxylic acids. In some embodiments, the saturated fatty acids are mono-esters.

In some embodiments, the retroviral vector or VLP can comprise one or more unsaturated fatty acids. In some embodiments, the unsaturated fatty acids have between C16 and C18 unsaturated fatty acids. In some embodiments, the unsaturated fatty acids include oleic acid, glycerol mono-oleate, glycerides, diacylglycerol, modified unsaturated fatty acids, and any combination thereof.

Without wishing to be bound by theory, in some embodiments negative curvature lipids promote membrane fusion. In some embodiments, the retroviral vector or VLP comprises one or more negative curvature lipids, e.g., exogenous negative curvature lipids, in the membrane. In embodiments, the negative curvature lipid or a precursor thereof is added to media comprising source cells, retroviral vector, or VLP. In embodiments, the source cell is engineered to express or overexpress one or more lipid synthesis genes. The negative curvature lipid can be, e.g., diacylglycerol (DAG), cholesterol, phosphatidic acid (PA), phosphatidylethanolamine (PE), or fatty acid (FA).

Without wishing to be bound by theory, in some embodiments positive curvature lipids inhibit membrane fusion. In some embodiments, the retroviral vector or VLP comprises reduced levels of one or more positive curvature lipids, e.g., exogenous positive curvature lipids, in the membrane. In embodiments, the levels are reduced by inhibiting synthesis of the lipid, e.g., by knockout or knockdown of a lipid synthesis gene, in the source cell. The positive curvature lipid can be, e.g., lysophosphatidylcholine (LPC), phosphatidylinositol (PtdIns), lysophosphatidic acid (LPA), lysophosphatidylethanolamine (LPE), or monoacylglycerol (MAG).

### Chemical Fusogens

In some embodiments, the retroviral vector or VLP may be treated with fusogenic chemicals. In some embodiments, the fusogenic chemical is polyethylene glycol (PEG) or derivatives thereof.

In some embodiments, the chemical fusogen induces a local dehydration between the two membranes that leads to unfavorable molecular packing of the bilayer. In some embodiments, the chemical fusogen induces dehydration of an area near the lipid bilayer, causing displacement of aqueous molecules between two membranes and allowing interaction between the two membranes together.

In some embodiments, the chemical fusogen is a positive cation. Some nonlimiting examples of positive cations include Ca2+, Mg2+, Mn2+, Zn2+, La3+, Sr3+, and H+.

In some embodiments, the chemical fusogen binds to the target membrane by modifying surface polarity, which alters the hydration-dependent intermembrane repulsion.

In some embodiments, the chemical fusogen is a soluble lipid soluble. Some nonlimiting examples include oleoylglycerol, dioleoylglycerol, trioleoylglycerol, and variants and derivatives thereof.

In some embodiments, the chemical fusogen is a water-soluble chemical. Some nonlimiting examples include polyethylene glycol, dimethyl sulphoxide, and variants and derivatives thereof.

In some embodiments, the chemical fusogen is a small organic molecule. A nonlimiting example includes n-hexyl bromide.

In some embodiments, the chemical fusogen does not alter the constitution, cell viability, or the ion transport properties of the fusogen or target membrane.

In some embodiments, the chemical fusogen is a hormone or a vitamin. Some nonlimiting examples include abscisic acid, retinol (vitamin A1), a tocopherol (vitamin E), and variants and derivatives thereof.

In some embodiments, the retroviral vector or VLP comprises actin and an agent that stabilizes polymerized actin. Without wishing to be bound by theory, stabilized actin in a retroviral vector or VLP can promote fusion with a target cell. In embodiments, the agent that stabilizes polymerized actin is chosen from actin, myosin, biotin-streptavidin, ATP, neuronal Wiskott-Aldrich syndrome protein (N-WASP), or formin. See, e.g., Langmuir. 2011 Aug. 16; 27(16):10061-71 and Wen et al., Nat Commun. 2016 Aug. 31; 7. In embodiments, the retroviral vector or VLP comprises exogenous actin, e.g., wild-type actin or actin comprising a mutation that promotes polymerization. In embodiments, the retroviral vector or VLP comprises ATP or phosphocreatine, e.g., exogenous ATP or phosphocreatine.

### Small Molecule Fusogens

In some embodiments, the retroviral vector or VLP may be treated with fusogenic small molecules. Some nonlimiting examples include halothane, nonsteroidal anti-inflammatory drugs (NSAIDs) such as meloxicam, piroxicam, tenoxicam, and chlorpromazine.

In some embodiments, the small molecule fusogen may be present in micelle-like aggregates or free of aggregates.

### Modifications to Protein Fusogens

Protein fusogens or viral envelope proteins may be re-targeted by mutating amino acid residues in a fusion protein or a targeting protein (e.g. the hemagglutinin protein). In some embodiments the fusogen is randomly mutated. In some embodiments the fusogen is rationally mutated. In some embodiments the fusogen is subjected to directed evolution. In some embodiments the fusogen is truncated and only a subset of the peptide is used in the retroviral vector or VLP. For example, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

Protein fusogens may be re-targeted by covalently conjugating a targeting-moiety to the fusion protein or targeting protein (e.g. the hemagglutinin protein). In some embodiments, the fusogen and targeting moiety are covalently conjugated by expression of a chimeric protein comprising the fusogen linked to the targeting moiety. A target includes any peptide (e.g. a receptor) that is displayed on a target cell. In some examples the target is expressed at higher levels on a target cell than non-target cells. For example, single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). For example, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). For example, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). A targeting protein can also include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Protein fusogens may be re-targeted by non-covalently conjugating a targeting moiety to the fusion protein or targeting protein (e.g. the hemagglutinin protein). For example, the fusion protein can be engineered to bind the Fc region of an antibody that targets an antigen on a target cell, redirecting the fusion activity towards cells that display the antibody's target (DOI: 10.1128/JV.75.17.8016-8020.2001, doi:10.1038/nml192). Altered and non-altered fusogens may be displayed on the same retroviral vector or VLP (doi: 10.1016/j.biomaterials.2014.01.051).

A targeting moiety may comprise, e.g., a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

Retroviral vectors or VLPs may display targeting moieties that are not conjugated to protein fusogens in order to redirect the fusion activity towards a cell that is bound by the targeting moiety, or to affect homing.

The targeting moiety added to the retroviral vector or VLP may be modulated to have different binding strengths. For example, scFvs and antibodies with various binding strengths may be used to alter the fusion activity of the retroviral vector or VLP towards cells that display high or low amounts of the target antigen (doi:10.1128/JV.01415-07, doi:10.1038/cgt.2014.25, DOI: 10.1002/jgm.1151). For example DARPins with different affinities may be used to alter the fusion activity of the retroviral vector or VLP towards cells that display high or low amounts of the target antigen (doi:10.1038/mt.2010.298). Targeting moieties may also be modulated to target different regions on the target ligand, which will affect the fusion rate with cells displaying the target (doi: 10.1093/protein/gzv005).

In some embodiments protein fusogens can be altered to reduce immunoreactivity, e.g., as described herein. For instance, protein fusogens may be decorated with molecules that reduce immune interactions, such as PEG (DOI: 10.1128/JVI.78.2.912-921.2004). Thus, in some embodiments, the fusogen comprises PEG, e.g., is a PEGylated polypeptide. Amino acid residues in the fusogen that are targeted by the immune system may be altered to be unrecognized by the immune system (doi: 10.1016/j.virol.2014.01.027, doi:10.1371/journal.pone.0046667). In some embodiments the protein sequence of the fusogen is altered to resemble amino acid sequences found in humans (humanized). In some embodiments the protein sequence of the fusogen is changed to a protein sequence that binds MHC complexes less strongly. In some embodiments, the protein fusogens are derived from viruses or organisms that do not infect humans (and which humans have not been vaccinated against), increasing the likelihood that a patient's immune system is naive to the protein fusogens (e.g., there is a negligible humoral or cell-mediated adaptive immune response towards the fusogen) (doi:10.1006/mthe.2002.0550, doi:10.1371/journal.ppat.1005641, doi:10.1038/gt.2011.209, DOI 10.1182/blood-2014-02-558163). In some embodiments, glycosylation of the fusogen may be changed to alter immune interactions or reduce immunoreactivity. Without wishing to be bound by theory, in some embodiments, a protein fusogen derived from a virus or organism that do not infect humans does not have a natural fusion targets in patients, and thus has high specificity.

### Positive Target Cell-Specific Regulatory Element

In some embodiments, a retroviral nucleic acid described herein comprises a positive target cell-specific regulatory element such as a tissue-specific promoter, a tissue-specific enhancer, a tissue-specific splice site, a tissue-specific site extending half-life of an RNA or protein, a tissue-specific mRNA nuclear export promoting site, a tissue-specific translational enhancing site, or a tissue-specific post-translational modification site.

A retroviral nucleic acid described herein can comprise regions, e.g., non-translated regions such as origins of replication, selection cassettes, promoters, enhancers, translation initiation signals (Shine Dalgarno sequence or Kozak sequence), introns, a polyadenylation sequence, 5′ and 3′ untranslated regions—which interact with host cellular proteins to carry out transcription and translation, and which are capable of directing, increasing, regulating, or controlling the transcription or expression of an operatively linked polynucleotide. Such elements may vary in their strength and specificity. Depending on the vector system and host utilized, any number of suitable transcription and translation elements, including ubiquitous promoters and inducible promoters may be used.

In particular embodiments, control elements are capable of directing, increasing, regulating, or controlling the transcription or expression of an operatively linked polynucleotide in a cell-specific manner. In particular embodiments, retroviral nucleic acids comprise one or more expression control sequences that are specific to particular cells, cell types, or cell lineages e.g., target cells; that is, expression of polynucleotides operatively linked to an expression control sequence specific to particular cells, cell types, or cell lineages is expressed in target cells and not (or at a lower level) in non-target cells.

In particular embodiments, a retroviral nucleic acid can include exogenous, endogenous, or heterologous control sequences such as promoters and/or enhancers.

In embodiments, the promoter comprises a recognition site to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

In embodiments, an enhancer comprises a segment of DNA which contains sequences capable of providing enhanced transcription and in some instances can function independent of orientation relative to another control sequence. An enhancer can function cooperatively or additively with promoters and/or other enhancer elements. In some embodiments, a promoter/enhancer segment of DNA contains sequences capable of providing both promoter and enhancer functions.

Illustrative ubiquitous expression control sequences include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus Orions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, β-actin promoter and a myeloproliferative sarcoma virus enhancer, negative control region deleted, d1587rev primer-binding site substituted (MND) promoter (Challita et al., J Virol. 69(2):748-55 (1995)).

In some embodiments, the promoter is a tissue-specific promoter, e.g., a promoter that drives expression in liver cells, e.g., hepatocytes, liver sinusoidal endothelial cells, cholangiocytes, stellate cells, liver-resident antigen-presenting cells (e.g., Kupffer Cells), liver-resident immune lymphocytes (e.g., T cell, B cell, or NK cell), or portal fibroblasts. Various suitable liver-specific promoters (e.g., hepatocyte-specific promoters and liver sinusoidal endothelial cell promoters) are described in Table 6 below. Table 6 also lists several ubiquitous promoters which are not specific to liver cells. In some embodiments, a fusosome (e.g., viral vector) described herein comprises, in its nucleic acid, a promoter having a sequence of Table 6, or transcriptionally active fragment thereof, or a variant having at least 70%, 75%, 80%, 85%, 90%, 95%, 96%, 97%, 98%, or 99% identity thereto. In some embodiments, a fusosome (e.g., viral vector) described herein comprises, in its nucleic acid, a promoter having transcription factor binding sites from the region within 3 kb of the transcriptional start site for the genes listed in Table 6. In some embodiments, the a fusosome (e.g., viral vector) described herein comprises, in its nucleic acid, a region within 2.5 kb, 2 kb, 1.5 kb, 1 kb, or 0.5 kb immediately upstream of the transcriptional start site of agene listed in Table 6,or a transcriptionally active fragment thereof, or a variant having at least 70%, 75%, 80%, 85%, 90%, 95%, 96%, 97%, 98%, or 99% identity thereto.

In some embodiments, a fusosome (e.g., viral vector) described herein comprises, in its nucleic acid, a promoter having a sequence set forth in any one of SEQ ID NOS:133-142 or 161-168, or transcriptionally active fragment thereof, or a variant having at least 70%, 75%, 80%, 85%, 90%, 95%, 96%, 97%, 98%, or 99% identity thereto.

TABLE 6
Exemplary promoters, e.g., hepatocyte-specific promoters

| | | Source of | | |
|---|---|---|---|---|
| | | cis- | | |
| | Promoter | regulatory | | SEQ ID |
| Specificity | Name | elements | Exemplary sequence | NO |
| Hepatocytes | hAAT | α1 | AGATCTTGCTACCAGTGGAACAGCCACTAAGG | 161 |
| | (Serpin | antitrypsin | ATTCTGCAGTGAGAGCAGAGGGCCAGCTAAGT | |
| | A1) | gene | GGTACTCTCCCAGAGACTGTCTGACTCACGCCA | |
| | | (Serpina1 | CCCCCTCCACCTTGGACACAGGACGCTGTGGTT | |
| | | gene) | TCTGAGCCAGGTACAATGACTCCTTTCGGTAAG | |
| | | | TGCAGTGGAAGCTGTACACTGCCCAGGCAAAG | |
| | | | CGTCCGGGCAGCGTAGGCGGGCGACTCAGATC | |
| | | | CCAGCCAGTGGACTTAGCCCCTGTTTGCTCCTC | |
| | | | CGATAACTGGGGTGACCTTGGTTAATATTCACC | |
| | | | AGCAGCCTCCCCCGTTGCCCCTCTGGATCCACT | |
| | | | GCTTAAATACGGACGAGGACAGGGCCCTGTCT | |
| | | | CCTCAGCTTCAGGCACCACCACTGACCTGGGA | |
| | | | CAGTGAATGTCCCCCTGATCTGCGGCCGTGACT | |
| | | | CTCTTAAGGTAGCCTTGCAGAAGTTGGTCGTGA | |
| | | | GGCACTGGGCAGGTAAGTATCAAGGTTACAAG | |
| | | | ACAGGTTTAAGGAGACCAATAGAAACTGGGCT | |
| | | | TGTCGAGACAGAGAAGACTCTTGCGTTTCTGAT | |
| | | | AGGCACCTATTGGTCTTACTGACATCCACTTTG | |
| | | | CCTTTCTCTCCACAGGTGTCCACTCCCAGTTCA | |
| | | | ATTACAGCT | |
| Hepatocytes | ApoE. | Apolipo- | gttaggctcagaggcacacaggagtttctgggctcaccctgcccccttccaac | 133 |
| | HCR- | protein | ccctcagttcccatcctccagcagctgtttgtgtgctgcctctgaagtccacact | |
| | hAAT | E/C-I | gaacaaacttcagcctactcatgtccctaaaatgggcaaacattgcaagcagc | |
| | | gene, α1 | aaacagcaaacacacagccctccctgcctgctgaccttggagctggggcag | |
| | | antitrypsin | aggtcagagacctctctgggcccatgccacctccaacatccactcgacccctt | |
| | | gene | ggaatttcggtggagaggagcagaggttgtcctggcgtggtttaggtagtgtg | |
| | | | agaggggtacccggggatcttgctaccagtggaacagccactaaggattctg | |
| | | | cagtgagagcagagggccagctaagtggtactctcccagagactgtctgact | |
| | | | cacgccaccccctccaccttggacacaggacgctgtggtttctgagccaggt | |
| | | | acaatgactcctttcggtaagtgcagtggaagctgtacactgcccaggcaaa | |
| | | | gcgtccgggcagcgtaggcgggcgactcagatcccagccagtggacttag | |
| | | | cccctgtttgctcctccgataactggggtgaccttggttaatattcaccagcagc | |
| | | | ctcccccgttgcccctctggatccactgcttaaatacggacgaggacagggc | |
| | | | cctgtctcctcagcttcaggcaccaccactgacctgggacagtgaatgatccc | |
| | | | cctgatctgcggcctcgacggtatcgataagcttgatatcgaattctagtcgtc | |
| | | | gaccactttcacaatctgctagcaacctgaggaggttatcgtacgaaattcgct | |
| | | | gtctgcgagggccagctgttggggtgagtactccctctcaaaagcgggcatg | |
| | | | acttctgcgctaagattgtcagtttccaaaaacgaggaggatttgatattcacct | |
| | | | ggcccgcggtgatgcctttgagggtggccgcgtccatctggtcagaaaaga | |
| | | | caatctttttgttgtcaagcttgaggtgtggcaggcttgagatcgatctgaccat | |
| | | | acacttgagtgacaatgacatccactttgcctttctctccacaggtgtccactcc | |
| | | | caggtccaac | |
| Hepatocytes | Enhanced | Transthyretin | CTCGAGGTCAATTCACGCGAGTTAATAATTACC | 162 |
| | trans- | gene | AGCGCGGGCCAAATAAATAATCCGCGAGGGGC | |
| | thyretin | | AGGTGACGTTTGCCCAGCGCGCGCTGGTAATT | |
| | | | ATTAACCTCGCGAATATTGATTCGAGGCCGCG | |
| | | | ATTGCCGCAATCGCGAGGGGCAGGTGACCTTT | |
| | | | GCCCAGCGCGCGTTCGCCCCGCCCCGGACGGT | |
| | | | ATCGATAAGCTTAGGAGCTTGGGCTGCAGGTC | |
| | | | GAGGGCACTGGGAGGATGTTGAGTAAGATGGA | |
| | | | AAACTACTGATGACCCTTGCAGAGACAGAGTA | |
| | | | TTAGGACATGTTTGAACAGGGGCCGGGCGATC | |
| | | | AGCAGGTAGCTCTAGAGGATCCCCGTCTGTCTG | |
| | | | CACATTTCGTAGAGCGAGTGTTCCGATACTCTA | |
| | | | ATCTCCCTAGGCAAGGTTCATATTTGTGTAGGT | |
| | | | TACTTATTCTCCTTTTGTTGACTAAGTCAATAAT | |
| | | | CAGAATCAGCAGGTTTGGAGTCAGCTTGGCAG | |
| | | | GGATCAGCAGCCTGGGTTGGAAGGAGGGGGTA | |
| | | | TAAAAGCCCCTTCACCAGGAGAAGCCGTCACA | |
| | | | CAGATCCACAAGCTCCTGCCACCATGG | |
| Hepatocyte | TTR | transthyretin | CCCACCTCCCGGAGTCCTCTCCTGCACATTCTC | 163 |
| | | | ATGTTCCTGAAAGGCTTTTCTGTCCCTTCCACT | |
| | | | ACTCCCTGTAAGCTCCTGTGCTTCACAATTTCT | |
| | | | TGTTGAATTTTTTCTAATCTGACTCTATCAGTTA | |
| | | | TGGGAATGTTCCCTCAATTCTTAGTGCTCCAAA | |
| | | | CCGGACTTGCTCTTGGCTTGTATTTGTCCAAAA | |
| | | | TATTTGTCTTCTCTATGTTTTCTACATGTTTGTC | |
| | | | TTATAAGGACAAAAACCTGCCTTAGTTTATCCA | |
| | | | TGAACAAAGCCACGCATGCTAGTGGACACACA | |
| | | | CACACATGCGCGTGCGCGCGCACACACACACA | |
| | | | CACACACATACACACAGAGACTTTGTATGTGA | |
| | | | GTAATGAATCATCAAATCATCATAATTTCTGGA | |
| | | | CTTGTATTAATAAGTCGGCCAGGAGGAAAAGA | |
| | | | ATCTGCTGTCAATCATGGCTTCTGGTTCTCACA | |
| | | | GTCATCTCTACTTTCTTCCAGCAAGTTTGGTTCT | |
| | | | GTCAAAAACCAGCTGTCAGCCTTGTTCCTGCAT | |
| | | | GCCCAATGCAGAAGAGTCAGTAAAGAAGATTT | |
| | | | GGTTCTCTGTATTTCAGGGGCATCAATGCCAGG | |
| | | | TTGAAATATGCCATTCTGGCCCAGCTCAGTGGC | |
| | | | TCACACGTGTAATCCCAGCACTTTGGAAGGCC | |
| | | | AAAGCGGGTGGATTGCTTGAGCTCAGGAGTTC | |
| | | | GAGACCAGCCTGGGCAAGAGGCTGAGGTGGGA | |
| | | | GGATGACCTGAGCCCGGGAGGTCAAGGCTGCA | |
| | | | GCGAGCTGTGATCGTGCCACTGCACTCGAGCC | |
| | | | AGGGCGTTGGAGTGAGACCCTGTCAAAAAAAA | |
| | | | AAAAAAAAAGGAAGGAAAAAAGGAAGGAAGG | |
| | | | AAGGGAGGGAGGGAAGATGCCATTCTTAGATT | |
| | | | GAAGTGGACTTTATCTGGGCAGAACACACACA | |
| | | | CACATACACACATGCACACACACATTGTGGAG | |
| | | | AAATTGCTGACTAAGCAAAGCTTCCAAATGAC | |
| | | | TTAGTTTGGCTAAAATGTAGGCTTTTAAAAATG | |
| | | | TGAGCACTGCCAAGGGTTTTTCCTTGTTGACCC | |
| | | | ATGGATCCATCAAGTGCAAACATTTTCTAATGC | |
| | | | ACTATATTTAAGCCTGTGCAGCTAGATGTCATT | |
| | | | CAACATGAAATACATTATTACAACTTGCATCTG | |
| | | | TCTAAAATCTTGCATCTAAAATGAGAGACAAA | |
| | | | AAATCTATAAAAATGGAAAACATGCATAGAAA | |
| | | | TATGTGAGGGAGGAAAAAATTACCCCCAAGAA | |
| | | | TGTTAGTGCACGCAGTCACACAGGGAGAAGAC | |
| | | | TATTTTTGTTTTGTTTTGATTGTTTTGTTTTGTTT | |
| | | | TGGTTGTTTTGTTTTGGTGACCTAACTGGTCAA | |
| | | | ATGACCTATTAAGAATATTTCATAGAACGAAT | |
| | | | GTTCCGATGCTCTAATCTCTCTAGACAAGGTTC | |
| | | | ATATTTGTATGGGTTACTTATTCTCTCTTTGTTG | |
| | | | ACTAAGTCAATAATCAGAATCAGCAGGTTTGC | |
| | | | AGTCAGATTGGCAGGGATAAGCAGCCTAGCTC | |
| | | | AGG | |
| Hepatocytes | Alb | Albumin | ccaccgcggtggcggccgctctagcttccttagcatgacgttccacttttttcta | 134 |
| | | gene | aggtggagcttacttctttgatttgatcttttgtgaaacttttggaaattacccatct | |
| | | | tcctaagcttctgcttctctcagttttctgcttgctcattccacttttccagctgacc | |
| | | | ctgccccctaccaacattgctccacaagcacaaattcatccagagaaaataaa | |
| | | | ttctaagttttatagttgtttggatcgcataggtagctaaagaggtggcaaccca | |
| | | | cacatccttaggcatgagcttgattttttttgatttagaaccttcccctctctgttcc | |
| | | | tagactacactacacattctgcaagcatagcacagagcaatgttctactttaatt | |
| | | | actttcattttcttgtatcctcacagcctagaaaataacctgcgttacagcatcca | |
| | | | ctcagtatcccttgagcatgaggtgacactacttaacatagggacgagatggt | |
| | | | actttgtgtctcctgctctgtcagcagggcactgtacttgctgataccagggaat | |
| | | | gtttgttcttaaataccatcattccggacgtgtttgccttggccagttttccatgta | |
| | | | catgcagaaagaagtaggactgatcaatacagtcctctgcattaaagcaata | |
| | | | ggaaaaggccaacttgtctacgtttagtatgtggctgtagaaagggtatagata | |
| | | | taaaaattaaaactaatgaaatggcagtcttacacatttttggcagcttatttaaa | |
| | | | gtcttggtgttaagtacgctggagctgtcacagctaccaatcaggcatgtctgg | |
| | | | gaatgagtacacggggaccataagttactgacattcgtttcccattccatttgaata | |
| | | | cacacttttgtcatggtattgcttgctgaaattgttttgcaaaaaaaaccccttcaa | |
| | | | attcatatatattattttaataaatgaattttaatttatctcaatgttataaaaaagt | |
| | | | caattttaataattaggtacttatatacccaataatatctaacaatcatttttaaaca | |
| | | | tttgtttattgagcttattatggatgaatctatctctatatactctatatactctaaa | |
| | | | aaagaagaaagaccatagacaatcatctatttgatatgtgtaaagtttacatgtga | |
| | | | gtagacatcagatgctccatttctcactgtaataccatttatagttacttgcaaaa | |
| | | | ctaactggaattctaggacttaaatattttaagttttagctgggtgactggttgga | |
| | | | aaattttaggtaagtactgaaaccaagagattataaaacaataaattctaaagttt | |
| | | | tagaagtgatcataatcaaatattaccctctaatgaaaatattccaaagttgagct | |
| | | | acagaaatttcaacataagataattttagctgtaacaatgtaatttgttgtctatttt | |
| | | | cttttgagatacagttttttctgtctagctttggctgtcctggaccttgctctgtaga | |
| | | | ccaggttggtcttgaactcagagatctgcttgcctctgccttgcaagtgctagg | |
| | | | attaaaagcatgtgccaccactgcctggctacaatctatgttttataagagattat | |
| | | | aaagctctggctttgtgacattaatctttcagataataagtcttttggattgtgtctg | |
| | | | gagaacatacagactgtgagcagatgttcagaggtatatttgcttaggggtga | |
| | | | attcaatctgcagcaataattatgagcagaattactgacacttccattttatacatt | |
| | | | ctacttgctgatctatgaaacatagataagcatgcaggcattcatcatagttttct | |
| | | | ttatctggaaaaacattaaatatgaaagaagcactttattaatacagtttagatgt | |
| | | | gttttgccatcttttaatttcttaagaaatactaagctgatgcagagtgaagagtg | |
| | | | tgtgaaaagcagtggtgcagcttggcttgaactcgttctccagcttgggatcga | |
| | | | cctgcaggcatgcttccatgccaaggcccacactgaaatgctcaaatgggag | |
| | | | acaaagagattaagctcttatgtaaaatttgctgttttacataactttaatgaatgg | |
| | | | acaaagtcttgtgcatgggggtgggggtggggttagaggggaacagctcca | |
| | | | gatggcaaacatacgcaagggatttagtcaaacaactttttggcaaagatggt | |
| | | | atgattttgtaatggggtaggaaccaatgaaatgcgaggtaagtatggttaatg | |
| | | | atctacagttattggttaaagaagtatattagagcgagtctttctgcacacagat | |
| | | | cacctttcctatcaaccccgggatcccccgggctgcaggaattcgatatcaag | |
| | | | cttatcgataccgtcgacctcgagggggggcccggtac | |
| Hepatocytes | Apoa2 | Apolipo- | CCGGGCGTGGTGGCGCATGTCTGTAATCCCAG | 164 |
| | | protein | CTACTTGGGATGCTGAGGCAGGAGAATCCTTG | |
| | | A-II | AACCCGGGAGGTGGAGGTTGCAGTGAGCCGAG | |
| | | gene | ATCATGCCATTACGCTCCAGCCTGAGCAACAA | |
| | | | GAGCAAAACTCCGTCTCAGGAAAACAAACAAA | |
| | | | AAAACCTGCACATATACTTCTGAATTTAAAACA | |
| | | | AAAGTTAAAAAACAAAGATTTCTTGGTCTCTG | |
| | | | GTCACTACCTCCCTCATCAGCTTTGCGCCTCCA | |
| | | | CTGTCACCCTCAGGAATGTTCCACATACTCAGC | |
| | | | GAGTATGCTTGGGGGGCAAAAGGGTGAAAGAT | |
| | | | ACAAAAGCTTCTGATATCTATTTAACTGATTTC | |
| | | | ACCCAAATGCTTTGAACCTGGGAATGTACCTCT | |
| | | | CCCCCTCCCCCACCCCCAACAGGAGTGAGACA | |
| | | | AGGGCCAGGGCTATTGCCCCTGCTGACTCAAT | |
| | | | ATTGGCTAATCACTGCCTAGAACTGATAAGGT | |
| | | | GATCAAATGACCAGGTGCCTTCAACCTTTACCC | |
| | | | TGGTAGAAGCCTCTTATTCACCTCTTTTCCTGC | |
| | | | CAGAGCCCTCCATTGGGAGGGGACGGGCGGAA | |
| | | | GCTGTTTTCTGAATTTGTTTTACTGGGGGTAGG | |
| | | | GTATGTTCAGTGATCAGCATCCAGGTCATTCTG | |
| | | | GGCTCTCCTGTTTTCTCCCCGTCTCATTACACAT | |
| | | | TAACTCAAAAACGGACAAGATCATTTACACTT | |
| | | | GCCCTCTTACCCGACCCTCATTCCCCTAACCCC | |
| | | | CATAGCCCTCAACCCTGTCCCTGATTTCAATTC | |
| | | | CTTTCTCCTTTCTTCTGCTCCCCAATATCTCTCT | |
| | | | GCCAAGTTGCAGTAAAGTGGGATAAGGTTGAG | |
| | | | AGATGAGATCTACCCATAATGGAATAAAGACA | |
| | | | CCATGAGCTTTCCATGGTATGATGGGTTGATGG | |
| | | | TATTCCATGGGTTGATATGTCAGAGCTTTCCAG | |
| | | | AGAAATAACTTGGAATCCTGCTTCCTGTTGCAC | |
| | | | TCAAGTCCAAGGACCTCAGATCTCAAAAGAAT | |
| | | | GAACCTCAAATATACCTGAAGTGTACCCCCTTA | |
| | | | GCCTCCACTAAGAGCTGTACCCCCTGCCTCTCA | |
| | | | CCCCATCACCATGAGTCTTCCATGTGCTTGTCC | |
| | | | TCTCCTCCCCCATTTCTCCAACTTGTTTATCCTC | |
| | | | ACATAATCCCTGCCCCACTGGGCCCATCCATAG | |
| | | | TCCCTGTCACCTGACAGGGGGTGGGTAAACAG | |
| | | | ACAGGTATATAGCCCCTTCCTCTCCAGCCAGGG | |
| | | | CAGGCACAGACACCAAGGACAGAGACGCTGGC | |
| | | | TAGGTAAGATAAGGAGGCAAGATGTGTGAGCA | |
| | | | GCATCCAAAGAGGCCTGGGCTTCAGTTGTGGA | |
| | | | GAGGGAGAGAGCCAGGTTGGAATGGGCAGCA | |
| | | | GGTAGGGAGATCCCTGGGGAGGAGCTGAAGCC | |
| | | | CATTTGGCTTCAGTGTCCCCCAAACCCCCACCA | |
| | | | CCCT | |
| Hepatocytes | Cyp3a4 | Cyp3a4 | AGCTCCTGGGGCCTGCCCTCCTCCCATTAGAAA | 165 |
| | | gene | ATCCTCCACTTGTCAAAAAGGAAGCCATTTGCT | |
| | | | TTGAACTCCAATTCCACCCCCAAGAGGCTGGG | |
| | | | ACCATCTTATTGGAGTCCTTGATGCTGTGTGAC | |
| | | | CTGCAGTGACCACTGCCCCATCATTGCTGGCTG | |
| | | | AGGTGGTTGGGGTCCATCTGGCTATCTGGGCA | |
| | | | GCTGTTCTCTTCTCTCCTTTCTCTCCTGTTTCCA | |
| | | | GACATGCAGTATTTCCAGAGAGAAGGGGCCAC | |
| | | | TCTTTGGCAAAGAACCTGTCTAACTTGCTATCT | |
| | | | ATGGCAGGACCTTTGAAGGGTTCACAGGAAGC | |
| | | | AGCACAAATTGATACTATTCCACCAAGCCATC | |
| | | | AGCTCCATCTCATCCATGCCCTGTCTCTCCTTTA | |
| | | | GGGGTCCCCTTGCCAACAGAATCACAGAGGAC | |
| | | | CAGCCTGAAAGTGCAGAGACAGCAGCTGAGGC | |
| | | | ACAGCCAAGAGCTCTGGCTGTATTAATGACCT | |
| | | | AAGAAGTCACCAGAAAGTCAGAAGGGATGAC | |
| | | | ATGCAGAGGCCCAGCAATCTCAGCTAAGTCAA | |
| | | | CTCCACCAGCCTTTCTAGTTGCCCACTGTGTGT | |
| | | | ACAGCACCCTGGTAGGGACCAGAGCCATGACA | |
| | | | GGGAATAAGACTAGACTATGCCCTTGAGGAGC | |
| | | | TCACCTCTGTTCAGGGAAACAGGCGTGGAAAC | |
| | | | ACAATGGTGGTAAAGAGGAAAGAGGACAATA | |
| | | | GGATTGCATGAAGGGGATGGAAAGTGCCCAGG | |
| | | | GGAGGAAATGGTTACATCTGTGTGAGGAGTTT | |
| | | | GGTGAGGAAAGACTCTAAGAGAAGGCTCTGTC | |
| | | | TGTCTGGGTTTGGAAGGATGTGTAGGAGTCTTC | |
| | | | TAGGGGGCACAGGCACACTCCAGGCATAGGTA | |
| | | | AAGATCTGTAGGTGTGGCTTGTTGGGATGAATT | |
| | | | TCAAGTATTTTGGAATGAGGACAGCCATAGAG | |
| | | | ACAAGGGCAGGAGAGAGGCGATTTAATAGATT | |
| | | | TTATGCCAATGGCTCCACTTGAGTTTCTGATAA | |
| | | | GAACCCAGAACCCTTGGACTCCCCAGTAACAT | |
| | | | TGATTGAGTTGTTTATGATACCTCATAGAATAT | |
| | | | GAACTCAAAGGAGGTCAGTGAGTGGTGTGTGT | |
| | | | GTGATTCTTTGCCAACTTCCAAGGTGGAGAAGC | |
| | | | CTCTTCCAACTGCAGGCAGAGCACAGGTGGCC | |
| | | | CTGCTACTGGCTGCAGCTCCAGCCCTGCCTCCT | |
| | | | TCTCTAGCATATAAACAATCCAACAGCCTCACT | |
| | | | GAATCACTGCTGTGCAGGGCAGGAAAGCTCCA | |
| | | | TGCA | |
| Hepatocytes | LP1B | Apolipo- | cggcctctagactcgagccctaaaatgggcaaacattgcaagcagcaaaca | 135 |
| | | protein | gcaaacacacagccctccctgcctgctgaccttggagctggggcagaggtc | |
| | | E/C-I | agagacctctctgggcccatgccacctccaacatccactcgaccccttggaat | |
| | | gene, α1 | ttcggtggagaggagcagaggttgtcctggcgtggtttaggtagtgtgagag | |
| | | antitrypsin | ggtggacacaggacgctgtggtttctgagccagggggcgactcagatccca | |
| | | gene | gccagtggacttagcccctgtttgctcctccgataactggggtgaccttggtta | |
| | | | atattcaccagcagcctcccccgttgcccctctggatccactgcttaaatacgg | |
| | | | acgaggacagggccctgtctcctcagcttcaggcaccaccactgacctggg | |
| | | | acagtgaatccggactctaaggtaaatataaaatttttaagtgtataatgtgttaa | |
| | | | actactgattctaattgtttctctcttttagattccaacctttggaactgaaccggt | |
| Hepatocytes | MIR122 | microRNA- | GAATGCATGGTTAACTACGTCAGAAATGACCA | 166 |
| | | 122 | GTTCAAGAGGAGAATGAGATTGGCTTCCAAAT | |
| | | | GTTGGTCAAGAGCTCTACGTAGCATGAGCCAA | |
| | | | GGATCTATTGAACTTAGTAGGCTCCTGTGACCG | |
| | | | GTGACTCTTCTGTCTCTAGAAATCTGGGGAGGT | |
| | | | GACCAGGTCATACATGGCAGTCTTCCCGTGAG | |
| | | | GAACGTTAAACTGGTTGGAAGTTGGGGTTCTG | |
| | | | AGGGGAAGATGTATTCACTAGGTGACCTGTCTT | |
| | | | CTCTGCCTCGGTGGCCTCCATGGCTGCCTGCTG | |
| | | | GCCGCACACCCCCACTCAGCAGAGGAATGGAC | |
| | | | TTTCCAATCTTGCTGAGTGTGTTTGACCAAAGG | |
| | | | TGGTGCTGACTTAGTGGCCTAAGGTCGTGCCCT | |
| | | | CCCTCCCCCACTGAATCGATAAATAATGCGACT | |
| | | | TATCAGAAAGAGAAAGAATTGTTTACTTTTAA | |
| | | | ACCCTGGATCCCATAAAGGGAGAGGGGAGAGG | |
| | | | CCTAAAGCCACAGAAGCTGTGGAAGGCGCCAT | |
| | | | CCTGCCTGCCACAGGAAGGGCCTTGGACTGAG | |
| | | | AGGACCGGAGCTGACTGGGGGTAAGTGCGGCT | |
| | | | CTCCCCCGGCGCCTGCCGACCCCCCTGAGTGAT | |
| | | | CAGGCCGTTCTTTGGGGTGGCCGCTGACCGAG | |
| | | | AAATGACGGGAGG | |
| | | | See Li et al., 2011, J. Hepatol., 55:602-611 | |
| Hepatocytes | hemopexin | Hemopexin | GCAGCTTTGGGAGTGGGCCCAGGAAGTACTGA | 167 |
| | | gene | GGATAGCAGGTGAGATCCCAGGAAGAGATGGA | |
| | | | TGTGGGGCCGAGACACTGGAGAGAGAAACAG | |
| | | | GACTGTCAGATAAAGGGCGTCTGTGACTCCTA | |
| | | | GATCTCATTATGCCTACTACCATAACCTACCCC | |
| | | | CAATTCCTAATATTCTCCTACCCTAGAGGGGGG | |
| | | | GAAATTGTCAGAAATTTGGCTGCAACACTAGC | |
| | | | AACACTACTCAGTACTTGAAATGCATTTTTGCA | |
| | | | TTTTTTTCATTCAACAAATATTTCTGGAACAAC | |
| | | | TCTTATATGCCAGGCACTATTTTAGGAGTCAGG | |
| | | | GATATATAATGGTAAACAAGACAGGCAAAACA | |
| | | | AAGCAAAGCAACAACAACCATCACCAGATAAG | |
| | | | TAGACAGATGAAAGAATTTCAAGTTTTAGTAA | |
| | | | GTAAAATAAAACAAGCAAGGGTCTGAAATGGC | |
| | | | TAGATAAGGTGGTCAAGAAAGGCTTCATTGAG | |
| | | | AAGGTAGCATTTAAGCAGGAGTCAGCTAGAAA | |
| | | | TATTGTGAAATTCCAGTTACAGTTCTATTTGTT | |
| | | | CTGGGTTGGTTAAATAAAGCTTTTTCCCCCAAG | |
| | | | GTGGAAACTACCAAGAAAGACTAATTACTAGT | |
| | | | AGTGGTGGTGCTCTCTGGAAGAGAGACACCTC | |
| | | | CTGTTTCTGCCTCATTACTGTCAACCCTTCACTT | |
| | | | CCAGGCACTTTTTGCAAAGCCCTTTGCCAGTCA | |
| | | | GGGAAGGCGAGAGGCTGGGCATGGGGCTTGGA | |
| | | | CATTTGACAACAGTGAGACATTATTGTCCCCAG | |
| | | | ACTCACTAGCCCAAGGGTAAAGCTGAAGAGGC | |
| | | | TTGGGCATGCCCCAGAAAGGCCCCTGATGAAG | |
| | | | CTTGGAAAAAGCTGTTCTCTGAGTATTTCTAAG | |
| | | | TAAGTTTATCTGTGTGTGTGGTTACTAAAAGTA | |
| | | | GTAAGTATTGCTGTCTCTAGCTGCCTTAGAGCA | |
| | | | GGGCTTGACACAGTACACAGCAATATTAGTTC | |
| | | | CCTCCTTTTCTCACCTCCCCCATTGTGGAGATA | |
| | | | AACTCAATCACAAAAGGTGATCCTCAGTCTACT | |
| | | | CACTTCCCTGACTTATGGATGCCTGGACCCATT | |
| | | | GCCAGTGTGAGAGTCACAGCTGGACGTCAGCA | |
| | | | GTGTAGCCCAGTTACTGCTTGAAAATTGCTGAA | |
| | | | GGGGGTTGGGGGGCAGCTGCCGGGAAAAAGG | |
| | | | AGTCTTGGATTCAGATTTCTGTCCAGACCCTGA | |
| | | | CCTTATTTGCAGTGATGTAATCAGCCAATATTG | |
| | | | GCTTAGTCCTGGGAGACAGCACATTCCCAGTA | |
| | | | GAGTTGGAGGTGGGGGTGGTGCTGCTGCCAAC | |
| | | | T | |
| Hepatocytes | HLP | Apolipo- | tgtttgctgcttgcaatgtttgcccattttagggtggacacaggacgctgtggttt | 136 |
| | | protein, | ctgagccagggggcgactcagatcccagccagtggacttagcccctgtttgc | |
| | | SERINA1 | tcctccgataactggggtgaccttggttaatattcaccagcagcctcccccgtt | |
| | | | gcccctctggatccactgcttaaatacggacgaggacagggccctgtctcctc | |
| | | | agcttcaggcaccaccactgacctgggacagtgaatc | |
| liver | VEC | Vascular | CCCCTGCCCTCCTCCTCTGCCCTCTCCTGGCATT | 168 |
| sinusoidal | | endothelial | CCTCCTTCATCATGGGACCCTCTTCTAATGGAT | |
| endothelial | | cadherin | CCCCAAATGTCAGAGGGTCCAAGTCCTCCCTCC | |
| cells | | gene | CTCCAAGCTCATCCATGCCCATGGCCTCAGATG | |
| | | | CCAGCCATAAGCTGTTGGGTTCCAAACCTCGAC | |
| | | | TCCAGGCTGGACTCACCCCTGTCTCCCCCACCA | |
| | | | GCCTGACACCTCCACCTGGGTATCTAACGAGC | |
| | | | ATCTCAAACTCAACCTGCCTGAGACAGAGGAA | |
| | | | TCACTATCCCCTCCTCCTCCAAAAATATCCTTC | |
| | | | CATCACACTCCCCATCTTGTGCTCTGATTTACT | |
| | | | AAACGGCCCTGGGCCCTCTCTTTCTCAGGGTCT | |
| | | | CTGCTTGCCCAGCTATATAATAAAACAAGTTTG | |
| | | | GGACTTCCCAACCATTCACCCATGGAAAAACA | |
| | | | GAAGCAACTCTTCAAAGGACAGATTCCCAGGA | |
| | | | TCTGCCCTGGGAGATTCCAAATCAGTTGATCTG | |
| | | | GGGTGAGCCCAGTCCTCTGTAGTTTTTAGAAGC | |
| | | | TCCTCCTATGTCTCTCCTGGTCAGCAGAATCTT | |
| | | | GGCCCCTCCCTTCCCCCCAGCCTCTTGGTTCTTC | |
| | | | TGGGCTCTGATCCAGCCTCAGCGTCACTGTCTT | |
| | | | CCACGCCCCTCTTTGATTCTCGTTTATGTCAAA | |
| | | | AGCCTTGTGAGGATGAGGCTGTGATTATCCCCA | |
| | | | TTTTACAGATGAGGAAACTGTGGCTCCAGGAT | |
| | | | GACACAACTGGCCAGAGGTCACATCAGAAGCA | |
| | | | GAGCTGGGTCACTTGACTCCACCCAATATCCCT | |
| | | | AAATGCAAACATCCCCTACAGACCGAGGCTGG | |
| | | | CACCTTAGAGCTGGAGTCCATGCCCGCTCTGAC | |
| | | | CAGGAGAAGCCAACCTGGTCCTCCAGAGCCAA | |
| | | | GAGCTTCTGTCCCTTTCCCATCTCCTGAAGCCT | |
| | | | CCCTGTCACCTTTAAAGTCCATTCCCACAAAGA | |
| | | | CATCATGGGATCACCACAGAAAATCAAGCTCT | |
| | | | GGGGCTAGGCTGACCCCAGCTAGATTTTTGGCT | |
| | | | CTTTTATACCCCAGCTGGGTGGACAAGCACCTT | |
| | | | AAACCCGCTGAGCCTCAGCTTCCCGGGCTATA | |
| | | | AAATGGGGGTGATGACACCTGCCTGTAGCATT | |
| | | | CCAAGGAGGGTTAAATGTGATGCTGCAGCCAA | |
| | | | GGGTCCCCACAGCCAGGCTCTTTGCAGGTGCTG | |
| | | | GGTTCAGAGTCCCAGAGCTGAGGCCGGGAGTA | |
| | | | GGGGTTCAAGTGGGGTGCCCCAGGCAGGGTCC | |
| | | | AGTGCCAGCCCTCTGTGGAGACAGCCATCCGG | |
| | | | GGCCGAGGCAGCCGCCCACCGCAGGGCCTGCC | |
| | | | TATCTGCAGCCAGCCCAGCCCTCACAAAGGAA | |
| | | | CAATAACAGGAAACCATCCCAGGGGGAAGTGG | |
| | | | GCCAGGGCCAGCTGGAAAACCTGAAGGGGAG | |
| | | | GCAGCCAGGCCTCCCTCGCCAGCGGGGTGTGG | |
| | | | CTCCCCTCCAAAGACGGTCGGCTGACAGGCTC | |
| | | | CACAGAGCTCCACTCACGCTCAGCCCTGGACG | |
| | | | GACAGGCAGTCCAACGGAACAGAAACATCCCT | |
| | | | CAGCCCACAGGCACGGTGAGTGGGGGCTCCCA | |
| | | | CACTCCCCTCCACCCCAAACCCGCCACCCTGCG | |
| ubiquitous | EF1a | EF1α gene | gggcagagcgcacatcgcccacagtccccgagaagttggggggaggggt | 137 |
| | core | | cggcaattgaacgggtgcctagagaaggtggcgcggggtaaactgggaaa | |
| | promoter | | gtgatgtcgtgtactggctccgcctttttcccgagggtgggggagaaccgtat | |
| | | | ataagtgcagtagtcgccgtgaacgttctttttcgcaacgggtttgccgccaga | |
| | | | acacag | |
| ubiquitous | EF1a | EF1α gene | ggctccggtgcccgtcagtgggcagagcgcacatcgcccacagtccccga | 138 |
| | | | gaagttggggggaggggtcggcaattgaaccggtgcctagagaaggtggc | |
| | | | gcggggtaaactgggaaagtgatgtcgtgtactggctccgcctttttcccgag | |
| | | | ggtgggggagaaccgtatataagtgcagtagtcgccgtgaacgttctttttcg | |
| | | | caacgggtttgccgccagaacacaggtaagtgccgtgtgtggttcccgcgg | |
| | | | gcctggcctctttacgggttatggcccttgcgtgccttgaattacttccacctgg | |
| | | | ctccagtacgtgattcttgatcccgagctggagccaggggcgggccttgcgc | |
| | | | tttaggagccccttcgcctcgtgcttgagttgaggcctggcctgggcgctggg | |
| | | | gccgccgcgtgcgaatctggtggcaccttcgcgcctgtctcgctgctttcgat | |
| | | | aagtctctagccatttaaaatttttgatgacctgctgcgacgctttttttctggcaa | |
| | | | gatagtcttgtaaatgcgggccaggatctgcacactggtatttcggtttttgggc | |
| | | | ccgcggccggcgacggggcccgtgcgtcccagcgcacatgttcggcgag | |
| | | | gcggggcctgcgagcgcggccaccgagaatcggacgggggtagtctcaa | |
| | | | gctggccggcctgctctggtgcctggcctcgcgccgccgtgtatcgccccgc | |
| | | | cctgggcggcaaggctggcccggtcggcaccagttgcgtgagcggaaaga | |
| | | | tggccgcttcccggccctgctccagggggctcaaaatggaggacgcggcg | |
| | | | ctcgggagagcgggcgggtgagtcacccacacaaaggaaaagggcctttc | |
| | | | cgtcctcagccgtcgcttcatgtgactccacggagtaccgggcgccgtccag | |
| | | | gcacctcgattagttctggagcttttggagtacgtcgtctttaggttgggggga | |
| | | | ggggttttatgcgatggagtttccccacactgagtgggtggagactgaagtta | |
| | | | ggccagcttggcacttgatgtaattctccttggaatttggcctttttgagtttggat | |
| | | | cttggttcattctcaagcctcagacagtggttcaaagtttttttcttccatttcaggt | |
| | | | gtcgtga | |
| ubiquitous | hPGK | PGK gene | ggggttggggttgcgccttttccaaggcagccctgggtttgcgcagggacgc | 139 |
| | | | ggctgctctgggcgtggttccgggaaacgcagcggcgccgaccctgggtct | |
| | | | cgcacattcttcacgtccgttcgcagcgtcacccggatcttcgccgctaccctt | |
| | | | gtgggccccccggcgacgcttcctgctccgcccctaagtcgggaaggttcct | |
| | | | tgcggttcgcggcgtgccggacgtgacaaacggaagccgcacgtctcacta | |
| | | | gtaccctcgcagacggacagcgccagggagcaatggcagcgcgccgacc | |
| | | | gcgatgggctgtggccaatagcggctgctcagcggggcgcgccgagagca | |
| | | | gcggccgggaaggggcggtgcgggaggcggggtgtggggcggtagtgt | |
| | | | gggccctgttcctgcccgcgcggtgttccgcattctgcaagcctccggagcg | |
| | | | cacgtcggcagtcggctccctcgttgaccgaatcaccgacctctctcccca | |
| ubiquitous | mCMV | Cytomegalo- | ggtaggcgtgtacggtgggaggcctatataagcagagct | 140 |
| | | virus | | |
| ubiquitous | Ubc | Ubiquitin C | gtctaacaaaaaagccaaaaacggccagaatttagcggacaatttactagtct | 141 |
| | | gene | aacactgaaaattacatattgacccaaatgattacatttcaaaaggtgcctaaaa | |
| | | | aacttcacaaaacacactcgccaaccccgagcgcatagttcaaaaccggag | |
| | | | cttcagctacttaagaagataggtacataaaaccgaccaaagaaactgacgc | |
| | | | ctcacttatccctcccctcaccagaggtccggcgcctgtcgattcaggagagc | |
| | | | ctaccctaggcccgaaccctgcgtcctgcgacggagaaaagcctaccgcac | |
| | | | acctaccggcaggtggccccaccctgcattataagccaacagaacgggtga | |
| | | | cgtcacgacacgacgagggcgcgcgctcccaaaggtacgggtgcactgcc | |
| | | | caacggcaccgccataactgccgcccccgcaacagacgacaaaccgagtt | |
| | | | ctccagtcagtgacaaacttcacgtcagggtccccagatggtgccccagccc | |
| | | | atctcacccgaataagagctttcccgcattagcgaaggcctcaagaccttggg | |
| | | | ttcttgccgcccaccatgccccccaccttgtttcaacgacctcacagcccgcct | |
| | | | cacaagcgtcttccattcaagactcgggaacagccgccattttgctgcgctcc | |
| | | | ccccaacccccagttcagggcaaccttgctcgcggacccagactacagccc | |
| | | | ttggcggtctctccacacgcttccgtcccaccgagcggcccggcggccacg | |
| | | | aaagccccggccagcccagcagcccgctactcaccaagtgacgatcacag | |
| | | | cgatccacaaacaagaaccgcgacccaaatcccggctgcgacggaactag | |
| | | | ctgtgccacacccggcgcgtccttatataatcatcggcgttcaccgccccacg | |
| | | | gagatccctccgcagaatcgccgagaagggactacttttcctcgcctgttccg | |
| | | | ctctctggaaagaaaaccagtgccctagagtcacccaagtcccgtcctaaaat | |
| | | | gtccttctgctgatactggggttctaaggccgagtcttatgagcagcgggccg | |
| | | | ctgtcctgagcgtccgggcggaaggatcaggacgctcgctgcgcccttcgtc | |
| | | | tgacgtggcagcgctcgccgtgaggaggggggcgcccgcgggaggcgc | |
| | | | caaaacccggcgcggaggcc | |
| ubiquitous | SFFV | Spleen | gtaacgccattttgcaaggcatggaaaaataccaaaccaagaatagagaagt | 142 |
| | | focus- | tcagatcaagggcgggtacatgaaaatagctaacgttgggccaaacaggata | |
| | | forming | tctgcggtgagcagtttcggccccggcccggggccaagaacagatggtcac | |
| | | virus | cgcagtttcggccccggcccgaggccaagaacagatggtccccagatatgg | |
| | | | cccaaccctcagcagtttcttaagacccatcagatgtttccaggctcccccaag | |
| | | | gacctgaaatgaccctgcgccttatttgaattaaccaatcagcctgcttctcgct | |
| | | | tctgttcgcgcgcttctgcttcccgagctctataaaagagctcacaacccctca | |
| | | | ctcggcgcgccagtcctccgacagactgagtcgcccggg | |

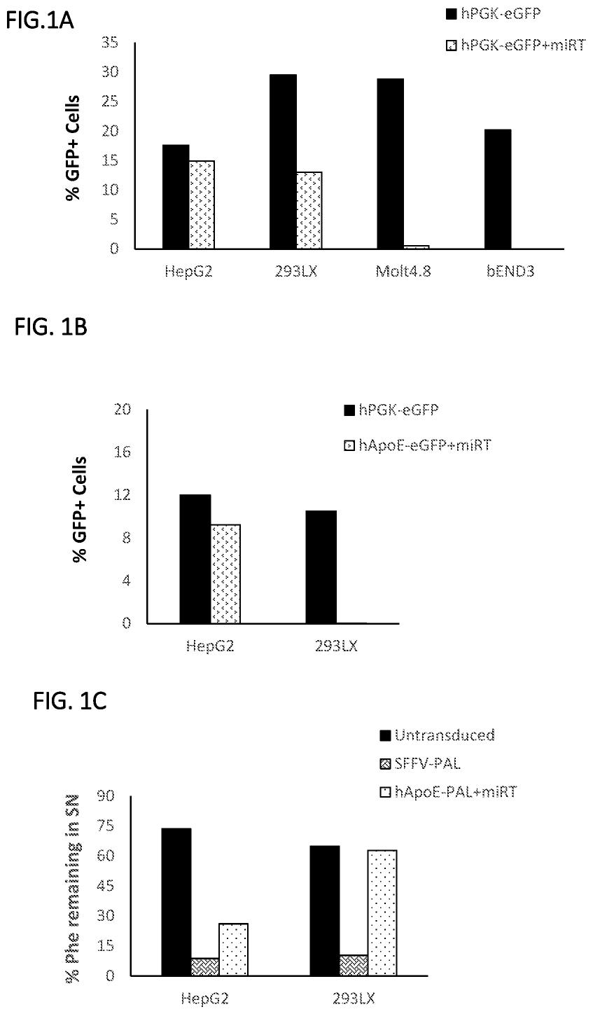

Various consensus sequences within liver-specific cis-regulatory modules (e.g., promoters) have been described. In some embodiments, a liver-specific cis-regulatory module comprises a binding site for one or more of HNF1α, C/EBP, LEF1, FOX, IRF, LEF1/TCF, Tall p/E47, and MyoD. In some embodiments, a liver-specific cis-regulatory module comprises a sequence set out in FIG. 1 or Table 1 of Chuah et al, “Liver-Specific Transcriptional Modules Identified by Genome-Wide In Silico Analysis Enable Efficient Gene Therapy in Mice and Non-Human Primates” Mol Ther. 2014 September; 22(9): 1605-1613, which is herein incorporated by reference in its entirety, including the sequences of FIG. 1 and Table 1 therein. In some embodiment, a liver-specific cis-regulatory module comprises a human sequence of HS-CRM1, HS-CRM2, HS-CRM3, HS-CRM4, HS-CRM5, HS-CRM6, HS-CRM7, HS-CRM8, HS-CRM9, HS-CRM10, HS-CRM11, HS-CRM12, HS-CRM13, or HS-CRM14 as described in Chuah et al supra.

An internal ribosome entry site (IRES) typically promotes direct internal ribosome entry to the initiation codon, such as ATG, of a cistron (a protein encoding region), thereby leading to the cap-independent translation of the gene. See, e.g., Jackson et al, (1990) Trends Biochem Sci 15(12):477-83) and Jackson and Kaminski. (1995) RNA 1 (10):985-1000. In particular embodiments, a vector includes one or more exogenous genes encoding one or more exogenous agents. In particular embodiments, to achieve efficient translation of each of the plurality of exogenous protein agents, the polynucleotide sequences can be separated by one or more IRES sequences or polynucleotide sequences encoding self-cleaving polypeptides.

The retroviral nucleic acids herein can also comprise one or more Kozak sequences, e.g., a short nucleotide sequence that facilitates the initial binding of mRNA to the small subunit of the ribosome and increases translation. The consensus Kozak sequence is (GCC)RCCATGG, where R is a purine (A or G) (Kozak, (1986) Cell. 44(2):283-92, and Kozak, (1987) Nucleic Acids Res. 15(20): 8125-48).

Promoters Responsive to a Heterologous Transcription Factor and Inducer

In some embodiments, a retroviral nucleic acid comprises an element allowing for conditional expression of the exogenous agent, e.g., any type of conditional expression including, but not limited to, inducible expression; repressible expression; cell type-specific expression, or tissue-specific expression. In some embodiments, to achieve conditional expression of the exogenous agent, expression is controlled by subjecting a cell, tissue, or organism to a treatment or condition that causes the exogenous agent to be expressed or that causes an increase or decrease in expression of the exogenous agent.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Transgene expression may be activated or repressed by the presence or absence of an inducer molecule. In some cases the inducer molecule activates or represses gene expression in a graded manner, and in some cases the inducer molecules activates or represses gene expression in an all-or-nothing manner.

A commonly used inducible promoter/system is tetracycline (Tet)-regulated system. The Tet system is based on the coexpression of two elements in the respective target cell: (i) the tetracycline response element containing repeats of the Tet-operator sequences (TetO) fused to a minimal promoter and connected to a gene of interest (e.g., a gene encoding the exogenous agent) and (ii) the transcriptional transactivator (tTA), a fusion protein of the Tet-repressor (TetR) and the transactivation domain of the herpes simplex virus derived VP16 protein. Whereas in the originally described version, transgene expression was active in the absence of tetracycline or its potent analogue doxycycline (Do), referred to as Tet-OFF system, modification of four amino acids within the transactivator protein resulted in a reverse tTA (rtTA), which only binds to TetO in the presence of Dox (Tet-ON system). In some embodiments, in the transactivator, the VP16 domain has been replaced by minimal activation domains, potential splice-donor and splice acceptor sites have been removed, and the protein has been codon optimization, resulting in the improved Transactivator variant rtTA2S-M2 with higher sensitivity to Dox and lower baseline activity. Furthermore, different Tet-responsive promoter elements have been generated, including modification in the TetO with 36-nucleotide spacing from neighboring operators to enhance regulation. Additional modifications may be useful to further reduce basal activity and increase the expression dynamic range. As an example, the pTet-T11 (short: TII) variant displays a high dynamic range and low background activity.

Conditional expression can also be achieved by using a site specific DNA recombinase. According to certain embodiments, the retroviral nucleic acid comprises at least one (typically two) site(s) for recombination mediated by a site specific recombinase, e.g., an excisive or integrative protein, enzyme, cofactor or associated protein that is involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases include, but are not limited to: Cre, Int, IHF, Xis, Flp, Fis, Hin, Gin, <DC31, Cin, Tn3 resolvase, TndX, XerC, XerD, TnpX, Hjc, Gin, SpCCE1, and ParA.

Riboswitches to Regulate Exogenous Agent Expression

Some of the compositions and methods provided herein include one or more riboswitches or polynucleotides that include one or more riboswitch. Riboswitches are a common feature in bacteria to regulate gene expression and are a means to achieve RNA control of biological functions. Riboswitches can be present in the 5′-untranslated region of mRNAs and can allow for regulatory control over gene expression through binding of a small molecule ligand that induces or suppresses a riboswitch activity. In some embodiments, the riboswitch controls a gene product involved in the generation of the small molecule ligand. Riboswitches typically act in a cis-fashion, although riboswitches have been identified that act in a trans-fashion. Natural riboswitches consist of two domains: an aptamer domain that binds the ligand through a three-dimensional folded RNA structure and a function switching domain that induces or suppresses an activity in the riboswitch based on the absence or presence of the ligand. Thus, there are two ligand sensitive conformations achieved by the riboswitch, representing on and off states (Garst et al., 2011). The function switching domain can affect the expression of a polynucleotide by regulating: an internal ribosome entry site, pre-mRNA splice donor accessibility in the retroviral gene construct, translation, termination of transcription, transcript degradation, miRNA expression, or shRNA expression (Dambach and Winkler 2009). The aptamer and function switching domains can be used as modular components allowing for synthetic RNA devices to control gene expression either as native aptamers, mutated/evolved native aptamers, or totally synthetic aptamers that are identified from screening random RNA libraries (McKeague et al 2016).

The purine riboswitch family represents one of the largest families with over 500 sequences found (Mandal et al 2003; US20080269258; and WO2006055351). The purine riboswitches share a similar structure consisting of three conserved helical elements/stem structures (PI, P2, P3) with intervening loop/junction elements (J1-2, L2, J2-3, L3, J3-1). The aptamer domains of the purine family of riboswitches naturally vary in their affinity/regulation by various purine compounds such as adenine, guanine, adenosine, guanosine, deoxyadenosine, deoxyguanosine, etc. due to sequence variation (Kim et al. 2007)

In some embodiments, a retroviral nucleic acid described herein comprises a polynucleotide encoding the exogenous agent operably linked to a promoter and a riboswitch. The riboswitch include one or more of, e.g., all of: a.) an aptamer domain, e.g., an aptamer domain capable of binding a nucleoside analogue antiviral drug and having reduced binding to guanine or 2′-deoxyguanosine relative to the nucleoside analogue antiviral drug; and b.) a function switching domain, e.g., a function switching domain capable of regulating expression of the exogenous agent, wherein binding of the nucleoside analogue by the aptamer domain induces or suppresses the expression regulating activity of the function switching domain, thereby regulating expression of the exogenous agent. In some embodiments, the exogenous agent can be a polypeptide, an miRNA, or an shRNA. For example, in an embodiment, the riboswitch is operably linked to a nucleic acid encoding a chimeric antigen receptor (CAR). In non-limiting illustrative examples provided herein, the exogenous gene encodes one or more engineered signaling polypeptides. For instance, the riboswitch and the target polynucleotide encoding one or more engineered signaling polypeptides can be found in the genome of a source cell, in a replication incompetent recombinant retroviral particle, in a T cell and/or in an NK cell.

The aptamer domains can be used, e.g., as modular components and combined with any of the function switching domains to affect the RNA transcript. In any of the embodiments disclosed herein, the riboswitch can affect the RNA transcript by regulating any of the following activities: internal ribosomal entry site (IRES), pre-mRNA splice donor accessibility, translation, termination of transcription, transcript degradation, miRNA expression, or shRNA expression. In some embodiments, the function switching domain can control binding of an anti-IRES to an IRES (see, e.g. Ogawa, RNA (2011), 17:478-488, the disclosure of which is incorporated by reference herein in its entirety). In any of the embodiments disclosed herein, the presence or absence of the small molecule ligand can cause the riboswitch to affect the RNA transcript. In some embodiments, the riboswitch can include a ribozyme. Riboswitches with ribozymes can inhibit or enhance transcript degradation of target polynucleotides in the presence of the small molecule ligand. In some embodiments, the ribozyme can be a pistol class of ribozyme, a hammerhead class of ribozyme, a twisted class of ribozyme, a hatchet class of ribozyme, or the HDV (hepatitis delta virus).

### Non-Target Cell-Specific Regulatory Element

In some embodiments, the non-target cell specific regulatory element or negative TCSRE comprises a tissue-specific miRNA recognition sequence, tissue-specific protease recognition site, tissue-specific ubiquitin ligase site, tissue-specific transcriptional repression site, or tissue-specific epigenetic repression site.

In some embodiments, a non-target cell comprises an endogenous miRNA. The retroviral nucleic acid (e.g., the gene encoding the exogenous agent) may comprise a recognition sequence for that miRNA. Thus, if the retroviral nucleic acid enters the non-target cell, the miRNA can downregulate expression of the exogenous agent. This helps achieve additional specificity for the target cell versus non-target cells.

In some embodiments, the miRNA is a small non-coding RNAs of 20-22 nucleotides, typically excised from ^{˜}70 nucleotide foldback RNA precursor structures known as pre-miRNAs. In general, miRNAs negatively regulate their targets in one of two ways depending on the degree of complementarity between the miRNA and the target. First, miRNAs that bind with perfect or nearly perfect complementarity to protein-coding mRNA sequences typically induce the RNA-mediated interference (RNAi) pathway. miRNAs that exert their regulatory effects by binding to imperfect complementary sites within the 3′ untranslated regions (UTRs) of their mRNA targets, typically repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs (e.g., naturally occurring miRNAs or artificially designed miRNAs) can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

Hundreds of distinct miRNA genes are differentially expressed during development and across tissue types. Several studies have suggested important regulatory roles for miRNAs in a broad range of biological processes including developmental timing, cellular differentiation, proliferation, apoptosis, oncogenesis, insulin secretion, and cholesterol biosynthesis. (See Bartel 2004 Cell 116:281-97; Ambros 2004 Nature 431:350-55; Du et al. 2005 Development 132:4645-52; Chen 2005 N. Engl. J. Med. 353:1768-71; Krutzfeldt et al. 2005 Nature 438:685-89.) Molecular analysis has shown that miRNAs have distinct expression profiles in different tissues. Computational methods have been used to analyze the expression of approximately 7,000 predicted human miRNA targets. The data suggest that miRNA expression broadly contributes to tissue specificity of mRNA expression in many human tissues. (See Sood et al. 2006 PNAS USA 103(8):2746-51.)

Thus, an miRNA-based approach may be used for restricting expression of the exogenous agent to a target cell population by silencing exogenous agent expression in non-target cell types by using endogenous microRNA species. MicroRNA induces sequence-specific post-transcriptional gene silencing in many organisms, either by inhibiting translation of messenger RNA (mRNA) or by causing degradation of the mRNA. See, e.g., Brown et al. 2006 Nature Med. 12(5):585-91., and WO2007/000668, each of which is herein incorporated by reference in its entirety. In some embodiments, the retroviral nucleic acid comprises one or more of (e.g., a plurality of) tissue-specific miRNA recognition sequences. In some embodiments, the tissue-specific miRNA recognition sequence is about 20-25, 21-24, or 23 nucleotides in length. In embodiments, the tissue-specific miRNA recognition sequence has perfect complementarity to an miRNA present in a non-target cell. In some embodiments, the exogenous agent does not comprise GFP, e.g., does not comprise a fluorescent protein, e.g., does not comprise a reporter protein. In some embodiments, the off-target cells are not hematopoietic cell and/or the miRNA is not present in hematopoietic cells.

In some embodiments, a method herein comprises tissue-specific expression of an exogenous agent in a target cell comprising contacting a plurality of retroviral vectors comprising a nucleotide encoding the exogenous agent and at least one tissue-specific microRNA (miRNA) target sequence with a plurality of cells comprising target cells and non-target cells, wherein the exogenous agent is preferentially expressed in, e.g., restricted, to the target cell.

For example, the retroviral nucleic acid can comprise at least one miRNA recognition sequence operably linked to a nucleotide sequence having a corresponding miRNA in a non-target cell, e.g., a hematopoietic progenitor cell (HSPC), hematopoietic stem cell (HSC), which prevents or reduces expression of the nucleotide sequence in the non-target cell but not in a target cell, e.g., differentiated cell. In some embodiments, the retroviral nucleic acid comprises at least one miRNA sequence target for a miRNA which is present in an effective amount (e.g., concentration of the endogenous miRNA is sufficient to reduce or prevent expression of a transgene) in the non-target cell, and comprises a transgene. In embodiments, the miRNA used in this system is strongly expressed in non-target cells, such as HSPC and HSC, but not in differentiated progeny of e.g. the myeloid and lymphoid lineage, preventing or reducing expression of a transgene in sensitive stem cell populations, while maintaining expression and therapeutic efficacy in the target cells.

In some embodiments, the negative TSCRE or NTSCRE comprises an miRNA recognition site, e.g., a miRNA recognition site that is bound by an miRNA endogenous to hematopoietic cells. For example, the negative TSCRE or NTSCRE is a sequence that is complementary to an miRNA endogenous to a hematopoietic cell. Exemplary miRNAs are provided in Table 7 below. In some embodiments, the nucleic acid (e.g., fusosome nucleic acid or retroviral nucleic acid) comprises a sequence that is complementary to a miRNA of Table 7, or has at least 50%, 60%, 70%, 75%, 80%, 85%, 90%, 95%, 96%, 97%, 98%, or 99% complementarity thereto. In some embodiments, the nucleic acid (e.g., fusosome nucleic acid or retroviral nucleic acid) comprises a sequence that is perfectly complementary to a seed sequence within an endogenous miRNA, e.g., miRNA of Table 7. In embodiments, the seed sequence is at least 6, 7, 8, 9, or 10 nucleotides in length.

In some embodiments, the nucleic acid (e.g., fusosome nucleic acid or retroviral nucleic acid) comprises sequence that is complementary to a miRNA set forth in any one of SEQ ID NOS: 143-160, or a sequence that has at least 50%, 60%, 70%, 75%, 80%, 85%, 90%, 95%, 96%, 97%, 98%, or 99% complementarity thereto. In some embodiments, the nucleic acid (e.g., fusosome nucleic acid or retroviral nucleic acid) includes a TSCRE or NTSCRE that comprises a sequence that is perfectly complementary to a seed sequence within an endogenous miRNA set forth in any one of SEQ ID NOS: 143-160. In embodiments, the seed sequence is at least 6, 7,8, 9, or 10nucleotides in length.

TABLE 7
Exemplary miRNA sequences.

| | | | | SEQ |
|---|---|---|---|---|
| Silenced cell | miRNA | | | ID |
| type | name | Mature miRNA | miRNA sequence | NO |
| hematopoietic | miR-142 | hsa-miR-142-3p | uguaguguuuccuacuuuaugga | 143 |
| cells | | | | |
| hematopoietic | miR-142 | hsa-miR-142-5p | cauaaaguagaaagcacuacu | 144 |
| cells | | | | |
| hematopoietic | mir-181a-2 | hsa-miR-181a-5p | aacauucaacgcugucggugagu | 145 |
| cells | | | | |
| hematopoietic | mir-181a-2 | hsa-miR-181a-2-3p | accacugaccguugacuguacc | 146 |
| cells | | | | |
| hematopoietic | mir-181b-1 | hsa-miR-181b-5p | aacauucauugcugucggugggu | 147 |
| cells | | | | |
| hematopoietic | mir-181b-1 | hsa-miR-181b-3p | cucacugaacaaugaaugcaa | 148 |
| cells | | | | |
| hematopoietic | mir-181c | hsa-miR-181c-5p | aacauucaaccugucggugagu | 149 |
| cells | | | | |
| hematopoietic | mir-181c | hsa-miR-181c-3p | aaccaucgaccguugaguggac | 150 |
| cells | | | | |
| hematopoietic | mir-181a-1 | hsa-miR-181a | aacauucaacgcugucggugagu | 151 |
| cells | | | | |
| hematopoietic | mir-181a-1 | hsa-miR-181a-3p | accaucgaccguugauuguacc | 152 |
| cells | | | | |
| hematopoietic | mir-181b-2 | hsa-miR-181b-5p | aacauucauugcugucggugggu | 153 |
| cells | | | | |
| hematopoietic | mir-181b-2 | hsa-miR-181b-2-3p | cucacugaucaaugaaugca | 154 |
| cells | | | | |
| hematopoietic | mir-181d | hsa-miR-181d-5p | aacauucauuguugucggugggu | 155 |
| cells | | | | |
| hematopoietic | mir-181d | hsa-miR-181d-3p | ccaccgggggaugaaugucac | 156 |
| cells | | | | |
| hematopoietic | miR-223 | hsa-miR-223-5p | cguguauuugacaagcugaguu | 157 |
| cells | | | | |
| hematopoietic | miR-223 | hsa-miR-223-3p | ugucaguuugucaaauacccca | 158 |
| cells | | | | |
| pDCs | miR-126 | hsa-miR-126-5p | cauuauuacuuuugguacgcg | 159 |
| pDCs | miR-126 | hsa-miR-126-3p | ucguaccgugaguaauaaugcg | 160 |

In some embodiments, the negative TSCRE or NTSCRE comprises an miRNA recognition site for an miRNA described herein. Exemplary miRNAs include those found in Griffiths-Jones et al. Nucleic Acids Res. 2006 Jan. 1, 34; Chen and Lodish, Semin Immunol. 2005 April; 17(2):155-65; Chen et al. Science. 2004 Jan. 2; 303(5654):83-6; Barad et al. Genome Res. 2004 December; 14(12): 2486-2494; Krichevsky et al., RNA. 2003 October; 9(10):1274-81; Kasashima et al. Biochem Biophys Res Commun. 2004 Sep. 17; 322(2):403-10; Houbaviy et al., Dev Cell. 2003 August; 5(2):351-8; Lagos-Quintana et al., Curr Biol. 2002 Apr. 30; 12(9):735-9; Calin et al., Proc Natl Acad Sci USA. 2004 Mar. 2; 101(9):2999-3004; Sempere et al. Genome Biol. 2004; 5(3): R13; Metzler et al., Genes Chromosomes Cancer. 2004 February; 39(2):167-9; Calin et al., Proc Natl Acad Sci USA. 2002 Nov. 26; 99(24):15524-9; Mansfield et al. Nat Genet. 2004 October; 36(10):1079-83; Michael et al. Mol Cancer Res. 2003 October; 1(12):882-91; and at www.miRNA.org.

In some embodiments, the negative TSCRE or NTSCRE comprises an miRNA recognition site for an miRNA selected from miR-1b, miR-189b, miR-93, miR-125b, miR-130, miR-32, miR-128, miR-22, miR124a, miR-296, miR-143, miR-15, miR-141, miR-143, miR-16, miR-127, miR99a, miR-183, miR-19b, miR-92, miR-9, miR-130b, miR-21, miR-30b, miR-16, miR-142-s, miR-99a, miR-212, miR-30c, miR-213, miR-20, miR-155, miR-152, miR-139, miR-30b, miR-7, miR-30c, miR-18, miR-137, miR-219, miR-1d, miR-178, miR-24, miR-122a, miR-215, miR-142-a, miR-223, miR-142, miR-124a, miR-190, miR-149, miR-193, miR-181, let-7a, miR-132, miR-27a, miR-9*, miR-200b, miR-266, miR-153, miR-135, miR-206, miR-24, miR-19a, miR-199, miR-26a, miR-194, miR-125a, miR-15a, miR-145, miR-133, miR-96, miR-131, miR-124b, miR-151, miR-7b, miR-103, and miR-208.

In some embodiments, the nucleic acid (e.g., fusosome nucleic acid or retroviral nucleic acid) comprises two or more miRNA recognition sites. In some embodiments, each of the two or more miRNA recognition sites are recognized by an miRNA as described herein, e.g., such as any set forth in of Table 7. In some embodiments, each of the two or more miRNA recognition sites are recognized by an miRNA set forth in any one of SEQ ID NOS: 143-160. In some embodiments, the two or more miRNA recognition sites can include 2, 3, 4, 5, 6, 7, 8, 9, 10 or more miRNA recognition sites. The two or more miRNA recognition sites can be positioned in tandem in the nucleic acid to provide multiple, tandem-binding sites for a miRNA.

In some embodiments, the two or more miRNA recognition sites can include at least one first miRNA recognition site, such as 1, 2, 3, 4, 5, 6 or more first miRNA recognition sites, and at least one second miRNA recognition site, such as 1, 2, 3, 4, 5, 6 or more second miRNA recognition sites. In some embodiments, the nucleic acid contains two or more first miRNA recognition site and each of the first miRNA recognition sites are present in tandem in the nucleic acid to provide multiple, tandem-binding sites for a first miRNA and/or the nucleic acid contains two more second miRNA recognition site and each of the second miRNA recognition sites are present in tandem in the nucleic acid to provide multiple, tandem-binding sites for a second miRNA. In some embodiments, the first miRNA recognition site and second miRNA recognition site are recognized by the same miRNA, and in some embodiments, the first miRNA recognition site and second miRNA recognition site are recognized by different miRNAs. In some embodiments, the first miRNA recognition site and second miRNA recognition site are recognized by miRNAs present in the same non-target cell, and in some embodiments, the first miRNA recognition site and second miRNA recognition site are recognized by miRNAs present in different non-target cells. In some embodiments, one or both of the first miRNA recognition site and second miRNA recognition site are recognized by miRNAs as described herein, such as any set forth in Table 7. In some embodiments, one or both of the first miRNA recognition site and second miRNA recognition site are recognized by an miRNAs set forth in any one of SEQ ID NOS: 143-160. In some embodiments, one or more of the miRNA recognition sites on the fusosome nucleic acid (e.g. retroviral nucleic acid) are transcribed in cis with the exogenous agent. In some embodiments, one or more of the miRNA recognition sites on the fusosome nucleic acid (e.g., retroviral nucleic acid) are situated downstream of the poly A tail sequence, e.g., between the poly A tail sequence and the WPRE. In some embodiments, one or more of the miRNA recognition sites on the fusosome nucleic acid (e.g., retroviral nucleic acid) are situated downstream of the WPRE.

### Immune Modulation

In some embodiments, a retroviral vector or VLP described herein comprises elevated CD47. See, e.g., U.S. Pat. No. 9,050,269, which is herein incorporated by reference in its entirety. In some embodiments, a retroviral vector or VLP described herein comprises elevated Complement Regulatory protein. See, e.g., ES2627445T3 and U.S. Pat. No. 6,790,641, each of which is incorporated herein by reference in its entirety. In some embodiments, a retroviral vector or VLP described herein lacks or comprises reduced levels of an MHC protein, e.g., an MHC-1 class 1 or class II. See, e.g., US20170165348, which is herein incorporated by reference in its entirety.

Sometimes retroviral vectors or VLPs are recognized by the subject's immune system. In the case of enveloped viral vector particles (e.g., retroviral vector particles), membrane-bound proteins that are displayed on the surface of the viral envelope may be recognized and the viral particle itself may be neutralised. Furthermore, on infecting a target cell, the viral envelope becomes integrated with the cell membrane and as a result viral envelope proteins may become displayed on or remain in close association with the surface of the cell. The immune system may therefore also target the cells which the viral vector particles have infected. Both effects may lead to a reduction in the efficacy of exogenous agent delivery by viral vectors.

A viral particle envelope typically originates in a membrane of the source cell. Therefore, membrane proteins that are expressed on the cell membrane from which the viral particle buds may be incorporated into the viral envelope.

### The Immune Modulating Protein CD47

The internalization of extracellular material into cells is commonly performed by a process called endocytosis (Rabinovitch, 1995, Trends Cell Biol. 5(3):85-7; Silverstein, 1995, Trends Cell Biol. 5(3):141-2; Swanson et al., 1995, Trends Cell Biol. 5(3):89-93; Allen et al., 1996, J. Exp. Med. 184(2):627-37). Endocytosis may fall into two general categories: phagocytosis, which involves the uptake of particles, and pinocytosis, which involves the uptake of fluid and solutes.

Professional phagocytes have been shown to differentiate from non-self and self, based on studies with knockout mice lacking the membrane receptor CD47 (Oldenborg et al., 2000, Science 288(5473):2051-4). CD47 is a ubiquitous member of the Ig superfamily that interacts with the immune inhibitory receptor SIRPa (signal regulatory protein) found on macrophages (Fujioka et al., 1996, Mol. Cell. Biol. 16(12):6887-99; Veillette et al., 1998, J. Biol. Chem. 273(35):22719-28; Jiang et al., 1999, J. Biol. Chem. 274(2):559-62). Although CD47-SIRPa interactions appear to deactivate autologous macrophages in mouse, severe reductions of CD47 (perhaps 90%) are found on human blood cells from some Rh genotypes that show little to no evidence of anemia (Mouro-Chanteloup et al., 2003, Blood 101(1):338-344) and also little to no evidence of enhanced cell interactions with phagocytic monocytes (Arndt et al., 2004, Br. J. Haematol. 125(3):412-4).

In some embodiments, a retroviral vector or VLP (e.g., a viral particle having a radius of less than about 1 m, less than about 400 nm, or less than about 150 nm), comprises at least a biologically active portion of CD47, e.g., on an exposed surface of the retroviral vector or VLP. In some embodiments, the retroviral vector (e.g., lentivirus) or VLP includes a lipid coat. In embodiments, the amount of the biologically active CD47 in the retroviral vector or VLP is between about 20-250, 20-50, 50-100, 100-150, 150-200, or 200-250 molecules/m². In some embodiments, the CD47 is human CD47.

A method described herein can comprise evading phagocytosis of a particle by a phagocytic cell. The method may include expressing at least one peptide including at least a biologically active portion of CD47 in a retroviral vector or VLP so that, when the retroviral vector or VLP comprising the CD47 is exposed to a phagocytic cell, the viral particle evades phacocytosis by the phagocytic cell, or shows decreased phagocytosis compared to an otherwise similar unmodified retroviral vector or VLP. In some embodiments, the half-life of the retroviral vector or VLP in a subject is extended compared to an otherwise similar unmodified retroviral vector or VLP.

MHC Deletion

The major histocompatibility complex class I (MHC-I) is a host cell membrane protein that can be incorporated into viral envelopes and, because it is highly polymorphic in nature, it is a major target of the body's immune response (McDevitt H. O. (2000) Annu. Rev. Immunol. 18: 1-17). MHC-I molecules exposed on the plasma membrane of source cells can be incorporated in the viral particle envelope during the process of vector budding. These MHC-I molecules derived from the source cells and incorporated in the viral particles can in turn be transferred to the plasma membrane of target cells. Alternatively, the MHC-I molecules may remain in close association with the target cell membrane as a result of the tendency of viral particles to absorb and remain bound to the target cell membrane.

The presence of exogenous MHC-I molecules on or close to the plasma membrane of transduced cells may elicit an alloreactive immune response in subjects. This may lead to immune-mediated killing or phagocytosis of transduced cells either upon ex vivo gene transfer followed by administration of the transduced cells to the subject, or upon direct in vivo administration of the viral particles. Furthermore, in the case of in vivo administration of MHC-I bearing viral particles into the bloodstream, the viral particles may be neutralised by pre-existing MHC-I specific antibodies before reaching their target cells.

Accordingly, in some embodiments, a source cell is modified (e.g., genetically engineered) to decrease expression of MHC-I on the surface of the cell. In embodiments, the source comprises a genetically engineered disruption of a gene encoding β2-microglobulin (β2M). In embodiments, the source cell comprises a genetically engineered disruption of one or more genes encoding an MHC-I a chain. The cell may comprise genetically engineered disruptions in all copies of the gene encoding β2-microglobulin. The cell may comprise genetically engineered disruptions in all copies of the genes encoding an MHC-I a chain. The cell may comprise both genetically engineered disruptions of genes encoding β2-microglobulin and genetically engineered disruptions of genes encoding an MHC-I a chain. In some embodiments, the retroviral vector or VLP comprises a decreased number of surface-exposed MHC-I molecules. The number of surface-exposed MHC-I molecules may be decreased such that the immune response to the MHC-I is decreased to a therapeutically relevant degree. In some embodiments, the enveloped viral vector particle is substantially devoid of surface-exposed MHC-I molecules.

HLA-G/E Overexpression

In some embodiments, a retroviral vector or VLP displays on its envelope a tolerogenic protein, e.g., an ILT-2 or ILT-4 agonist, e.g., HLA-E or HLA-G or any other ILT-2 or ILT-4 agonist. In some embodiments, a retroviral vector or VLP has increased expression of HLA-E, HLA-G, ILT-2 or ILT-4 compared to a reference retrovirus, e.g., an unmodified retrovirus otherwise similar to the retrovirus.

In some embodiments, a retrovirus composition has decreased MHC Class I compared to an unmodified retrovirus and increased HLA-G compared to an unmodified retrovirus.

In some embodiments, the retroviral vector or VLP has an increase in expression of HLA-G or HLA-E, e.g., an increase in expression of 1%, 5%, 10%, 20%, 30%, 40%, 50%, 60%, 70%, 80%, 90%, or more of HLA-G or HLA-E, compared to a reference retrovirus, e.g., an unmodified retrovirus otherwise similar to the retrovirus, wherein expression of HLA-G or HLA-E is assayed in vitro using flow cytometry, e.g., FACS.

In some embodiments, the retrovirus with increased HLA-G expression demonstrates reduced immunogenicity, e.g., as measured by reduced immune cell infiltration, in a teratoma formation assay.

Complement Regulatory Proteins

Complement activity is normally controlled by a number of complement regulatory proteins (CRPs). These proteins prevent spurious inflammation and host tissue damage. One group of proteins, including CD55/decay accelerating factor (DAF) and CD46/membrane cofactor protein (MCP), inhibits the classical and alternative pathway C3/C5 convertase enzymes. Another set of proteins including CD59 regulates MAC assembly. CRPs have been used to prevent rejection of xenotransplanted tissues and have also been shown to protect viruses and viral vectors from complement inactivation.

Membrane resident complement control factors include, e.g., decay-accelerating factor (DAF) or CD55, factor H (FH)-like protein-1 (FHL-1), C4b-binding protein (C4BP), Complement receptor 1 (CD35), membrane cofactor protein (MCP) or CD46, and CD59 (protectin) (e.g., to prevent the formation of membrane attack complex (MAC) and protect cells from lysis).

Albumin Binding Protein

In some embodiments the lentivirus binds albumin. In some embodiments the lentivirus comprises on its surface a protein that binds albumin. In some embodiments the lentivirus comprises on its surface an albumin binding protein. In some embodiments the albumin binding protein is streptococcal Albumin Binding protein. In some embodiments the albumin binding protein is streptococcal Albumin Binding Domain.

Expression of Non-Fusogen Proteins on the Lentiviral Envelope

In some embodiments the lentivirus is engineered to comprise one or more proteins on its surface. In some embodiments the proteins affect immune interactions with a subject. In some embodiments the proteins affect the pharmacology of the lentivirus in the subject. In some embodiments the protein is a receptor. In some embodiments the protein is an agonist. In some embodiments the protein is a signaling molecule. In some embodiments, the protein on the lentiviral surface comprises an anti-CD3 antibody (e.g., OKT3) or IL7.

In some embodiments, a mitogenic transmembrane protein and/or a cytokine-based transmembrane protein is present in the source cell, which can be incorporated into the retrovirus when it buds from the source cell membrane. The mitogenic transmembrane protein and/or a cytokine-based transmembrane protein can be expressed as a separate cell surface molecule on the source cell rather than being part of the viral envelope glycoprotein.

In some embodiments of any of the aspects described herein, the retroviral vector, VLP, or pharmaceutical composition is substantially non-immunogenic. Immunogenicity can be quantified, e.g., as described herein.

In some embodiments, a retroviral vector or VLP fuses with a target cell to produce a recipient cell. In some embodiments, a recipient cell that has fused to one or more retroviral vectors or VLPs is assessed for immunogenicity. In embodiments, a recipient cell is analyzed for the presence of antibodies on the cell surface, e.g., by staining with an anti-IgM antibody. In other embodiments, immunogenicity is assessed by a PBMC cell lysis assay. In embodiments, a recipient cell is incubated with peripheral blood mononuclear cells (PBMCs) and then assessed for lysis of the cells by the PBMCs. In other embodiments, immunogenicity is assessed by a natural killer (NK) cell lysis assay. In embodiments, a recipient cell is incubated with NK cells and then assessed for lysis of the cells by the NK cells. In other embodiments, immunogenicity is assessed by a CD8+ T-cell lysis assay. In embodiments, a recipient cell is incubated with CD8+ T-cells and then assessed for lysis of the cells by the CD8+ T-cells.

In some embodiments, the retroviral vector or VLP comprises elevated levels of an immunosuppressive agent (e.g., immunosuppressive protein) as compared to a reference retroviral vector or VLP, e.g., one produced from an unmodified source cell otherwise similar to the source cell, or a HEK293 cell. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the retroviral vector or VLP comprises an immunosuppressive agent that is absent from the reference cell. In some embodiments, the retroviral vector or VLP comprises reduced levels of an immunostimulatory agent (e.g., immunostimulatory protein) as compared to a reference retroviral vector or VLP, e.g., one produced from an unmodified source cell otherwise similar to the source cell, or a HEK293 cell. In some embodiments, the reduced level is at least 5%, 10%, 20%, 30%, 40%, 50%, 60%, 70%, 80%, 90%, 95%, 98%, or 99% compared to the reference retroviral vector or VLP. In some embodiments, the immunostimulatory agent is substantially absent from the retroviral vector or VLP.

In some embodiments, the retroviral vector or VLP, or the source cell from which the retroviral vector or VLP is derived, has one, two, three, four, five, six, seven, eight, nine, ten, eleven, twelve, or more of the following characteristics:
a. less than 50%, 40%, 30%, 20%, 15%, 10%, or 5% or lesser expression of MHC class I or MHC class II, compared to a reference retroviral vector or VLP, e.g., an unmodified retroviral vector or VLP from a source cell otherwise similar to the source cell, or a HeLa cell, or a HEK293 cell;
b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference retroviral vector or VLP, e.g., an unmodified retroviral vector or VLP from a cell otherwise similar to the source cell, or a HEK cell, or a reference cell described herein;
c. expression of surface proteins which suppress macrophage engulfment e.g., CD47, e.g., detectable expression by a method described herein, e.g., more than 1.5-fold, 2-fold, 3-fold, 4-fold, 5-fold, 10-fold, or more expression of the surface protein which suppresses macrophage engulfment, e.g., CD47, compared to a reference retroviral vector or VLP, e.g., an unmodified retroviral vector or VLP from a cell otherwise similar to the source cell, a Jurkat cell, or a HEK293 cell;
d. expression of soluble immunosuppressive cytokines, e.g., IL-10, e.g., detectable expression by a method described herein, e.g., more than 1.5-fold, 2-fold, 3-fold, 4-fold, 5-fold, 10-fold, or more expression of soluble immunosuppressive cytokines, e.g., IL-10, compared to a reference retroviral vector or VLP, e.g., an unmodified retroviral vector or VLP from a cell otherwise similar to the source cell, or a HEK293 cell;
e. expression of soluble immunosuppressive proteins, e.g., PD-L1, e.g., detectable expression by a method described herein, e.g., more than 1.5-fold, 2-fold, 3-fold, 4-fold, 5-fold, 10-fold, or more expression of soluble immunosuppressive proteins, e.g., PD-L1, compared to a reference retroviral vector or VLP, e.g., an unmodified retroviral vector or VLP from a cell otherwise similar to the source cell, or a HEK293 cell;
f. less than 50%, 40%, 30%, 20%, 15%, 10%, or 5% or lesser expression of soluble immune stimulating cytokines, e.g., IFN-gamma or TNF-α, compared to a reference retroviral vector or VLP, e.g., an unmodified retroviral vector or VLP from a cell otherwise similar to the source cell, or a HEK293 cell or a U-266 cell;
g. less than 50%, 40%, 30%, 20%, 15%, 10%, or 5% or lesser expression of endogenous immune-stimulatory antigen, e.g., Zg16 or Hormad1, compared to a reference retroviral vector or VLP, e.g., an unmodified retroviral vector or VLP from a cell otherwise similar to the source cell, or a HEK293 cell or an A549 cell, or a SK-BR-3 cell;
h. expression of, e.g., detectable expression by a method described herein, HLA-E or HLA-G, compared to a reference retroviral vector or VLP, e.g., an unmodified retroviral vector or VLP from a cell otherwise similar to the source cell, a HEK293 cell, or a or a Jurkat cell;
i. surface glycosylation profile, e.g., containing sialic acid, which acts to, e.g., suppress NK cell activation;
j. less than 50%, 40%, 30%, 20%, 15%, 10%, or 5% or lesser expression of TCRα/β, compared to a reference retroviral vector or VLP, e.g., an unmodified retroviral vector or VLP from a cell otherwise similar to the source cell, a HEK293 cell, or a Jurkat cell;
k. less than 50%, 40%, 30%, 20%, 15%, 10%, or 5% or lesser expression of ABO blood groups, compared to a reference retroviral vector or VLP, e.g., an unmodified retroviral vector or VLP from a cell otherwise similar to the source cell, a HEK293 cell, or a HeLa cell;
l. less than 50%, 40%, 30%, 20%, 15%, 10%, or 5% or lesser expression of Minor Histocompatibility Antigen (MHA), compared to a reference retroviral vector or VLP, e.g., an unmodified retroviral vector or VLP from a cell otherwise similar to the source cell, a HEK293 cell, or a Jurkat cell; or
m. has less than 10%, 9%, 8%, 7%, 6%, 5%, 4%, 3%, 2%, 1%, or less, of mitochondrial MHAs, compared to a reference retroviral vector or VLP e.g., an unmodified retroviral vector or VLP from a cell otherwise similar to the source cell, a HEK293 cell, or a Jurkat cell, or has no detectable mitochondrial MHAs.

In embodiments, the co-stimulatory protein is 4-1BB, B7, SLAM, LAG3, HVEM, or LIGHT, and the reference cell is HDLM-2. In some embodiments, the co-stimulatory protein is BY-H3 and the reference cell is HeLa. In some embodiments, the co-stimulatory protein is ICOSL or B7-H4, and the reference cell is SK-BR-3. In some embodiments, the co-stimulatory protein is ICOS or OX40, and the reference cell is MOLT-4. In some embodiments, the co-stimulatory protein is CD28, and the reference cell is U-266. In some embodiments, the co-stimulatory protein is CD30L or CD27, and the reference cell is Daudi.

In some embodiments, the retroviral vector, VLP, or pharmaceutical composition does not substantially elicit an immunogenic response by the immune system, e.g., innate immune system. In embodiments, an immunogenic response can be quantified, e.g., as described herein. In some embodiments, the an immunogenic response by the innate immune system comprises a response by innate immune cells including, but not limited to NK cells, macrophages, neutrophils, basophils, eosinophils, dendritic cells, mast cells, or gamma/delta T cells. In some embodiments, an immunogenic response by the innate immune system comprises a response by the complement system which includes soluble blood components and membrane bound components.

In some embodiments, the retroviral vector, VLP, or pharmaceutical composition does not substantially elicit an immunogenic response by the immune system, e.g., adaptive immune system. In some embodiments, an immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes. In some embodiments, an immunogenic response by the adaptive immune system includes increased levels of soluble blood components including, but not limited to a change, e.g., increase, in number or activity of cytokines or antibodies (e.g., IgG, IgM, IgE, IgA, or IgD).

In some embodiments, the retroviral vector, VLP, or pharmaceutical composition is modified to have reduced immunogenicity. In some embodiments, the retroviral vector, VLP, or pharmaceutical composition has an immunogenicity less than 5%, 10%, 20%, 30%, 40%, or 50% lesser than the immunogenicity of a reference retroviral vector or VLP, e.g., an unmodified retroviral vector or VLP from a cell otherwise similar to the source cell, a HEK293 cell, or a Jurkat cell.

In some embodiments of any of the aspects described herein, the retroviral vector, VLP, or pharmaceutical composition is derived from a source cell, e.g., a mammalian cell, having a modified genome, e.g., modified using a method described herein, to reduce, e.g., lessen, immunogenicity. Immunogenicity can be quantified, e.g., as described herein.

In some embodiments, the retroviral vector, VLP, or pharmaceutical composition is derived from a mammalian cell depleted of, e.g., with a knock out of, one, two, three, four, five, six, seven or more of the following:
a. MHC class I, MHC class II or MHA;
b. one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4;
c. soluble immune-stimulating cytokines e.g., IFN-gamma or TNF-α;
d. endogenous immune-stimulatory antigen, e.g., Zg16 or Hormad1;
e. T-cell receptors (TCR);
f. The genes encoding ABO blood groups, e.g., ABO gene;
g. transcription factors which drive immune activation, e.g., NFκB;
h. transcription factors that control MHC expression e.g., class II trans-activator (CIITA), regulatory factor of the Xbox 5 (RFX5), RFX-associated protein (RFXAP), or RFX ankyrin repeats (RFXANK; also known as RFXB); or
i. TAP proteins, e.g., TAP2, TAP1, or TAPBP, which reduce MHC class I expression.

In some embodiments, the retroviral vector or VLP is derived from a source cell with a genetic modification which results in increased expression of an immunosuppressive agent, e.g., one, two, three or more of the following (e.g., wherein before the genetic modification the cell did not express the factor):
a. surface proteins which suppress macrophage engulfment, e.g., CD47; e.g., increased expression of CD47 compared to a reference retroviral vector or VLP, e.g., an unmodified retroviral vector or VLP from a cell otherwise similar to the source cell, a HEK293 cell, or a Jurkat cell;
b. soluble immunosuppressive cytokines, e.g., IL-10, e.g., increased expression of IL-10 compared to a reference retroviral vector or VLP, e.g., an unmodified retroviral vector or VLP from a cell otherwise similar to the source cell, a HEK293 cell, or a Jurkat cell;
c. soluble immunosuppressive proteins, e.g., PD-1, PD-L1, CTLA4, or BTLA; e.g., increased expression of immunosuppressive proteins compared to a reference retroviral vector or VLP, e.g., an unmodified retroviral vector or VLP from a cell otherwise similar to the cell source, a HEK293 cell, or a Jurkat cell;
d. a tolerogenic protein, e.g., an ILT-2 or ILT-4 agonist, e.g., HLA-E or HLA-G or any other endogenous ILT-2 or ILT-4 agonist, e.g., increased expression of HLA-E, HLA-G, ILT-2 or ILT-4 compared to a reference retroviral vector or VLP, e.g., an unmodified retroviral vector or VLP from a cell otherwise similar to the source cell, a HEK293 cell, or a Jurkat cell; or
e. surface proteins which suppress complement activity, e.g., complement regulatory proteins, e.g. proteins that bind decay-accelerating factor (DAF, CD55), e.g. factor H (FH)-like protein-1 (FHL-1), e.g. C4b-binding protein (C4BP), e.g. complement receptor 1 (CD35), e.g. Membrane cofactor protein (MCP, CD46), eg. Profectin (CD59), e.g. proteins that inhibit the classical and alternative complement pathway CD/C5 convertase enzymes, e.g. proteins that regulate MAC assembly; e.g. increased expression of a complement regulatory protein compared to a reference retroviral vector or VLP, e.g. an unmodified retroviral vector or VLP from a cell otherwise similar to the source cell, a HEK293 cell, or a Jurkat cell.

In some embodiments, the increased expression level is at least 5%, 10%, 20%, 30%, 40%, 50%, 60%, 70%, 80%, 90%, 2-fold, 3-fold, 5-fold, 10-fold, 20-fold, 50-fold, or 100-fold higher as compared to a reference retroviral vector or VLP.

In some embodiments, the retroviral vector or VLP is derived from a source cell modified to have decreased expression of an immunostimulatory agent, e.g., one, two, three, four, five, six, seven, eight or more of the following:
a. less than 50%, 40%, 30%, 20%, 15%, 10%, or 5% or lesser expression of MHC class I or MHC class II, compared to a reference retroviral vector or VLP, e.g., an unmodified retroviral vector or VLP from a cell otherwise similar to the source cell, a HEK293 cell, or a HeLa cell;
b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference retroviral vector or VLP, e.g., an unmodified retroviral vector or VLP from a cell otherwise similar to the source cell, a HEK293 cell, or a reference cell described herein;
c. less than 50%, 40%, 30%, 20%, 15%, 10%, or 5% or lesser expression of soluble immune stimulating cytokines, e.g., IFN-gamma or TNF-α, compared to a reference retroviral vector or VLP, e.g., an unmodified retroviral vector or VLP from a cell otherwise similar to the source cell, a HEK293 cell, or a U-266 cell;
d. less than 50%, 40%, 30%, 20%, 15%, 10%, or 5% or lesser expression of endogenous immune-stimulatory antigen, e.g., Zg16 or Hormad1, compared to a reference retroviral vector or VLP, e.g., an unmodified retroviral vector or VLP from a cell otherwise similar to the source cell, a HEK293 cell, or an A549 cell or a SK-BR-3 cell;
e. less than 50%, 40%, 30%, 20%, 15%, 10%, or 5% or lesser expression of T-cell receptors (TCR) compared to a reference retroviral vector or VLP, e.g., an unmodified retroviral vector or VLP from a cell otherwise similar to the source cell, a HEK293 cell, or a Jurkat cell;
f. less than 50%, 40%, 30%, 20%, 15%, 10%, or 5% or lesser expression of ABO blood groups, compared to a reference retroviral vector or VLP, e.g., an unmodified retroviral vector or VLP from a cell otherwise similar to the source cell, a HEK293 cell, or a HeLa cell;
g. less than 50%, 40%, 30%, 20%, 15%, 10%, or 5% or lesser expression of transcription factors which drive immune activation, e.g., NFkB; compared to a reference retroviral vector or VLP, e.g., an unmodified retroviral vector or VLP from a cell otherwise similar to the source cell, a HEK293 cell, or a Jurkat cell
h. less than 50%, 40%, 30%, 20%, 15%, 10%, or 5% or lesser expression of transcription factors that control MHC expression, e.g., class II trans-activator (CIITA), regulatory factor of the Xbox 5 (RFX5), RFX-associated protein (RFXAP), or RFX ankyrin repeats (RFXANK; also known as RFXB) compared to a reference retroviral vector or VLP, e.g., an unmodified retroviral vector or VLP from a cell otherwise similar to the source cell, a HEK293 cell, or a Jurkat cell; or
i. less than 50%, 40%, 30%, 20%, 15%, 10%, or 5% or lesser expression of TAP proteins, e.g., TAP2, TAP1, or TAPBP, which reduce MHC class I expression compared to a reference retroviral vector or VLP, e.g., an unmodified retroviral vector or VLP from a cell otherwise similar to the source cell, a HEK293 cell, or a HeLa cell.

In some embodiments, a retroviral vector, VLP, or pharmaceutical composition derived from a mammalian cell, e.g., a HEK293, modified using shRNA expressing lentivirus to decrease MHC Class I expression, has lesser expression of MHC Class I compared to an unmodified retroviral vector or VLP, e.g., a retroviral vector or VLP from a cell (e.g., mesenchymal stem cell) that has not been modified. In some embodiments, a retroviral vector or VLP derived from a mammalian cell, e.g., a HEK293, modified using lentivirus expressing HLA-G to increase expression of HLA-G, has increased expression of HLA-G compared to an unmodified retroviral vector or VLP, e.g., from a cell (e.g., a HEK293) that has not been modified.

In some embodiments, the retroviral vector, VLP, or pharmaceutical composition is derived from a source cell, e.g., a mammalian cell, which is not substantially immunogenic, wherein the source cells stimulate, e.g., induce, T-cell IFN-gamma secretion, at a level of 0 μg/mL to >0 μg/mL, e.g., as assayed in vitro, by IFN-gamma ELISPOT assay.

In some embodiments, the retroviral vector, VLP, or pharmaceutical composition is derived from a source cell, e.g., a mammalian cell, wherein the mammalian cell is from a cell culture treated with an immunosuppressive agent, e.g., a glucocorticoid (e.g., dexamethasone), cytostatic (e.g., methotrexate), antibody (e.g., Muromonab (OKT3)-CD3), or immunophilin modulator (e.g., Ciclosporin or rapamycin).

In some embodiments, the retroviral vector, VLP, or pharmaceutical composition is derived from a source cell, e.g., a mammalian cell, wherein the mammalian cell comprises an exogenous agent, e.g., a therapeutic agent.

In some embodiments, the retroviral vector, VLP, or pharmaceutical composition is derived from a source cell, e.g., a mammalian cell, wherein the mammalian cell is a recombinant cell.

In some embodiments, the retroviral vector, VLP, or pharmaceutical is derived from a mammalian cell genetically modified to express viral immunoevasins, e.g., hCMV US2, or US11.

In some embodiments, the surface of the retroviral vector or VLP, or the surface of the source cell, is covalently or non-covalently modified with a polymer, e.g., a biocompatible polymer that reduces immunogenicity and immune-mediated clearance, e.g., PEG.

In some embodiments, the surface of the retroviral vector or VLP, or the surface of the source cell is covalently or non-covalently modified with a sialic acid, e.g., a sialic acid comprising glycopolymers, which contain NK-suppressive glycan epitopes.

In some embodiments, the surface of the retroviral vector or VLP, or the surface of the source cell is enzymatically treated, e.g., with glycosidase enzymes, e.g., α-N-acetylgalactosaminidases, to remove ABO blood groups In some embodiments, the surface of the retroviral vector or VLP, or the surface of the source cell is enzymatically treated, to give rise to, e.g., induce expression of, ABO blood groups which match the recipient's blood type.

### Parameters for Assessing Immunogenicity

In some embodiments, the retroviral vector or VLP is derived from a source cell, e.g., a mammalian cell which is not substantially immunogenic, or modified, e.g., modified using a method described herein, to have a reduction in immunogenicity. Immunogenicity of the source cell and the retroviral vector or VLP can be determined by any of the assays described herein.

In some embodiments, the retroviral vector or VLP has an increase, e.g., an increase of 1%, 5%, 10%, 20%, 30%, 40%, 50%, 60%, 70%, 80%, 90%, or more, in in vivo graft survival compared to a reference retroviral vector or VLP, e.g., an unmodified retroviral vector or VLP from a cell otherwise similar to the source cell.

In some embodiments, the retroviral vector or VLP has a reduction in immunogenicity as measured by a reduction in humoral response following one or more implantation of the retroviral vector or VLP into an appropriate animal model, e.g., an animal model described herein, compared to a humoral response following one or more implantation of a reference retroviral vector or VLP, e.g., an unmodified retroviral vector or VLP from a cell otherwise similar to the source cell, into an appropriate animal model, e.g., an animal model described herein. In some embodiments, the reduction in humoral response is measured in a serum sample by an anti-cell antibody titre, e.g., anti-retroviral or anti-VLP antibody titre, e.g., by ELISA. In some embodiments, the serum sample from animals administered the retroviral vector or VLP has a reduction of 1%, 5%, 10%, 20%, 30%, 40%, 50%, 60%, 70%, 80%, 90%, or more of an anti-retroviral or anti-VLP antibody titer compared to the serum sample from animals administered an unmodified retroviral vector or VLP. In some embodiments, the serum sample from animals administered the retroviral vector or VLP has an increased anti-retroviral or anti-VLP antibody titre, e.g., increased by 1%, 2%, 5%, 10%, 20%, 30%, or 40% from baseline, e.g., wherein baseline refers to serum sample from the same animals before administration of the retroviral vector or VLP.

In some embodiments, the retroviral vector or VLP has a reduction in macrophage phagocytosis, e.g., a reduction of 1%, 5%, 10%, 20%, 30%, 40%, 50%, 60%, 70%, 80%, 90%, or more in macrophage phagocytosis compared to a reference retroviral vector or VLP, e.g., an unmodified retroviral vector or VLP from a cell otherwise similar to the source cell, wherein the reduction in macrophage phagocytosis is determined by assaying the phagocytosis index in vitro, e.g., as described in Example 8. In some embodiments, the retroviral vector or VLP has a phagocytosis index of 0, 1, 10, 100, or more, e.g., as measured by an assay of Example 8, when incubated with macrophages in an in vitro assay of macrophage phagocytosis.

In some embodiments, the source cell or recipient cell has a reduction in cytotoxicity mediated cell lysis by PBMCs, e.g., a reduction of 1%, 5%, 10%, 20%, 30%, 40%, 50%, 60%, 70%, 80%, 90%, or more in cell lysis compared to a reference cell, e.g., an unmodified cell otherwise similar to the source cell, or a recipient cell that received an unmodified retroviral vector or VLP, or a mesenchymal stem cells, e.g., using an assay of Example 17. In embodiments, the source cell expresses exogenous HLA-G.

In some embodiments, the source cell or recipient cell has a reduction in NK-mediated cell lysis, e.g., a reduction of 1%, 5%, 10%, 20%, 30%, 40%, 50%, 60%, 70%, 80%, 90%, or more in NK-mediated cell lysis compared to a reference cell, e.g., an unmodified cell otherwise similar to the source cell, or a recipient cell that received an unmodified retroviral vector or VLP, wherein NK-mediated cell lysis is assayed in vitro, by a chromium release assay or europium release assay, e.g., using an assay of Example 18.

In some embodiments, the source cell or recipient cell has a reduction in CD8+ T-cell mediated cell lysis, e.g., a reduction of 1%, 5%, 10%, 20%, 30%, 40%, 50%, 60%, 70%, 80%, 90%, or more in CD8 T cell mediated cell lysis compared to a reference cell, e.g., an unmodified cell otherwise similar to the source cell, or a recipient cell that received an unmodified retroviral vector or VLP, wherein CD8 T cell mediated cell lysis is assayed in vitro, by an assay of Example 19.

In some embodiments, the source cell or recipient cell has a reduction in CD4+ T-cell proliferation and/or activation, e.g., a reduction of 1%, 5%, 10%, 20%, 30%, 40%, 50%, 60%, 70%, 80%, 90%, or more compared to a reference cell, e.g., an unmodified cell otherwise similar to the source cell, or a recipient cell that received an unmodified retroviral vector or VLP, wherein CD4 T cell proliferation is assayed in vitro (e.g. co-culture assay of modified or unmodified mammalian source cell, and CD4+ T-cells with CD3/CD28 Dynabeads).

In some embodiments, the retroviral vector or VLP causes a reduction in T-cell IFN-gamma secretion, e.g., a reduction of 1%, 5%, 10%, 20%, 30%, 40%, 50%, 60%, 70%, 80%, 90%, or more in T-cell IFN-gamma secretion compared to a reference retroviral vector or VLP, e.g., an unmodified retroviral vector or VLP from a cell otherwise similar to the source cell, wherein T-cell IFN-gamma secretion is assayed in vitro, e.g., by IFN-gamma ELISPOT.

In some embodiments, the retroviral vector or VLP causes a reduction in secretion of immunogenic cytokines, e.g., a reduction of 1%, 5%, 10%, 20%, 30%, 40%, 50%, 60%, 70%, 80%, 90%, or more in secretion of immunogenic cytokines compared to a reference retroviral vector or VLP, e.g., an unmodified retroviral vector or VLP from a cell otherwise similar to the source cell, wherein secretion of immunogenic cytokines is assayed in vitro using ELISA or ELISPOT.

In some embodiments, the retroviral vector or VLP results in increased secretion of an immunosuppressive cytokine, e.g., an increase of 1%, 5%, 10%, 20%, 30%, 40%, 50%, 60%, 70%, 80%, 90%, or more in secretion of an immunosuppressive cytokine compared to a reference retroviral vector or VLP, e.g., an unmodified retroviral vector or VLP from a cell otherwise similar to the source cell, wherein secretion of the immunosuppressive cytokine is assayed in vitro using ELISA or ELISPOT.

In some embodiments, the retroviral vector or VLP has an increase in expression of HLA-G or HLA-E, e.g., an increase in expression of 1%, 5%, 10%, 20%, 30%, 40%, 50%, 60%, 70%, 80%, 90%, or more of HLA-G or HLA-E, compared to a reference retroviral vector or VLP, e.g., an unmodified retroviral vector or VLP from a cell otherwise similar to the source cell, wherein expression of HLA-G or HLA-E is assayed in vitro using flow cytometry, e.g., FACS. In some embodiments, the retroviral vector or VLP is derived from a source cell which is modified to have an increased expression of HLA-G or HLA-E, e.g., compared to an unmodified cell, e.g., an increased expression of 1%, 5%, 10%, 20%, 30%, 40%, 50%, 60%, 70%, 80%, 90%, or more of HLA-G or HLA-E, wherein expression of HLA-G or HLA-E is assayed in vitro using flow cytometry, e.g., FACS. In some embodiments, the retroviral vector or VLP derived from a modified cell with increased HLA-G expression demonstrates reduced immunogenicity.

In some embodiments, the retroviral vector or VLP has or causes an increase in expression of T cell inhibitor ligands (e.g. CTLA4, PD1, PD-L1), e.g., an increase in expression of 1%, 5%, 10%, 20%, 30%, 40%, 50%, 60%, 70%, 80%, 90%, or more of T cell inhibitor ligands as compared to a reference retroviral vector or VLP, e.g., an unmodified retroviral vector or VLP from a cell otherwise similar to the source cell, wherein expression of T cell inhibitor ligands is assayed in vitro using flow cytometry, e.g., FACS.

In some embodiments, the retroviral vector or VLP has a decrease in expression of co-stimulatory ligands, e.g., a decrease of 1%, 5%, 10%, 20%, 30%, 40%, 50%, 60%, 70%, 80%, 90%, or more in expression of co-stimulatory ligands compared to a reference retroviral vector or VLP, e.g., an unmodified retroviral vector or VLP from a cell otherwise similar to the source cell, wherein expression of co-stimulatory ligands is assayed in vitro using flow cytometry, e.g., FACS.

In some embodiments, the retroviral vector or VLP has a decrease in expression of MHC class I or MHC class II, e.g., a decrease in expression of 1%, 5%, 10%, 20%, 30%, 40%, 50%, 60%, 70%, 80%, 90%, or more of MHC Class I or MHC Class II compared to a reference retroviral vector or VLP, e.g., an unmodified retroviral vector or VLP from a cell otherwise similar to the source cell or a HeLa cell, wherein expression of MHC Class I or II is assayed in vitro using flow cytometry, e.g., FACS.

In some embodiments, the retroviral vector or VLP is derived from a cell source, e.g., a mammalian cell source, which is substantially non-immunogenic. In some embodiments, immunogenicity can be quantified, e.g., as described herein. In some embodiments, the mammalian cell source comprises any one, all or a combination of the following features:
a. wherein the source cell is obtained from an autologous cell source; e.g., a cell obtained from a recipient who will be receiving, e.g., administered, the retroviral vector or VLP;
b. wherein the source cell is obtained from an allogeneic cell source which is of matched, e.g., similar, gender to a recipient, e.g., a recipient described herein who will be receiving, e.g., administered; the retroviral vector or VLP;
c. wherein the source cell is obtained is from an allogeneic cell source is which is HLA matched with a recipient's HLA, e.g., at one or more alleles;
d. wherein the source cell is obtained is from an allogeneic cell source which is an HLA homozygote;
e. wherein the source cell is obtained is from an allogeneic cell source which lacks (or has reduced levels compared to a reference cell) MHC class I and II; or
f. wherein the source cell is obtained is from a cell source which is known to be substantially non-immunogenic including but not limited to a stem cell, a mesenchymal stem cell, an induced pluripotent stem cell, an embryonic stem cell, a sertoli cell, or a retinal pigment epithelial cell.

In some embodiments, the subject to be administered the retroviral vector or VLP has, or is known to have, or is tested for, a pre-existing antibody (e.g., IgG or IgM) reactive with a retroviral vector or VLP. In some embodiments, the subject to be administered the retroviral vector or VLP does not have detectable levels of a pre-existing antibody reactive with the retroviral vector or VLP. Tests for the antibody are described, e.g., in Example 13.

In some embodiments, a subject that has received the retroviral vector or VLP has, or is known to have, or is tested for, an antibody (e.g., IgG or IgM) reactive with a retroviral vector or VLP. In some embodiments, the subject that received the retroviral vector or VLP (e.g., at least once, twice, three times, four times, five times, or more) does not have detectable levels of antibody reactive with the retroviral vector or VLP. In embodiments, levels of antibody do not rise more than 1%, 2%, 5%, 10%, 20%, or 50% between two timepoints, the first timepoint being before the first administration of the retroviral vector or VLP, and the second timepoint being after one or more administrations of the retroviral vector or VLP. Tests for the antibody are described, e.g., in Example 14.

### Exogenous Agents

In some embodiments, a retroviral vector, VLP, or pharmaceutical composition described herein encodes an exogenous agent.

### Exogenous Protein Agents

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell.

In some embodiments, the exogenous agent comprises a nucleic acid, e.g., RNA, intron(s), exon(s), mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), IncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid or a mutant nucleic acid. In some embodiments the nucleic acid is a fusion or chimera of multiple nucleic acid sequences.

In some embodiments, the exogenous agent comprises a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein or a mutant protein. In some embodiments the protein is a fusion or chimeric protein.

### Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the membrane protein comprises a sequence of SEQ ID NOs: 8144-16131 of U.S. Patent Publication No. 2016/0289674, which are herein incorporated by reference in their entireties. In some embodiments, the membrane protein comprises a fragment, variant, or homolog of a sequence of SEQ ID NOs: 8144-16131 of U.S. Patent Publication No. 2016/0289674. In some embodiments, the membrane protein comprises a nucleic acid encoding a protein comprising a sequence of SEQ ID NOs: 8144-16131 of U.S. Patent Publication No. 2016/0289674. In some embodiments, the membrane protein comprises a nucleic acid encoding a protein comprising a fragment, variant, or homolog of a sequence of SEQ ID NOs: 8144-16131 of U.S. Patent Publication No. 2016/0289674.

In some embodiments, the membrane protein comprises a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the antigen binding domain targets an antigen characteristic of a neoplastic cell. In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphAl, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (SlPlR), NMDA channel, transmembrane protein, multispan transmembrane protein, T-cell receptor motifs; T-cell alpha chains; T-cell Rchains; T-cell γ chains; T-cell 6 chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Thl; Th2; Thl7; Th40; Th22; Th9; Tfh, Canonical Treg. FoxP3+; Trl; Th3; Treg17; TREG; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-72, VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-10, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LCAM, LeY, MSLN, IL13R□1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-iRa), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCRi, ADRB3, PANX3, GPR20, LY6K, OR51E2, TARP, WT1, NY-ESO-1, LAGE-la, MAGE-A1, legumain, HPV E6, E7, ETV6-AML, sperm protein 17, XAGE1, Tie 2, MAD-CT-1, MAD-CT-2, Fos-related antigen 1, p53, p⁵³ mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin Bi, MYCN, RhoC, TRP-2, CYPIB I, BORIS, SART3, PAX5, OY-TES1, LCK, AKAP-4, SSX2, RAGE-1, human telomerase reverse transcriptase, RUi, RU2, intestinal carboxyl esterase, mut hsp70-2, CD79a, CD79b, CD72, LAIR1, FCAR, LILRA2, CD300LF, CLECi2A, BST2, EMR2, LY75, GPC3, FCRL5, IGLL, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the CAR transmembrane domain comprises at least a transmembrane region of the alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8a, CD80, 4-1BB/CD137, CD28, CD34, CD4, FcεRIγ, CD16, OX40/CD134, CD3ζ, CD3F, CD37, CD36, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCLB; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

miRNA and siRNA

In embodiments, the retroviral genome encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155.

In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target.

In some embodiments, the exogenous agent comprises a shRNA. A shRNA (short hairpin RNA) can comprise a double-stranded structure that is formed by a single self-complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1 RNA promoter and the human tRNA-val promoter, or a strong constitutive pol II promoter.

### Fusogen Receptors and Methods of Preventing Source Cell Fusion

In some embodiments, a source cell is modified (e.g., using siRNA, miRNA, shRNA, genome editing, or other methods) to have reduced expression (e.g., no expression) of a fusogen receptor that binds a fusogen expressed by the source cell. In some embodiments, the fusogen is a re-targeted fusogen, e.g., the fusogen may comprise a target-binding domain, e.g., an antibody, e.g., an scFv. In some embodiments, the fusogen receptor is bound by the antibody.

### Insulator Elements

In some embodiments, a retroviral or lentiviral vector or VLP further comprises one or more insulator elements, e.g., an insulator element described herein. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (e.g., position effect; see, e.g., Burgess-Beusse et al, 2002, Proc. Natl. Acad. Sci., USA, 99: 16433; and Zhan et al, 2001, Hum. Genet., 109:471) or deregulated expression of endogenous sequences adjacent to the transferred sequences. In some embodiments, transfer vectors comprise one or more insulator element the 3′ LTR and upon integration of the provirus into the host genome, the provirus comprises the one or more insulators at the 5′ LTR and/or 3′ LTR, by virtue of duplicating the 3′ LTR. Suitable insulators include, but are not limited to, the chicken β-globin insulator (see Chung et al, 1993. Cell 74:505; Chung et al, 1997. N4S 94:575; and Bell et al., 1999. Cell 98:387, incorporated by reference herein) or an insulator from a human β-globin locus, such as chicken HS4. In some embodiments the insulator binds CCCTC binding factor (CTCF). In some embodiments the insulator is a barrier insulator. In some embodiments the insulator is an enhancer-blocking insulator. See, e.g., Emery et al., Human Gene Therapy, 2011, and in Browning and Trobridge, Biomedicines, 2016, both of which are included in their entirety by reference.

In some embodiments, insulators in the retroviral nucleic acid reduce genotoxicity in recipient cells. Genotoxicity can be measured, e.g., as described in Cesana et al, “Uncovering and dissecting the genotoxicity of self-inactivating lentiviral vectors in vivo” Mol Ther. 2014 April; 22(4):774-85. doi: 10.1038/mt.2014.3. Epub 2014 Jan. 20.

Pharmaceutical Compositions and Methods of Making them

In some embodiments, one or more transducing units of retroviral vector are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per kg are administered to the subject. In some embodiments at least 1, 10, 100, 1000, 10⁴, 10⁵, 10⁶, 10⁷, 10⁸, 10⁹,10¹⁰, 10¹¹, 10¹², 10, or 10¹⁴, transducing units per target cell per ml of blood are administered to the subject.

### Concentration and Purification of Lentivirus

In some embodiments, a retroviral vector formulation described herein can be produced by a process comprising one or more of, e.g., all of, the following steps (i) to (vi), e.g., in chronological order:
(i) culturing cells that produce retroviral vector;
(ii) harvesting the retroviral vector containing supernatant;
(iii) optionally clarifying the supernatant;
(iv) purifying the retroviral vector to give a retroviral vector preparation;
(v) optionally filter-sterilization of the retroviral vector preparation; and
(vi) concentrating the retroviral vector preparation to produce the final bulk product.

In some embodiments the process does not comprise the clarifying step (iii). In other embodiments the process does include the clarifying step (iii). In some embodiments, step (vi) is performed using ultrafiltration, or tangential flow filtration, more preferably hollow fiber ultrafiltration. In some embodiments, the purification method in step (iv) is ion exchange chromatography, more preferably anion exchange chromatography. In some embodiments, the filter-sterilisation in step (v) is performed using a 0.22 m or a 0.2 m sterilising filter. In some embodiments, step (iii) is performed by filter clarification. In some embodiments, step (iv) is performed using a method or a combination of methods selected from chromatography, ultrafiltration/diafiltration, or centrifugation. In some embodiments, the chromatography method or a combination of methods is selected from ion exchange chromatography, hydrophobic interaction chromatography, size exclusion chromatography, affinity chromatography, reversed phase chromatography, and immobilized metal ion affinity chromatography. In some embodiments, the centrifugation method is selected from zonal centrifugation, isopycnic centrifugation and pelleting centrifugation. In some embodiments, the ultrafiltration/diafiltration method is selected from tangential flow diafiltration, stirred cell diafiltration and dialysis. In some embodiments, at least one step is included into the process to degrade nucleic acid to improve purification. In some embodiments, said step is nuclease treatment.

In some embodiments, concentration of the vectors is done before filtration. In some embodiments, concentration of the vectors is done after filtration. In some embodiments, concentration and filtrations steps are repeated.

In some embodiments, the final concentration step is performed after the filter-sterilisation step. In some embodiments, the process is a large scale-process for producing clinical grade formulations that are suitable for administration to humans as therapeutics. In some embodiments, the filter-sterilisation step occurs prior to a concentration step. In some embodiments, the concentration step is the final step in the process and the filter-sterilisation step is the penultimate step in the process. In some embodiments, the concentration step is performed using ultrafiltration, preferably tangential flow filtration, more preferably hollow fiber ultrafiltration. In some embodiments, the filter-sterilisation step is performed using a sterilising filter with a maximum pore size of about 0.22 μm. In another preferred embodiment the maximum pore size is 0.2 μm

In some embodiments, the vector concentration is less than or equal to about 4.6×10¹¹ RNA genome copies per ml of preparation prior to filter-sterilisation. The appropriate concentration level can be achieved through controlling the vector concentration using, e.g. a dilution step, if appropriate. Thus, in some embodiments, a retroviral vector preparation is diluted prior to filter sterilisation.

Clarification may be done by a filtration step, removing cell debris and other impurities. Suitable filters may utilize cellulose filters, regenerated cellulose fibers, cellulose fibers combined with inorganic filter aids (e.g. diatomaceous earth, perlite, fumed silica), cellulose filters combined with inorganic filter aids and organic resins, or any combination thereof, and polymeric filters (examples include but are not limited to nylon, polypropylene, polyethersulfone) to achieve effective removal and acceptable recoveries. A multiple stage process may be used. An exemplary two or three-stage process would consist of a coarse filter(s) to remove large precipitate and cell debris followed by polishing second stage filter(s) with nominal pore sizes greater than 0.2 micron but less than 1 micron. The optimal combination may be a function of the precipitate size distribution as well as other variables. In addition, single stage operations employing a relatively small pore size filter or centrifugation may also be used for clarification. More generally, any clarification approach including but not limited to dead-end filtration, microfiltration, centrifugation, or body feed of filter aids (e.g. diatomaceous earth) in combination with dead-end or depth filtration, which provides a filtrate of suitable clarity to not foul the membrane and/or resins in the subsequent steps, will be acceptable to use in the clarification step of the present invention.

In some embodiments, depth filtration and membrane filtration is used. Commercially available products useful in this regard are for instance mentioned in WO 03/097797, p. 20-21. Membranes that can be used may be composed of different materials, may differ in pore size, and may be used in combinations. They can be commercially obtained from several vendors. In some embodiments, the filter used for clarification is in the range of 1.2 to 0.22 μm. In some embodiments, the filter used for clarification is either a 1.2/0.45 μm filter or an asymmetric filter with a minimum nominal pore size of 0.22 μm

In some embodiments, the method employs nuclease to degrade contaminating DNA/RNA, i.e. mostly host cell nucleic acids. Exemplary nucleases suitable for use in the present invention include Benzonase® Nuclease (EP 0229866) which attacks and degrades all forms of DNA and RNA (single stranded, double stranded linear or circular) or any other DNase and/or RNase commonly used within the art for the purpose of eliminating unwanted or contaminating DNA and/or RNA from a preparation. In preferred embodiments, the nuclease is Benzonase® Nuclease, which rapidly hydrolyzes nucleic acids by hydrolyzing internal phosphodiester bonds between specific nucleotides, thereby reducing the size of the polynucleotides in the vector containing supernatant. Benzonase® Nuclease can be commercially obtained from Merck KGaA (code W214950). The concentration in which the nuclease is employed is preferably within the range of 1-100 units/ml.

In some embodiments, the vector suspension is subjected to ultrafiltration (sometimes referred to as diafiltration when used for buffer exchange) at least once during the process, e.g. for concentrating the vector and/or buffer exchange. The process used to concentrate the vector can include any filtration process (e.g., ultrafiltration (UF)) where the concentration of vector is increased by forcing diluent to be passed through a filter in such a manner that the diluent is removed from the vector preparation whereas the vector is unable to pass through the filter and thereby remains, in concentrated form, in the vector preparation. UF is described in detail in, e.g., Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A suitable filtration process is Tangential Flow Filtration (“TFF”) as described in, e.g., MILLIPORE catalogue entitled “Pharmaceutical Process Filtration Catalogue” pp. 177-202 (Bedford, Mass., 1995/96). TFF is widely used in the bioprocessing industry for cell harvesting, clarification, purification and concentration of products including viruses. The system is composed of three distinct process streams: the feed solution, the permeate and the retentate. Depending on application, filters with different pore sizes may be used. In some embodiments, the retentate contains the product (lentiviral vector).The particular ultrafiltration membrane selected may have a pore size sufficiently small to retain vector but large enough to effectively clear impurities. Depending on the manufacturer and membrane type, for retroviral vectors nominal molecular weight cutoffs (NMWC) between 100 and 1000 kDa may be appropriate, for instance membranes with 300 kDa or 500 kDa NMWC. The membrane composition may be, but is not limited to, regenerated cellulose, polyethersulfone, polysulfone, or derivatives thereof. The membranes can be flat sheets (also called flat screens) or hollow fibers. A suitable UF is hollow fibre UF, e.g., filtration using filters with a pore size of smaller than 0.1 m. Products are generally retained, while volume can be reduced through permeation (or be kept constant during diafiltration by adding buffer with the same speed as the speed with which the permeate, containing buffer and impurities, is removed at the permeate side).

The two most widely used geometries for TFF in the biopharmaceutical industry are plate & frame (flat screens) and hollow fiber modules. Hollow fiber units for ultrafiltration and microfiltration were developed by Amicon and Ramicon in the early 1970s (Cheryan, M. Ultrafiltration Handbook), even though now there are multiple vendors including Spectrum and GE Healthcare. The hollow fiber modules consist of an array of self-supporting fibers with a dense skin layer. Fiber diameters range from 0.5 mm-3 mm. In certain embodiments, hollow fibers are used for TFF. In certain embodiments, hollow fibers of 500 kDa (0.05 m) pore size are used. Ultrafiltration may comprise diafiltration (DF). Microsolutes can be removed by adding solvent to the solution being ultrafiltered at a rate equal to the UF rate. This washes microspecies from the solution at a constant volume, purifying the retained vector.

UF/DF can be used to concentrate and/or buffer exchange the vector suspensions in different stages of the purification process. The method can utilize a DF step to exchange the buffer of the supernatant after chromatography or other purification steps, but may also be used prior to chromatography.

In some embodiments, the eluate from the chromatography step is concentrated and further purified by ultrafiltration-diafiltration. During this process the vector is exchanged into formulation buffer. Concentration to the final desired concentration can take place after the filter-sterilisation step. After said sterile filtration, the filter sterilised substance is concentrated by aseptic UF to produce the bulk vector product.

In embodiments, the ultrafiltration/diafiltration may be tangential flow diafiltration, stirred cell diafiltration and dialysis.

Purification techniques tend to involve the separation of the vector particles from the cellular milieu and, if necessary, the further purification of the vector particles. One or more of a variety of chromatographic methods may be used for this purification. Ion exchange, and more particularly anion exchange, chromatography is a suitable method, and other methods could be used. A description of some chromatographic techniques is given below.

Ion-exchange chromatography utilises the fact that charged species, such as biomolecules and viral vectors, can bind reversibly to a stationary phase (such as a membrane, or else the packing in a column) that has, fixed on its surface, groups that have an opposite charge. There are two types of ion exchangers. Anion exchangers are stationary phases that bear groups having a positive charge and hence can bind species with a negative charge. Cation exchangers bear groups with a negative charge and hence can bind species with positive charge. The pH of the medium has an influence on this, as it can alter the charge on a species. Thus, for a species such as a protein, if the pH is above the pI, the net charge will be negative, whereas below the pI, the net charge will be positive.

Displacement (elution) of the bound species can be effected by the use of suitable buffers. Thus commonly the ionic concentration of the buffer is increased until the species is displaced through competition of buffer ions for the ionic sites on the stationary phase. An alternative method of elution entails changing the pH of the buffer until the net charge of the species no longer favours biding to the stationary phase. An example would be reducing the pH until the species assumes a net positive charge and will no longer bind to an anion exchanger.

Some purification can be achieved if impurities are uncharged, or else if they bear a charge of opposite sign to that of the desired species, but the same sign to that on the ion exchanger. This is because uncharged species and those having a charge of the same sign to that an ion exchanger, will not normally bind. For different bound species, the strength of the binding varies with factors such as the charge density and the distribution of charges on the various species. Thus by applying an ionic or pH gradient (as a continuous gradient, or as a series of steps), the desired species might be eluted separately from impurities.

Size exclusion chromatography is a technique that separates species according to their size. Typically it is performed by the use of a column packed with particles having pores of a well-defined size. For the chromatographic separation, particles are chosen that have pore sizes that are appropriate with regard to the sizes of the species in the mixture to be separated. When the mixture is applied, as a solution (or suspension, in the case of a virus), to the column and then eluted with buffer, the largest particles will elute first as they have limited (or no) access to the pores. Smaller particles will elute later as they can enter the pores and hence take a longer path through the column. Thus in considering the use of size exclusion chromatography for the purification of viral vectors, it would be expected that the vector would be eluted before smaller impurities such as proteins.

Species, such as proteins, have on their surfaces, hydrophobic regions that can bind reversibly to weakly hydrophobic sites on a stationary phase. In media having a relatively high salt concentration, this binding is promoted. Typically in HIC the sample to be purified is bound to the stationary phase in a high salt environment. Elution is then achieved by the application of a gradient (continuous, or as a series of steps) of decreasing salt concentration. A salt that is commonly used is ammonium sulphate. Species having differing levels of hydrophobicity will tend to be eluted at different salt concentrations and so the target species can be purified from impurities. Other factors, such as pH, temperature and additives to the elution medium such as detergents, chaotropic salts and organics can also influence the strength of binding of species to HIC stationary phases. One, or more, of these factors can be adjusted or utilised to optimise the elution and purification of product.

Viral vectors have on their surface, hydrophobic moieties such as proteins, and thus HIC could potentially be employed as a means of purification.

Like HIC, RPC separates species according to differences in their hydrophobicities. A stationary phase of higher hydrophobicity than that employed in HIC is used. The stationary phase often consists of a material, typically silica, to which are bound hydrophobic moieties such as alkyl groups or phenyl groups. Alternatively the stationary phase might be an organic polymer, with no attached groups. The sample-containing the mixture of species to be resolved is applied to the stationary phase in an aqueous medium of relatively high polarity which promotes binding. Elution is then achieved by reducing the polarity of the aqueous medium by the addition of an organic solvent such as isopropanol or acetonitrile. Commonly a gradient (continuous, or as a series of steps) of increasing organic solvent concentration is used and the species are eluted in order of their respective hydrophobicities.

Other factors, such as the pH of the elution medium, and the use of additives, can also influence the strength of binding of species to RPC stationary phases. One, or more, of these factors can be adjusted or utilised to optimise the elution and purification of product. A common additive is trifluororacetic acid (TFA). This suppresses the ionisation of acidic groups such as carboxyl moieties in the sample. It also reduces the pH in the eluting medium and this suppresses the ionisation of free silanol groups that may be present on the surface of stationary phases having a silica matrix. TFA is one of a class of additives known as ion pairing agents. These interact with ionic groups, present on species in the sample, that bear an opposite charge. The interaction tends to mask the charge, increasing the hydrophobicity of the species. Anionic ion pairing agents, such as TFA and pentafluoropropionic acid interact with positively charged groups on a species. Cationic ion pairing agents such, as triethylamine, interact with negatively charged groups.

Viral vectors have on their surface, hydrophobic moieties such as proteins, and thus RPC, potentially, could be employed as a means of purification.

Affinity chromatography utilises the fact that certain ligands that bind specifically with biomolecules such as proteins or nucleotides, can be immobilised on a stationary phase. The modified stationary phase can then be used to separate the relevant biomolecule from a mixture. Examples of highly specific ligands are antibodies, for the purification of target antigens and enzyme inhibitors for the purification of enzymes. More general interactions can also be utilised such as the use of the protein A ligand for the isolation of a wide range of antibodies.

Typically, affinity chromatography is performed by application of a mixture, containing the species of interest, to the stationary phase that has the relevant ligand attached. Under appropriate conditions this will lead to the binding of the species to the stationary phase. Unbound components are then washed away before an eluting medium is applied. The eluting medium is chosen to disrupt the binding of the ligand to the target species. This is commonly achieved by choice of an appropriate ionic strength, pH or by the use of substances that will compete with the target species for ligand sites. For some bound species, a chaotropic agent such as urea is used to effect displacement from the ligand. This, however, can result in irreversible denaturation of the species.

Viral vectors have on their surface, moieties such as proteins, that might be capable of binding specifically to appropriate ligands. This means that, potentially, affinity chromatography could be used in their isolation.

Biomolecules, such as proteins, can have on their surface, electron donating moieties that can form coordinate bonds with metal ions. This can facilitate their binding to stationary phases carrying immobilised metal ions such as Ni²⁺, Cu²⁺, Zn²⁺ or Fe³⁺. The stationary phases used in IMAC have chelating agents, typically nitriloacetic acid or iminodiacetic acid covalently attached to their surface and it is the chelating agent that holds the metal ion. It is necessary for the chelated metal ion to have at least one coordination site left available to form a coordinate bond to a biomolecule. Potentially there are several moieties on the surface of biomolecules that might be capable of bonding to the immobilised metal ion. These include histidine, tryptophan and cysteine residues as well as phosphate groups. For proteins, however, the predominant donor appears to be the imidazole group of the histidine residue. Native proteins can be separated using IMAC if they exhibit suitable donor moieties on their surface. Otherwise IMAC can be used for the separation of recombinant proteins bearing a chain of several linked histidine residues.

Typically, IMAC is performed by application of a mixture, containing the species of interest, to the stationary phase. Under appropriate conditions this will lead to the coordinate bonding of the species to the stationary phase. Unbound components are then washed away before an eluting medium is applied. For elution, gradients (continuous, or as a series of steps) of increasing salt concentration or decreasing pH may be used. Also a commonly used procedure is the application of a gradient of increasing imidazole concentration. Biomolecules having different donor properties, for example having histidine residues in differing environments, can be separated by the use of gradient elution.

Viral vectors have on their surface, moieties such as proteins, that might be capable of binding to IMAC stationary phases. This means that, potentially, IMAC could be used in their isolation.

Suitable centrifugation techniques include zonal centrifugation, isopycnic ultra and pelleting centrifugation.

Filter-sterilisation is suitable for processes for pharmaceutical grade materials. Filter-sterilisation renders the resulting formulation substantially free of contaminants. The level of contaminants following filter-sterilisation is such that the formulation is suitable for clinical use. Further concentration (e.g. by ultrafiltration) following the filter-sterilisation step may be performed in aseptic conditions. In some embodiments, the sterilising filter has a maximum pore size of 0.22 m.

The retroviral vectors herein can also be subjected to methods to concentrate and purify a lentiviral vector using flow-through ultracentrifugation and high-speed centrifugation, and tangential flow filtration. Flow through ultracentrifugation can be used for the purification of RNA tumor viruses (Toplin et al, Applied Microbiology 15:582-589, 1967; Burger et al., Journal of the National Cancer Institute 45: 499-503, 1970). Flow-through ultracentrifugation can be used for the purification of Lentiviral vectors. This method can comprise one or more of the following steps. For example, a lentiviral vector can be produced from cells using a cell factory or bioreactor system. A transient transfection system can be used or packaging or producer cell lines can also similarly be used. A pre-clarification step prior to loading the material into the ultracentrifuge could be used if desired. Flow-through ultracentrifugation can be performed using continuous flow or batch sedimentation. The materials used for sedimentation are, e.g.: Cesium chloride, potassium tartrate and potassium bromide, which create high densities with low viscosity although they are all corrosive. CsCl is frequently used for process development as a high degree of purity can be achieved due to the wide density gradient that can be created (1.0 to 1.9 g/cm³). Potassium bromide can be used at high densities, e.g., at elevated temperatures, such as 25° C., which may be incompatible with stability of some proteins. Sucrose is widely used due to being inexpensive, non-toxic and can form a gradient suitable for separation of most proteins, sub-cellular fractions and whole cells. Typically the maximum density is about 1.3 g/cm³. The osmotic potential of sucrose can be toxic to cells in which case a complex gradient material can be used, e.g. Nycodenz. A gradient can be used with 1 or more steps in the gradient. An embodiment is to use a step sucrose gradient. The volume of material can be from 0.5 liters to over 200 liters per run. The flow rate speed can be from 5 to over 25 liters per hour. A suitable operating speed is between 25,000 and 40,500 rpm producing a force of up to 122,000×g. The rotor can be unloaded statically in desired volume fractions. An embodiment is to unload the centrifuged material in 100 ml fractions. The isolated fraction containing the purified and concentrated Lentiviral vector can then be exchanged in a desired buffer using gel filtration or size exclusion chromatography. Anionic or cationic exchange chromatography could also be used as an alternate or additional method for buffer exchange or further purification. In addition, Tangential Flow Filtration can also be used for buffer exchange and final formulation if required. Tangential Flow Filtration (TFF) can also be used as an alternative step to ultra or high speed centrifugation, where a two step TFF procedure would be implemented. The first step would reduce the volume of the vector supernatant, while the second step would be used for buffer exchange, final formulation and some further concentration of the material. The TFF membrane can have a membrane size of between 100 and 500 kilodaltons, where the first TFF step can have a membrane size of 500 kilodaltons, while the second TFF can have a membrane size of between 300 to 500 kilodaltons. The final buffer should contain materials that allow the vector to be stored for long term storage.

In embodiments, the method uses either cell factories that contains adherent cells, or a bioreactor that contains suspension cells that are either transfected or transduced with the vector and helper constructs to produce lentiviral vector. Non limiting examples or bioreactors, include the Wave bioreactor system and the Xcellerex bioreactors. Both are disposable systems. However non-disposable systems can also be used. The constructs can be those described herein, as well as other lentiviral transduction vectors. Alternatively the cell line can be engineered to produce Lentiviral vector without the need for transduction or transfection. After transfection, the lentiviral vector can be harvested and filtered to remove particulates and then is centrifuged using continuous flow high speed or ultra centrifugation. A preferred embodiment is to use a high speed continuous flow device like the JCF-A zonal and continuous flow rotor with a high speed centrifuge. Also preferably is the use of Contifuge Stratus centrifuge for medium scale Lentiviral vector production. Also suitable is any continuous flow centrifuge where the speed of centrifugation is greater than 5,000×g RCF and less than 26,000×g RCF. Preferably, the continuous flow centrifugal force is about 10,500×g to 23,500×g RCF with a spin time of between 20 hours and 4 hours, with longer centrifugal times being used with slower centrifugal force. The lentiviral vector can be centrifuged on a cushion of more dense material (a non limiting example is sucrose but other reagents can be used to form the cushion and these are well known in the art) so that the Lentiviral vector does not form aggregates that are not filterable, as sometimes occurs with straight centrifugation of the vector that results in a viral vector pellet. Continuous flow centrifugation onto a cushion allows the vector to avoid large aggregate formation, yet allows the vector to be concentrated to high levels from large volumes of transfected material that produces the Lentiviral vector. In addition, a second less-dense layer of sucrose can be used to band the Lentiviral vector preparation. The flow rate for the continuous flow centrifuge can be between 1 and 100 ml per minute, but higher and lower flow rates can also be used. The flow rate is adjusted to provide ample time for the vector to enter the core of the centrifuge without significant amounts of vector being lost due to the high flow rate. If a higher flow rate is desired, then the material flowing out of the continuous flow centrifuge can be re-circulated and passed through the centrifuge a second time. After the virus is concentrated using continuous flow centrifugation, the vector can be further concentrated using Tangential Flow Filtration (TFF), or the TFF system can be simply used for buffer exchange. A non-limiting example of a TFF system is the Xampler cartridge system that is produced by GB-Healthcare. Preferred cartridges are those with a MW cut-off of 500,000 MW or less. Preferably a cartridge is used with a MW cut-off of 300,000 MW. A cartridge of 100,000 MW cut-off can also be used. For larger volumes, larger cartridges can be used and it will be easy for those in the art to find the right TFF system for this final buffer exchange and/or concentration step prior to final fill of the vector preparation. The final fill preparation may contain factors that stabilize the vector sugars are generally used and are known in the art.

Protein Content

In some embodiments the retroviral particle includes various source cell genome-derived proteins, exogenous proteins, and viral-genome derived proteins. In some embodiments the retroviral particle contains various ratios of source cell genome-derived proteins to viral-genome-derived proteins, source cell genome-derived proteins to exogenous proteins, and exogenous proteins to viral-genome derived proteins.

In some embodiments, the viral-genome derived proteins are GAG polyprotein precursor, HIV-1 Integrase, POL polyprotein precursor, Capsid, Nucleocapsid, p17 matrix, p6, p2, VPR, Vif.

In some embodiments, the source cell-derived proteins are Cyclophilin A, Heat Shock 70 kD, Human Elongation Factor-1 Alpha (EF-1R), Histones H1, H2A, H3, H4, beta-globin, Trypsin Precursor, Parvulin, Glyceraldehyde-3-phosphate dehydrogenase, Lck, Ubiquitin, SUMO-1, CD48, Syntenin-1, Nucleophosmin, Heterogeneous nuclear ribonucleoproteins C1/C2, Nucleolin, Probable ATP-dependent helicase DDX48, Matrin-3, Transitional ER ATPase, GTP-binding nuclear protein Ran, Heterogeneous nuclear ribonucleoprotein U, Interleukin enhancer binding factor 2, Non-POU domain containing octamer binding protein, RuvB like 2, HSP 90-b, HSP 90-a, Elongation factor 2, D-3-phosphoglycerate dehydrogenase, α-enolase, C-1-tetrahydrofolate synthase, cytoplasmic, Pyruvate kinase, isozymes M1/M2, Ubiquitin activating enzyme E1, 26S protease regulatory subunit S10B, 60S acidic ribosomal protein P2, 60S acidic ribosomal protein PO, 40S ribosomal protein SA, 40S ribosomal protein S2, 40S ribosomal protein S3, 60S ribosomal protein L4, 60S ribosomal protein L3, 40S ribosomal protein S3a, 40S ribosomal protein S7, 60S ribosomal protein L7a, 60S acidic ribosomal protein L31, 60S ribosomal protein L10a, 60S ribosomal protein L6, 26S proteasome non-ATPase regulatory subunit 1, Tubulin b-2 chain, Actin, cytoplasmic 1, Actin, aortic smooth muscle, Tubulin α-ubiquitous chain, Clathrin heavy chain 1, Histone H2B.b, Histone H4, Histone H3.1, Histone H3.3, Histone H2A type 8, 26S protease regulatory subunit 6A, Ubiquitin-4, RuvB like 1, 26S protease regulatory subunit 7, Leucyl-tRNA synthetase, cytoplasmic, 60S ribosomal protein L19, 26S proteasome non-ATPase regulatory subunit 13, Histone H2B.F, U5 small nuclear ribonucleoprotein 200 kDa helicase, Poly[ADP-ribose]polymerase-1, ATP-dependent DNA helicase II, DNA replication licensing factor MCM5, Nuclease sensitive element binding protein 1, ATP-dependent RNA helicase A, Interleukin enhancer binding factor 3, Transcription elongation factor B polypeptide 1, Pre-mRNA processing splicing factor 8, Staphylococcal nuclease domain containing protein 1, Programmed cell death 6-interacting protein, Mediator of RNA polymerase II transcription subunit 8 homolog, Nucleolar RNA helicase II, Endoplasmin, DnaJ homolog subfamily A member 1, Heat shock 70 kDa protein 1L, T-complex protein 1 e subunit, GCN1-like protein 1, Serotransferrin, Fructose bisphosphate aldolase A, Inosine-5′monophosphate dehydrogenase 2, 26S protease regulatory subunit 6B, Fatty acid synthase, DNA-dependent protein kinase catalytic subunit, 40S ribosomal protein S17, 60S ribosomal protein L7, 60S ribosomal protein L12, 60S ribosomal protein L9, 40S ribosomal protein S8, 40S ribosomal protein S4 X isoform, 60S ribosomal protein L11, 26S proteasome non-ATPase regulatory subunit 2, Coatomer a subunit, Histone H2A.z, Histone H1.2, Dynein heavy chain cytosolic. See: Saphire et al., Journal of Proteome Research, 2005, and Wheeler et al., Proteomics Clinical Applications, 2007.

In some embodiments the retroviral vector is pegylated.

### Particle Size

In some embodiments the median retroviral vector diameter is between 10 and 1000 nM, 25 and 500 nm 40 and 300 nm, 50 and 250 nm, 60 and 225 nm, 70 and 200 nm, 80 and 175 nm, or 90 and 150 nm.

In some embodiments, 90% of the retroviral vectors fall within 50% of the median diameter of the retrovirus. In some embodiments, 90% of the retroviral vectors fall within 25% of the median diameter of the retrovirus. In some embodiments, 90% of the retroviral vectors fall within 20% of the median diameter of the retrovirus. In some embodiments, 90% of the retroviral vectors fall within 15% of the median diameter of the retrovirus. In some embodiments, 90% of the retroviral vectors fall within 10% of the median diameter of the retrovirus.

### Indications and Uses

In some embodiments, the fusosome, e.g. retroviral vectors or particles, or pharmaceutical compositions thereof as described herein can be administered to a subject, e.g. a mammal, e.g. a human. In some aspects, provided herein are retroviral vectors, VLPs, or pharmaceutical compositions, such as any as described herein, that can be administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease. In some embodiments, the fusosome, e.g. retroviral vectors or particles, contains nucleic acid sequences encoding an exogenous agent for treating the disease or condition in the subject. For example, the exogenous agent is one that targets or is specific for a protein of a neoplastic cells and the fusosome is administered to a subject for treating a tumor or cancer in the subject. In another example, the exogenous agent is an inflammatory mediator or immune molecule, such as a cytokine, and the fusosome is administered to a subject for treating any condition in which it is desired to modulate (e.g. increase) the immune response, such as a cancer or infectious disease.

Thus, also provided, in some aspects, are methods of administering and uses, such as therapeutic and prophylactic uses, of the provided fusosomes, e.g., retroviral vectors and particles, such as lentiviral vectors and particles, and/or compositions comprising the same. Such methods and uses include therapeutic methods and uses, for example, involving administration of the fusosomes, e.g., retroviral vectors or particles, such as lentiviral vectors or particles, or compositions containing the same, to a subject having a disease, condition, or disorder for delivery of an exogenous agent for treatment of the disease, condition or disorder. In some embodiments, the fusosome (e.g., retroviral vector or particle, such as lentiviral vector or particle) is administered in an effective amount or dose to effect treatment of the disease, condition or disorder. Provided herein are uses of any of the provided fusosomes (e.g. retroviral vector or particle, such as lentiviral vector or particle) in such methods and treatments, and in the preparation of a medicament in order to carry out such therapeutic methods. In some embodiments, the methods are carried out by administering the fusosomes (e.g. retroviral vector or particle, such as lentiviral vector or particle), or compositions comprising the same, to the subject having, having had, or suspected of having the disease or condition or disorder. In some embodiments, the methods thereby treat the disease or condition or disorder in the subject. Also provided herein are use of any of the compositions, such as pharmaceutical compositions provided herein, for the treatment of a disease, condition or disorder associated with a particular gene or protein targeted by or provided by the exogenous agent.

Cancers include, for example, leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Target cells from mammalian (e.g., human) tissue include cells from epithelial, connective, muscular, or nervous tissue or cells, and combinations thereof. Target mammalian (e.g., human) cells and organ systems include the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)’; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In some embodiments, a non-target cells or organ system is chosen from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)’; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

The administration of a pharmaceutical composition described herein may be, for example, by way of oral, inhaled, transdermal or parenteral (including intravenous, intratumoral, intraperitoneal, intramuscular, intracavity, and subcutaneous) administration. The fusosomes may, in some embodiments, be administered alone or formulated as a pharmaceutical composition.

In embodiments, the fusosome composition mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the fusosome composition comprises an exogenous protein), the effect lasts for less than 1, 2, 3, 4, 5, 6, or 7 days, 2, 3, or 4 weeks, or 1, 2, 3, 6, or 12 months.

In embodiments, the fusosome composition described herein is delivered ex-vivo to a cell or tissue, e.g., a human cell or tissue.

The fusosome compositions described herein can, in some embodiments, be administered to a subject, e.g., a mammal, e.g., a human. In certain embodiments, the subject may be at risk of, may have a symptom of, or may be diagnosed with or identified as having, a particular disease or condition (e.g., a disease or condition described herein).

In some embodiments, the source of fusosomes are from the same subject that is administered a fusosome composition. In other embodiments, they are different. For example, the source of fusosomes and recipient tissue may be autologous (from the same subject) or heterologous (from different subjects). In either case, the donor tissue for fusosome compositions described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

In some embodiments, the fusosome is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 DOI: 10.1038/srep01462). Thus, in some embodiments, the fusosome is co-administered with an inhibitor of sypressyn, e.g., a siRNA or inhibitory antibody.

Compositions described herein may, in some embodiments, be used to similarly modulate the cell or tissue function or physiology of a variety of other organisms, including but not limited to: farm or working animals (horses, cows, pigs, chickens etc.), pet or zoo animals (cats, dogs, lizards, birds, lions, tigers and bears etc.), aquaculture animals (fish, crabs, shrimp, oysters etc.), plants species (trees, crops, ornamentals flowers etc), fermentation species (saccharomyces etc.). Fusosome compositions described herein can be made, in some embodiments, from such non-human sources and administered to a non-human target cell or tissue or subject.

Fusosome compositions can be autologous, allogeneic or xenogeneic to the target.

### Additional Therapeutic Agents

In some embodiments, the fusosome composition is co-administered with an additional agent, e.g., a therapeutic agent, to a subject, e.g., a recipient, e.g., a recipient described herein. In some embodiments, the co-administered therapeutic agent is an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin). In embodiments, the immunosuppressive agent decreases immune mediated clearance of fusosomes. In some embodiments the fusosome composition is co-administered with an immunostimulatory agent, e.g., an adjuvant, an interleukin, a cytokine, or a chemokine.

In some embodiments, the fusosome composition and the immunosuppressive agent are administered at the same time, e.g., contemporaneously administered. In some embodiments, the fusosome composition is administered before administration of the immunosuppressive agent. In some embodiments, the fusosome composition is administered after administration of the immunosuppressive agent.

In some embodiments, the immunosuppressive agent is a small molecule such as ibuprofen, acetaminophen, cyclosporine, tacrolimus, rapamycin, mycophenolate, cyclophosphamide, glucocorticoids, sirolimus, azathriopine, or methotrexate.

In some embodiments, the immunosuppressive agent is an antibody molecule, including but not limited to: muronomab (anti-CD3), Daclizumab (anti-IL12), Basiliximab, Infliximab (Anti-TNFa), or rituximab (Anti-CD20).

In some embodiments, co-administration of the fusosome composition with the immunosuppressive agent results in enhanced persistence of the fusosome composition in the subject compared to administration of the fusosome composition alone. In some embodiments, the enhanced persistence of the fusosome composition in the co-administration is at least 10%, 20%, 30%, 40%, 50%, 60%, 70%, 80%, 90% or longer, compared to persistence of the fusosome composition when administered alone. In some embodiments, the enhanced persistence of the fusosome composition in the co-administration is at least 1, 2, 3, 4, 5, 6, 7, 10, 15, 20, 25, or 30 days or longer, compared to survival of the fusosome composition when administered alone.

### EXAMPLES

The Examples below are set forth to aid in the understanding of the inventions, but are not intended to, and should not be construed to, limit its scope in any way.

### Example 1. Assaying Off-Target Cells to Detect Specificity of Retroviral Nucleic Acid Delivery

This Example describes quantification of a nucleic acid in off-target recipient cells by measuring vector copy number in single cells.

In an embodiment, treated mice have a similar vector copy number in off-target cells as those from untreated mice, e.g., no vector or a vector number similar to negative control levels. In an embodiment, treated mice have a similar percent of off-target cells that contain the vector as those from untreated mice, e.g., no cells or a cell number similar to negative control levels.

In this example, the off-target recipient cell is a CD11c+ cell. However, this protocol may be adapted to any cell type for which suitable surface markers exist and which can be isolated from the subject. Notably, the methods described herein may be equally applicable to humans, rats, monkeys with optimization to the protocol.

Mice are treated with retroviral vector produced as described herein or with PBS (negative control). 28 days following treatment, peripheral blood is collected from mice that received retroviral vector and mice that received PBS treatment. Blood is collected into 1 ml PBS containing 5 μM EDTA and mixed immediately to prevent clotting. The tubes are kept on ice and red blood cells are removed using a buffered ammonium chloride (ACK) solution. Cells are stained with a murine CD11c:APC-Cy7 antibody (Biolegend Catalog #: 117323) or an isotype control APC-Cy7 antibody (Biolegend Catalog #: 400230) at 4° C. for 30 minutes in the dark, after being Fc blocked (Biolegend Catalog #: 101319) in cell staining buffer (Biolegend Catalog #: 420201) for 10 minutes. After being washed two times with PBS, cells are analyzed on a FACS Aria (BD Biosciences, San Jose, Calif.) with 640 nm laser excitation and emission collected at 780−/+60 nm running the FACSDiva™ software (BD Biosciences, San Jose, Calif.) to set negative gates using the isotype control APC-Cy7 antibody labeled cells. APC-Cy7 positive cells are sorted into single wells of plate for vector copy number analysis.

Vector copy number is assessed using single-cell nested PCR. PCR is performed with qPCR using primers and probes specific to the vector and an endogenous control gene. Vector copy number is determined by dividing the amount of vector qPCR signal by the amount of the endogenous control gene qPCR signal. A cell that received the vector will have a vector copy number of at least 1.0. Vector copy number is assessed across the population by averaging the vector copy number of the plurality of cells

In some embodiments, mice treated with retroviral vectors have a similar average vector copy number in off-target cells as those from mice treated with vehicle. In some embodiments, mice treated with treated with retroviral vectors have a similar percent of off-target cells that received the vector as those from mice treated with vehicle.

### Example 2. Assaying Off-Target Cells to Detect Specificity of Delivery of an Exogenous Protein Agent

This Example describes quantification of the expression of an exogenous agent in off-target recipient cells by exogenous agent expression in single cells.

In an embodiment, treated mice have similar exogenous agent expression in off-target cells as those from untreated mice. In an embodiment, treated mice have a similar percent of off-target cells that express the exogenous agent as those from untreated mice.

In this example, the off-target recipient cell is a CD11c+ cell. However, this protocol may be adapted to any cell type for which suitable surface markers exist and which can be isolated from the subject. Notably, the methods described herein may be equally applicable to humans, rats, monkeys with optimization to the protocol. In this example the exogenous agent is a fluorescent protein and expression is measured via flow cytometry. In other embodiments, the expression of an exogenous protein agent may be measured with immunostaining for the protein. In other embodiments expression of the exogenous protein agent may be measured via microscopy or western blot.

Mice are treated with retroviral vector with a tdtomato fluorescent protein agent produced via any of the methods described in this application or with PBS (negative control). 28 days following treatment, peripheral blood is collected from mice that received retroviral vector and mice that received PBS treatment. Blood is collected into 1 ml PBS containing 5 μM EDTA and mixed immediately to prevent clotting. The tubes are kept on ice and red blood cells are removed using a buffered ammonium chloride (ACK) solution. Cells are stained with a murine CD11c:APC-Cy7 antibody (Biolgend Catalog #: 117323) or isotype controls APC-Cy7 antibody (Biolegend Catalog #: 400230) at 4° C. for 30 minutes in the dark, after being Fc blocked (Biolegend Catalog #: 101319) in cell staining buffer (Biolegend Catalog #: 420201) for 10 minutes. After being washed two times with PBS, cells are analyzed on a FACS Aria (BD Biosciences, San Jose, Calif.) running the FACSDiva™ software (BD Biosciences, San Jose, Calif.). A negative gate for CD11c is set using the isotype control APC-Cy7 antibody labeled cells and with a 640 nm laser excitation and emission collected at 780−/+60. A negative gate for tdtomato expression is set with cells isolated from mice treated with vehicle and with a 552 nm laser excitation and an emission collected at 585−/+42 nm.

The percent of CD11c+ cells that are tdtomato positive is measured. In some embodiments, the percent of CD11c+ cells that are tdtomato positive is similar in cells from treated and untreated mice. The median tdtomato fluorescence level is measured in CD11c+ cells. In some embodiments, the median tdtomato fluorescence level in CD11c+ cells is similar in cells from treated and untreated mice.

### Example 3. Assaying Target Cells to Detect Specificity of Retroviral Nucleic Acid Delivery

This Example describes quantification of a nucleic acid in target recipient cells by measuring vector copy number in single cells.

In an embodiment, treated mice have a greater vector copy number in target cells than those from untreated mice. In an embodiment, treated mice have a greater percent of target cells that contain the vector than those from untreated mice.

In this example, the target recipient cell is a CD3+ cell. However, this protocol may be adapted to any cell type for which suitable surface markers exist and which can be isolated from the subject. Notably, the methods described herein may be equally applicable to humans, rats, monkeys with optimization to the protocol.

Mice are treated with retroviral vector and a blood sample is collected as described above in Example 1. Cells are stained with a murine CD3:APC-Cy7 antibody (Biolegend Catalog #: 100330) or an isotype control using the protocol described above in Example 1. Vector copy number is assessed using single-cell nested PCR as described in Example 1.

In some embodiments, mice treated with retroviral vectors have a greater average vector copy number in target cells than those from mice treated with vehicle. In some embodiments, mice treated with treated with retroviral vectors have a greater percent of target cells that received the vector than those from mice treated with vehicle.

### Example 4. Assaying Target Cells to Detect Specificity of Delivery of an Exogenous Protein Agent

This Example describes quantification of the expression of an exogenous protein agent in target recipient cells by exogenous protein agent expression in single cells.

In an embodiment, treated mice have greater exogenous protein agent expression in target cells than those from untreated mice. In an embodiment, treated mice have a greater percent of target cells that express the exogenous protein agent than those from untreated mice.

In this example, the target recipient cell is a CD3+ cell. However, this protocol may be adapted to any cell type for which suitable surface markers exist and which can be isolated from the subject. Notably, the methods described herein may be equally applicable to humans, rats, monkeys with optimization to the protocol. In this example the exogenous protein agent is a fluorescent protein and expression is measured via flow cytometry. In other embodiments, the expression of an exogenous protein agent may be measured with immunostaining for the protein. In other embodiments expression of the exogenous protein agent may be measured via microscopy or western blot.

Mice are treated with retroviral vector and a blood sample is collected as described above in Example 2. Cells are stained with a murine CD3:APC-Cy7 antibody (Biolegend Catalog #: 100330) or isotype controls and analyzed by flow cytometry using the protocol described in Example 2.

The percent of CD3+ cells that are tdtomato positive is measured. In some embodiments, the percent of CD3+ cells that are tdtomato positive is greater in cells from treated than untreated mice. The median tdtomato fluorescence level is measured in CD3+ cells. In some embodiments, the median tdtomato fluorescence level in CD3+ cells is greater in cells from treated than untreated mice.

### Example 5. Modification of Retroviral Vector with HLA-G or HLA-E for Decreased Cytotoxicity Mediated by PBMC Cell Lysis

This Example describes retroviral vectors derived from cells modified to have decreased cytotoxicity due to cell lysis by peripheral blood mononuclear cells (PBMCs).

In an embodiment, cytotoxicity mediated cell lysis of retroviral vectors by PBMCs is a measure of immunogenicity of retroviral vectors, as lysis will reduce, e.g., inhibit or stop, the activity of a retroviral vector.

Retroviral vectors are created from: unmodified cells (hereinafter NMCs, positive control), cells that are transfected with HLA-G or HLA-E cDNA (hereinafter NMC-HLA-G), and cells transfected with an empty vector control (hereinafter NMC-empty vector, negative control).

PBMC mediated lysis of a retroviral vector is determined by europium release assays as described in Bouma, et al. Hum. Immunol. 35(2):85-92; 1992 & van Besouw et al. Transplantation 70(1):136-143; 2000. PBMCs (hereinafter effector cells) are isolated from an appropriate donor, and stimulated with allogeneic gamma irradiated PMBCs and 200 IU/mL IL-2 (proleukin, Chiron BV Amsterdam, The Netherlands) in a round bottom 96 well plate for 7 days at 37° C. The retroviral vectors are labeled with europium-diethylenetriaminepentaacetate (DTPA) (sigma, St. Louis, Mo., USA).

At day 7 cytotoxicity-mediated lysis assays is performed by incubating ⁶³Eu-labelled retroviral vector with effector cells in a 96-well plate for 1, 2, 3, 4, 5, 6, 8, 10, 15, 20, 24, or 48 hours after plating at effector/target ratios ranging from 1000:1-1:1 and 1:1.25-1:1000. After incubation, the plates are centrifuged and a sample of the supernatant is transferred to 96-well plates with low background fluorescence (fluoroimmunoplates, Nunc, Roskilde, Denmark).

Subsequently, enhancement solution (PerkinElmer, Groningen, The Netherlands) is added to each well. The released europium is measured in a time-resolved fluorometer (Victor 1420 multilabel counter, LKB-Wallac, Finland). Fluorescence is expressed in counts per second (CPS). Maximum percent release of europium by a target retroviral vector is determined by incubating an appropriate number (1×10²-1×10⁸) of retroviral vectors with 1% triton (sigma-aldrich) for an appropriate amount of time. Spontaneous release of europium by target retroviral vector is measured by incubation of labeled target retroviral vector without effector cells. Percentage leakage is then calculated as: (spontaneous release/maximum release)×100%. The percentage of cytotoxicity mediated lysis is calculated as % lysis=[(measured lysis-spontaneous lysis-spontaneous release)/(maximum release-spontaneous release)]×100%. The data is analyzed by looking at the percentage of lysis as a function of different effector target ratios.

In an embodiment, retroviral vectors generated from NMC-HLA-G cells will have a decreased percentage of lysis by target cells at specific timepoints as compared to retroviral vectors generated from NMCs or NMC-empty vector.

### Example 6. Modification of Retroviral Vector with HLA-G or HLA-E for Decreased NK Lysis Activity

This Example describes the generation of a retroviral vector composition derived from a cell source which has been modified to decrease cytotoxicity mediated cell lysis by NK cells. In an embodiment cytotoxicity mediated cell lysis of retroviral vectors by NK cells is a measure of immunogenicity for retroviral vectors.

Retroviral vectors are created from: unmodified cells (hereinafter NMCs, positive control), cells that are transfected with HLA-G or HLA-E cDNA (hereinafter NMC-HLA-G), and cells transfected with an empty vector control (hereinafter NMC-empty vector, negative control).

NK cell mediated lysis of a retroviral vector is determined by europium release assays as described in Bouma, et al. Hum. Immunol. 35(2):85-92; 1992 & van Besouw et al. Transplantation 70(1):136-143; 2000. NK cells (hereinafter effector cells) are isolated from an appropriate donor according to the methods in Crop et al. Cell transplantation (20):1547-1559; 2011, and stimulated with allogeneic gamma irradiated PMBCs and 200 IU/mL IL-2 (proleukin, Chiron BV Amsterdam, The Netherlands) in a round bottom 96 well plate for 7 days at 37° C. The retroviral vectors are labeled with europium-diethylenetriaminepentaacetate (DTPA) (sigma, St. Louis, Mo., USA). Cytotoxicity-mediated lysis assays and data analysis are performed as described above in Example 5.

In an embodiment, retroviral vectors generated from NMC-HLA-G cells will have a decreased percentage of lysis by target cells at specific timepoints as compared to retroviral vectors generated from NMCs or NMC-empty vector.

### Example 7. Modification of Retroviral Vector with HLA-G or HLA-E for Decreased CD8 Killer T Cell Lysis

This Example describes the generation of a retroviral vector composition derived from a cell source which has been modified to decrease cytotoxicity mediated cell lysis by CD8+ T-cells. In an embodiment, cytotoxicity mediated cell lysis of retroviral vector by CD8+ T-cells is a measure of immunogenicity for retroviral vectors.

Retroviral vectors are created from: unmodified cells (hereinafter NMCs, positive control), cells that are transfected with HLA-G or HLA-E cDNA (hereinafter NMC-HLA-G), and cells transfected with an empty vector control (hereinafter NMC-empty vector, negative control).

CD8+ T cell mediated lysis of a retroviral vector is determined by europium release assays as described in Bouma, et al. Hum. Immunol. 35(2):85-92; 1992 & van Besouw et al. Transplantation 70(1):136-143; 2000. CD8+ T-cells (hereinafter effector cells) are isolated from an appropriate donor according to the methods in Crop et al. Cell transplantation (20):1547-1559; 2011, and stimulated with allogeneic gamma irradiated PMBCs and 200 IU/mL IL-2 (proleukin, Chiron BV Amsterdam, The Netherlands) in a round bottom 96 well plate for 7 days at 37° C. The retroviral vectors are labeled with europium-diethylenetriaminepentaacetate (DTPA) (sigma, St. Louis, Mo., USA). Cytotoxicity-mediated lysis assays and data analysis are performed as described above in Example 5.

In an embodiment, retroviral vectors generated from NMC-HLA-G cells will have a decreased percentage of lysis by target cells at specific timepoints as compared to retroviral vectors generated from NMCs or NMC-empty vector.

### Example 8: Modification of Retroviral Vector with CD47 to Evade Macrophage Phagocytosis

This Example describes quantification of the evasion of phagocytosis by modified retroviral vector. In an embodiment, modified retroviral vector will evade phagocytosis by macrophages.

Cells engage in phagocytosis, engulfing particles, enabling the sequestration and destruction of foreign invaders, like bacteria or dead cells. In some embodiments, phagocytosis of lentiviral vectors by macrophages would reduce their activity. In some embodiments, phagocytosis of lentiviral vectors is a measure of immunogenicity of retroviral vectors.

Retroviral vectors are produced from cells which lack CD47 (hereinafter NMC, positive control), cells that are transfected with CD47 cDNA (hereinafter NMC-CD47), and cells transfected with an empty vector control (hereinafter NMC-empty vector, negative control). Prior to retroviral vector production, the cells are labeled with CSFE.

Reduction of macrophage mediated immune clearance is determined with a phagocytosis assay according to the following protocol. Macrophages are plated immediately after harvest in confocal glass bottom dishes. Macrophages are incubated in DMEM+10% FBS+1% P/S for 1h to attach. An appropriate number of retroviral vectors produced from NMC, NMC-CD47, NMC-empty vector are added to the macrophages as indicated in the protocol, and are incubated for 2h, tools.thennofisher.com/content/sfs/manuals/mp06694.pdf.

After 2h, the dish is gently washed and intracellular fluorescence is examined. Intracellular fluorescence emitted by engulfed retroviral particles is imaged by confocal microscopy at 488 excitation. The number of phagocytotic positive macrophage is quantified using imaging software. The data is expressed as the phagocytic index=(total number of engulfed cells/total number of counted macrophages)×(number of macrophages containing engulfed cells/total number of counted macrophages)×100.

In an embodiment, the phagocytic index will be reduced when macrophages are incubated with retroviral vectors derived from NMC-CD47, versus those derived from NMC, or NMC-empty vector.

### Example 9: Modification of Retroviral Vector with Complement Regulatory Proteins to Evade Complement

This Example describes quantification of complement activity against a retroviral vector using an in vitro assay. In some embodiments a modified retroviral vector described herein will have reduced complement activity compared to an unmodified retroviral vector.

In this Example, serum from a mouse is assessed for complement activity against a retroviral vector. The example measures the level of complement C3a, which is a central node in all complement pathways. The methods described herein may be equally applicable to humans, rats, monkeys with optimization to the protocol.

In this example, retroviral vectors are generated from HEK293 cells transfected with a cDNA coding for complement regulatory protein DAF (HEK293-DAF retroviral vector) or HEK 293 cells not expressing a complementary regulatory protein (HEK293 retroviral vector). In other embodiments, other complement regulatory proteins may be used, such as proteins that bind decay-accelerating factor (DAF, CD55), e.g. factor H (FH)-like protein-1 (FHL-1), e.g. C4b-binding protein (C4BP), e.g. complement receptor 1 (CD35), e.g. Membrane cofactor protein (MCP, CD46), eg. Profectin (CD59), e.g. proteins that inhibit the classical and alternative complement pathway CD/C5 convertase enzymes, e.g. proteins that regulate MAC assembly.

Serum is recovered from naive mice, mice that are administered HEK293-DAF retroviral vector, or mice that are administered HEK293 retroviral vector. Sera are collected from mice by collecting fresh whole blood and allowing it to clot completely for several hours. Clots are pelleted by centrifugation and the serum supernatants are removed. A negative control is heat inactivated mouse serum. Negative control samples are heated at 56 degrees Celsius for 1 hour. Serum may be frozen in aliquots.

The different retroviral vectors are tested for the dose at which 50% of cells in a target cell population receive the exogenous agent in the retroviral vector. The retroviral vector may contain any of the exogenous agents described herein. Many methods for assaying retroviral delivery of an exogenous agent to recipient cells are also described herein. In this particular example, the exogenous agent is Cre protein (encoded by the retroviral nucleic acid) and the target cells are RPMI8226 cells which stably-express a “LoxP-GFP-stop-LoxP-RFP” cassette under a CMV promoter, which upon recombination by Cre switches from GFP to RFP expression, as a marker of delivery. The identified dose at which 50% of the recipient cells are RFP positive is used for further experiments. In some embodiments, the identified dose at which 50% of recipient cells receive the exogenous agent will be similar across retroviral vectors.

Two-fold dilutions in phosphate-buffered saline (PBS, pH 7.4) of the retroviral vectors, starting at the dose of retroviral vectors at which 50% of the target cells receive the exogenous agent, are mixed with a 1:10 dilution of the sera from mice treated with the same retroviral vectors or naive mice (assay volume, 20 l) and incubated for 1 h at 37° C. The samples are further diluted 1:500 and used in an enzyme-linked immunosorbent assay (ELISA) specific for C3a. The ELISA is mouse complement C3a ELISA Kit product LS-F4210 sold by LifeSpan BioSciences Inc, which measures the concentration of C3a in a sample. The dose of retroviral vector at which 200 μg/ml of C3a is present is compared across sera isolated from mice.

In some embodiments, the dose of retroviral vector at which 200 μg/ml of C3a is present will be greater for HEK293-DAF retroviral vector incubated with HEK-293 DAF mouse sera than for HEK293 retroviral vector incubated with HEK293 mouse sera, indicating that complement activity targeting retroviral vector is greater in mice treated with HEK293 retroviral vector than HEK293-DAF retroviral vector. In some embodiments, the dose of retroviral vector at which 200 μg/ml of C3a is present will be greater for HEK293-DAF retroviral vector incubated with naive mouse sera than for HEK293 retroviral vector incubated with naive mouse sera, indicating that complement activity targeting retroviral vector is greater in mice treated with HEK293 retroviral vector than HEK293-DAF retroviral vector.

### Example 10: Modification of Retroviral Vector to Knockdown Immunogenic Protein to Reduce Immunogenicity

This Example describes the generation of a retroviral vector composition derived from a cell source which has been modified to reduce expression of a molecule which is immunogenic, and quantification of the reduced expression. In an embodiment, a retroviral vector can be derived from a cell source, which has been modified to reduce expression of a molecule which is immunogenic.

Therapies that stimulate an immune response can reduce the therapeutic efficacy or cause toxicity to the recipient. Thus, immunogenicity is an important property for a safe and effective therapeutic retroviral vectors. Expression of certain immune activating agents can create an immune response. MHC class I represents one example of an immune activating agent.

Retroviral vectors are produced from unmodified cells which normally express MHC-1 (hereinafter NMC, positive control), cells that are transfected with a DNA coding for a shRNA targeting MHC class I (hereinafter NMC-shMHC class I), and cells transfected with a DNA coding for non-targeted scrambled shRNA vector control (hereinafter NMC-vector control, negative control). Prior to retroviral production, the cells are labeled with CSFE.

Retroviral vectors are assayed for expression of MHC class I using flow cytometry. An appropriate number of retroviral vectors are washed and resuspended in PBS, held on ice for 30 minutes with 1: 10-1: 4000 dilution of fluorescently conjugated monoclonal antibodies against MHC class I (Harlan Sera-Lab, Belton, UK). Retroviral vectors are washed three times in PBS and resuspended in PBS. Nonspecific fluorescence is determined, using equal aliquots of retroviral vector preparation incubated with and appropriate fluorescently conjugated isotype control antibody at equivalent dilutions. Retroviral vectors are assayed in a flow cytometer (FACSort, Becton-Dickinson) and the data is analyzed with flow analysis software (Becton-Dickinson).

The mean fluorescence data of the retroviral vectors derived from NMCs, NMC-shMHC class I, and NMC-vector control, is compared. In an embodiment, retroviral vectors derived from NMC-shMHC class I will have lower expression of MHC class I compared to NMCs and NMC-vector control.

### Example 11: Measuring Pre-Existing Serum Inactivation of Retroviral Vectors

This Example describes quantification of pre-existing serum inactivation of retroviral vectors using an in vitro delivery assay.

In some embodiments, a measure of immunogenicity for retroviral vectors is serum inactivation. Serum inactivation of retroviral vectors may be due to antibody-mediated neutralization or complement mediated degradation. In an embodiment, some recipients of a retroviral vectors described herein will have factors in their serum which bind to and inactivate retroviral vectors.

In this Example, a retroviral vector naive mouse is assessed for the presence of factors that inactivate retroviral vectors in serum. Notably, the methods described herein may be equally applicable to humans, rats, monkeys with optimization to the protocol.

The negative control is heat inactivated mouse serum and the positive control is serum derived from a mouse that has received multiple injections of retroviral vector generated from a xenogeneic source cell. Sera are collected from mice by collecting fresh whole blood and allowing it to clot completely for several hours. Clots are pelleted by centrifugation and the serum supernatants are removed. Negative control samples are heated at 56 degrees Celsius for 1 hour. Serum may be frozen in aliquots.

The retroviral vectors are tested for the dose at which 50% of cells in a target cell population receive the exogenous agent in the retroviral vector, as described above in Example 9.

To assess serum inactivation of retroviral vectors, retroviral vectors are diluted 1:5 into normal or heat-inactivated serum (or medium containing 10% heat-inactivated FBS as the no-serum control) and the mixture is incubated at 37° C. for 1 h. Following the incubation, medium is added to the reaction for an additional 1:5 dilution and then serially diluted twice at a 1:10 ratio. Following this step, the retroviral vectors should be present at the previously identified dose at which 50% of the recipient cells have received the exogenous agent (e.g. are RFP positive).

Retroviral vectors that have been exposed to serum are then incubated with target cells. The percent of cells which receive the exogenous agent, and thus are RFP positive, is calculated. In some embodiments, the percent of cells which receive the exogenous agent will not be different between retroviral vector samples that have been incubated with serum and heat-inactivated serum from retroviral vector naive mice, indicating that there is not serum inactivation of retroviral vector. In some embodiments, the percent of cells which receive the exogenous agent will not be different between retroviral vector samples that have been incubated with serum from retroviral vector naive mice and no-serum control incubations, indicating that there is not serum inactivation of retroviral vectors. In some embodiments, the percent of cells which receive the exogenous agent will be less in retroviral vector samples that have been incubated with positive control serum than in retroviral vector samples that have been incubated with serum from retroviral vector naive mice, indicating that there is not serum inactivation of retroviral vectors.

### Example 12: Measuring Serum Inactivation of Retroviral Vectors after Multiple Administrations

This Example describes quantification of serum inactivation of retroviral vectors using an in vitro delivery assay following multiple administrations of the retroviral vectors. In an embodiment, a modified retroviral vector, e.g., modified by a method described herein, will have a reduced (e.g., reduced compared to administration of an unmodified retroviral vector) serum inactivation following multiple (e.g., more than one, e.g., 2 or more) administrations of the modified retroviral vector. In an embodiment, a retroviral vector described herein will not be inactivated by serum following multiple administrations.

In some embodiments, a measure of immunogenicity for retroviral vector is serum inactivation. In an embodiment, repeated injections of a retroviral vector can lead to the development of anti-retroviral vector antibodies, e.g., antibodies that recognize retroviral vectors. In an embodiment, antibodies that recognize retroviral vectors can bind in a manner that can limit retroviral vector activity or longevity and mediate complement degradation.

In this Example, serum inactivation is examined after one or more administrations of retroviral vectors. Retroviral vectors are produced by any one of the previous Examples. In this example, retroviral are created from: cells that are transfected with HLA-G or HLA-E cDNA (hereinafter NMC-HLA-G), and cells transfected with an empty vector control (hereinafter NMC-empty vector, negative control). In some embodiments, retroviral vectors are derived from cells that are expressing other immunoregulatory proteins.

Serum is drawn from different cohorts: mice injected systemically and/or locally with 1, 2, 3, 5, or 10 injections of vehicle (retroviral vector naive group), HEK293-HLA-G retroviral vector, or HEK293 retroviral vector. Sera are collected from mice by collecting fresh whole blood and allowing it to clot completely for several hours. Clots are pelleted by centrifugation and the serum supernatants are removed. A negative control is heat inactivated mouse serum. Negative control samples are heated at 56 degrees Celsius for 1 hour. Serum may be frozen in aliquots.

The retroviral vectors are tested for the dose at which 50% of cells in a target cell population receive the exogenous agent in the retroviral vector, as described above in Example 9.

To assess serum inactivation of retroviral vectors, retroviral vectors are exposed to serum and incubated with target cells as described in Example 11 above.

The percent of cells which receive the exogenous agent, and thus are RFP positive, is calculated. In some embodiments, the percent of cells which receive the exogenous agent will not be different between retroviral vector samples that have been incubated with serum and heat-inactivated serum from mice treated with HEK293-HLA-G retroviral vectors, indicating that there is not serum inactivation of retroviral vectors or an adaptive immune response. In some embodiments, the percent of cells which receive the exogenous agent will not be different between retroviral vector samples that have been incubated from mice treated 1, 2, 3, 5 or 10 times with HEK293-HLA-G retroviral vectors, indicating that there is not serum inactivation of retroviral vectors or an adaptive immune response. In some embodiments, the percent of cells which receive the exogenous agent will not be different between retroviral vector samples that have been incubated with serum from mice treated with vehicle and from mice treated with HEK293-HLA-G retroviral vectors, indicating that there is not serum inactivation of retroviral vectors or an adaptive immune response. In some embodiments, the percent of cells which receive the exogenous agent will be less for retroviral vectors derived from HEK293 than for HEK293-HLA-G retroviral vectors indicating that there is not serum inactivation of HEK293-HLA-G retroviral vectors or an adaptive immune response.

### Example 13: Measuring Pre-Existing IgG and IgM Antibodies Reactive Against Retroviral Vectors

This Example describes quantification of pre-existing anti-retroviral vector antibody titers measured using flow cytometry.

In some embodiments, a measure of immunogenicity for a retroviral vector is antibody responses. Antibodies that recognize retroviral vector can bind in a manner that can limit retroviral vector activity or longevity. In an embodiment, some recipients of a retroviral vector described herein will have pre-existing antibodies which bind to and recognize retroviral vector.

In this Example, anti-retroviral vector antibody titers are tested using retroviral vector produced using a xenogeneic source cell. In this Example, a retroviral vector naive mouse is assessed for the presence of anti-retroviral vector antibodies. Notably, the methods described herein may be equally applicable to humans, rats, monkeys with optimization to the protocol.

The negative control is mouse serum which has been depleted of IgM and IgG, and the positive control is serum derived from a mouse that has received multiple injections of retroviral vector generated from a xenogeneic source cell.

To assess the presence of pre-existing antibodies which bind to retroviral vector, sera from retroviral vector-naïve mice is first decomplemented by heating to 56° C. for 30 min and subsequently diluted by 33% in PBS containing 3% FCS and 0.1% NaN₃. Equal amounts of sera and retroviral vector (1×10²-1×108 retroviral vectors per mL) suspensions are incubated for 30 min at 4° C. and washed with PBS through a calf-serum cushion.

IgM xenoreactive antibodies are stained by incubation of the retroviral vector with PE-conjugated goat antibodies specific for the Fc portion of mouse IgM (BD Bioscience) at 4° C. for 45 min. Notably, anti-mouse IgG1 or IgG2 secondary antibodies may also be used. Retroviral vector from all groups are washed twice with PBS containing 2% FCS and then analyzed on a FACS system (BD Biosciences). Fluorescence data are collected by use of logarithmic amplification and expressed as mean fluorescent intensity.

In an embodiment, the negative control serum will show negligible fluorescence comparable to the no serum or secondary alone controls. In an embodiment, the positive control will show more fluorescence than the negative control, and more than the no serum or secondary alone controls. In an embodiment, in cases where immunogenicity occurs, serum from retroviral vector-naive mice will show more fluorescence than the negative control. In an embodiment, in cases where immunogenicity does not occur, serum from retroviral vector-naive mice will show similar fluorescence compared to the negative control.

### Example 14: Measuring IgG and IgM Antibody Responses after Multiple Administrations of Retroviral Vectors

This Example describes quantification of the humoral response of a modified retroviral vector following multiple administrations of the modified retroviral vector. In an embodiment, a modified retroviral vector, e.g., modified by a method described herein, will have a reduced (e.g., reduced compared to administration of an unmodified retroviral vector) humoral response following multiple (e.g., more than one, e.g., 2 or more), administrations of the modified retroviral vector.

In some embodiments, a measure of immunogenicity for a retroviral vector is the antibody responses. In an embodiment, repeated injections of a retroviral vector can lead to the development of anti-retroviral vector antibodies, e.g., antibodies that recognize retroviral vector. In an embodiment, antibodies that recognize retroviral vector can bind in a manner that can limit retroviral vector activity or longevity.

In this Example, anti-retroviral vector antibody titers are examined after one or more administrations of retroviral vector. Retroviral vector is produced by any one of the previous Examples. In this example, retroviral are created from: cells that are not transfected with an immunomodulatory protein (NMCs), cells that are transfected with HLA-G or HLA-E cDNA (hereinafter NMC-HLA-G), and cells transfected with an empty vector control (hereinafter NMC-empty vector, negative control). In some embodiments, retroviral vectors are derived from cells that are expressing other immunoregulatory proteins.

Serum is drawn from different cohorts: mice injected systemically and/or locally with 1, 2, 3, 5, 10 injections of vehicle (retroviral vector naive group), NMC retroviral vector, NMC-HLA-G retroviral vector, or NMC-empty vectors retroviral vector.

To assess the presence and abundance of anti-retroviral vector antibodies, sera from the mice is first decomplemented by heating to 56° C. for 30 min and subsequently diluted by 33% in PBS with 3% FCS and 0.1% NaN₃. Equal amounts of sera and retroviral vector (1×10²-1×10⁸ retroviral vector per mL) are incubated for 30 min at 4° C. and washed with PBS through a calf-serum cushion.

Retroviral vector reactive IgM antibodies are stained by incubation of the retroviral vector with PE-conjugated goat antibodies specific for the Fc portion of mouse IgM (BD Bioscience) at 4° C. for 45 min. Notably, anti-mouse IgG1 or IgG2 secondary antibodies may also be used. Retroviral vector from all groups are washed twice with PBS containing 2% FCS and then analyzed on a FACS system (BD Biosciences). Fluorescence data are collected by use of logarithmic amplification and expressed as mean fluorescent intensity.

In an embodiment, NMC-HLA-G retroviral vectors will have decreased anti-viral IgM (or IgG1/2) antibody titers (as measured by fluorescence intensity on FACS) after injections, as compared to NMC retroviral vectors or NMC-empty retroviral vectors.

### Example 15: Measuring IgG and IgM Titers Antibody Responses to Retroviral Vector Recipient Cells

This Example describes quantification of antibody titers against recipient cells (cells that have fused with retroviral vectors) using flow cytometry. In some embodiments, a measure of the immunogenicity of recipient cells is the antibody response. Antibodies that recognize recipient cells can bind in a manner that can limit cell activity or longevity. In an embodiment, recipient cells will not be targeted by an antibody response, or an antibody response will be below a reference level.

In this Example, anti-recipient cell antibody titers in a subject (e.g., human, rat, or monkey) are tested. In addition, the protocol may be adapted to any cell type for which suitable surface markers exist. In this example, the target recipient cell is a CD3+ cell.

Mice are treated with retroviral vectors produced via any of the methods described in this application or with PBS (negative control) daily for 5 days. 28 days following the final treatment, peripheral blood is collected from mice that received retroviral vectors and mice that received PBS treatment. Blood is collected into 1 ml PBS containing 5 μM EDTA and mixed immediately to prevent clotting. The tubes are kept on ice and red blood cells are removed using a buffered ammonium chloride (ACK) solution. Cells are stained with a murine CD3-FITC antibody (Thermo Fisher Catalog #:11-0032-82), at 4° C. for 30 minutes in the dark, after being blocked with bovine serum albumin for 10 minutes. After being washed two times with PBS, cells are analyzed on a LSR II (BD Biosciences, San Jose, Calif.) with 488 nm laser excitation and emission collected at 530+/−30 nm running the FACSDiva™ software (BD Biosciences, San Jose, Calif.). CD3+ cells are sorted.

The sorted CD3+ cells are then stained with IgM antibodies by incubation of the reaction mixture with PE-conjugated goat antibodies specific for the Fc portion of mouse IgM (BD Bioscience) at 4° C. for 45 min. Notably, anti-mouse IgG1 or IgG2 secondary antibodies may also be used. Cells from all groups are washed twice with PBS containing 2% FCS and then analyzed on a FACS system (BD Biosciences). Fluorescence data are collected by use of logarithmic amplification and expressed as mean fluorescent intensity. The mean fluorescence intensity is calculated for the sorted CD3 cells from mice treated with retroviral vectors and the mice treated with PBS.

A low mean fluorescence intensity is indicative of a low humoral response against the recipient cells. Mice treated with PBS are expected to have low mean fluorescence intensity. In an embodiment, the mean fluorescence intensity will be similar for recipient cells from mice treated with retroviral vectors and mice treated with PBS.

### Example 16: Measuring Phagocytic Response to Retroviral Vector Recipient Cells

This Example describes quantification of macrophage response against recipient cells with a phagocytosis assay.

In some embodiments, a measure of the immunogenicity of recipient cells is the macrophage response. Macrophages engage in phagocytosis, engulfing cells and enabling the sequestration and destruction of foreign invaders, like bacteria or dead cells. In some embodiments, phagocytosis of recipient cells by macrophages would reduce their activity.

In an embodiment, recipient cells are not targeted by macrophages. In this Example, the macrophage response against recipient cells in a subject is tested. In addition, the protocol may be adapted to any cell type for which suitable surface markers exist. In this example, the target recipient cell is a CD3+ cell.

Mice are treated with retroviral vectors produced via any of the methods described in this application or with PBS (negative control) daily for 5 days. 28 days following the final treatment, peripheral blood is collected from mice that received retroviral vectors and mice that received PBS treatment. Blood is collected into 1 ml PBS containing 5 μM EDTA and mixed immediately to prevent clotting. The tubes are kept on ice and red blood cells are removed using a buffered ammonium chloride (ACK) solution.

Cells are stained with a murine CD3-FITC antibody (Thermo Fisher Catalog #:11-0032-82), at 4° C. for 30 minutes in the dark, after being blocked with bovine serum albumin for 10 minutes. After being washed two times with PBS, cells are analyzed on a LSR II (BD Biosciences, San Jose, Calif.) with 488 nm laser excitation and emission collected at 530+/−30 nm running the FACSDiva™ software (BD Biosciences, San Jose, Calif.). CD3+ cells are then sorted.

A phagocytosis assay is run to assess macrophage mediated immune clearance according to the following protocol. Macrophages are plated immediately after harvest in confocal glass bottom dishes. Macrophages are incubated in DMEM+10% FBS+1% P/S for 1h to attach. An appropriate number of sorted and FITC-stained CD3+ cells derived from mice that received retroviral vectors and PBS are added to the macrophages as indicated in the protocol, and are incubated for 2h, e.g., as described in the Vybrant™ Phagocytosis Assay Kit product information insert (Molecular Probes, revised 18 μMar. 2001, found at tools.thermofisher.com/content/sfs/manuals/mp06694.pdf).

After 2h, the dish is gently washed and intracellular fluorescence is examined. To identify macrophages, cells are first incubated with Fc-receptor blocking antibody (eBioscence cat. no. 14-0161-86, clone 93) for 15 min on ice to block the binding of labeled mAbs to Fc receptors, which are abundantly expressed on macrophages. Following this step anti-F4/80-PE (ThermoFisher cat. No. 12-4801-82, clone BM8) and anti-CD11b-PerCP-Cy5.5 (BD Biosciences cat. No. 550993, clone M1/70) conjugated antibodies are added to stain macrophage surface antigens. Cells are incubated for 30 min in the dark at 4 C followed by centrifugation and washing in PBS. The cells are then resuspended in PBS. Flow cytometry of samples is then performed and macrophages are identified via positive fluorescence signal for F4/80-PE and CD11b-PerCP-Cy5.5 using 533 nm and 647 nm laser excitation, respectively. After gating for macrophages, intracellular fluorescence emitted by engulfed recipient cells is assessed by 488 nm laser excitation. The number of phagocytotic positive macrophage is quantified using imaging software. The data is expressed as the phagocytic index=(total number of engulfed cells/total number of counted macrophages)×(number of macrophages containing engulfed cells/total number of counted macrophages)×100.

A low phagocytic index is indicative of low phagocytosis and targeting by macrophages. Mice treated with PBS are expected to have a low phagocytic index. In an embodiment, the phagocytic index will be similar for recipient cells derived from mice treated with retroviral vectors and mice treated with PBS.

### Example 17: Measuring PBMC Response to Retroviral Vector Recipient Cells

This Example describes quantification of a PBMC response against recipient cells with a cell lysis assay.

In some embodiments, a measure of the immunogenicity of recipient cells is the PBMC response. In an embodiment, cytotoxicity mediated cell lysis of recipient cells by PBMCs is a measure of immunogenicity, as lysis will reduce, e.g., inhibit or stop, the activity of a retroviral vector.

In an embodiment, recipient cells do not elicit a PBMC response. In this Example, the PBMC response against recipient cells in a subject is tested.

In addition, the protocol may be adapted to any cell type for which suitable surface markers exist. In this example, the target recipient cell is a CD3+ cell.

Mice are treated with retroviral vector produced via any of the methods described in this application or with PBS (negative control) daily for 5 days. 28 days following the final treatment, peripheral blood is collected from mice that received retroviral vector and mice that received PBS treatment. Blood is collected into 1 ml PBS containing 5 μM EDTA and mixed immediately to prevent clotting. The tubes are kept on ice and red blood cells are removed using a buffered ammonium chloride (ACK) solution. Cells are stained with a murine CD3:APC-Cy7 antibody (Biolgend Catalog #: 100330) or an isotype control APC-Cy7 (IC:APC-Cy7) antibody (Biolgend Catalog #: 400230) at 4° C. for 30 minutes in the dark, after being Fc blocked (Biolgend Catalog #: 101319) in cell staining buffer (Biolgend Catalog #: 420201) for 10 minutes. After being washed two times with PBS, cells are analyzed on a FACS Aria (BD Biosciences, San Jose, Calif.) with 640 nm laser excitation and emission collected at 780−/+60 nm running the FACSDiva™ software (BD Biosciences, San Jose, Calif.) to set negative gates using the isotype control APC-Cy7 antibody labelled cells and then APC-Cy7 positive cells are sorted and collected. Sorted CD3+ cells are then labelled with either CellMask™ Green Plasma membrane Stain (CMG, ThermoFisher Catalog #: C37608) or DMSO as the negative control.

7 days prior to the isolation of CD3+ cells from the mice treated with retroviral vector or PBS, PBMCs are isolated from mice treated with retroviral vector or PBS according to the methods in Crop et al. Cell transplantation (20):1547-1559; 2011 and simulated in the presence of IL-2 recombinant mouse protein (R&D Systems Catalog #: 402-ML-020) and CD3/CD28 beads (ThermoFisher Catalog #: 11456D) in a round bottom 96 well plate for 7 days at 37 C. At day 7, the stimulated PBMCs are co-incubated with CD3+/CMG+ or CD3+/DMSO control cells for 1, 2, 3, 4, 5, 6, 8, 10, 15, 20, 24, 48 hours at a plating ratio of PBMC:CD3+/CMG+ or PBMC: CD3+/DMSO control cells ranging from 1000:1-1:1 and 1:1.25-1:1000. As a negative control a set of wells would receive CD3+/CMG+ and CD3+/DMSO control cells only, no PBMCs. After incubation, the plates are centrifuged and processed so that they are labelled with either murine CD3:APC-Cy7 antibody or an IC:APC-Cy7 antibody as per above. After being washed two times with PBS, cells are re-suspended in PBS and analyzed on a FACS Aria (APC-Cy7: 640 nm laser excitation/emission collected at 780−/+60 nm and CMG 561 nm laser excitation/emission collected at 585−/+16 nm) running the FACSDiva™ software (BD Biosciences, San Jose, Calif.). The FSC/SSC event data would then be used initially to set the gate for events labelled “cells”. This “cells” gate would be then used to display events to set the PMT voltage for the 640 nm and 561 nm laser analyzing samples labelled with IC:APC-Cy7/DMSO only. This sample would also be used to set the gates for negative cells for both APC-Cy7 and CMG. The CD3+/CMG+ cells that did not receive any PBMCs would then used to set the positive gates for CD3+ and CMG+ cells.

The data is analyzed by looking at the percentage of CD3+/CMG+ cells in the population of total cells. When comparing treatment groups, a relatively lower percentage of CD3+/CMG+ cells at any given assay ratio of PBMC:CD3+/CMG+ cells is indicative of recipient cell lysis. In an embodiment, the percent of CD3+/CMG+ will be similar for recipient cells derived from mice treated with retroviral vector and mice treated with PBS.

### Example 18: Measuring NK Cell Response to Retroviral Vector Recipient Cells

This Example describes quantification of a natural killer cell response against recipient cells with a cell lysis assay.

In some embodiments, a measure of the immunogenicity of recipient cells is the natural killer cell response. In an embodiment, cytotoxicity mediated cell lysis of recipient cells by natural killer cells is a measure of immunogenicity, as lysis will reduce, e.g., inhibit or stop, the activity of a retroviral vector.

In an embodiment, recipient cells do not elicit a natural killer cell response. In this Example, the natural killer response against recipient cells in a subject is tested. In addition, the protocol may be adapted to any cell type for which suitable surface markers exist. In this example, the target recipient cell is a CD3+ cell.

Mice are treated with retroviral vector, a blood sample is drawn, and CD3+ cells are sorted as described above in Example 17. NK cells are isolated, cultured with the CD3+ cells, and analyzed by FACS according to the protocol described above in Example 17 except that NK cells are used in place of the PBMC cells used in Example 17.

The data is analyzed by looking at the percentage of CD3+/CMG+ cells in the population of total cells. When comparing treatment groups, a relatively lower percentage of CD3+/CMG+ cells at any given assay ratio of NK cells:CD3+/CMG+ cells is indicative of recipient cell lysis. In an embodiment, the percent of CD3+/CMG+ will be similar for recipient cells derived from mice treated with retroviral vector and mice treated with PBS.

### Example 19: Measuring CD8 T Cell Response to Retroviral Vector Recipient Cells

This Example describes quantification of a CD8+ T cell response against recipient cells (cells that have fused with retroviral vectors) with a cell lysis assay.

In some embodiments, a measure of the immunogenicity of recipient cells is the CD8+ T cell response. In an embodiment, cytotoxicity mediated cell lysis of recipient cells by CD8+ T cells is a measure of immunogenicity, as lysis will reduce, e.g., inhibit or stop, the activity of a retroviral vector.

In an embodiment, recipient cells do not elicit a CD8+ T cell response. In this Example, the CD8+ T cell response against recipient cells in a subject is tested. In addition, the protocol may be adapted to any cell type for which suitable surface markers exist. In this example, the target recipient cell is a CD3+ cell.

Mice are treated with retroviral vector, a blood sample is drawn, and CD3+ cells are sorted as described above in Example 17. CD8+ T cells are isolated, cultured with the CD3+ cells, and analyzed by FACS according to the protocol described above in Example 17 except that CD8+ T cells are used in place of the PBMC cells used in Example 17.

The data is analyzed by looking at the percentage of CD3+/CMG+ cells in the population of total cells. When comparing treatment groups, a relatively lower percentage of CD3+/CMG+ cells at any given assay ratio of CD8+ cells:CD3+/CMG+ cells is indicative of recipient cell lysis. In an embodiment, the percent of CD3+/CMG+ will be similar for recipient cells derived from mice treated with retroviral vectors and mice treated with PBS.

### Example 20. Creating a Fusogen-Resistant Source Cell

This example describes a source cell that cannot be targeted (or is targeted at a reduced level) by a retroviral vector because the source cell has been modified so that the receptor that the retroviral vector targets is absent or at a reduced level.

In this Example the fusogen is Syncytin-1 (HERV-W), the receptor is ASCT2, and receptor expression is reduced in source cells using genome editing with Cas9 and a guide RNA targeting the ASCT2 locus. It is understood that a variety of other receptors can be downregulated by a variety of methods, e.g., using RNA interference.

A HEK293T source line is transfected with mRNA coding for Cas9 and a guide RNA targeting ASCT2 (HEK293T-ASCT2) or with control mRNA that does not express Cas9 (HEK293T-control). Cells are cultured for 3 weeks and then stained for ASCT2 expression using immunostaining and flow cytometry. In some embodiments, at least 90% of cells in the HEK293T-ASCT2 population will stain negative for ASCT2 and at least 90% of cells in the HEK293T-control population will stain positive for ASCT2. These cells are then used for subsequent experiments.

The cells are then transfected with lentiviral packaging vectors (Gag, Pol, and Rev), a Syncytin-1 envelope vector, and a payload vector encoding GFP. After 24 hours, the cells are imaged using microscopy to assay syncytia formation and then viral particles are collected from the supernatant.

The amount of syncytia in the source cell population can be assayed by quantifying the number of nuclei per syncytium microscopically after staining with DAPI. In some embodiments, the percent of nuclei per syncytium is less in HEK293T-ASCT2 source cells than in HEK293T-control source cells.

The amount of functional viral particles from HEK293T-ASCT2 source cells and HEK293T-control source cells is quantified by culturing HEK293T cells with the viral particles collected from the supernatant. The cells are cultured for 5 days after culturing with the viral particles and the percent of GFP positive cells is assayed via flow cytometry. In some embodiments, the percent of GFP positive cells will be greater in HEK293T cells treated with HEK293T-ASCT2 source cell-derived viral particles than in HEK293T-control source cell-derived viral particles.

### Example 21: Assessing Median Expression Level in Target Cells Over Time

This Example describes quantification of the expression of an exogenous agent in target cells by measuring exogenous agent levels in single cells.

In an embodiment, the exogenous agent level is greater than a house keeping gene. In an embodiment, payload expression is similar across target cells.

In this example, the target cell is a CD3+ cell. However, this protocol may be adapted to any cell type for which suitable surface markers exist and which can be isolated from the subject. Notably, the methods described herein may be equally applicable to humans, rats, monkeys with optimization to the protocol. In this example the payload is a fluorescent protein and expression is measured via flow cytometry. In other embodiments, the expression of a protein payload may be measured with immunostaining for the protein. In other embodiments expression of the protein payload may be measured via microscopy or Western blot.

Mice are treated with retroviral vector with a tdtomato fluorescent protein agent produced via any of the methods described in this application or with PBS (negative control). 7 days, 14 days, 28 days, 56 days, 112 days, 365 days, 730 days, and 1095 days following treatment, peripheral blood is collected from mice that received retroviral vector and mice that received PBS treatment. Blood is collected as described above in Example 2. Cells are stained with a murine CD3:APC-Cy7 antibody (Biolegend Catalog #: 100330) or isotype controls and analyzed by flow cytometry using the protocol described in Example 2.

The percent of CD3+ cells that are tdtomato positive is measured. In some embodiments, the percent of CD3+ cells that are tdtomato positive will be greater in cells from treated than untreated mice. In some embodiments, the percent of CD3+ cells that are tdtomato positive will be similar across cells collected at 7 days, 14 days, 28 days, 56 days, 112 days, 365 days, 730 days, or 1095 days. The median tdtomato fluorescence level is measured in CD3+ Tdtomato+ cells. In some embodiments, the median tdtomato fluorescence level in CD3+ Tdtomato+ cells will be similar in cells collected at 7 days, 14 days, 28 days, 56 days, 112 days, 365 days, 730 days, or 1095 days.

### Example 22: Assessment of Specificity of Transgene Expression Using Tissue-Specific Promoters and miRNA Mediated Gene Silencing

This Example describes quantification of an exogenous agent in target human hepatoma cell line (HepG2) and compared to non-target (non-hepatic) cell lines. Cell lines were transduced with lentiviruses (LV) containing positive TCSREs (e.g. tissue-specific promoter) or a combination of positive TCSREs and NTCSREs (e.g. miRNA-mediated gene silencing with a tissue-specific miRNA recognition sequence). Target and non-target cells lines were transduced with generated lentiviral particles containing the positive and negative regulatory elements and the effect of transgene expression in the cells lines was assessed.

### A. EFFECT OF MIRNA-MEDIATED GENE REGULATION ON SPECIFICITY OF TRANSGENE EXPRESSION

In addition to hepatocytes, major cell populations that line liver sinusoids include endothelial cells and Kupffer cells (resident macrophages derived from the hematopoietic lineage), which express mir-126-3p and mir-142-3p, respectively. These miRNAs are not substantially expressed in hepatocytes. Lentiviral vectors were constructed to contain an enhanced green fluorescent protein (eGFP) expression cassette under the control of the constitutively active promoter phosphoglycerate kinase (hPGK, positive TCSRE; see e.g. SEQ ID NO:139, with or without four tandem copies each of sequences complementary to mir-142-3p (e.g. Table 7, SEQ ID NO:143) and miR-126-3p (e.g. Table 7, SEQ ID NO:160) as an NTCSRE. Lentiviral vector (LV) constructs with the NTCSRE were designated hPGK-eGFP+miRT and constructs without the NTCSRE are designated hPGK-eGFP.

Lentiviruses (LVs) generated from these hPGK-eGFP+miRT and hPGK-eGFP vectors, respectively, were used to transduce a target human hepatoma cell line (HepG2), human embryonic kidney cell line (293LX), human T-cell line of hematopoietic origin (Molt4.8), or an endothelial cell line derived from mouse brain (bEND.3). Seven days post-transduction, GFP expression was measured by flow cytometry.

As shown in FIG. 1A, 18-30% of all cell types transduced with hPGK-eGFP LV expressed GFP. Following transduction of LVs containing mirT sequences (hPGK-eGFP+miRT) in non-target cells, only 0.6% GFP expression was observed in Molt4.8 cells (express mir-142-3p) and no expression was observed in bEND.3 cells (express mir-126-3p). Mild reduction of GFP expression was observed in 293LX cells, which may have very low level expression of one or both of these miRNAs. No effect on GFP expression was observed in HepG2 target cells that had been transduced with LVs containing mirT sequences (hPGK-eGFP+miRT). These results demonstrated that incorporation of miRT sequences in lentiviral vectors resulted in at least a 50-fold reduction in transgene expression in cells of the hematopoietic and endothelial lineages, while maintaining robust expression in hepatic cells.

### B. COMBINED EFFECT OF MIRNA-MEDIATED GENE REGULATION AND TISSUE-SPECIFIC PROMOTERS ON TRANSGENE EXPRESSION

Additional lentiviral vectors (LVs) were generated substantially as described above, but with either an eGFP or with a transgene encoding the enzyme phenylalanine ammonia lyase (PAL) with an N-terminal flag tag (nucleotide sequence shown below, under the control of a hepatocyte-specific human (ApoE.HCR-hAAT (hApoE) promoter (e.g., as shown in Table 6, SEQ ID NO:133) as a tissue-specific regulatory promoter as a positive TCSREs or a constitutively active Spleen-focus-forming Virus (SFFV) promoter (e.g., as shown in Table 6, SEQ ID NO:142). Expression of PAL in liver cells using the provided nucleic acid constructs is representative of expression of a desired exogenous agent in target cells. For example, endogenous PAH deficiency in human liver cells can result in toxic accumulation of Phe in the blood leading to phenylketonuria (PKU), a clinical condition characterized by severe neurological disorders and stunted growth. In some aspects, early administration of PAL to PKU patients has been shown to successfully decrease blood Phe levels and alleviate symptoms.

The nucleotide sequence of the PAL gene is shown (SEQ ID NO:169):

ATGGACTACAAAGACGATGACGACAAGGCCAAGACACTGTCTCAGGCC
CAGAGCAAGACCAGCAGCCAGCAGTTTAGCTTCACCGGCAACAGCAGC
GCCAACGTGATCATCGGCAACCAGAAGCTGACCATCAACGACGTGGCC
AGAGTGGCCCGGAATGGCACACTGGTGTCCCTGACCAACAACACCGAT
ATCCTGCAGGGCATCCAGGCCAGCTGCGACTACATCAACAACGCCGTG
GAAAGCGGCGAGCCCATCTACGGCGTGACATCTGGCTTTGGCGGCATG
GCTAATGTGGCCATCAGCAGAGAGCAGGCCAGCGAGCTGCAGACCAAT
CTCGTGTGGTTCCTGAAAACCGGCGCTGGCAACAAACTGCCCCTGGCT
GATGTTCGGGCTGCCATGCTGCTGAGAGCCAACTCTCACATGAGAGGC
GCCAGCGGCATCCGGCTGGAACTGATCAAGCGGATGGAAATCTTCCTG
AACGCTGGCGTGACCCCTTACGTGTACGAGTTTGGCTCTATCGGCGCC
TCCGGCGATCTGGTGCCTCTGTCTTACATCACCGGCAGCCTGATCGGC
CTGGATCCTAGCTTCAAGGTGGACTTCAACGGCAAAGAGATGGACGCC
CCTACCGCTCTGAGACAGCTGAATCTGAGCCCTCTGACACTGCTGCCC
AAAGAAGGCCTGGCCATGATGAATGGCACCAGCGTGATGACAGGGATC
GCCGCCAATTGCGTGTACGACACCCAGATCCTGACCGCCATTGCCATG
GGAGTGCACGCCCTGGATATTCAGGCCCTGAACGGCACCAACCAGAGC
TTTCACCCCTTCATCCACAACAGCAAGCCCCATCCTGGACAGCTGTGG
GCCGCTGATCAGATGATTAGCCTGCTGGCCAACAGCCAGCTCGTGCGG
GATGAGCTGGATGGCAAGCACGACTACAGAGATCACGAGCTGATCCAG
GACCGGTACAGCCTGAGATGCCTGCCTCAGTATCTGGGCCCTATCGTG
GATGGCATCTCTCAGATCGCCAAGCAGATCGAGATTGAGATCAACAGC
GTGACCGACAATCCCCTGATCGACGTGGACAACCAGGCCTCTTATCAC
GGCGGCAACTTTCTGGGCCAGTACGTCGGCATGGGCATGGACCACCTG
AGGTACTATATCGGCCTCCTGGCCAAGCACCTGGACGTGCAAATTGCC
CTGCTGGCAAGCCCCGAGTTCAGCAATGGACTGCCTCCTAGCCTGCTC
GGCAACCGCGAGAGAAAAGTGAACATGGGCCTGAAGGGCCTGCAGATC
TGTGGCAACTCCATCATGCCCCTGCTGACCTTCTACGGCAACTCTATC
GCCGACAGATTCCCCACACACGCCGAGCAGTTCAACCAGAACATCAAC
TCCCAGGGCTACACCAGCGCCACACTGGCTAGAAGAAGCGTGGACATC
TTCCAGAACTACGTGGCAATCGCCCTGATGTTTGGAGTGCAGGCCGTG
GACCTGCGGACCTACAAGAAAACAGGCCACTACGACGCCAGAGCCAGC
CTGTCTCCTGCCACCGAGAGACTGTATTCTGCCGTGCGGCATGTCGTG
GGCCAGAAGCCTACAAGCGACAGACCCTACATCTGGAACGACAACGAG
CAGGGCCTCGACGAGCACATTGCCAGAATCTCTGCCGATATCGCTGCC
GGCGGAGTGATTGTGCAGGCTGTGCAAGACATCCTGCCAAGCCTGCAC
TGA

As shown in FIG. 1B, transduction with LVs containing hPGK-eGFP or LVs containing 25 mirT sequences and GFP under the control of the hepatocyte specific promoter (hApoE-eGFP+miRT), resulted in greater than 250-fold repression of GFP expression in 293LX cells, with no substantial effect in HepG2 cells, as measured by flow cytometry.

HepG2 and 293LX cells were transduced with LVs containing the PAL transgene under the control of the SFFV promoter (SFFV-PAL), or LVs containing PAL transgene along with 30 mirT sequences under the control of the hApoE promoter (hApoE-PAL+miRT). PAL catalyzes the conversion of phenylalanine (Phe) to ammonia and cinnamic acid, and has been used in enzyme replacement therapy in patients with an inborn deficiency of phenylalanine hydroxylase (PAH). Specificity of PAL transgene expression was measured by reduction in Phe levels in culture supernatant (SN) relative to fresh medium.

As shown in FIG. 1C, expression from a constitutive promoter (SFFV) resulted in substantial reduction in Phe levels in SN collected from both cell types. However, the hApoE-PAL+miRT construct led to substantial Phe reduction only in HepG2 cells; Phe levels in SN collected from 293LX cells transduced with the hApoE-PAL+miRT LVs were indistinguishable from the untransduced controls. This result is consistent with high expression of the exemplary transgene PAL in HepG2 target cells when transduced with LV constructs containing a positive TSCRE and a NTSCRE but not in non-target 293LX cells.

### C. CONCLUSION

Together, these data are supportive of the finding that the use of a tissue-specific promoter in conjunction with miRNA target sites can impart substantial specificity to transgene expression.

What is claimed is:

1. A fusosome comprising:
a) a lipid bilayer comprising a fusogen; and
b) a nucleic acid that comprises or encodes:
(i) a positive target cell-specific regulatory element operatively linked to a nucleic acid encoding an exogenous agent, wherein the positive tissue-specific regulatory element increases expression of the exogenous agent in a target cell or tissue relative to an otherwise similar fusosome lacking the positive tissue-specific regulatory element; or
(ii) a non-target cell-specific regulatory element operatively linked to the nucleic acid encoding the exogenous agent, wherein the non-target cell-specific regulatory element decreases expression of the exogenous agent in a non-target cell or tissue relative to an otherwise similar fusosome lacking the non-target cell-specific regulatory element.

2. A fusosome comprising:
a) a lipid bilayer comprising a fusogen;
b) a nucleic acid that comprises or encodes:
(i) a positive target cell-specific regulatory element operatively linked to a nucleic acid encoding an exogenous agent, wherein the positive tissue-specific regulatory element increases expression of the exogenous agent in a target cell or tissue relative to an otherwise similar fusosome lacking the positive tissue-specific regulatory element; and
(ii) a non-target cell-specific regulatory element operatively linked to the nucleic acid encoding the exogenous agent, wherein the non-target cell-specific regulatory element decreases expression of the exogenous agent in a non-target cell or tissue relative to an otherwise similar fusosome lacking the non-target cell-specific regulatory element.

3. The fusosome of claim 1 or claim 2, wherein the fusosome comprises the exogenous agent or a nucleic acid encoding the exogenous agent.

4. The fusosome of any of the preceding claims, wherein one or more of:
i) the fusosome fuses at a higher rate with a target cell than with a non-target cell, optionally wherein the higher rate is by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
ii) the fusosome fuses at a higher rate with a target cell than with another fusosome, optionally wherein the higher rate is by at least 10%, 20%, 30%, 40%, 50%, 60%, 70%, 80%, or 90%, 2-fold, 3-fold, 4-fold, 5-fold, 10-fold, 20-fold, 50-fold, or 100-fold;
iii) the fusosome fuses with target cells at a rate such that the exogenous agent in the fusosome is delivered to at least 10%, 20%, 30%, 40%, 50%, 60%, 70%, 80%, or 90%, of target cells after 24, 48, or 72 hours;
iv) the fusosome delivers the nucleic acid to a target cell at a higher rate than to a non-target cell, optionally wherein the higher rate is by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
v) the fusosome delivers the nucleic acid to a target cell at a higher rate than to another fusosome, optionally wherein the higher rate is by at least 10%, 20%, 30%, 40%, 50%, 60%, 70%, 80%, or 90%, 2-fold, 3-fold, 4-fold, 5-fold, 10-fold, 20-fold, 50-fold, or 100-fold; or
vi) the fusosome delivers the nucleic acid to a target cell at a rate such that the exogenous agent in the fusosome is delivered to at least 10%, 20%, 30%, 40%, 50%, 60%, 70%, 80%, or 90%, of target cells after 24, 48, or 72 hours.

5. The fusosome of any of the preceding claims, wherein, when the fusosome is administered to a subject, one or more of:
i) less than 10%, 5%, 4%, 3%, 2%, or 1% of the exogenous agent detectably present in the subject is in non-target cells;
ii) at least 90%, 95%, 96%, 97%, 98%, or 99% of the cells of the subject that detectably comprise the exogenous agent, are target cells, optionally wherein the target cells are of a single cell type, optionally wherein the target cells are T cells;
iii) less than 1,000,000, 500,000, 200,000, 100,000, 50,000, 20,000, or 10,000 cells of the cells of the subject that detectably comprise the exogenous agent are non-target cells;
iv) average levels of the exogenous agent in all target cells in the subject are at least 100-fold, 200-fold, 500-fold, or 1,000-fold higher than average levels of the exogenous agent in all non-target cells in the subject; or
v) the exogenous agent is not detectable in any non-target cell in the subject.

6. The fusosome of any of the preceding claims, wherein the fusogen is a re-targeted fusogen.

7. The fusosome of claim 6, wherein the re-targeted fusogen comprises a sequence chosen from Nipah virus F and G proteins, measles virus F and H proteins, tupaia paramyxovirus F and H proteins, paramyxovirus F and G proteins or F and H proteins or F and HN proteins, Hendra virus F and G proteins, Henipavirus F and G proteins, Morbilivirus F and H proteins, respirovirus F and HN protein, a Sendai virus F and HN protein, rubulavirus F and HN proteins, or avulavirus F and HN proteins, or a derivative thereof, or any combination thereof.

8. The fusosome of any of the preceding claims, wherein the fusogen comprises a domain of at least 100 amino acids in length having at least 80%, 85%, 90%, 95%, 96%, 97%, 98%, or 99% sequence identity to a wild-type paramyxovirus fusogen, optionally wherein the wild-type paramyxovirus fusogen is set forth in any one of SEQ ID NOS: 1-132.

9. The fusosome of claim 8, wherein the wild-type paramyxovirus is a Nipah virus, optionally wherein the Nipah virus is a henipavirus.

10. The fusosome of any of the preceding claims, wherein the positive target cell-specific regulatory element comprises a tissue-specific promoter, a tissue-specific enhancer, a tissue-specific splice site, a tissue-specific site extending half-life of an RNA or protein, a tissue-specific mRNA nuclear export promoting site, a tissue-specific translational enhancing site, or a tissue-specific post-translational modification site.

11. The fusosome of any of the preceding claims, wherein the positive target cell-specific regulatory element comprises a tissue-specific promoter.

12. The fusosome of any of the preceding claims, wherein the non-target cell specific regulatory element comprises a tissue-specific miRNA recognition sequence, tissue-specific protease recognition site, tissue-specific ubiquitin ligase site, tissue-specific transcriptional repression site, or tissue-specific epigenetic repression site.

13. The fusosome of any of the preceding claims, wherein the non-target cell specific regulatory element comprises a tissue-specific miRNA recognition sequence.

14. The fusosome of claim 13, wherein the non-target cell specific regulatory element is situated or encoded within a transcribed region encoding the exogenous agent, optionally wherein an RNA produced by the transcribed region comprises the tissue-specific miRNA recognition sequence within a UTR or coding region.

15. The fusosome of any of the preceding claims, wherein the target cell is a cancer cell and the non-target cell is a non-cancerous cell.

16. The fusosome of any of the preceding claims, wherein the exogenous agent is an exogenous polypeptide or exogenous RNA, optionally wherein the exogenous agent is a therapeutic agent.

17. The fusosome of any of the preceding claims, wherein the fusosome further comprises:
i) a first exogenous or overexpressed immunosuppressive protein on the lipid bilayer and a second exogenous or overexpressed immunosuppressive protein on the lipid bilayer;
ii) a first exogenous or overexpressed immunosuppressive protein on the lipid bilayer and a second immunostimulatory protein that is absent or present at reduced levels, optionally wherein the reduced level is by at least 10%, 20%, 30%, 40%, 50%, 60%, 70%, 80%, or 90%, compared to a fusosome generated from an otherwise similar, unmodified source cell; or
iii) a first immunostimulatory protein that is absent or present at reduced levels, optionally wherein the reduced level by at least 10%, 20%, 30%, 40%, 50%, 60%, 70%, 80%, or 90%, compared to a fusosome generated from an otherwise similar, unmodified source cell and a second immunostimulatory protein that is absent or present at reduced levels, optionally wherein the reduced level is by at least 10%, 20%, 30%, 40%, 50%, 60%, 70%, 80%, or 90%, compared to a fusosome generated from an otherwise similar, unmodified source cell.

18. The method of claim 17, wherein, when administered to a subject, one or more of:
i) the fusosome does not produce a detectable antibody response, or antibodies against the fusosome are present at a level of less than 10%, 5%, 4%, 3%, 2%, or 1% above a background level;
ii) the fusosome does not produce a detectable cellular immune response, or a cellular immune response against the fusosome is present at a level of less than 10%, 5%, 4%, 3%, 2%, or 1% above a background level;
iii) the fusosome does not produce a detectable innate immune response, or the innate immune response against the fusosome is present at a level of less than 10%, 5%, 4%, 3%, 2%, or 1% above a background level;
iv) less than 10%, 5%, 4%, 3%, 2%, or 1% of fusosomes are inactivated by serum;
v) a target cell that has received the exogenous agent from the fusosome does not produce a detectable antibody response, or antibodies against the target cell are present at a level of less than 10%, 5%, 4%, 3%, 2%, or 1% above a background level; or
vi) a target cell that has received the exogenous agent from the fusosome does not produce a detectable cellular immune response, or a cellular response against the target cell is present at a level of less than 10%, 5%, 4%, 3%, 2%, or 1% above a background level.

19. A fusosome comprising:
a) a lipid bilayer comprising a fusogen, and
b) an exogenous agent or a nucleic acid encoding an exogenous agent; and
c) one or more of:
i) a first exogenous or overexpressed immunosuppressive protein on the lipid bilayer and a second exogenous or overexpressed immunosuppressive protein on the lipid bilayer;
ii) a first exogenous or overexpressed immunosuppressive protein on the lipid bilayer and a second immunostimulatory protein that is absent or present at reduced levels, optionally wherein the reduced level is by at least 10%, 20%, 30%, 40%, 50%, 60%, 70%, 80%, or 90%, compared to a fusosome generated from an otherwise similar, unmodified source cell; or
iii) a first immunostimulatory protein that is absent or present at reduced levels, optionally wherein the reduced level is reduced by at least 10%, 20%, 30%, 40%, 50%, 60%, 70%, 80%, or 90%, compared to a fusosome generated from an otherwise similar, unmodified source cell and a second immunostimulatory protein that is absent or present at reduced levels, optionally wherein the reduced level is reduced by at least 10%, 20%, 30%, 40%, 50%, 60%, 70%, 80%, or 90%, compared to a fusosome generated from an otherwise similar, unmodified source cell;
wherein, when administered to a subject, optionally wherein the subject is a human subject or a mouse, one or more of:
i) the fusosome does not produce a detectable antibody response, or antibodies against the fusosome are present at a level of less than 10%, 5%, 4%, 3%, 2%, or 1% above a background level;
ii) the fusosome does not produce a detectable cellular immune response, or a cellular immune response against the fusosome is present at a level of less than 10%, 5%, 4%, 3%, 2%, or 1% above a background level;
iii) the fusosome does not produce a detectable innate immune response, or the innate immune response against the fusosome is present at a level of less than 10%, 5%, 4%, 3%, 2%, or 1% above a background level;
iv) less than 10%, 5%, 4%, 3%, 2%, or 1% of fusosomes are inactivated by serum;
v) a target cell that has received the exogenous agent from the fusosome does not produce a detectable antibody response, or antibodies against the target cell are present at a level of less than 10%, 5%, 4%, 3%, 2%, or 1% above a background level; or
vi) a target cell that has received the exogenous agent from the fusosome does not produce a detectable cellular immune response, or a cellular response against the target cell is present at a level of less than 10%, 5%, 4%, 3%, 2%, or 1% above a background level.

20. The fusosome of claim 18 or claim 19, wherein the background level is the corresponding level in the same subject prior to administration of the fusosome.

21. The fusosome of any one of claims 17-20, wherein the immunosuppressive protein is a complement regulatory protein or CD47.

22. The fusosome of any one of claims 18-21, wherein the immunostimulatory protein is an MHC, optionally wherein the MHC is an HLA, protein.

23. A fusosome, comprising:
a) a lipid bilayer comprising a fusogen, and
b) a nucleic acid encoding an exogenous agent;
c) an exogenous or overexpressed MHC, optionally wherein the MHC is an HLA, optionally wherein the HLA is an HLA-G or HLA-E, or a combination thereof, on the lipid bilayer.

24. The fusosome of any one of claims 19-23, wherein the fusogen comprises a domain of at least 100 amino acids in length having at least 80%, 85%, 90%, 95%, 96%, 97%, 98%, or 99% sequence identity to a wild-type paramyxovirus fusogen set forth in any one of SEQ ID NOS: 1-132.

25. A fusosome comprising:
a) a lipid bilayer comprising a fusogen, wherein the fusogen comprises a domain of at least 100 amino acids in length having at least 80%, 85%, 90%, 95%, 96%, 97%, 98%, or 99% sequence identity to a wild-type paramyxovirus fusogen set forth in any one of SEQ ID NOS: 1-132;
b) a nucleic acid encoding an exogenous agent; and
c) an exogenous or overexpressed CD47 or a complement regulatory protein, or a combination thereof, on the lipid bilayer.

26. A fusosome comprising:
a) a lipid bilayer comprising a fusogen, wherein the fusogen comprises a domain of at least 100 amino acids in length having at least 80%, 85%, 90%, 95%, 96%, 97%, 98%, or 99% sequence identity to a wild-type paramyxovirus fusogen set forth in any one of SEQ ID NOS: 1-132, and
b) a nucleic acid encoding an exogenous agent; and
c) MHC I, optionally wherein the MHC I is HLA-A, HLA-B, or HLA-C, or MHC II, optionally wherein MHC II is HLA-DP, HLA-DM, HLA-DOA, HLA-DOB, HLA-DQ, or HLA-DR, that is absent or present at reduced levels, optionally wherein the reduced level is, reduced by at least 10%, 20%, 30%, 40%, 50%, 60%, 70%, 80%, or 90%, compared to a fusosome generated from an otherwise similar, unmodified source cell.

27. A fusosome comprising:
a) a lipid bilayer comprising a fusogen, wherein the fusogen comprises a domain of at least 100 amino acids in length having at least 80%, 85%, 90%, 95%, 96%, 97%, 98%, or 99% sequence identity to a wild-type paramyxovirus fusogen set forth in any one of SEQ ID NOS: 1-132; and
b) a nucleic acid encoding an exogenous agent; and
c) one or both of an exogenous or overexpressed immunosuppressive protein or an immunostimulatory protein that is absent or present at reduced levels, optionally wherein the reduced level is reduced by at least 10%, 20%, 30%, 40%, 50%, 60%, 70%, 80%, or 90%, compared to a fusosome generated from an otherwise similar, unmodified source cell.

28. The fusosome of any of the preceding claims, wherein the nucleic acid comprises w one or more insulator elements.

29. A fusosome comprising:
a) a lipid bilayer comprising a fusogen, wherein the fusogen comprises a domain of at least 100 amino acids in length having at least 80%, 85%, 90%, 95%, 96%, 97%, 98%, or 99% sequence identity to a wild-type paramyxovirus fusogen set forth in any one of SEQ ID NOS: 1-132; and
b) a nucleic acid encoding an exogenous agent, wherein the nucleic acid comprises one or more insulator elements.

30. The fusosome of claim 28 or claim 29, wherein the nucleic acid comprises two insulator elements, optionally wherein the two insulator elements comprise a first insulator element upstream of a region encoding the exogenous agent and a second insulator element downstream of a region encoding the exogenous agent, optionally wherein the first insulator element and second insulator element comprise the same or different sequences.

31. The fusosome of any one of claims 28-30, wherein variation in the median exogenous agent level in a sample of cells isolated after administration of the fusosome to the subject at a first timepoint is at least, less than, or about 10,000%, 5,000%, 2,000%, 1,000%, 500%, 200%, 100%, 50%, 20%, 10%, or 5% of the median exogenous agent level in a sample of cells isolated after administration of the fusosome to the subject at a second, later timepoint.

32. The fusosome of any one of claims 28-31, wherein at least 10%, 20%, 30%, 40%, 50%, 60%, 70%, 80%, or 90% of target cells in the subject detectably comprise the exogenous agent.

33. The fusosome of any one of claims 28-32, wherein at least 10%, 20%, 30%, 40%, 50%, 60%, 70%, 80%, or 90% of target cells in the subject that detectably comprised the exogenous agent at a first time point still detectably comprise the exogenous agent at a second, later timepoint.

34. The fusosome of any one of claims 28-33, which is not genotoxic or does not increase the rate of tumor formation in target cells.

35. The fusosome of any one of claims 19-34, wherein the exogenous agent is an exogenous polypeptide or an exogenous RNA, optionally wherein the exogenous agent is a therapeutic agent.

36. A method of delivering an exogenous agent to a subject comprising administering to the subject a fusosome of any of the preceding claims, thereby delivering the exogenous agent to the subject, optionally wherein the subject is a human subject.

37. A method of modulating a function, in a subject, target tissue or target cell, comprising contacting the target tissue or the target cell a fusosome of any of the preceding claims, optionally wherein the subject is a human subject.

38. The method of claim 37, wherein the target tissue or the target cell is present in a subject.

39. The method of claim 37 or claim 38, wherein the contacting is carried out by administering the fusosome to the subject.

40. A method of treating or preventing a disorder, in a subject, comprising administering to the subject a fusosome of any one of the preceding claims, optionally wherein the subject is a human subject.

41. A method of making a fusosome of any one of the preceding claims, comprising:
a) providing a cell that comprises the nucleic acid and the fusogen (e.g., re-targeted fusogen);
b) culturing the cell under conditions that allow for production of the fusosome, and
c) separating, enriching, or purifying the fusosome from the cell, thereby making the fusosome.

42. A source cell for producing a fusosome, comprising:
a) a nucleic acid;
b) structural proteins that can package the nucleic acid, wherein at least one structural protein comprises a fusogen that binds a fusogen receptor; and
c) a fusogen receptor that is absent or present at reduced levels (e.g., reduced by at least 10%, 20%, 30%, 40%, 50%, 60%, 70%, 80%, or 90%) compared to an otherwise similar, unmodified source cell.

43. The source cell of claim 42, wherein the fusogen causes fusion of the fusosome with the target cell upon binding to the fusogen receptor.

44. The source cell of claim 42 or 43, which binds to the second similar source cell, e.g., the fusogen of the source cell binds to the fusogen receptor on the second source cell.

45. A population of source cells of any one of claims 42-44.

46. The population of source cells of claim 45, wherein less than 10%, 5%, 4%, 3%, 2%, or 1% of cells in the population are multinucleated.
